(19) United States
(12) Patent Application Publication
(10) Pub. No.: US 2025/0349399 A1
(43) Pub. Date: Nov. 13, 2025

(54) PERSONAL HEALTH DATABASE PLATFORM WITH SPATIOTEMPORAL MODELING AND SIMULATION

(71) Applicant: QOMPLX LLC, Reston, VA (US)

(72) Inventors: Jason Crabtree, Vienna, VA (US); Richard Kelley, Woodbridge, VA (US)

(21) Appl. No.: 18/801,361

(22) Filed: Aug. 12, 2024

Related U.S. Application Data
(63) Continuation-in-part of application No. 18/662,988, filed on May 13, 2024, now Pat. No. 12,373,600.

(51) Int. Cl. G16H 10/60; G16H 20/00; G16H 50/30; G16H 50/50

(52) U.S. Cl. CPC G16H 10/60; G16H 20/00; G16H 50/30; G16H 50/50

(57) ABSTRACT

A spatiotemporal modeling system for Personal Health Database (PHDB) platforms integrates diverse health data types into a comprehensive 4D model of an individual's health status. By combining genomic, imaging, clinical, and real-time health data, the system creates a dynamic, time-based representation of the user's anatomy and physiology. This model enables real-time analysis, pattern recognition, and predictive forecasting of health outcomes. The system preprocesses and aligns data from various sources, constructs a detailed spatial framework, and continuously updates the model with new inputs. Through interactive visualizations, it provides users and healthcare providers with intuitive, personalized insights for improved health management and decision-making.

### CROSS-REFERENCE TO RELATED APPLICATIONS

Priority is claimed in the application data sheet to the following patents or patent applications, each of which is expressly incorporated herein by reference in its entirety:
Ser. No. 18/662,988

### BACKGROUND OF THE INVENTION

### Field of the Art

The present invention is in the field of genomic data analysis, and more particularly is directed to the problem of modeling the human body using a robust medical database.

### Discussion of the State of the Art

Today's computer aided biology endeavors are rapidly seeking to incorporate omics data including genomics, proteomics, metabolomics, metagenomics, epigenomics, and transcriptomics as well as machine learning, modeling simulation and broader artificial intelligence techniques. This is particularly true when viewed from the most pragmatic lens, functional genomics, that focuses on describing gene and protein interactions beyond just static DNA sequences or structures inclusive of concepts such as gene translation and transcription, gene expression, protein-protein interactions in situ. One of the tremendous challenges with linking such work at scale stems from the challenges resulting from security, privacy and regulatory compliance efforts associated with such data and the practical elements of considering not just basic bioinformatics workflows around observation, collection, persistence, aggregation, correlation, queries, statistical modeling, and machine learning but also integration of more advanced simulation modeling and artificial intelligence (AI) enabled planning efforts. This is not only important because this broader definition of bioinformatics not only encompasses the current state of data analytics and computational biology concepts but extends them to look at more elements of potential temporal, environmental, and experiential elements impacting a functional understanding of omics data and its inclusion in practical wellness and healthcare. This requires consideration of both nature and nurture as well as broader contextual and social elements of health.

Genetic carrier screening is a relevant exemplary diagnostic procedure that delves into an individual's DNA to ascertain whether they bear an elevated risk of having offspring afflicted with specific genetic disorders, often this is evaluated given the individual's data as well as a prospective mate's DNA profile. Within our genetic makeup, we harbor alterations referred to as variants that possess the potential to trigger genetic conditions or expressions and a proactive understanding of such predispositions can aid in personal and medical decision-making and planning. Fortunately, most potentially problematic variants which increase likelihood of disease remain dormant, avoiding significant impact on our personal health or the well-being of our progeny. In the future, such considerations may also inform gene therapies both pre- and post-conception or throughout an individual's life. When they do manifest, awareness of genetic predispositions to disease can speed up diagnosis, treatment, and resolution—with the advent of CRISPR/Cas9 based gene editing therapies such knowledge may also be the gateway for genetic alteration via in vivo, in vitro or ex vivo processes targeting Deoxyribonucleic Acid (DNA) or as demonstrated more recently, Ribonucleic Acid (RNA) and mitochondrial DNA (mtDNA). Genetic carrier screening at present is often primarily concerned with conditions that are hereditary in an autosomal recessive, X-linked disorders, or other potential screening approaches.

In the case of autosomal recessive disorders, both the person who contributes the egg and the one who provides the sperm must carry variants within the same gene to give rise to a child afflicted by that particular condition. Notable instances of autosomal recessive conditions include cystic fibrosis, spinal muscular atrophy, sickle cell disease, and Tay-Sachs disease. For X-linked conditions, carriers contributing their eggs are more likely to have children affected by the condition. It is noteworthy that individuals with XY chromosomes, such as most cisgender males and transgender females, are typically not predisposed to having offspring affected by X-linked conditions such as in disorders like Fragile X Syndrome. Consequently, many laboratories do not routinely conduct screening for X-linked genes in such cases. Prominent examples of X-linked conditions encompass Fragile X syndrome and Duchenne muscular dystrophy. Single gene disorders such as cystic fibrosis, hemochromatosis, Tay-Sachs, and sickle cell anemia can also be tested for.

With the vast reduction in whole genome sequencing costs, we note that whole genome sequencing and democratized access to associated bioinformatics files such as BAM, VCF, FASTA, FASTQ, or SAM files, is rapidly changing the potential for consumer, community, and medical professional access to genomics information and downstream analytical capabilities. Since our phones, watches, wearables, instrumented homes and offices also provide data, there is now potential for consideration of gene expression and response to environmental and lifestyle factors that can further aid in our understanding of gene and protein interactions at scale and over time.

Best practices for science-driven couples wishing to have children now includes genetic carrier screening. For couples undergoing in-vitro fertilization (IVF) or other fertility assistance, this is often mandatory. Additional preimplantation genetic testing, very recently including whole genome sequencing, can also aid in reproductive, life, and medical decision-making processes. Unfortunately, prior to being ready for children, very few couples understand the degree to which a prospective progeny would be susceptible to material genetically based disease risks and costs. It can be very tragic to learn that some genetic issue raises the prospect of serious risk of fatal birth defects or potential medical conditions pre or post conception. Clearly, if it were technically and socially feasible for couples to understand their genetic compatibility earlier in the process, such difficult situations might be avoided or better managed. In fact, performing genetic screening when initially dating rather than waiting until years later when planning to have children offers several important advantages such as:

Early Awareness: Genetic screening at the beginning of a relationship allows couples to gain awareness of their genetic compatibility and any potential risks long before they decide to have children. This early awareness can help them make informed decisions about their future together or apart—especially when different risk factors may impact the nature, timing or potential for reproduction of a potential partner.

Informed Decision-Making: Knowing their genetic status enables couples to make informed decisions about their relationship and family planning. If both individuals are carriers for a specific genetic condition, they can discuss their options and potential challenges from the outset. Since cost is a leading factor in reproductive decision-making, probabilistic weightings of disease expression, treatments and outcomes may also aid couples or broader families in their consideration of starting a “next” generation.

Emotional Preparation: Genetic screening early in a relationship provides couples with the opportunity to emotionally prepare for any potential challenges related to their genetic compatibility. This can reduce the shock and stress that may occur when discovering genetic risks later on. It may also help avoid statistically improbable, but potentially devastating, issues like determining shared genetic lineage whether through natural or assisted reproduction since some extreme cases indicate upwards of 550 offspring from single individuals.

Time for Evaluation and Counseling: Couples who undergo genetic screening during dating have more time to seek genetic and medical counseling, consult with healthcare professionals, and explore their reproductive options or discuss potential challenges with family or spiritual support systems that may be impacted. This allows for a comprehensive understanding of the implications of their genetic status in major personal, family, community, and financial decisions.

Potential Alternatives: Early screening may reveal that one or both partners are carriers of certain genetic conditions. This knowledge can prompt discussions about alternative family planning methods, such as adoption or assisted reproductive technologies (including more advanced screening of potential embryos during IVF or offspring in vivo), which may be preferable for some couples.

Relationship Planning: Genetic screening results can also influence long-term relationship planning. Couples can consider whether they are prepared to navigate potential challenges associated with genetic risks and whether they want to invest in the necessary support and care needed.

Reduced Stress: Couples who have already addressed potential genetic concerns can experience reduced stress and anxiety when they eventually decide to have children, as they have already taken steps to understand and manage any risks or costs.

Supportive Environment: Openly discussing genetic screening early in a relationship fosters an environment of trust and communication. Couples can work together to make decisions that align with their values and goals. Additionally, genetic information might be optionally persisted for the availability of any future offspring to aid them in their own healthcare decisions if needed.

Potential Reduced Fertility Treatment Costs: Reducing the potential for infertility, miscarriage or complications and better leverage and incorporate preimplantation genetic testing (PGT; a screening test that can be performed on embryos created via in vitro fertilization IVF to genetically analyze the embryos prior to transfer) or in vivo genetic sampling and testing.

Genetic screening during the early stages of dating allows couples to make informed choices about their future, consider alternatives, and emotionally prepare for any challenges related to their genetic compatibility. It promotes open communication and proactive decision-making, ultimately leading to a healthier and more supportive relationship dynamic and mitigating or potentially avoiding painful situations like an inability to have children without catastrophic risks or costs after years of investment in a relationship. In a much more pedestrian example, it may also be used to aid in refining things as mundane as grocery shopping, suggested recipes, or travel planning (e.g., for a date or a trip) by noting potential indicators of allergies, lactose intolerance, or other factors that might necessitate or encourage alternate decisions. For example, not going to fondue and ice cream if lactose intolerance is likely based on genetic indicators around LCT gene for lactase enzyme production.

Similarly, ongoing treatment for disease like cancer requires ongoing monitoring of omics data to include emergent elements such as algorithm based RNA sequencing, Homologous Recombination Deficiency (HRD) identification and Tumor Origin (TO) identification among others in order to develop and field more personalized and predictive treatment options as well as to support alternative financial and remuneration models such as value-based healthcare.

What is needed are methods and systems for spatio-temporal health records for individuals and groups that leverage omics data alongside environmental and lifestyle data risk factors, holistic health tracking, and supporting of predictive and collaborative medical advances and research. In order to achieve this, what is also needed is a scalable platform for creating and using personal health databases (PHDBs) in support of capturing discrete observations of relevant health events, supporting omics based analysis, and developing evaluating potential health risks, treatments and insurance or remuneration schemes for individuals, providers, payers and/or their employers or government entities to the prospective individuals, their communities their offspring.

### SUMMARY OF THE INVENTION

Accordingly, the inventor has conceived and reduced to practice methods and systems for a personal spatiotemporal health database platform with integrated modeling and simulation and artificial intelligence and machine learning capabilities.

According to a preferred embodiment, a computer-implemented method platform for a personal health database platform with spatiotemporal modeling, the computer-implemented method comprising: collecting a plurality of data that include a plurality of data types from multiple sources; preprocessing the plurality of data by converting the plurality of data into a common format and temporally aligning data points from different data sources along a common timeline; creating a multi-dimensional spatial framework representing a desired subject, wherein the spatial framework comprises a detailed digital representation of a subject's anatomy; generating contextualized insight data from raw observational and sensor data with spatiotemporal tagging; combining the common timeline and spatial framework to create a plurality of multi-dimensional time-based models or simulations, wherein the models or simulations integrate the preprocessed data with the spatial framework to construct a comprehensive 4D representation of the subject's health status; performing batch, microbatched or streaming near real-time analysis on the plurality of multi-dimensional time-based models or simulations to identify patterns, anomalies, potential health issues, and corresponding therapies or treatments; generating predictions and forecasts of the subject's future anatomical and physiological states based on the analysis of the plurality of multi-dimensional time-based models or simulations; displaying the plurality of multi-dimensional time-based models or simulations and analysis results through an audible, haptic, or visual interface connected to a user device, wherein the display includes interactive visualizations or descriptions of health data, insights, or scenarios derived from the plurality of multi-dimensional time-based models or simulations; and updating the plurality of multi-dimensional time-based models or simulations with new data inputs to maintain an accurate and current representation of the subject's health status over time, is disclosed.

According to another preferred embodiment, a computing system for a personal health database platform with spatiotemporal modeling, the computing system comprising: one or more hardware processors configured for: collecting a plurality of data that include a plurality of data types from multiple sources; preprocessing the plurality of data by converting the plurality of data into a common format and temporally aligning data points from different data sources along a common timeline; creating a multi-dimensional spatial framework representing a desired subject, wherein the spatial framework comprises a detailed digital representation of a subject's anatomy; generating contextualized insight data from raw observational and sensor data with spatiotemporal tagging; combining the common timeline and spatial framework to create a plurality of multi-dimensional time-based models or simulations, wherein the models or simulations integrate the preprocessed data with the spatial framework to construct a comprehensive 4D representation of the subject's health status; performing batch, microbatched or streaming near real-time analysis on the plurality of multi-dimensional time-based models or simulations to identify patterns, anomalies, potential health issues, and corresponding therapies or treatments; generating predictions and forecasts of the subject's future anatomical and physiological states based on the analysis of the plurality of multi-dimensional time-based models or simulations; displaying the plurality of multi-dimensional time-based models or simulations and analysis results through an audible, haptic, or visual interface connected to a user device, wherein the display includes interactive visualizations or descriptions of health data, insights, or scenarios derived from the plurality of multi-dimensional time-based models or simulations; and updating the plurality of multi-dimensional time-based models or simulations with new data inputs to maintain an accurate and current representation of the subject's health status over time, is disclosed.

According to another preferred embodiment, a system for a personal health database platform with spatiotemporal modeling, comprising one or more computers with executable instructions that, when executed, cause the system to: collect a plurality of data that include a plurality of data types from multiple sources; preprocess the plurality of data by converting the plurality of data into a common format and temporally aligning data points from different data sources along a common timeline; create a multi-dimensional spatial framework representing a desired subject, wherein the spatial framework comprises a detailed digital representation of a subject's anatomy; generate contextualized insight data from raw observational and sensor data with spatiotemporal tagging; combine the common timeline and spatial framework to create a plurality of multi-dimensional time-based models or simulations, wherein the models or simulations integrate the preprocessed data with the spatial framework to construct a comprehensive 4D representation of the subject's health status; perform batch, microbatched or streaming near real-time analysis on the plurality of multi-dimensional time-based models or simulations to identify patterns, anomalies, potential health issues, and corresponding therapies or treatments; generate predictions and forecasts of the subject's future anatomical and physiological states based on the analysis of the plurality of multi-dimensional time-based models or simulations; display the plurality of multi-dimensional time-based models or simulations and analysis results through an audible, haptic, or visual interface connected to a user device, wherein the display includes interactive visualizations or descriptions of health data, insights, or scenarios derived from the plurality of multi-dimensional time-based models or simulations; and update the plurality of multi-dimensional time-based models or simulations with new data inputs to maintain an accurate and current representation of the subject's health status over time, is disclosed.

According to another preferred embodiment, non-transitory, computer-readable storage media having computer instructions embodied thereon that, when executed by one or more processors of a computing system employing a system for a personal health database platform with spatiotemporal modeling, cause the computing system to: collect a plurality of data that include a plurality of data types from multiple sources; preprocess the plurality of data by converting the plurality of data into a common format and temporally aligning data points from different data sources along a common timeline; create a multi-dimensional spatial framework representing a desired subject, wherein the spatial framework comprises a detailed digital representation of a subject's anatomy; generate contextualized insight data from raw observational and sensor data with spatiotemporal tagging; combine the common timeline and spatial framework to create a plurality of multi-dimensional time-based models or simulations, wherein the models or simulations integrate the preprocessed data with the spatial framework to construct a comprehensive 4D representation of the subject's health status; perform batch, microbatched or streaming near real-time analysis on the plurality of multi-dimensional time-based models or simulations to identify patterns, anomalies, potential health issues, and corresponding therapies or treatments; generate predictions and forecasts of the subject's future anatomical and physiological states based on the analysis of the plurality of multi-dimensional time-based models or simulations; display the plurality of multi-dimensional time-based models or simulations and analysis results through an audible, haptic, or visual interface connected to a user device, wherein the display includes interactive visualizations or descriptions of health data, insights, or scenarios derived from the plurality of multi-dimensional time-based models or simulations; and update the plurality of multi-dimensional time-based models or simulations with new data inputs to maintain an accurate and current representation of the subject's health status over time, is disclosed.

According to an aspect of the embodiment, the plurality of data types comprise genomic data, proteomic data, metabolomics data, metagenomics data, epigenomic data, imaging data, clinical data, and real-time health metrics.

### BRIEF DESCRIPTION OF THE DRAWING FIGURES

FIG. 1 is a high-level architecture diagram of an exemplary system for discretely analyzing one or more human genomes for risk management or health outcome improvement using personal health databases (PHDBs), according to an aspect of the invention.

FIG. 2 is a diagram showing an exemplary arrangement of a PHDB, according to an aspect of the invention.

FIG. 3 is a system diagram showing an exemplary arrangement of cloud-based PHDB-related computing, according to an aspect of the invention.

FIG. 4 is a system diagram showing an exemplary arrangement of mobile device-resident PHDB-related computing, according to an aspect of the invention.

FIG. 5 is a system diagram showing an exemplary arrangement of a PHDB computing server or service with registration controls and third-party service integration, according to an aspect of the invention.

FIG. 6 is a system diagram showing an exemplary arrangement of a PHDB orchestration service according to an aspect of the invention.

FIG. 7 is a system diagram showing an exemplary arrangement of a federated PHDB architecture, according to an aspect of the invention.

FIG. 8 is a system diagram showing an exemplary arrangement of biometric computing used for biometric sensors located on a user's endpoint device or with separate biometric sensor devices, according to an aspect of the invention.

FIG. 9 is a system diagram showing an exemplary arrangement of an encryption platform for PHDB computing, according to an aspect of the invention.

FIG. 10 is a system diagram showing an exemplary arrangement of a PHDB computing system using distributed ledger technologies, according to an aspect of the invention.

FIG. 11 is a system diagram showing an exemplary arrangement of a PHDB computing system using geospatial computing, according to an aspect of the invention.

FIG. 12 is a system diagram showing an exemplary arrangement of a PHDB computing system using geospatial and community-based computing techniques and user grouping techniques, according to an aspect of the invention.

FIG. 13 is a system diagram showing an exemplary arrangement of federated computing, according to an aspect of the invention.

FIG. 14 is a system diagram showing an exemplary arrangement of a service prediction mechanism using a multidimensional time-series database, according to an aspect of the invention.

FIG. 15 is a system diagram illustrating an exemplary architecture of a machine learning engine.

FIG. 16A is a diagram illustrating an exemplary architecture of a neural network.

FIG. 16B is a system diagram showing an exemplary arrangement of a PHDB system utilizing Large Language Model (LLM) machine learning technology, according to an aspect of the invention.

FIG. 17 is a system diagram showing an exemplary arrangement of a PHDB computing system utilizing Augmented Reality (AR) or Virtual Reality (VR) or haptic or brain interface technologies, according to an aspect of the invention.

FIG. 18 is a system diagram showing an exemplary arrangement of a PHDB computing system utilizing a variety of possible third-party services and servers, according to an aspect of the invention.

FIG. 19 is a system diagram showing an exemplary arrangement of a PHDB computing system utilizing Internet-of-Things (IoT) devices and associated technology, according to an aspect of the invention.

FIG. 20 is a method diagram showing exemplary steps taken for a user to register and establish a PHDB, according to an aspect of the invention.

FIG. 21 is a method diagram showing exemplary steps taken for a user to update their data or information in a PHDB, according to an aspect of the invention.

FIG. 22 is a method diagram showing exemplary steps taken for homomorphic encryption to be utilized with a PHDB, according to an aspect of the invention.

FIG. 23 is a method diagram showing exemplary steps taken for a user to authenticate themselves for other services using a PHDB, according to an aspect of the invention.

FIG. 24 is a method diagram showing exemplary steps taken for genome screening of a pair of individuals using their PHDBs, according to an aspect of the invention.

FIG. 25 is a method diagram showing exemplary steps taken for groups to be formed and users to be screened based on their group memberships via their PHDBs, according to an aspect of the invention.

FIG. 26 is a method diagram showing exemplary steps taken on an Oracle database to mitigate the risk of exploitation.

FIG. 27 is a method diagram showing exemplary steps taken for a PHDB service to alert or warn users based on geohazards and government or NGO alerts based on geographical regions, according to an aspect of the invention.

FIG. 28 is a method diagram showing exemplary steps taken for PHDB platform users to be paired for peer-to-peer communications, according to an aspect of the invention.

FIG. 29 is a method diagram showing exemplary steps taken for a pair of users to be screened for peer-to-peer communications, according to an aspect of the invention.

FIG. 30 is a method diagram showing exemplary steps taken for individuals to be screened and scored for risk and risk mitigation factors using their PHDB and the PHDB computing platform, according to an aspect of the invention.

FIG. 31 is a method diagram showing exemplary steps taken for a PHDB computing platform to be utilized alongside AR or VR technologies, according to an aspect of the invention.

FIG. 32 is a method diagram showing exemplary steps taken for a PHDB computing platform to be used in tandem with third party services such as gaming services or software, according to an aspect of the invention.

FIG. 33 is a method diagram showing exemplary steps taken for a user to enhance their diet by utilizing IoT devices with their PHDB and a PHDB computing platform, according to an aspect of the invention.

FIG. 34 is a block diagram showing an exemplary arrangement of a PHDB, where the PHDB system is configured for real time spatiotemporal modeling.

FIG. 35 is a diagram showing an exemplary subsystem of a PHDB system configured for real time spatiotemporal modeling, a spatiotemporal modeling subsystem.

FIG. 36 is a block diagram showing an exemplary arrangement of a PHDB, where the PHDB system includes a Neurosymbolic AI system to combine symbolic and connectionist approaches.

FIG. 37 is a diagram showing an exemplary subsystem of a PHDB system including a Neurosymbolic AI system, a Neurosymbolic AI Subsystem.

FIG. 38 is a block diagram showing an exemplary arrangement of a PHDB, where the PHDB system is configured for multimodal integration.

FIG. 39 is a diagram showing an exemplary subsystem of a PHDB system configured for multimodal integration, a multimodal integrator.

FIG. 40 is a diagram showing an exemplary embodiment of a PHDB system configured for multimodal integration, wherein the multimodal integrator generates a 3D model from incoming data.

FIG. 41 is a block diagram showing an exemplary arrangement of a PHDB, where the PHDB system is configured for predictive analysis.

FIG. 42 is a diagram showing an exemplary subsystem of a PHDB system configured for predictive analysis, a predictive analysis subsystem.

FIG. 43 is a diagram showing an exemplary subsystem of a PHDB system is configured for predictive analysis, a machine learning training system.

FIG. 44 is a block diagram showing an exemplary arrangement of a PHDB, where the PHDB system is configured to generate AR/VR content.

FIG. 45 is a diagram showing an exemplary subsystem of a PHDB system configured for AR/VR content generation, an AR/VR content generator.

FIG. 46 is a block diagram showing an exemplary arrangement of a PHDB, where the PHDB system is configured for federated learning.

FIG. 47 is a diagram showing an exemplary subsystem of a PHDB system configured for federated learning, a federated learning subsystem.

FIG. 48 is a diagram showing an exemplary subsystem of a PHDB system configured for federated learning, a federated training subsystem.

FIG. 49 is a block diagram showing an exemplary arrangement of a PHDB, where the PHDB system is secured using a blockchain security system.

FIG. 50 is a diagram showing an exemplary subsystem of a PHDB with blockchain security, a blockchain security system.

FIG. 51 is a block diagram showing an exemplary arrangement of a PHDB, where the PHDB system is configured with an IoT data processing hub.

FIG. 52 is a diagram showing an exemplary subsystem of a PHDB with IoT processing, an IoT Processing Hub.

FIG. 53 is a diagram showing exemplary IoT devices that may be connected to an IoT processing hub.

FIG. 54 is a block diagram showing an exemplary arrangement of a PHDB, where the PHDB system is configured with a Large Language Model.

FIG. 55 is a diagram showing exemplary neural network architecture for a Large Language Model that has been integrated into the PHDB system.

FIG. 56 is a block diagram showing an exemplary arrangement of a PHDB, where the PHDB system is configured for maintaining ethical AI usage and transparent machine learning models.

FIG. 57 is a diagram showing an exemplary subsystem of a PHDB, an ethics and transparency subsystem.

FIG. 58 is a flow diagram showing exemplary method for configuring a PHDB system for real time spatiotemporal modeling.

FIG. 59 is a flow diagram showing exemplary method for configuring a PHDB system with a Neurosymbolic AI system.

FIG. 60 is a flow diagram showing exemplary method for configuring a PHDB system with multimodal integration capabilities.

FIG. 61 is a flow diagram showing exemplary method for configuring a PHDB system for enhanced predictive analysis.

FIG. 62 is a flow diagram showing exemplary method for configuring a PHDB system for generating AR/VR content.

FIG. 63 is a flow diagram showing exemplary method for configuring a PHDB system for federated learning and edge device computing.

FIG. 64 is a flow diagram showing exemplary method for configuring a PHDB system with a blockchain security system.

FIG. 65 is a flow diagram showing exemplary method for configuring a PHDB system with an IoT processing hub.

FIG. 66 is a flow diagram showing exemplary method for configuring a PHDB system with an integrated Large Language Model.

FIG. 67 is a flow diagram showing exemplary method for configuring a PHDB system for ethical AI detection and transparent AI model use.

FIG. 68 illustrates an exemplary computing environment on which an embodiment described herein may be implemented.

### DETAILED DESCRIPTION OF THE INVENTION

The inventor has conceived, and reduced to practice, methods, and systems for a personal health database platform with spatiotemporal modeling, simulation, and analytics. Such systems and methods need to be able to accommodate selective degrees of “opting in” to analytics or screening processes for group or cloud or aggregate modeling and analysis (e.g. a specific drug study) and the ability for multiple applications to engage with shared data. Setting and enforcing of user-specific preferences should also be available (e.g., specific kinds of “deal breaker” criteria or scores or preferences for a party or group or provider if “screening mode” is enabled to aid user in identifying potential dates, mates, friends, providers et cetera). The invention may use cloud-based, private data center, content distribution network, or edge-based processing (e.g. on a local mobile device). The invention is robust to periodic or sporadic connectivity (e.g., available during a remote hike or a network outage), and to the complexity of the issues associated with identifying a given individual or counterparty both digitally and physically linking them to a specific persona and health record (e.g. their own or doctor for a patient that has authorized their access). The invention may enable both attempts at automated recognition or identification of potential mates, friends, or service providers with probabilistic confidence (i.e., suggest a partner and “silently” screen) as well as proactive use cases (e.g., “can we check compatibility” or “am I eligible for the study” or “will my insurance cover this procedure”).

According to various aspects of the invention, user declarations of preferences about genetic, emotional, religious, or behavioral kinds of preferences or constraints can be stored and leveraged by computer-enabled processes to aid users in real-world interactions as well as in augmented reality and/or metaverse engagement (i.e. simulated worlds or gaming or simulated health scenarios). This may be through reports, suggestions regarding prospective interactions, in-app or “anti-app” dynamically generated user interface (UI) prompts, device feedback (e.g. haptics that buzz when close to a prospect that is compatible), interaction with implants or medical devices, or engagement with other applications, services, wearables, or hardware. It is worth noting that user preferences, regulations, laws, or application/community rules may also set conditions for different “visibility” conditions and how such identified matches or actions (e.g. you need to take a break because it is too hot out and you are experiencing an unsustainable level of effort or risking a cardiac event) may be presented to the user (for example, a “negative match” warning may be preferred in some settings as opposed to a positive match encouragement). For example, a negative only warning configuration could avert a future “incompatibility” disaster (e.g. common parent or unknown cousin status) behind the scenes without otherwise shaping/encouraging prospective relationship development. Similarly, overtraining could be identified to the user with an award/badge for stopping a workout when prudent vs just for “more calories achieved”.

The ability to change the nature, location, and specificity of potential alerts (positive or negative) is also based on the degree to which data sharing is enabled directly from others (e.g. mobile devices), intermediaries (e.g. Epic Systems, Cerner, Department of Defense or Veterans Affairs medical records, MATCH.COM, BUMBLE, TINDER, or LINKEDIN or Peloton or Reddit), but also from public data (e.g. public writings, persona, photos, etc.). According to an aspect, direct disclosure (e.g. of medical condition or ethnicity or toxic exposure) might also be estimated by some statistical or artificial intelligence or machine learning (AI/ML) or simulation methods. For example, generative AI (GenAI) might “guess” at prospective profiles (even genetic ones) based on indicators (e.g. specific ethnic background, food eaten, activities, localities, etc.) that may be available to the system from private or public sources (including web scraping). This can be used to probabilistically profile others, even if they are not opting into such a system to aid at least one user. This can be further enhanced by incorporating other models or data sets or other research publications that might be available for licensure in some fashion (e.g., 23ANDME or ANCESTRY.COM or proprietary datasets such as from drug companies or health care providers or payers doing studies on specific drugs or molecules or therapies) for genotyping or whole genome processing/guessing about a whole genome, other previously enumerated omics data, or elements including but not limited to SNPs, STRs, mtDNA, RAW data. Various aspects might also generate profiles that are more limited (e.g. genotype guesses vs whole genome) based on cost or on the stage of a relationship or the current level of medical risk or decision being considered. Systems and methods of the invention might also be used for identification of prospective organ donors or tissue donors or blood donors during normal human interactions. This could enable much more efficient search processes within “normal” community interactions for bone marrow, organs, other tissues and so forth, or for potential “directed” organ donation options for families/friends. This could also enable opt-in solicitation for organ and tissue donations as this may vastly improve potential for either direct use in medical procedures or may serve as a basis or seed for other advanced treatments or therapies that rely on emerging technologies like 3d printing of cellular tissue. It may also aid in study construction or in confirming efficacy of various treatments and drugs once rolled out to broader groups.

According to an aspect of the invention, the secure and privacy-preserving genetic compatibility or characteristic assessment and other analytics or simulation routines conducted by the system may be optionally enhanced with partial or full homomorphic encryption. In such aspects, the system can leverage collected omics information to calculate encrypted risk or susceptibility or compatibility scores for groups or pairs of individuals without revealing sensitive details. These scores, reflecting the predicted compatibility or risk factors based on analyzed omics or lifestyle or environmental or social factors, provide users with additional data point(s) to guide their decision making and life choices. These scores (or suggestions, reports, dashboards, risk factors or predictions) based on analyzed genetic factors or simulations or models or potential health states and scenarios without revealing raw data, provide users with another valuable piece of information for deciding whether to reveal their full identities and genetic information to promising (high scoring) matches and deepen their connection. This same approach may enable faster and more efficient sourcing of potential candidates for medical studies of drugs, therapeutics, surgeries or other treatments (including emerging CRISPR/Cas9) where new studies, approvals (e.g. drug or gene therapy or radiation) might receive feeds of relevant academic, regulatory, legal, commercial or government actions related to omics factors identified in their personal health database or those of others with whom they have visibility (e.g. spouse, child, parent, friend, or group) or especially of combinations of other lifestyle (e.g. fitness or nutritional) or environmental data (e.g. exposure to heavy metals or toxins).

According to an aspect of an embodiment, the platform can support “search: operations on (user) authorized data for analysis privately i.e., some people might enable homomorphic studies on their data by only want to receive “blind” approaches based on their data being of interest. This may be applied to user data such as tissue/blood/etc. donations as well as potential fits for treatments and/or therapies. This “inbound” queue may be optionally routed to an AI engine for recommendation/evaluation and also to their primary care physician or other medical team members as configured in their health records in the personal health database or their broader existing medical chart system like Epic System's MyChart. The platform can support such functionality by allowing individuals to authorize the use of their data for analysis privately. This can include using techniques like homomorphic encryption to allow for analysis without revealing the raw data. Platform may implement an inbound queue where data requests or queries can be submitted. This queue should be able to handle requests for analysis based on authorized data and route them to the appropriate destination.

According to another embodiment, user declarations of preferences and regulatory conditions, offering a flexible and adaptive approach to genetic and omics screening in the real-world, augmented reality, and metaverse contexts on an ongoing basis. The user also can change the nature, location, and specificity of potential alerts which is also based on the degree to which data sharing is enabled from others, intermediaries, and from public data. Direct disclosure might be estimated by statistical, artificial intelligence or machine learning methods and made available for review or sent via communications including but not limited to on-device push notifications, text messages, chat messages, voicemails, emails, physical mail, or engagement in the physical world with meetings or mailings or digital content (e.g. ads on computer or television or devices).

According to an aspect of the embodiment is that the systems and methods of the invention may be used for identification of prospective organ donors or blood or tissue donors during normal human interactions or via public (or private) listings which could enable much more efficient search processes within normal community interactions for tissue, blood, bone marrow, or organs or genetic material for healthcare or reproductive purposes. It should be noted that system may also be configured to have reference contracts for common elements (e.g. blood donor, tissue donor, marrow donor, sperm or egg donor, surrogate services) that might be aid counterparties in arranging for additional verification or transacting (conditional on medical professional approvals) directly in the PHDB or via sharing the PHDB with another application or service.

It should be appreciated that while a human genome and omics data is generally used throughout the specification when referring to genomic data and analysis thereof, it does not limit the disclosed systems and methods to the processing of human omic data. Such systems and methods may be directed to the omic data of other animals and not limited to human omic data. Such systems and methods may also find utility in the field of veterinary services.

According to an aspect of the embodiment is that the systems and methods of the invention may be used for personalized computational fluid dynamics modeling with both traditional numerical methods or with AI/ML enhanced approaches based on the spatiotemporal representation and user context observed by the system, with optional consideration of space-time stabilized representations of patient body. Specialized physics models trained on numerical simulations and experimental data across a variety of users under different conditions can support rapid and contextualized analysis of cardiovascular and other body systems where fluid dynamics models can enhance treatment or diagnosis. Physics machine learning models integrated with the system can enable real-time CFD to aid in diagnostics and analysis and in visualization for the user or for medical or emergency personnel. This can also enable specific exploration of potential medical devices (e.g. which stent is most appropriate? or what would a stent do for my condition?) to be assessed by the system and optionally explained by medical personnel or by an LLM or other AI agent to allow users to have more productive and specific discussions with ultimate healthcare providers or payers impacting their care.

One or more different aspects may be described in the present application. Further, for one or more of the aspects described herein, numerous alternative arrangements may be described; it should be appreciated that these are presented for illustrative purposes only and are not limiting of the aspects contained herein or the claims presented herein in any way. One or more of the arrangements may be widely applicable in numerous aspects, as may be readily apparent from the disclosure. In general, arrangements are described in sufficient detail to enable those skilled in the art to practice one or more of the aspects, and it should be appreciated that other arrangements may be utilized and that structural, logical, software, electrical and other changes may be made without departing from the scope of the particular aspects. Particular features of one or more of the aspects described herein may be described with reference to one or more particular aspects or figures that form a part of the present disclosure, and in which are shown, by way of illustration, specific arrangements of one or more of the aspects. It should be appreciated, however, that such features are not limited to usage in one or more particular aspects or figures with reference to which they are described. The present disclosure is neither a literal description of all arrangements of one or more of the aspects nor a listing of features of one or more of the aspects that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects and in order to more fully illustrate one or more aspects. Similarly, although process steps, method steps, algorithms or the like may be described in a sequential order, such processes, methods, and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method, or algorithm is carried out or executed. Some steps may be omitted in some aspects or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of more than one device or article.

The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

### Definitions

As used herein, “homomorphic encryption” refers to the cryptographic technique that allows computations to be performed on encrypted data without decrypting it first. It enables certain operations to be carried out on encrypted data while in ciphertext as if it were still in its original form, and the results are obtained in encrypted form.

### Conceptual Architecture

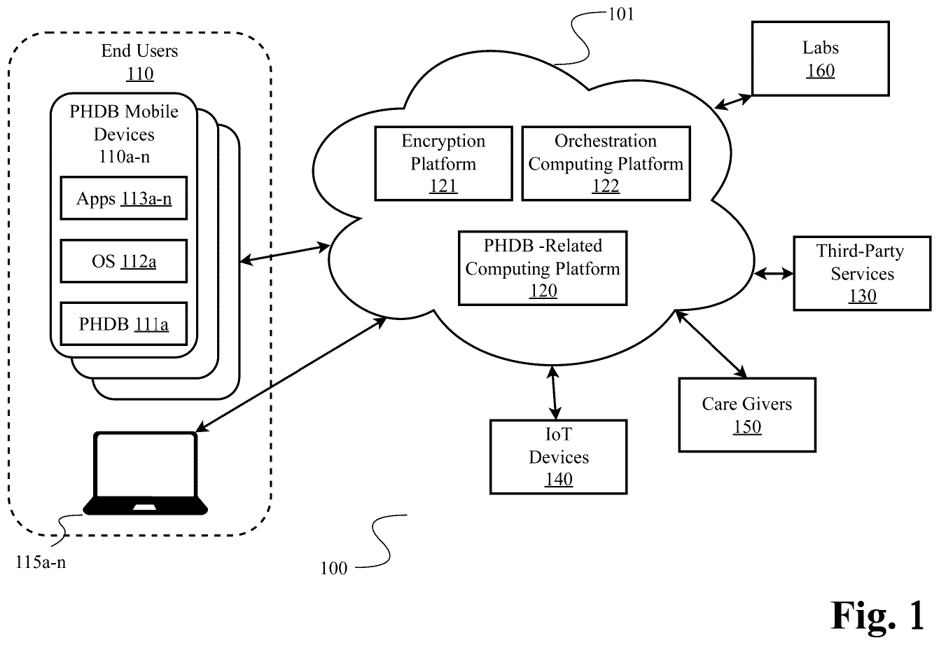

FIG. 1 is a high-level architecture diagram of an exemplary system for discretely filtering two or more human genomes for compatibility and risk using personal health databases (PHDBs), according to an aspect of the invention. As shown in FIG. 1, system 100 offers accessibility to a variety of entities including end users 110, Internet of Things (IoT) devices 140, Care Givers 150, Third-Party Services 130, and Labs 160 by connecting to various cloud-based 101 platforms (e.g., systems, subsystems, and/or services) via a suitable communication network such as the Internet. End Users 110 have flexibility, choosing to engage in cloud-based processing through either their Personal Computers 115a-n which may connect to the cloud-based platforms 101 via a browser-based website or web application, or PHDB-enabled Mobile Devices 110a-n (e.g., smart phone, tablet, smart wearable clothing or glasses, headsets etc.). These mobile devices may comprise a PHDB 111a, an operating system (OS) 112a, and various applications (Apps) 113a-n, creating a comprehensive environment for users to manage and interact with their health and preference data. The ability to have authorized disclosure rules and suggestions or delegate sharing and visibility for personal health records or conditions can also vastly simplify medical procedures and improve outcomes for patients. Current systems force patients into cumbersome manual and often paper disclosure certifications (e.g., outpatient surgery procedure) but could instead be configured to send appropriate status and visibility (even for physical visitation rights in hospital) data to family and friends. This can also better enable post-operative and non-medical facility care by enabling family and friend and personal uploads to the PHDB of photos, interactions, observations, sensor data which can be made available to PHDB processes or to medical staff supporting outcomes.

A user of the system may collect various personal consumption, environment, activity, and other health-related data from a plurality of sources and store the data in their personal health database. Personal health-related data can include genetic information and medical information associated with the user, as well as other types of biometric, behavioral, and/or physiological information. Personal health-related data may be obtained from various sources including, but not limited to, labs 160, third-party services 130, care givers 150, and IoT devices 140. For example, genetic information may be obtained from a lab 160 that conducts genetic carrier screening (e.g., autosomal dominant, autosomal recessive, X-linked dominant, X-linked recessive, mitochondrial, etc.) for a user. Ongoing urine data may be fed from Withings new urine sensor kit, body scan data from an at home body scanner/scale, temperature data from thermal cameras or thermometers, sleep data from smart mattress covers, snoring and sleep quality and sleep apnea indicators from wearable microphones along with heart rate and blood oxygen levels, et cetera. Best practices for individuals or couples wishing to improve personal health outcomes or shared goals such as having children now can include genetic indicator monitoring (e.g. for new papers and research) as well as lived experiences and exposures that may enhance or reduce their risk of adverse health outcomes.

Genetic testing can play a significant role in medical treatment. Some common types of genetic tests that can produce genetic information that can be stored in an individual's PHDB can include diagnostic testing, carrier testing, prenatal testing, newborn screening, pharmacogenetic testing, predictive and presymptomatic testing, forensic testing, and research genetic testing. Diagnostic testing is used to identify or rule out a specific genetic or chromosomal condition. It is done when there is a suspicion based on symptoms or family history. Carrier testing is used to determine if a person carries a gene for a genetic disorder. This type of testing is often done in people with a family history of genetic disorder or in specific ethnic groups with a higher risk. Prenatal testing is conducted during pregnancy to detect genetic abnormalities in the fetus. Examples include amniocentesis, chorionic villus sampling (CVS), and non-invasive prenatal testing (NIPT). Newborn screening involves a series of tests performed on newborns to detect certain genetic disorders early, allowing for early intervention and treatment. Pharmacogenetic testing analyzes how an individual's genes affect their response to certain medications. This information can help personalize medication dosages and selection. Predictive and presymptomatic testing is used to identify genetic mutations associated with conditions data develop later in life, such as certain types of cancer. Presymptomatic testing is done in individuals who do not yet have symptoms but have a family history of a genetic disorder. Forensic testing is used for identification purposes, such as in criminal investigations or paternity testing. Research genetic testing is conducted as part of research studies to better understand the roles of genetics in health and disease. These tests can provide valuable information for healthcare providers, care givers, individuals, and prospective mates.

In some implementations, labs 160 may comprise a plurality of types of labs and facilities that could gather genetic, biometric, behavioral, and/or physiological data on a user. Exemplary labs/facilities can include, but are not limited to, research laboratories (e.g., often affiliated with universities or research institutions and conduct studies to gather various types of data), biotechnology companies, healthcare facilities (e.g., hospitals, clinics, and other healthcare facilities may gather data as part of patient care or research studies. This data could include information from medical tests, imaging studies, and patient questionnaires), tech companies (e.g., wearable technology industry), government agencies, and consumer research firms.

According to the embodiment, caregivers 150 may also provide information to PHDB about the individual which they are providing care for. A caregiver, depending on their role and the context of care, may be responsible for a wide range of medical information. Some common types of medical information that a caregiver might know about or be responsible for include, but are not limited to, patient history (e.g., information about past illnesses, surgeries, medications, allergies, and family medical history), current health status (e.g., information about the patient's current health, including any ongoing medical conditions, symptoms, and vital signs such as blood pressure, heart rate, and temperature), medications (e.g., information about the medications the patient is taking, including dosage, frequency, and any special instructions), treatment plans (e.g., information about the patient's treatment plan, including any medications, therapies, or procedures that have been prescribed), progress notes (e.g., notes on the patient's progress, including any changes in their condition, response to treatment, or other relevant information), diagnostic tests (e.g., information about any diagnostic tests that have been performed, such as blood tests, imaging studies, or biopsies, and the results of those tests), care plan (e.g., information about the overall plan of care for the patient, including goals, interventions, and follow-up care), patient education (e.g., information about legal and ethical issues related to the patient's care such as advance directives, consent for treatment, and confidentiality), and coordination care (e.g., information about coordination of care with other healthcare providers, including referrals, consultations, and care transitions). The specific medical information that a caregiver is responsible for and can provide to the PHDB of their patient will vary depending on the setting and scope of their practice, as well as the needs of the patient.

According to the embodiment, cloud-based platforms 101 may integrate with various third-party services 130 to obtain information related to a user's genetics, biometrics, behavior, and/or physiological characteristics. For example, platform 101 may obtain an electronic health record (EHR), or a subset thereof, associated with the user for inclusion in the user's PHDB.

Additionally, a PHDB mobile device 110a-n may comprise a plurality of sensors which may be used to monitor and capture various biometric, behavioral, and/or physiological data associated with the owner (end user) of the PHDB mobile device. Captured sensors data may be stored in PHDB 111a either in raw data form, or in a format suitable for storage after one or more data processing operations (e.g., transformation, normalization, etc.) has been performed on the sensor data. In some embodiments, a purpose-built software application 113a-n configured to collect, process, and store various sensor data (e.g., biometric, behavioral, physiological, etc.) obtained by sensors embedded into or otherwise integrated with PHDB mobile devices 110a-n. Some exemplary sensors that may be embedded/integrated with PHDB mobile device can include, but are not limited to, fingerprint sensor, facial recognition sensor, heart rate sensor, accelerometer, gyroscope, continuous glucose monitor (CGM), Global Positioning System (GPS), microphone, camera, light sensor, electromagnetic sensors, barometer, pedometer/step counter, galvanic skin response (GSR) sensor (e.g., measures skin's electrical conductivity, which can vary with emotional arousal, stress, or excitement), temperature sensor, lidar, and infrared sensor. More advanced sensors might include Raman-based real-time analytics, gas chromatography mass spectrometry, liquid chromatography mass spectrometry, capillary electrophoresis mass spectrometry, which may be of particular use in environmental exposure considerations in health conditions and lived gene expression. These sensors can be used individually or in combination to gather a wide range of data about the user's biometric, behavioral, and physiological characteristics, enabling various applications such as health monitoring, fitness tracking, personalized user experiences, and human genome filtering for compatibility, to name a few. It is important to note that when combined with temporal and graph representations of interactions in the individual's life, this can feed into a much more nuanced biological monitoring, modeling and simulation aid available for personal, family, or medical use. Users who gather such data fastidiously may also be of particular interest to researchers in support of uncertainty reduction and isolation of particular genetic linkages to this litany of more comprehensive lived factors commonly excluded from static genomics analysis.

In some embodiments, PHDB 111a may be stored in the memory of PHDB mobile or wearable device 110a. In some embodiments, PHDB 111a may be implemented as an encrypted database wherein the plurality of personal health data stored therein is cryptographically encrypted to protect the personal and sensitive data stored therein.

End users 110 may also engage in edge-based processing referring to computing devices that process data closer to the source of data generation instead of relying solely on a centralized server. Edge devices are situated close to the point where data is generated, such as sensors, cameras, or other Internet of Things devices. The Internet of Things (IoT) 140 devices refer to physical objects embedded with sensors, software, and other technologies that enable them to connect and exchange data over the internet. These devices are part of the broader concept of the Internet of Things, which involves the interconnection of everyday objects to the Internet, allowing them to collect and share data for various purposes. Internet of Things devices find applications in various domains, including smart homes, healthcare, industrial automation, agriculture, transportation, and more. Examples include smart thermostats, wearable health monitors, industrial sensors, and connected vehicles. According to the embodiment, a plurality of IoT devices 140 may be deployed to collect and transmit various types of information related to a user's genetics, biometrics, behavior, and/or physiological characteristics. In some implementations, IoT devices 140 can include a plurality of sensors, devices, systems, and/or the like configured to collect and transmit data to cloud-based platforms 101 for inclusion in the user's PHDB. Some exemplary IoT devices 140 can include fitness trackers, smart scales, smart clothing, smart home devices, genetic testing kits, sleep monitors, health monitoring devices (e.g., devices that measure health parameters such as blood pressure, glucose levels, and oxygen saturation, etc.), and wearable cameras.

To facilitate proactive filtering across multiple platforms during interactions with prospective mates, the cloud 101 integrates an optional encryption platform 121, an orchestration computing platform 122, and a PHDB-related computing platform 120.

According to the embodiment, an optional encryption platform 121 may be configured and deployed to provide strong encryption to protect data from unauthorized access. In addition to using strong encryption algorithms, encryption platform 121 is configured to follow best practices for key management, such as using strong, randomly generated encryption keys, and regularly rotating keys to minimize the risk of unauthorized access. In an embodiment, encryption platform 121 may implement advanced encryption standard (AES) for encrypting the various data stored in PHDB. AES is a symmetric encryption algorithm that is widely used and considered to be very secure. It is often used to encrypt data at rest, such as files stored on PHDB. In an embodiment, encryption platform 121 may utilize RSA which is an asymmetric encryption algorithm commonly used for encrypting data in transit, such as data sent over the Internet. In another embodiment, elliptic curve cryptography (ECC) may be implemented which is an asymmetric encryption algorithm that is known for its efficiency and security. In some embodiments, ECC may be used to encrypt data obtained and transmitted by IoT devices 140 to cloud-based platforms 101. In some implementations, a combination of encryption schemes may be utilized to provide secure data storage and transmission. For example, personal-health data may be encrypted in the cloud using RSA and then sent to an end user mobile device 110a wherein it may be encrypted using AES for storage on PHDB 111a of the mobile device.

In some embodiments, encryption platform 121 may implement homomorphic encryption when processing or otherwise analyzing personal health information. In this way, the system can provide processing of encrypted data without having to decrypt and potentially leak personal information.

An orchestration platform 122 is present and configured to provide automated management, coordination, and execution of complex tasks or workflows. This can involve deploying and managing software applications, provisioning and managing resources, and coordinating interactions between different components of system 100. Orchestration platform 122 may automate the deployment and management of virtual machines, containers, and other resources. This can include tasks such as provisioning servers, configuring networking, and scaling resources up or down based on demand. For example, orchestration platform 122 may define and execute a workflow related to the collection, encryption, and distribution (to the appropriate PHDB) of user health-related information. Of particular importance is the ability of the platform to interact with AI/ML systems to aid in explaining, modeling, extracting models, or generating potential items of interest for consideration by medical experts or users. This is further enhanced by the ability for automated planning and modeling simulation services to consider forward scenario analysis of factors (e.g., what if I stopped eating bacon every morning and walked a minimum of 15,000 steps per day instead of my current activity level). This may also help generate financial models to aid users in their personal decision-making and potentially for insurers or medical professionals in theirs. This is becoming more important in the emerging CRISPR/Cas9 and with the sudden emergence of Ozempic and Zapbound era. This is likely to become more challenging for payers, patients and providers if the expected multireceptor agonists such as LY3437943 (a novel triple agonist peptide at the glucagon receptor (GCGR), glucose-dependent insulinotropic polypeptide receptor (GIPR), and glucagon-like peptide-1 receptor (GLP-1R)), emerge and potentially offer large benefits to at risk populations with severe disease and broad-based disease risk factor reductions. Such financial “what if” scenarios will become important when considering the lifetime value of treatment options and potential payment models and is critical to improving patient outcome and better managing continuity of care for healthier and ultimately cheaper patients.

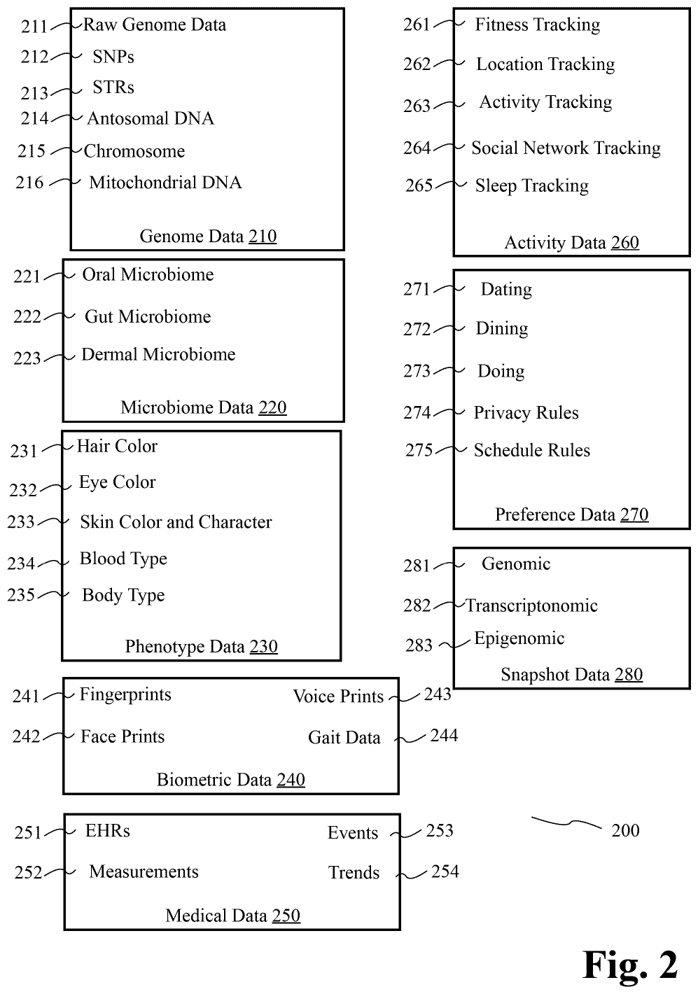

FIG. 2 is a diagram showing an exemplary arrangement of a personal health database (PHDB), according to an aspect of the invention.

In at least one aspect of an embodiment, a personal health database may be implemented as a cloud-based storage system for storing personal health-related information designed for security, scalability, and privacy.

In at least one aspect of an embodiment, a personal health database may be implemented as a database for storing personal health-related information stored in the memory (or some other storage system) of a PHDB mobile device 110a.

Regardless of the implementation, the system may require secure authentication mechanisms (e.g., username/password, two-factor authentication) to ensure that only authorized users can access the data. Role-based access control can be used to manage permissions for different types of users (e.g., patients, healthcare providers). Each individual can set their PHDB permissions with respect to other individuals or entities (e.g., that is who can access the data, other people, other applications or services, etc.) and scope (e.g., how much data can permit individuals access). All data stored in the system may be encrypted (such as by encryption platform 121) both in transit and at rest to protect it from unauthorized access. This can include using strong encryption algorithms such as AES, and in some implementations, homomorphic encryption. Personal health information can be segmented into categories (e.g., medical records, lab results, prescriptions, genome data, microbiome data, phenotype data, biometric data, activity data, preference data, etc.) to facilitate access control and data management. The system can be designed to handle a plurality of users and a large volume of data. This involves leveraging scalable cloud infrastructure and database technologies. In some implementations, mechanisms may be put in place to ensure the integrity and availability of the data, including regular backups and redundancy. The storage system may be configured to comply with relevant regulations and standards for health information privacy and security, such as the Health Insurance Portability and Accountability Act (HIPAA) in the United States. Additionally, the storage system may maintain detailed audit logs of all access and modifications to the data for accountability and compliance purposes.

According to some embodiments, the PHDB system comprises a user-friendly interface (e.g., graphic user interface) for users to access and manage their health information, as well as for healthcare providers to view and update patient records, if applicable. For example, a software application stored and operating on PHDB mobile device 110a may provide a user interface where PHDB mobile device users can manage their stored personal-health information including uploading new information, editing existing information, and setting permissions/managing access control over their stored personal health information. Overall, the PHDB system provides a secure and reliable platform for storing personal health information while ensuring that it remains accessible to authorized users when needed for personal, family, group or medical purposes.

Personal health information can be segmented into content categories 200 to facilitate access control and data management to be able to accommodate selective degrees of “opting in” to screening processes and the ability for multiple applications to engage with common data. This may further enable user-specific preferences such as, for example, specific “deal breakers” for either party if a “screening mode” is enabled. Similar users may authorize their data for evaluation by third parties with designated filters via rules, criteria or models (AI/ML or statistical) for approaching them or their medical providers with prospective requests, data use asks, or as a candidate for studies, treatments, or therapies.

PHDB content categories 200 may include, but are not limited to, genome data 210 which encompasses a diverse set of genetic information critical for understanding an individual's biological makeup. The subcategories can include the raw genome data 211, which is the complete genomic sequence of an individual, the single nucleotide polymorphisms (SNPs) 212, which are variations at the level of a single nucleotide, the short tandem repeats (STRs) 213, which are tandemly repeated DNA sequences, autosomal DNA 214, which are non-sex chromosome DNA, chromosome 215, which are individual chromosomes contributing to the genome, and mitochondrial DNA 216 which is genetic information from mitochondria. Additionally, genome data 210 can comprise an individual's RNA, DNA, and/or mtDNA data. A use case for such data is genetic alteration via in vivo, in vitro, or ex vivo processes targeting RNA, DNA, and/or mtDNA. Furthermore, genomic data 210 may comprise multiple instances of genomes in an individual's life. For example, if an individual gets a CRISPR/Cas9 therapy then the individual's genome be a v1Individual vs. v2Individiual vs. v3Individual etc, wherein each genome represents discrete genomic measurements of the same individual but that yield different results. This may involve analyzing a user's snapshot data 280, if available, and analyzing the users bioinformatic data over time to identify changes in genomic information.

Genome data 210 may be obtained from suitable third-party services 130 such as direct-to-consumer genetic testing companies, from various labs 160 which performed genetic testing, or any other suitable source of genetic information.

Microbiome data 220 explores the composition of microbial communities residing in various parts of the body. The human body is home to trillions of microorganisms, including bacteria, viruses, fungi, and other microbes, collectively known as the microbiota or microbiome. These microbial communities play a crucial role in human health and wellness in several ways. The gut microbiota, located primarily in the intestines, plays a key role in digestion and nutrient absorption. It helps break down complex carbohydrates, produces vitamins (such as vitamin K and some B vitamins), and metabolizes dietary compounds that are otherwise indigestible. The microbiota plays an important role in training and modulating the immune system. It helps distinguish between harmful pathogens and harmless antigens, and it assists in the development of immune tolerance. The gut-brain axis refers to the bidirectional communication between the gut and the brain. The gut microbiota can influence this axis and has been linked to conditions such as anxiety, depression, and stress. Imbalances in the microbiota, referred to as dysbiosis, have been associated with a variety of health conditions, including inflammatory bowel diseases (such as Crohn's disease and ulcerative colitis), allergies, asthma, autoimmune diseases, and even certain cancers. The gut microbiota can affect how medications are metabolized and their effectiveness. It can also influence the side effects of certain medications. Therefore, it would be beneficial and useful to have readily available microbiome data related to a user for various purposes such as matching with and vetting potential romantic partners, purchasing and/or prescribing medications or treatments, and purchasing food (either at a restaurant or at a grocery store).

Some exemplary subcategories of microbiome data 220 include the oral microbiome 221, which are microbial flora in the oral cavity (e.g., the mouth), the gut microbiome 222 which are the microorganisms in the gastrointestinal tract. The dermal microbiome 223 which are the microbes associated with the skin. Microbiome data 220 may be obtained in several ways. One method may involve the use of a third-party service 130 such as a direct-to-consumer microbiome testing, which typically requires an individual to provide a stool sample which is then analyzed to provide information about the composition of the individual's microbiome. Similarly, a medical provider may order some tests which require an individual to provide a saliva or urine or stool (or other bio-sample) for lab 160 analysis of the individual's microbiome. In some implementations, one or more IoT devices 140 may be deployed and configured to measure and transmit data related to one or more bio-samples of an individual to cloud-based platforms 101 for processing.

Phenotype data 230 describes observable traits and characteristics influenced by both genetic and environmental factors. Phenotype data can include physical characteristics, physiological traits (e.g., blood pressure, cholesterol levels, metabolism, etc.), and behavioral traits such as personality traits, cognitive function, and disease susceptibility. Some exemplary subcategories include hair color 231, eye color 232, skin color and character 233, blood type 234, and body type 235.

Generally, phenotype data is obtained through self-observation and recording of various traits and characteristics. Some physical characteristics may be obtained from linked electronic health record data such as height, weight, body mass index (BMI), eye color, hair color, etc. In some instances, IoT 140 or other sensor data may be used to obtain phenotype data related to physiological traits e.g., blood pressure, heart rate, etc. A user may submit phenotype data about themselves via their PHDB mobile device 110a or their computer 115a. For example, a user can submit information about their daily activities, habits, and behaviors including sleep patterns, exercise routines, dietary habits, and any notable behaviors or tendencies. Additionally, or alternatively, a user may optionally choose to link third-party services or applications with their PHDB so that personal health information about the user that is captured by said services or applications may be sent to PHDB for storage. For instance, a user may integrate their dietary data from a nutritional application, stored on their PHDB mobile device, which tracks the user's meals and macro-nutrients consumption as well as other eating habits. As another example, a user may choose to allow third-party services which provide cognitive tests, personality assessments, or psychological evaluations to gather data on the user's cognitive abilities, personality traits, and mental health, and provide this information for storage in the user's PHDB. By systematically collecting and recording this information over time, the system can build a comprehensive profile of phenotype data, which can be valuable for personal health management, understanding genetic predispositions, and participating in research studies or clinical trials.

Biometric data 240 involves distinctive physical and behavioral characteristics unique to an individual. Some exemplary subcategories include, but are not limited to, fingerprints 241, face prints 242, voice prints 243, and gait data 244. Biometric data 240 may be obtained from various sources such as, for example, IoT devices 140 or sensors and/or third-party services 130. There are several biometric sensors available for obtaining biometric data. Such sensors may be embedded hardware such as embedded into a PHDB mobile device 110a (e.g., a fingerprint scanner, microphone, facial recognition system, etc.). Biometric sensors may be separate devices (e.g., stand-alone fingerprint scanner) which may be connected to (wired or wireless) a computing device such as a computer 115a or mobile device and which may obtain and transmit biometric data. Some exemplary biometric sensors which may be used to obtain biometric data 240 can include heart rate monitors, electrocardiogram sensors, blood pressure monitors, pulse oximeters, temperature sensors, electrodermal activity sensors, microphones, facial recognition systems, accelerometers and gyroscopes, and bioimpedance sensors, to name a few.

Medical data 250 encompasses a range of health-related information. The subcategories may include electronic health records (EHRs) 251, measurements 252, events 253, and trends 254. Medical data 250 may be obtained from a user's linked EHR (e.g., Provider specific, or Insurer/Payer) or connected devices (e.g., phone, watch, wearables) or a combination. Medical measurements 252 encompass a wide range of quantitative data collected during the assessment, diagnosis, and monitoring of health and disease. Some common medical measurements include vital signs (e.g., body temperature, pulse rate, blood pressure, respiratory rate, etc.) body measurements (e.g., height, weight, BMI, waist circumference, etc.), laboratory tests (e.g., complete blood count, blood chemistry tests, urinalysis, sexually transmitted infection analysis, etc.), imaging studies (e.g., x-rays, ultrasound, magnetic resonance imaging, computed tomography scan, etc.), electrocardiogram, and various biometric measurements (e.g., heart rate variability, electrodermal activity, pulse oximetry, etc.).

A medical event 253 may refer to a significant health-related incident that may require medical attention or intervention. Medical events can vary widely in nature and severity, and they can be acute or chronic. Some examples of medical events include, but are not limited to, acute illness, injury, allergic reaction, surgery, hospitalization, medical procedure, mental health crisis, cardiovascular event, and/or neurological event (e.g., seizure or transient ischemic attack).

Activity data 260 captures information related to an individual's physical and social activities. The subcategories include fitness tracking 261, location tracking 262, activity tracking 263, social network tracking 264, and sleep tracking 265. A user may optionally select which services and/or applications they wish to integrate with their PHDB. In this way, the user can manage which data is uploaded to their PHDB.

Preference data 270 reflects an individual's preferences in various aspects of life. The subcategories include dating 271, dining 272, doing 273, privacy rules 274, and schedule rules 275. Dating preferences 271 may include a desired attributes in a potential romantic partner. These desired attributes may be physiological attributes (e.g., height, weight, ethnicity, etc.), character attributes (e.g., humor, kind, intelligent, etc.), behavioral attributes (e.g., mental health), or any other type of preference an individual may have as it relates to dating. Dining preferences 272 may refer to types of food (e.g., Chinese, Italian, Mexican, Indian, Vegan, etc.), specific or general restaurants, eating habits or restrictions, price-point limits (e.g., no restaurants with $70 entrees), dining ambiance preferences, and/or the like. Doing preferences 273 may be closely related to activity data and may refer to activities the individual prefers to do or perform while on a date. For example, an individual may input (or have an AI/ML assistant input for them based on historical activities/data) that they enjoy hiking, long distance bike riding, and sailing, but they are also open to try new activities like snorkeling or paddle boarding. Privacy rules 274 may refer to guidelines which an individual can set to manage the collection, use, disclosure, and protection of their personal health information store in their PHDB. Food sensitivities like Lactose, Gluten or other allergies (e.g., nuts) may also be encoded. Privacy rules can vary depending on the jurisdiction, user, and the type of information being protected. They can include provisions for obtaining consent for the collection and use of personal information, maintaining the confidentiality of that information, and providing individuals with access to their own information. Schedule rules 275 may refer to rules set by an individual which govern the scheduling of events (e.g., prospective dates with matched individuals), activities, or resources. In general, schedule rules may comprise (but not limited to) availability rules, priority rules, constraints, and synchronization rules.

Snapshot data 280 refers to an individual's various bioinformatic information at a given point in time. In bioinformatics, a “snapshot” of data typically refers to a specific point-in-time representation of biological data, often used for analysis or storage purposes. As illustrated, snapshot data 280 may comprise genomic data snapshots 281 which may capture the genetic information of an individual (e.g., human or animal) at a specific moment. This can include DNA sequences, variations (SNPs), and structural variants. Transcriptonomic data snapshot 282 may represent the gene expression levels in a cell or tissue at a specific time. This can include RNA sequencing data, which provides information about which genes are active and at what levels. Epigenomic data snapshots 283 may capture the epigenetic modifications (e.g., DNA methylation, histone modifications) in a cell or tissue at a specific time. This can provide insights into gene regulation and cell differentiation. Additional exemplary snapshot data can include, but is not limited to, proteomic data snapshots which capture the proteins present in a cell or tissue at a specific time and can include data on protein abundance, modifications, and interactions; metabolomic data snapshots which represent the small molecule metabolites present in a cell or tissue at a specific time and can provide insights into metabolic pathways and cellular processes; phylogenetic data snapshots which may represent the evolutionary relationships between different species or populations at a specific time and can include phylogenetic trees based on genetic or genomic data; and structural biology data snapshots which capture the three-dimensional structures of biological molecules (e.g., proteins, nucleic acids) at a specific time and can include data from X-ray crystallography, NMR spectroscopy, or cryo-electron microscopy.

This exemplary arrangement of PHDB content categories establishes a robust foundation for comprehensive health and preference profiling. This structure allows for effective management, analysis, and utilization of diverse data types to enhance personalized insights and applications within the described system.

According to some aspects, a PHDB or a subset of the data stored therein may be configured as a graph database. Such a graph database may be leveraged for various analytic, predictive, and/or scoring processes described herein. According to some aspects, platform 120 implements contact tracing and health notification with a focus on decentralized tracing and anonymity options. For example, the system may use a graph database to store and analyze the plurality of data collected by platform 120. Graph databases are well-suited for modeling complex relationships, making them ideal for contact tracing, where connections between individuals are important. Platform 120 may utilize one or more algorithms configured for contact tracing based on the collected data. For example, the algorithm could consider proximity between individuals, duration of contact, and other relevant factors to determine potential exposure to diseases. The system may be configured to send health notifications to individuals who may have been exposed to a disease. This can include information about testing, quarantine guidelines, and other relevant instructions. According to an aspect, the platform allows for decentralized tracing, meaning that individuals have control over their data and can choose whether to share it for contact tracing purposes. This may be accomplished by providing options for individuals to remain anonymous while still participating in contact tracing. This can be achieved through pseudonymization or other privacy-preserving techniques. Furthermore, platform contact tracing can integrate with healthcare providers to ensure that individuals receive appropriate care and follow-up based on their exposure status.

According to some embodiments, PHDB may further comprise Transposon data. Transposon, referred to as “jumping genes” are DNA segments that can move within the genosome to influence gene expression including transcription (synthesis of RNA from DNA) and translation (synthesis of proteins from RNA). Transposon placement can modify a gene's expression pattern via mechanisms such as providing for or disruption regulatory sequences like enhancers (which increase gene transcription) or silencers which decrease transcription. They can also rearrange genomic structure to create new gene combinations or duplicate existing genes as part of evolutionary processes. They can also insert themselves into a gene, which may be problematic and linked to disease but may also provide evolutionary benefits via novel gene functions or expression patterns.

According to some embodiments, PHDB may further comprise spatial omics data. The PHDB can store a 3-dimensional (3d) mesh (plus time which makes its 4-dimensional) of the body with different resolution levels (i.e., mesh shapes and densities) to support analytics and simulation modeling. Where available, platform may tag multi-omics data to any given “cell” in the 3d mesh. For example, both digital and physical samples (and associated extracted information) can be spatially and temporally located to the body to provide a spatially resolved view of the biological molecules with a tissue or organism. Examples of spatial omics data can include but is not limited to, spatial transcriptomics, spatial proteomics, spatial metabolomics, spatial genomics, and spatial multi-omics (e.g., refers to the integration of multiple omics data types such as transcriptomics, proteomics, metabolomics, etc.) with spatial information. According to an embodiment, a user may select spatiotemporal restrictions on what part of the electronic medical record (e.g., which mesh or subset of a mesh) can be viewed by a given party.

A particular function of platform 120 and PHDB is the capability to analyze genome and gene expression over time and in different places in the body. To assess genome and gene expression over time, the platform may utilize one or more techniques. A first technique may involve conducting longitudinal studies to track changes in the genome over time. This can involve sequencing DNA samples from the same individual at multiple time points to identify genetic variations, including those caused by transposon movement. Another technique may use spatial transcriptomics techniques to analyze gene expression patterns in specific regions or tissues of the body. This can provide insights into how gene expression varies spatially and how it changes over time. A transposon analysis technique may be used to study the movement and impact of transposons on gene expression over time and in different tissues. This can involve identifying transposon insertion sites and their effects on nearby genes. This may also involve the use of evolutionary analysis to study how transposon movement has contributed to genetic diversity and the evolution of new gene functions. This can involve comparing transposon sequences across different species or populations. Stored 3d and 4d mesh information may be analyzed to analyze genome and gene expression over time.

According to an embodiment, the platform may allow for multiple simulation paths based on “seeds” extracted from data stored in PHDB. Because there is uncertainty with respect to sampling, the system can engage in express parametric studies (e.g., initial seed manipulation) to look at potential impacts of various factors/constraints such as imaging, sampling, extraction, errors and uncertainty for statistical, ML/AI, or modelling simulation processes (e.g., diagnostics, treatment advisory, treatment calibration, etc.).

According to an embodiment, the platform may leverage ML/AI processes that classify individual cells and/or identify cellular neighborhoods. The results of such processes may be used to create an enhanced 3d (or 4d) mesh for anchoring all data available to analysis functions. This is useful because finite element analysis, fluid modeling, fluid structure interaction modeling and broader biological simulation models are strongly intertwined with the selection and determination of such boundaries. It should be noted that such meshes can shift over time. According to an aspect, a stabilized space-time process can enable more accurate and advanced modeling simulation and ML/AI basis as a reference “atlas” for single cell, cell groups (i.e., tissue/cellular neighborhoods), and other multi-omics information as well as observations, medical data, fitness data, telemetry, imagery, etc. This may involve simulating from an average mesh that is viewed as a stable point, then minimizing the dynamism inside the mesh over a finite time. This is useful because it moves medical records data into the equivalent of a (optionally) stabilized space time mesh projection (think of it like a GPS system for the body) to provide spatiotemporal locality of data. The system may be configured to support single cell/tissue, multiple tissue groups, or whole-body simulations and can parallelize parametric studies of different “assumptions” and perturbations of said models.

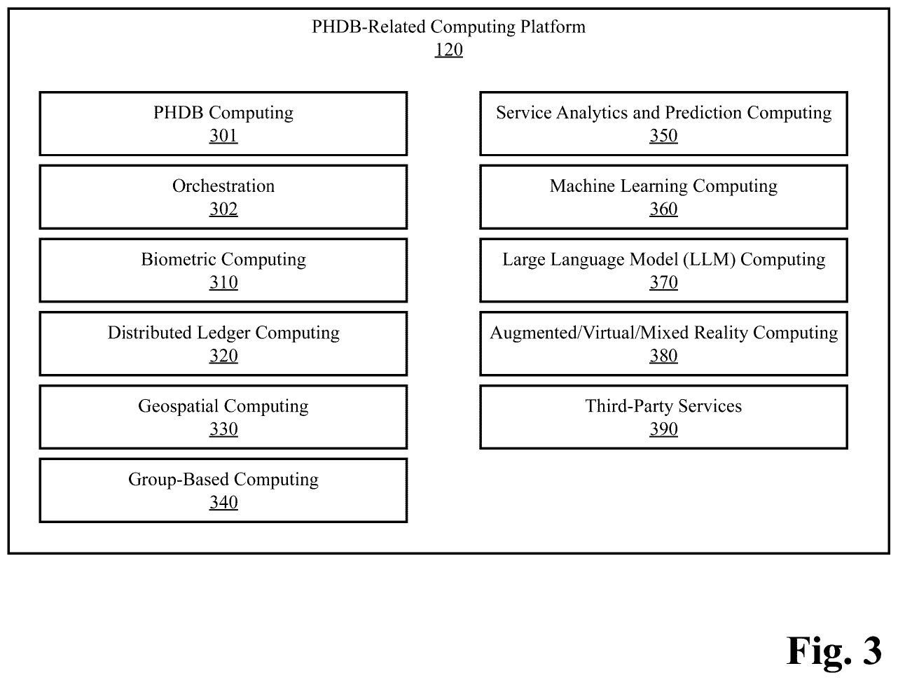

FIG. 3 is a system diagram showing an exemplary arrangement of a cloud-based PHDB-related computing platform, according to an aspect of the invention. According to the aspect, the PHDB computing platform 120 may be implemented as a computing system comprising one or more hardware processors configured for providing the various functionality described herein. According to another aspect, the PHDB computing platform 120 may be implemented as non-transitory, computer-readable storage media having computer-executable instructions embodied thereon and executed by one or more hardware processors configured for providing the various functionality described herein. According to another aspect, the PHDB computing platform 120 may be a computer-implemented method executed by the platform.

According to the embodiment, PHDB-related computing platform 120 comprises various computing components configured to provide computing functionality directed to various categories to support the discrete filtering of two or more human genomes for compatibility using personal health databases. These exemplary computing components may be implemented as servers or services which provide on-demand computing when queried by a system or process. For example, a PHDB-enabled mobile computing device may query geospatial computing 330 to locate and map other PHDB users in the location of mobile device user, and responsive to the query, geospatial computing 330 can send the identified and mapped locations of the other users to the mobile device for display in an application.

PHDB computing 301 is specialized in managing and processing PHDB data, ensuring efficient access and retrieval of diverse health and preference information. Orchestration 302 is responsible for coordinating and managing the flow of data and processes within the system, ensuring seamless interactions between different computing components.

Biometric computing 310 focuses on the analysis and interpretation of biometric data, including fingerprints, face prints, voice prints, and gait data.

Distributed Ledger Computing 320 utilizes distributed ledger technology for secure and transparent record-keeping of sensitive health and preference data, ensuring data integrity and privacy.

Geospatial computing 330 offers ready-to-use demographic datasets and map/imagery layers that allow users to gain immediate context to applications of all types. Group-based computing 340 offers the use of collaboration tools and software that enable multiple users to work together on shared tasks. Service analytics and prediction computing 350 involves leveraging data analytics and predictive modeling techniques to enhance the delivery and optimization of services. Machine learning computing 360 refers to the use of computing systems and algorithms to enable machines to learn and make predictions or decisions based on data.

Large Language Model (LLM) computing 370 refers to the utilization of advanced computational systems to train, deploy, and utilize large language models. These models are typically built using deep learning techniques and have the capability to understand, generate, and process human language at a sophisticated level.

Augmented/Virtual/Mixed Reality computing 380 involves integrating digital information, such as graphics, audio, and other sensory environments, with the user's real-world environment in real-time. Enhances the user's perception of the physical world by overlaying computer-generated content onto it.

Third-Party Services 390 engages external entities to provide additional services, expanding the functionality and capabilities of the PHDB-related computing platform.

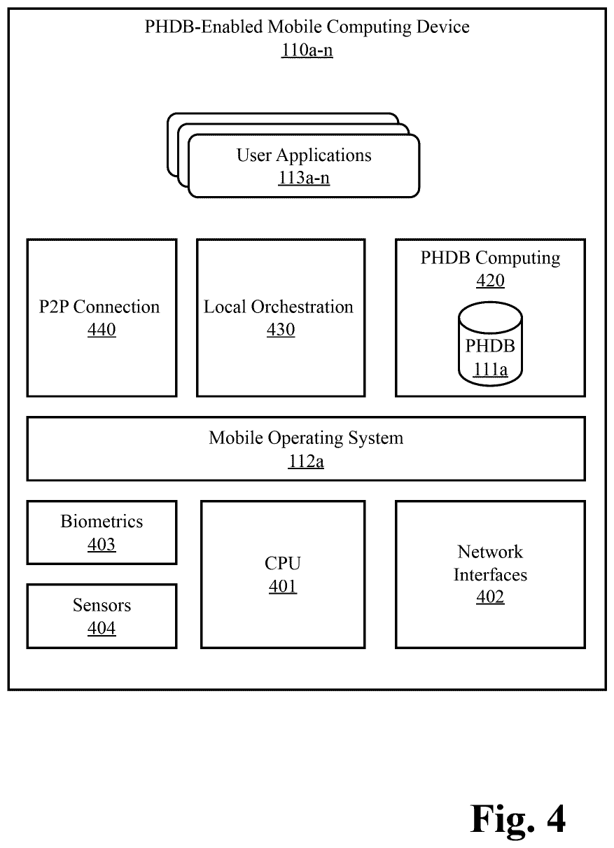

FIG. 4 is a system diagram showing an exemplary arrangement of mobile device configured to support PHDB-related computing, according to an aspect of the invention. The PHDB-enabled mobile computing device 110a-n comprises several key components such as various User Applications 113a-n which are Applications on the mobile device that interface with the PHDB. P2P Connection 440 which establishes a peer-to-peer (P2P) connection facilitating direct communication between PHDB-enabled mobile devices. Local orchestration 430 which manages and coordinates local processes and data flow within the mobile device. PHDB computing 420 which is responsible for securely storing PHDB 111a and is specialized in managing and processing PHDB data, ensuring efficient access and retrieval of diverse health and preference information. PHDB computing 420 may also be configured to perform edge-based computing, in certain embodiments. The PHDB-enabled mobile device 110a-n includes a central processing unit (CPU) 401 for processing, network interfaces 402 for connectivity with various networks (i.e., the Internet, a cellular network), a plurality of sensors 404 for data input, biometrics detection 403 (e.g., fingerprint scanner, microphone, etc.) and a mobile operating system 112a.

The PHDB mobile computing device 420 may be implemented as a smart phone, tablet, or smart wearable device. The PHDB computing device 420 securely stores PHDB 111a. The mobile device, denoted as PHDB-enabled mobile device 110a-n, is capable of sending requests for PHDB-based services to the cloud 101 and receiving requests for PHDB-related access from cloud-services.

The PHDB-enabled mobile device 110a-n establishes a P2P connection 440, enabling secure peer-to-peer PHDB operations. This ensures direct and secure communication between mobile devices for PHDB-related functionalities. For example, if prospective romantic partners have been identified in an area, say a music venue, then identified and matched potential partners may discover and communicate with each other via a P2P connection established between their respective PHDB enabled mobile device 110a.

This configuration enhances the PHDB-enabled mobile device's 110a-n capabilities by enabling local storage and processing of PHDB data, reducing reliance on external cloud services while maintaining secure P2P communication for PHDB operations.

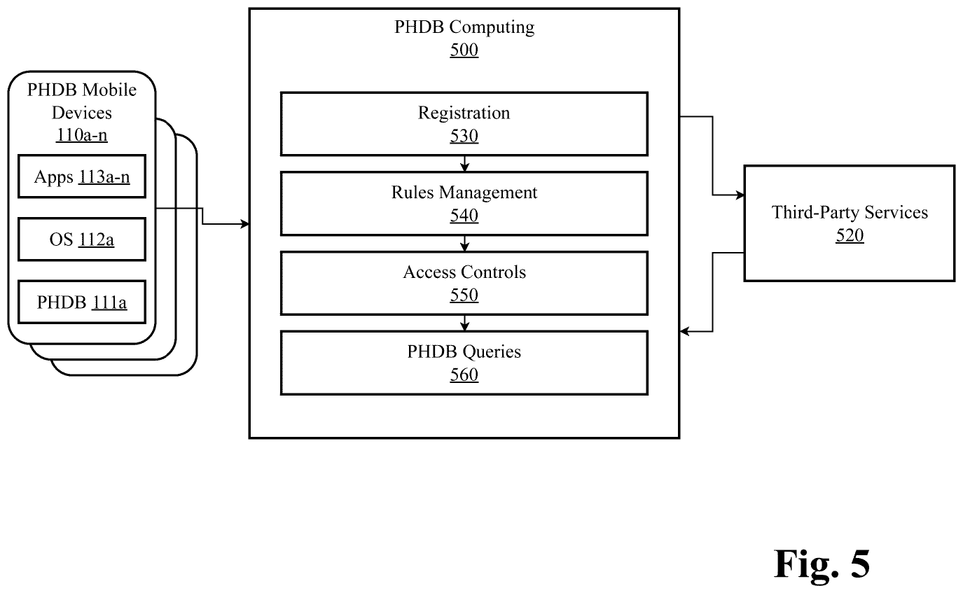

FIG. 5 is a system diagram showing an exemplary arrangement of a PHDB computing server or service with registration controls and third-party service 520 integration, according to an aspect of the invention. The components of PHDB computing 500 initiate a process that commences at the PHDB mobile device 110a-n, encompassing apps 113a-n, an OS 112a, and PHDB 111a. The process then progresses through the stages of PHDB computing 500 which acts as an intermediary in the process flow, facilitating seamless interactions between the PHDB mobile device 110a-n and various third-party services 520. For example, a user of PHDB mobile device 110a may grant permission for social media data related to their likes, check-ins, and subscribed pages data to be uploaded to their PHDB. In this example, PHDB computing 500 may query a social media server to retrieve the social media data, process the retrieved social media data (e.g., encrypt, transform, format, etc.), and then send the processed data to the PHDB mobile device 110a for storage in PHDB 111a.

Registration 530 initiates the registration process for PHDB services to users, ensuring proper onboarding and authentication. Registration may store login credentials of a user for various third-party services 520 to facilitate data exchange. Rules management 540 manages and enforces rules governing the access and usage of PHDB services, contributing to secure and compliant operations. Rules management 540 may be aware of or acquire user-defined access rules for their PHDB and apply those rules to adhere to the user's set permissions regarding access to their personal health information. Access Controls 550 implements controls (e.g., based on user defined rules, or governing rules and regulations such as HIPAA) to regulate access to PHDB services, safeguarding sensitive data and ensuring that users adhere to established rules. PHDB Queries 560 facilitates queries and requests related to the PHDB, enabling users to retrieve specific information or perform actions within the PHDB ecosystem.

In some implementations, personal health information within the PHDB may be shared with the third-party services 520. This integration ensures that the PHDB ecosystem can seamlessly interact with and contribute to third-party services.

This exemplary arrangement offers a comprehensive system for managing PHDB services, from user registration 530, to access controls 550, and facilitates the sharing of genomic data with external third-party services 520, thereby expanding the functionality and reach of the PHDB ecosystem.

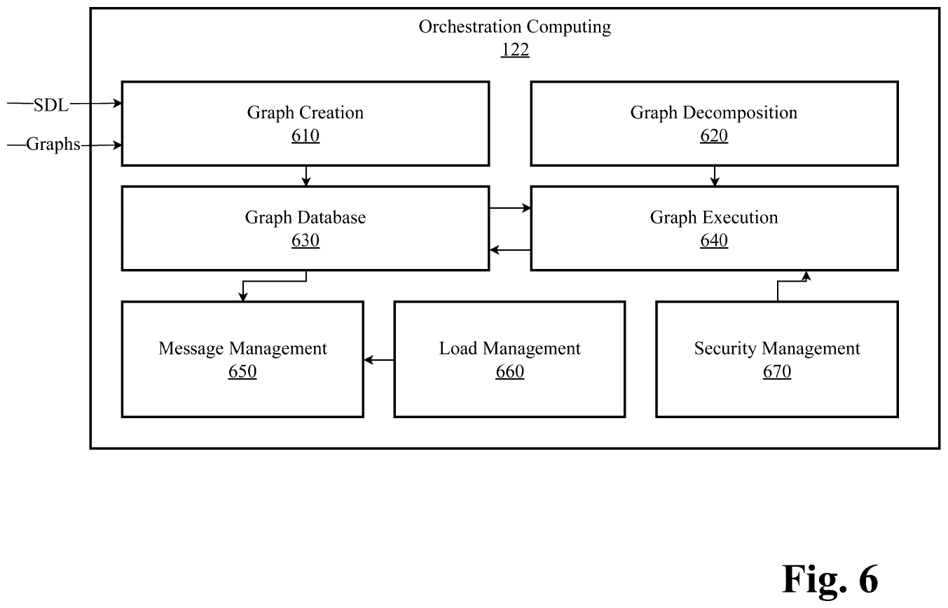

FIG. 6 is a system diagram showing an exemplary arrangement of a PHDB orchestration service according to an aspect of the invention. The orchestration computing 122 initiates a process that involves graph creation 610 which occurs when a user requests a service, the orchestration service receives the service description and creates a service graph. As shown, graph creation 610 may receive as input service description language (SDL) data. It is a language used to describe the services, their dependencies, and the workflow of a distributed system. SDL is used to create a formal description of the services and their interactions, which can then be used by orchestration computing 122 to automate the deployment, scaling, and management of the services. Additionally, or alternatively, graph creation 610 may receive as input a graph to be executed by orchestration computing 122.

The graph creation process results in the establishment of a graph database 630 wherein created graphs may be stored and retrieved for use. For example, a graph may represent various services that need to act on some data to perform the requested process. Graphs are often used in orchestration computing to represent the relationships between different components or tasks in a system. These graphs, known as orchestration graphs or workflow graphs, can help visualize the dependencies between tasks and the flow of data or control through the system.

Graphs can represent the workflow or sequence of tasks that need to be executed to complete a job or process. Each node in the graph represents a task, and the edges represent the dependencies between tasks. Graph decomposition 620 is a process where the created graph undergoes decomposition, breaking it down into subgraphs. This step is integral to optimizing service delivery and resource utilization. For example, each subgraph may represent a single service and the data processing required thereof. Graph execution 640 occurs when the decomposed graph is executed, leading to the activation of subgraphs. This stage ensures that the intended service actions are carried out effectively. The graph database 630 stores and manages the created graph, serving as a repository for orchestrating service delivery. Message management 650 is the process where messages are exchanged between subsystems to orchestrate the delivery of messages. Message may comprise data that is to be operated on or can include instructions for processing. During graph execution, security management 670 ensures the integrity and confidentiality of the controls and graph. This includes implementing measures to protect against unauthorized access or manipulation of genomic data. Load management 660 oversees the distribution of workloads during graph execution, optimizing system performance and resource allocation. This can involve deploying, scaling, and managing complex applications or services across a distributed environment. Graphs can be used to allocate resources dynamically based on the requirements of different tasks or components. For example, a graph can help determine where to deploy a new instance of a service based on current resource availability and workload.

This orchestration service provides a systematic and efficient approach to coordinating the delivery of services within the PHDB ecosystem. By decomposing and executing service graphs, managing messages, ensuring security, and optimizing load distribution, orchestration computing service 122 enhances the overall efficiency and reliability of service delivery.

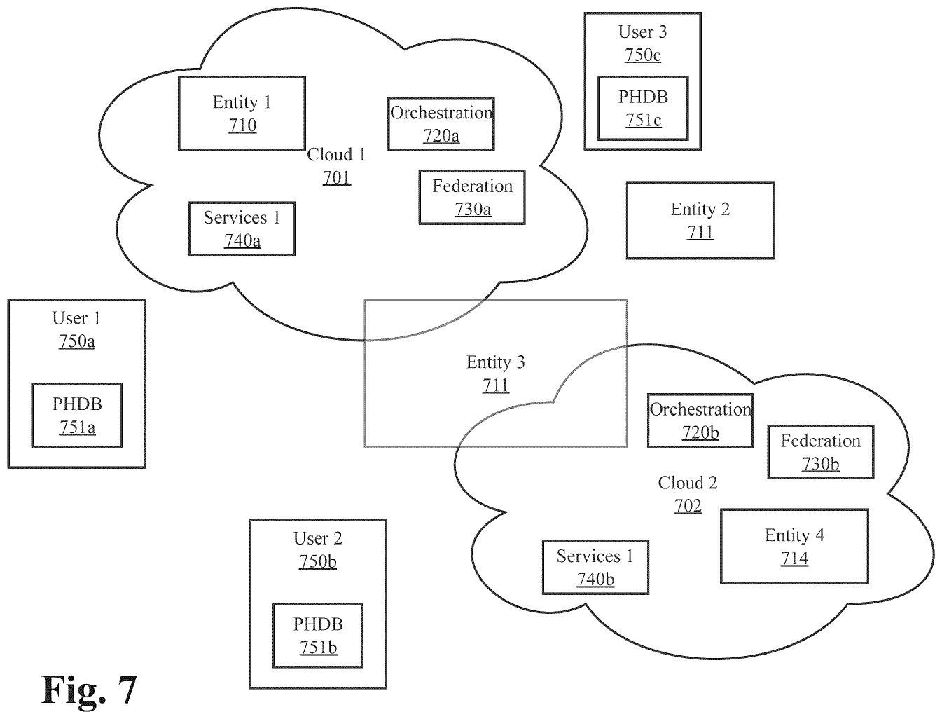

FIG. 7 is a system diagram showing an exemplary arrangement of a federated PHDB architecture, according to an aspect of the invention. A federated cloud architecture is a form of computing where multiple cloud computing environments are connected and integrated to work together as a unified, distributed system. In this federated cloud model, various cloud service providers (CSPs) 701, 702 collaborate, establishing a seamless and interoperable infrastructure for users. Federated cloud computing enables interoperability between different cloud providers or regions, allowing users to seamlessly access and use resources across multiple platforms.

According to the embodiment, each CSP may comprise entity 710, 714, an orchestration component 720a-b, a federation component 730a-b, and various services 740a-b. Orchestration components provide functionality directed to the execution of various tasks using one or more services provided by the CSP or other services provided by a different CSP. A CSP may comprise a group (e.g., federation 730a-b) or a collection of autonomous entities (such as organizations, departments, or systems) that cooperate and collaborate to achieve a common goal. Each entity in the federation retains control over its own resources and operations, while participating in a large, coordinated system. For example,

According to the embodiment, CSPs may have entities that operate fully within the CSP such as entity 1 710 of CSP 701. There are also entities that can exist between and among various CSPs such as entity 3 712 which operates within both cloud-based service providers. Also, there may be entities that exist outside of a CSP such as entity 2 711 which may interact with cloud 1 701 and cloud 2 702. Furthermore, the federated architecture further comprises a plurality of users 750a-c which are operating a PHDB enabled mobile device with a PHDB 751a-c stored on the user's mobile device.

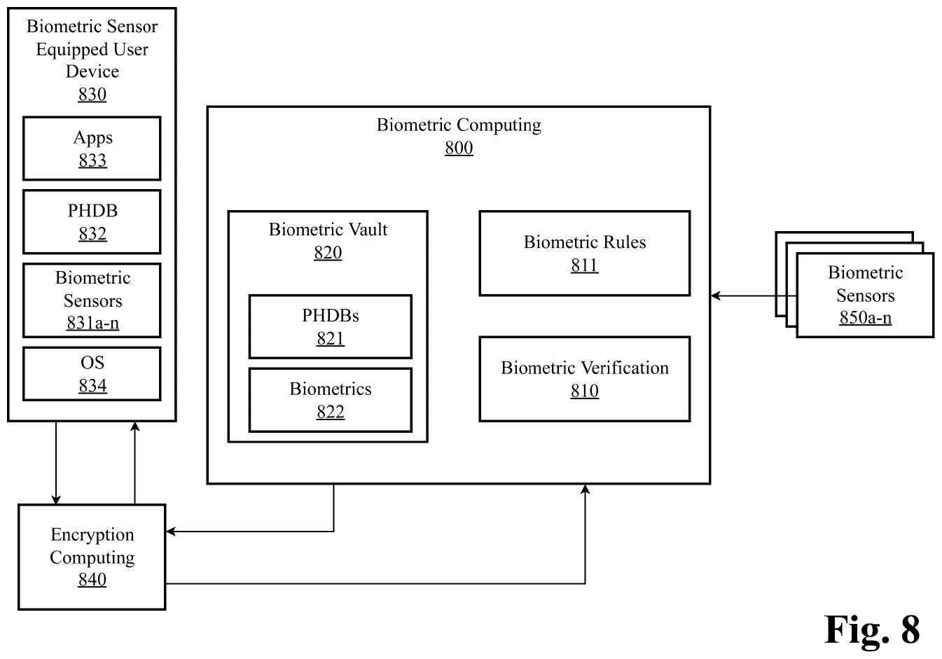

FIG. 8 illustrates a system diagram showcasing an exemplary configuration of biometric computing utilized for biometric sensors situated either on a user's endpoint device or on separate biometric sensor devices. Biometric computing 800 entails the utilization of biometric technology within computing systems. This technology involves the measurement and statistical analysis of distinctive physical and behavioral characteristics of individuals. A plurality of biometric sensors 850a-n collect the biometric data, which is subsequently computed, encrypted 840, and then shared with the biometric sensor equipped user device 830. The Biometric Sensor Equipped User Device 830 denotes a device incorporating biometric sensors 831a-n designed to capture and analyze physical or behavioral traits for functions such as fingerprint scanning, facial recognition, iris scanning, voice recognition, heart rate monitoring, and gesture recognition. Additionally, the biometric sensor equipped user device 830 includes applications (apps) 833, PHDB 832, and the operating system 834.

The data obtained from the biometric sensor equipped user device 830 is shared with encryption computing 840, which encrypts the obtained data to thwart unauthorized access. Encryption schemes such as AES or homomorphic encryption may be implemented to protect the obtained biometric data. Subsequently, the obtained data is sent to a biometric vault 820. The biometric vault securely stores biometric authentication methods for access control. The vault may store an instance of PHDB 821 which may only comprise encrypted biometric data such as biometric templates 822. It encompasses biometric rules 811 denoting privacy policies, guidelines, or principles pertaining to biometric technology use, and biometric verification 810, a process confirming identity by comparing unique physiological or behavioral characteristics against stored templates. The biometric vault 820 additionally includes the PHDBs 821 and biometrics 822. In addition to biometric sensors in the equipped user device 830, external biometric sensors 850a-n may be configured to capture and transmit biometric data to biometric computing 800. Biometric sensor data may be encrypted 840 and shared with the biometric sensor equipped user device 830 for storage in PHDB 832.

In an embodiment, biometric computing 800 may be used to provide biometric authentication of PHDB mobile device users. Biometric data gathered by user device 830 or biometric sensors 850a-n may be sent to biometric computing 800 where it may be processed and compared against stored biometrics 822 to authenticate a PHDB mobile device user. For example, if a user wishes to set new access permissions for third-party applications to access their PHDB, the system may prompt the user to provide biometric authentication to ensure that the user is the one managing access to their personal health information.

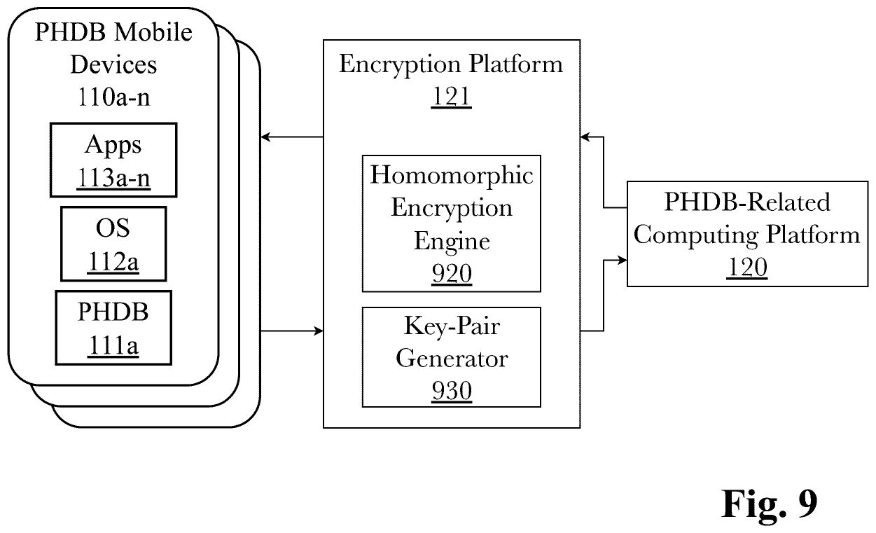

FIG. 9 is a system diagram showing an exemplary arrangement of an encryption platform for PHDB computing, according to an aspect of the invention. The PHDB-related computing platform 120 platform facilitates the sharing of personal health information (e.g., genomic data) with the encryption platform 121. Encryption platform possesses the capability to perform both regular and homomorphic encryption. The encrypted data is then securely transmitted to the requester on the PHDB mobile devices 110a-n, which consist of applications (apps) 113a-n, an operating system (OS) 112a, and PHDB 111a.

Homomorphic encryption engine (HEE) 920 functions as a cryptographic system providing homomorphic encryption of personal health information or other data requested from PHDB cloud services 101. The use of a homomorphic encryption engine enables computations that can be executed on encrypted data without requiring decryption. In contrast to regular encryption, which necessitates decryption before operations, homomorphic encryption allows computations directly on encrypted data, safeguarding its confidentiality. The homomorphic encryption engine 920 employs robust encryption algorithms to encrypt data. The encrypted data retains a format that enables specific computations without revealing the original information. In an implementation, HEE 920 may use partially homomorphic encryption, which is a type of homomorphic encryption that allows for computations on either the ciphertext or the plaintext, but not both. Examples include the RSA encryption method and the EIGamal method. In an implementation, HEE 920 may use a fully homomorphic encryption, which allows for arbitrary computations to be performed on encrypted data, including both additions and multiplications. Examples of fully homomorphic encryption include the Gentry-Halevi-Smart (GHS) scheme and the Brakerski-Gentry-Vaikuntanathan (BGV) scheme.

The key pair generator 930 component generates a public-private key pair crucial for the asymmetric encryption process. The public key is openly shared and is employed for encrypting data. The private key is kept confidential and is used for decrypting data and certain operations. Some common asymmetric encryption algorithms that may be implemented can include RSA, ECC, and Diffie-Hellman key exchange. The integration of key pair generation with homomorphic encryption enhances the platform's capabilities for privacy-preserving data analytics, secure cloud computing, and collaborative processing of confidential information.

In some embodiments, key pair generator 930 may utilize symmetric encryption techniques. In symmetric encryption, the same key is used for both encryption and decryption. This means that both the sender and the receiver need to know the same key. Symmetric encryption is typically faster and more efficient than asymmetric encryption, but it requires a secure way to share the key between the sender and receiver. Common symmetric encryption algorithms that may be implemented can include, but are not limited to, AES, data encryption standard (DES), and triple DES.

This system ensures that sensitive personal health data (e.g., genomic data) can be securely processed and analyzed while maintaining the privacy of the information through the utilization of both regular and homomorphic encryption techniques.

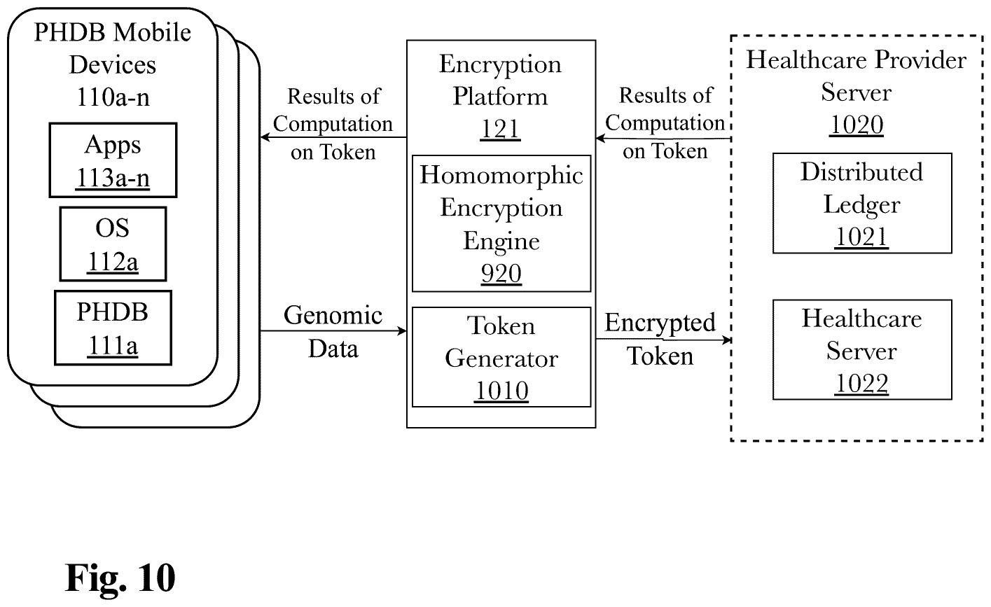

FIG. 10 is a system diagram showing an exemplary arrangement of a PHDB computing system using distributed ledger technologies, according to an aspect of the invention. The PHDB mobile devices 110a-n serves as the initiator by sharing genomic data through an encryption platform. The encryption platform 121 encompasses a homomorphic encryption engine 920 and a token generator 1010 powered by blockchain technology, enhancing traceability. The homomorphic encryption engine 920 facilitates secure computations on encrypted data without requiring decryption, preserving data confidentiality. The token generator 1010 employs blockchain technology to generate encrypted tokens, ensuring the traceability and secure data transfer. The encrypted token is then shared with the healthcare provider server 1020, comprising a healthcare server 1022 and a distributed ledger 1020. The healthcare server 1020 manages and processes the token encryption of data received from the encryption platform and the distributed ledger 1022 utilizes blockchain technology to maintain an immutable record of transactions and ensure transparency in data handling. The healthcare provider 1020 may process the encrypted genomic data or otherwise perform some computation on the genomic data. Examples of the types of processing that may be performed on genomic data by healthcare server 1022 can include, but are not limited to, whole genome sequencing, whole exome sequencing, genetic testing for specific conditions, pharmacogenomic testing, carrier screening, genomic tumor profiling, and general genomic counseling. Healthcare server 1022 can perform these types of computations and store the results on distributed ledger 1021. The results of the computations may be stored on a blockchain token.

Encryption platform 121 receives the tokenized computation results and can transmit them back to the requester on the PHDB Mobile devices 110a-n, completing the secure and traceable data exchange.

This system integrates homomorphic encryption, blockchain-powered token generation, and distribution ledger technologies to ensure secure, private, and transparent transactions in the context of personal health data (PHI) exchange. The use of encrypted tokens and distributed ledgers enhances traceability and accountability in the handling of sensitive genomic information.

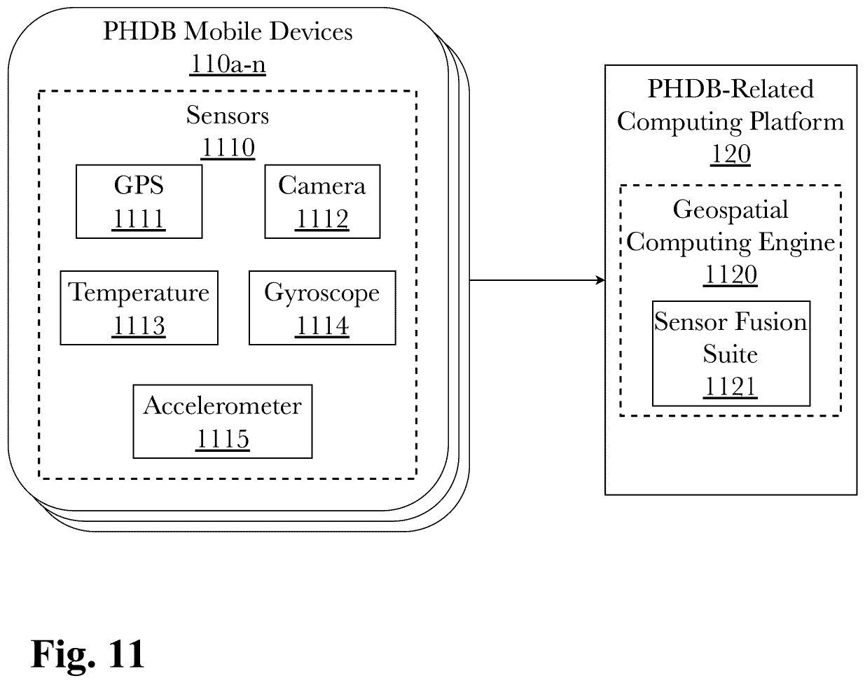

FIG. 11 is a system diagram showing an exemplary arrangement of a PHDB computing system using geospatial computing, according to an aspect of the invention. The PHDB mobile devices 110a-n is equipped with a set of sensors 1110, including, but not limited to, GPS 1111, Camera 1112, Temperature 1113, Gyroscope 1114, and Accelerometer 1115. The PHDB mobile devices share sensor data with the PHDB-related computing platform 120. Existing within the PHDB-related computing platform, the geospatial computing engine 1120 is responsible for processing geospatial data and leveraging a sensor fusion suite 1121. The geospatial computing engine 1120 receives position (e.g., location) updates from users, receives environmental events and obtains or generates local environmental data.

The geospatial computing engine 1120 utilizes a sensor fusion suite 1121 to integrate and analyze data from various sensors, including GPS 1111, Camera 1112, Temperature 1113, Gyroscope 1114, and Accelerometer 1115. According to an embodiment, sensor fusion suite 1121 may be implemented as a collection of algorithms and technologies used to integrate data from multiple sensors to provide a more accurate and comprehensive understanding of a system, user, or environment. Common sensor fusion algorithms include Kalman filters, particle filters, and Bayesian inference technique. Sensor fusion is particularly important in applications where multiple sensors are used to gather information about the same phenomenon such as when collecting information about a user's current environment. For example, a user's GPS coordinates may indicate that they are at a restaurant, but GPS cannot indicate if the user is dining inside or is seated outside on a balcony. Sensor fusion suite 1121 may be able to use ambient temperature sensor readings collected from the user's mobile device 110a to determine that the user is dining outside. As a result, the PHDB system may identify potential romantic partners who are also dining outside or that may be passing by on the street nearby the restaurant.

This system enables the integration of geospatial computing into the PHDB computing environment, leveraging sensor data from PHDB mobile devices equipped with GPS 1111, Camera 1112, Temperature 1113, and Gyroscope 1114. The Geospatial Computing Engine 1120, with its sensor fusion suite 1121, processes and analyzes the data to enhance the capabilities of the PHDB computing system 120.

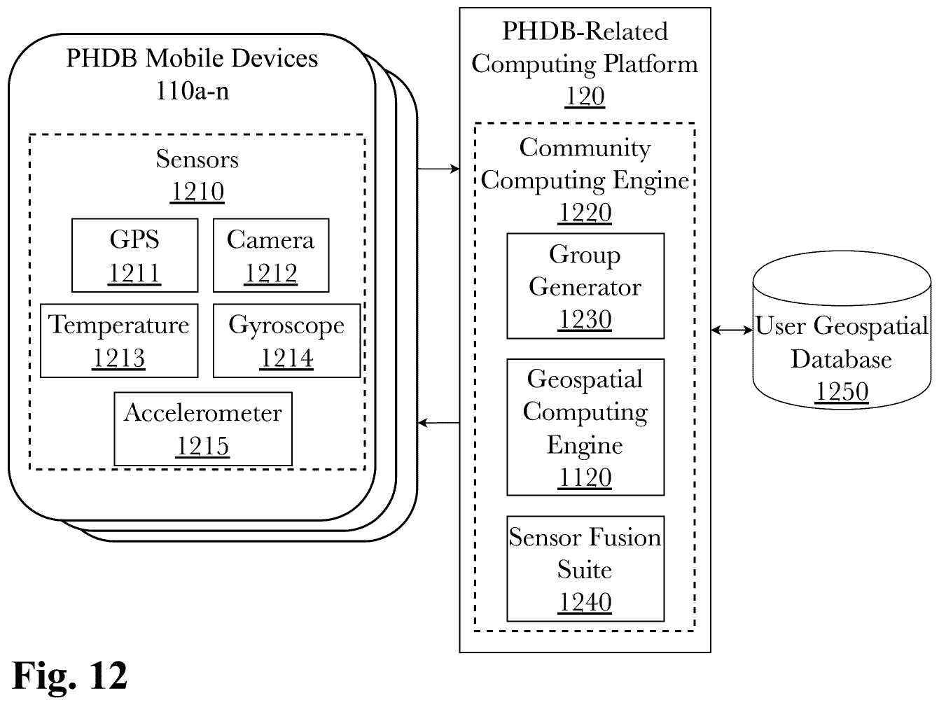

FIG. 12 is a system diagram showing an exemplary arrangement of a PHDB computing system using geospatial and community-based computing techniques and user grouping techniques, according to an aspect of the invention. User geospatial database 1250 actively receives and stores real-time geospatial data from subscribed users. This stored data may be retrieved by PHDB-related computing platform 120 to facilitate various community computing processes. These users utilize PHDB mobile devices 110a-n equipped with various sensors, such as GPS 1211, Camera 1212, Temperature 1213, Gyroscope 1214, and Accelerometer 1215. The data collected by these sensors is then transmitted to the PHDB-related computing platform 120, housing the community computing engine 1220.

According to the embodiment, community computing engine 1220 may comprise both a group generator 1230 and a geospatial computing engine 1120. The community computing engine 1220 facilitates the dynamic creation of ad hoc local groups, enables the establishment of persistent groups by users or third parties, and performs oracle detection. The computed data resulting from these operations is subsequently stored with user geospatial database 1250.

Specifically, group generator 1230 within the community computing engine 1220 is responsible for creating ad hoc local groups, allowing users to collaborate seamlessly based on real-time contextual factors. For example, groups may be created wherein members of the group are selected based on subsets of information stored in their individual PHDBs. For instance, groups may be based on shared common activities. Groups may be formed based on shared genetic information. Groups may be formed based on a score associated with human compatibility based on filtering of human genomes. Groups may be formed based on virtual environments or actual environments. Additionally, it provides functionality for users or third parties to establish and maintain persistent groups, fostering ongoing collaboration and data sharing.

Simultaneously, the geospatial computing engine 1120, also part of the community computing engine, processes the geospatial data received from the users. This engine performs intricate calculations and analyses to derive valuable insights, contributing to enhanced situational awareness and decision-making capabilities.

The overall system also incorporates an oracle detection mechanism within the community computing engine 1120. This feature enhances the system's ability to identify and respond to critical events or anomalies based on the processed data.

The PHDB-related computing platform 120 acts as a centralized hub where the data from various users and computing engines is consolidated and processed. Subsequently, the refined and computed data is stored in user geospatial database 1250. The PHDB computing system effectively utilizes geospatial and community-based computing techniques, coupled with advanced user grouping capabilities, to enhance real-time collaboration, data sharing, and decision-making within a dynamic and interconnected environment.

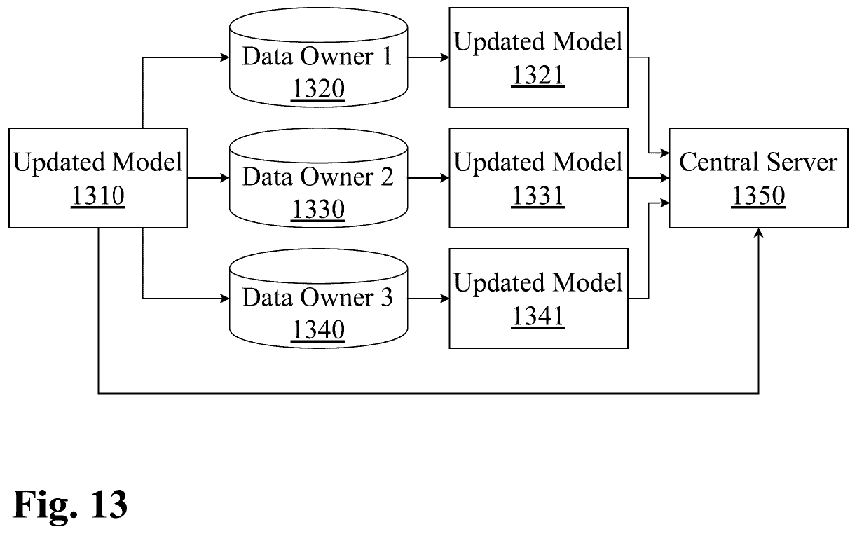

FIG. 13 is a system diagram showing an exemplary arrangement of federated computing, according to an aspect of the invention. The updated model 1310 is designed to share data seamlessly within a federated environment, demonstrating the flexibility of data federation. In this configuration, the updated model 1310 can directly communicate with the central server 1350, employing data federation protocols for efficient data exchange.

According to the embodiment, a plurality of data owners 1320, 1330, and 1340 are present and each in possession of some data. For instance, each data owner may represent PHDB user with a PHDB-enabled mobile device. The different data owned by the different people may be related, but different. For example, each data owner may store a plurality of personal health information. On each federated device there is a model 1321, 1331, and 1341 that can be stored and operated on the device. These models may function by processing local data. Periodically, each federated device may send updated models or model parameters to a central server 1350. The central server can perform model update tasks using the received models or model parameters from each of the federated devices. For example, a central server could aggregate the received model parameters to create a single set of model parameters with which to update a shared model. This updated model 1310 may be distributed to each of the federated device data owners, where it may be stored and operated.

Alternatively, the updated model 1310 can utilize data owner 1 1320, data owner 2 1330, and data owner 3 1340 as federation proxies. These proxies serve as intermediaries or agents, facilitating the exchange, retrieval, or processing of data between federated systems or networks. Their role extends to managing and securing data transactions within the federated environment.

In this federated setup, data owner 1 1320 acts as a proxy for data exchange with updated model 1321, data owner 2 1330 with updated model 1331, and data owner 3 1340 with updated model 1341. Each data owner serves as a localized point of contact, streamlining the data flow and ensuring efficient communication between the Updated Models and the corresponding Data Owners.

Subsequently, the data shared between each data owner and their respective updated models can be aggregated and transmitted to the central server 1350. This approach allows for a distributed and collaborative data-sharing model within the federated computing system.

The use of federation proxies not only enhances the efficiency of data transactions but also contributes to the overall security and management of the federated environment. By incorporating these intermediaries, the system ensures that data is exchanged seamlessly and securely across the federated network, promoting enhanced interoperability and collaboration.

This type or arrangement may also be used to represent edge computing wherein mobile devices representing data owners may train and maintain models on the device which process and analyze stored data. Periodically, these edge devices can submit their models to central server 1350 where they may be used to form a single updated model which can then be deployed back to the edge devices for further training and operation.

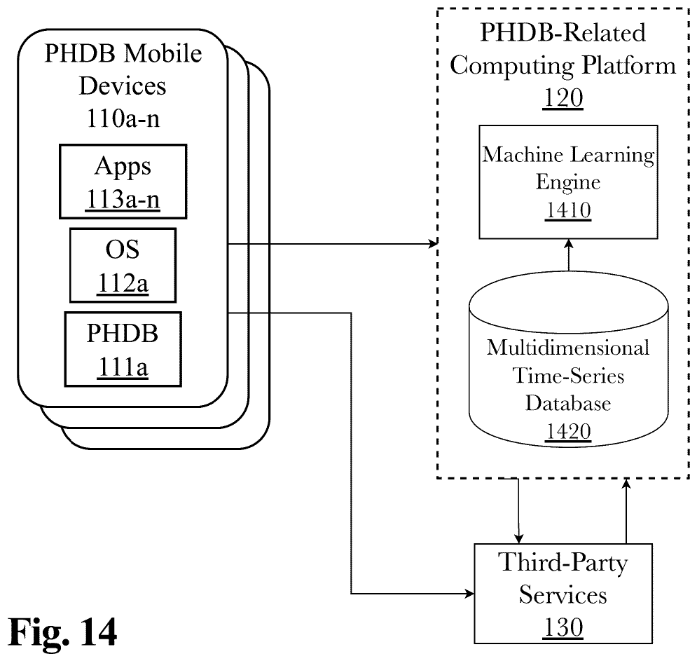

FIG. 14 is a system diagram showing an exemplary arrangement of a service prediction mechanism using a multidimensional time-series database, according to an aspect of the invention. Users equipped with PHDB mobile devices 110a-n, comprising applications 113a-n, an operating system (OS) 112a, and the PHDB application 111a, have the capability to share data directly with the PHDB-related computing platform 120. This platform encompasses a Multidimensional Time-Series Database (MTSDB) 1420, serving as a pivotal component in the system's predictive capabilities. The MTSDB 1420 can provide a source for creating training, validation, and test datasets from the shared data to a machine learning engine 1410.

Alternatively, users may opt-in to share their data with third-party services 130, which then relay the data to the PHDB-related computing platform 120. The MTSDB 1420 receives events from various related services, continuously updating with real-time events and aggregate data. This dynamic database is instrumental in conducting simulations aimed at predicting service demand and generating optimization recommendations for services.

The predictive capabilities of the system are harnessed through simulations facilitated by the Multidimensional Time-Series Database 1420. By analyzing real-time data, the system can anticipate service demands, allowing for proactive decision-making and resource optimization. Furthermore, the generated optimization recommendations contribute to refining the performance of the associated services.

The Multidimensional Time-Series Database 1420 plays a central role in the generation of real-time screening candidates and world-scale simulations. It acts as a comprehensive repository of temporal data, providing a foundation for accurate predictions and informed decision-making within the various services. Temporal data may be ingested or otherwise obtained by PHDB-related computing platform 120. Temporal data related to a person's omics information can include changes in gene expression levels over time, variations in metabolite levels in response to diet or medication, fluctuations in epigenetic markers like DNA methylation patterns, and alterations in the microbiome composition. Tracking how the expression of specific genes changes over time can provide insights into disease progression or response to treatment. Monitoring the levels of metabolites in the body over time can reveal patterns related to metabolic health, response to diet, or drug metabolism. Studying changes in DNA methylation patterns over time can help understand how environmental factors influence gene expression and disease risk. Observing how the composition of the gut microbiome changes over time can offer insights into digestive health, immune function, and even mental health. Analyzing changes in protein expression and modification patterns over time can provide information about cellular processes and disease mechanisms.

According to the embodiment, machine learning engine 1410 may be configured to create, store, and maintain one or more predictive or analytical models developed using machine and/or deep learning techniques. The one or more predictive or analytical models may be directed to generating a score that represents the compatibility of two or more people based on analysis of declared preferences such as, for example, a preferred genomic profile or attribute. Preferences may be directed to, for example (and not limited to), genetic, emotional, religious, or behavioral kinds of preferences or constraints. For example, assume Person-A set in their profile that they want to screen out anyone with a high potential for cystic fibrosis. Person-A walks into a room (at some venue—concert hall, convention center, restaurant, etc.) and their device begins the screening process to score everyone (that opts-in) in the room. The device can show a score for each individual (that opts-in) which reflects the relative match between Person-A and the other individuals based on each person's individual criteria (e.g., preferences).

According to the embodiment, machine learning engine 1410 may train one or more machines and/or deep learning algorithms to create a model. The data used for model training purposes may comprise subsets of data stored in a plurality of PHDBs. The training data may further comprise PHDB-enabled device-specific data or metadata such as mobile device location information, a device IP address, and/or the like. Training data may further be sourced from MTSDB 1420 or third-party services 130 such as, for example, social media servers, medical services providers, data centers, and medical/behavioral/therapeutic/etc. labs. Training data may further be sourced from various IoT devices 140.

In summary, the disclosed system leverages a multidimensional time-series database as a core component in a service prediction mechanism. By integrating predictive analytics and machine learning, the system enhances its ability to forecast service demands, generate optimization recommendations, and facilitate real-time screening candidates and large-scale simulations.

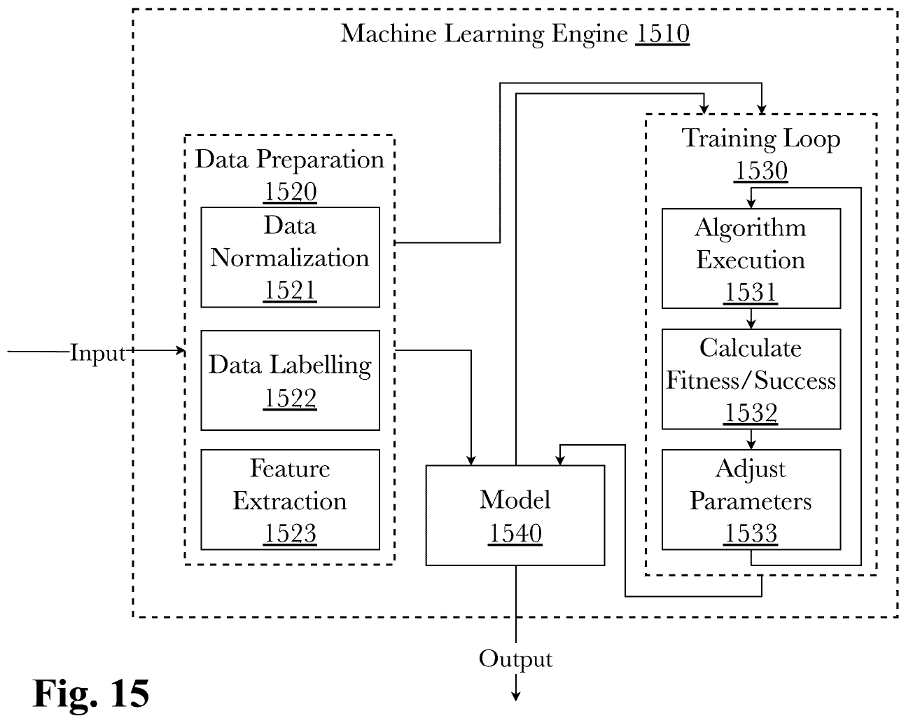

FIG. 15 is a system diagram illustrating an exemplary architecture of a machine learning engine. A machine learning engine 1510 may be a software component, standalone software library, system on a chip, application-specific integrated circuit (“ASIC”), or some other form of digital computing device or system capable of interacting with and receiving data from other digital or software systems. It may be connected over a network, or connected within a system or computer, and may be utilized by software processes or communicate with them as a separate application or process instance. The basic components within a machine learning engine, broadly, are a data preparation 1520 loop or algorithm, which may contain some combination of steps, commonly including data normalization 1521, data labeling 1522, and feature extraction 1523, depending on the exact implementation or configuration of a machine learning engine 1510. A key feature of a machine learning engine 1510, is the existence of some form of a training loop 1530 in their software or chip design, a series of steps taken to take input data and learn how to process it and produce some desired output. A machine learning engine 1510 may be configured or implemented poorly merely as a matter of execution, and may have trouble learning efficiently or at all, or have difficulty learning usefully from certain knowledge areas or domains, but all machine learning systems contain a training loop of some kind, and they frequently contain the subcomponents or steps of having algorithm execution perform over the set of input data 1531, calculating the fitness or success states or success rate of the algorithm with a current model 1540, and adjusting the parameters of the model to attempt to output better or more useful data for a given input data.

A model 1540 is a software or mathematical representation of data that impacts how an algorithm operates. An algorithm may be any set of concrete steps taken to attempt to process data or arrive at some solution to a problem, such as a basic search algorithm which tries to find a specified value in apparently unsorted numeric data. A basic attempt at such a search algorithm might be to simply jump around randomly in the dataset and look for the value being searched for. If machine learning were applied to such an algorithm, there might be a model of parameters for the algorithm to operate with, such as how far from the current index being examined in the input dataset, to be capable of jumping. For instance, in a set of 1,000 numbers in no readily apparent ordering or sorting scheme, the algorithm to randomly pick numbers until it finds the desired number may have a parameter that specifies that if you are currently at index x in the dataset being searched, you may only jump to a value between x−50 and x+50. This algorithm may then be executed 1531 over a training dataset, and have its fitness calculated 1532, in this example, as the number of computing cycles required to find the number in question. The lower the number, the higher the fitness score.

Using one of many possible parameter adjustment 1533 techniques, including linear regression, genetic variation or evolutionary programming, simulated annealing or other metaheuristic methods, gradient descent, or other mathematical methods for changing parameters in a function to try and approach desired values for specified inputs. Machine learning training method, that is, the way they adjust parameters 1533, may be deterministic or stochastic, as in evolutionary or genetic programming, or metaheuristics in general. Examples of genetic programming include the concept of genetic variation, whereby several different models of an algorithm are run over the same input data, compared for fitness, and a selection function determines which models to use for “breeding” the next “generation” of the model population, at which point a crossover function is used to recombine the “genes” (the word used in genetic programming to refer to function or model parameters) into different arrangements for each new member of the next generation, lastly applying a mutation function to alter (either randomly or statistically) some selection of genes from some selection of the newly bred models, before the process is repeated with the hope of finding some combinations of parameters or “genes” that are better than others and produce successively better generations of models.

Several machine learning methodologies may be combined, as with NeuroEvolution of Augmenting Topologies (“NEAT”), whereby a genetic algorithm is used to breed and recombine various arrangements of neurons and hidden layers and the parameters of neurons, in a neural network, reducing the use of human judgement in the design or topology of a neural network (which otherwise often requires a fair amount of trial and error and human judgement). These situations may be thought of either as multiple different training loops 1530 occurring with multiple models 1540, or may be thought of as multiple machine learning engines 1510 entirely, operating together.

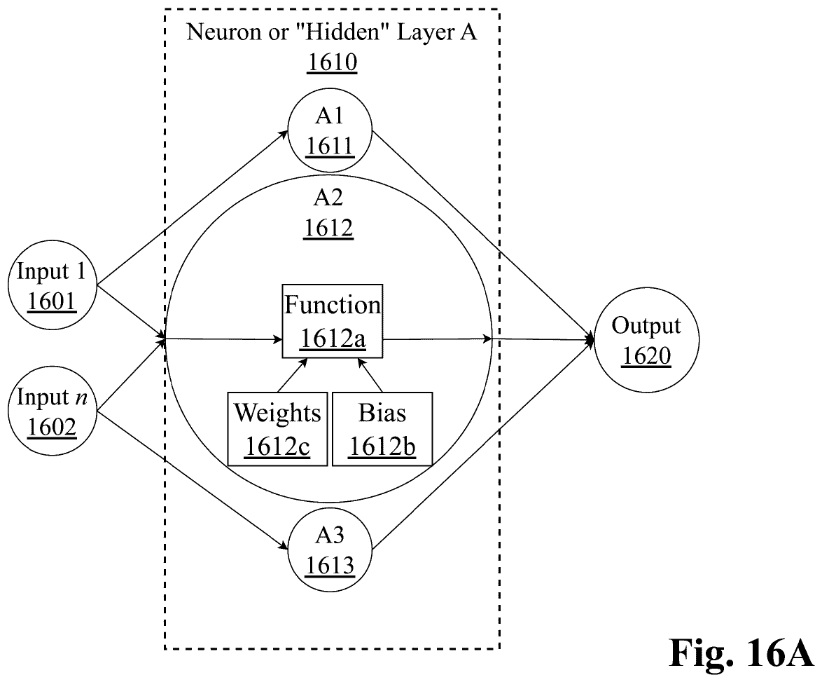

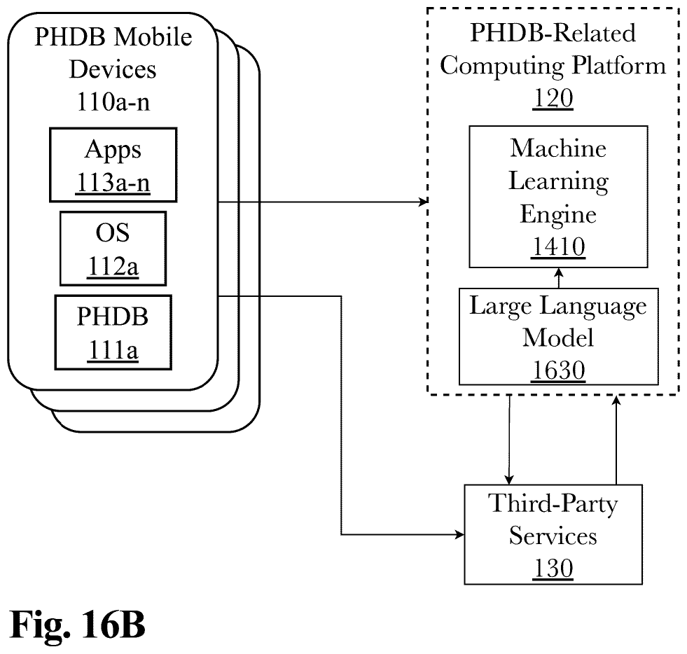

FIG. 16A is a diagram illustrating an exemplary architecture of a neural network. A neural network is a software system that may be used to attempt to learn or improve an algorithm at a task or set of tasks, using mathematical models and approximations of biological neurons with artificial neurons. The kinds of tasks that may be used in combination with a neural network are potentially unlimited so long as the problem is deterministic, but common applications include classification problems, labeling problems, compression or algorithm parameter tuning problems, image or audio recognition, and natural language processing. Neural networks may be used as part of a machine learning engine, as the method by which training is done and a model is generated. A neural network contains at least one input, here labeled as input 1 1601, but may have multiple inputs, labeled input n 1602, that feed into a neuron layer or hidden layer 1610 which contains at least one artificial neuron, here shown with A1 1611, A2 1612, and A3 1613. Inside of each neuron are three components, an activation function 1612a, a bias 1612b value, and a weight for each input that feeds into the neuron 1612c. An activation function 1612a is the function that determines the output of the neuron, and frequently follows a sigmoidal distribution or pattern, but may be any mathematical function, including piecewise functions, identity, binary step, and many others. The activation function 1612a is influenced not only by the inputs into a neuron 1601, 1602, but the weight assigned to each input 1612c, which multiplies an input value by itself, and a bias 1612b, which is a flat value added to the input of the activation function 1612a. For instance, with a single input value of 17, a weight of 0.3, and a bias of 0.5, a neuron would run its activation function with an input of 5.6 (17*0.3+0.5). The actual output of the activation function 1612a, for each neuron, then may proceed to be output 1620 in some format, usually numeric, before being interpreted by the system utilizing the neural network. There may be multiple output values, representing confidence values in different predictions or classifications, or other multi-valued results.

Various forms and variations of neural networks exist which may be more or less applicable to certain knowledge domains or certain problem sets, including image recognition, data compression, or weather prediction. Some examples of different types of neural networks include recurrent neural networks (including variants thereof such as Long Short Term Memory recurrent neural networks), convolutional neural networks, deep learning networks, and feed forward neural networks, the last of which is regarded by many as the “standard” or most basic usable form of an artificial neural network.

FIG. 16B is a system diagram showing an exemplary arrangement of a PHDB system utilizing Large Language Model (LLM) machine learning technology, according to an aspect of the invention. Users equipped with PHDB mobile devices 110a-n, comprising applications 113a-n, an operating system (OS) 112a, and the PHDB application 111a, possess the capability to share data directly with the PHDB-related computing platform 120.

It should be appreciated that the illustrated LLM 1630 is merely an exemplary machine learning model that may be utilized according to various aspects, and that other AI/ML systems may be used in various embodiments. In some aspects, a neuro-symbolic AI system may be implemented which blends LLMs or other Connectivist models with symbolic models. Such a neuro-symbolic model may be used to provide AI planning solutions and simulation to enable a much more complex reasoning/planning and operations/optimization with awareness of resources. A neuro-symbolic model that incorporates Connectivist principles combines elements of neural networks and symbolic reasoning to create a hybrid approach to AI. According to an aspect, a neural network component processes raw data, such as images, text, or sensor inputs, to extract relevant features and learn patterns. This component is responsible for tasks like image recognition, natural language processing, or sensor data analysis. The symbolic reasoning component manipulates abstract symbols and rules to perform tasks that require logical reasoning or explicit knowledge representation. This component is responsible for tasks like logical inference, planning, or knowledge representation. Connectivism emphasizes the importance of connections and networks in learning. In a neuro-symbolic model, Connectivist principles might be used to guide how the neural and symbolic components interact and learn from each other. This could involve feedback loops where symbolic reasoning helps guide the learning of the neural network, and the neural network's outputs inform symbolic reasoning. The integration of neural and symbolic components can happen at various levels. For example, the output of a neural network might be fed as input to a symbolic reasoning system, which then uses logical rules to make decisions or generate new knowledge. Conversely, the output of symbolic reasoning might be used to guide the training or behavior of the neural network.

According to the embodiment, platform 120 incorporates an LLM 1630, which may be trained and maintained by machine learning engine 1410. The user can transfer data directly to the platform, or alternatively, choose to share data with a third-party service 130, which then facilitates the relay of data to the LLM machine learning technology on the PHDB-Related Computing Platform 120. According to an aspect, a user of mobile device 110a may be able to submit a prompt via PHDB app 111a to LLM 1630 which can process the prompt and provide a response to the user of mobile device 110a. In some implementations, an instance of LLM 1630 may be deployed locally on a mobile device 110a. In such implementations, platform 120 may leverage federated learning with edge computing via PHDB mobile devices 110a-n.

The shared data plays a pivotal role in establishing a baseline for the LLM 1630. This model, equipped with machine learning capabilities, is not only capable of receiving requests (e.g., prompts) from other computing services but also adept at generating appropriate responses to fulfill the requirements of these services. In some implementations, multiple LLMs may be developed, wherein each of the multiple LLMs may be configured to specific domain or preference. For example, a first LLM may be trained to be a resource for use cases involving genomic assistance, and a second LLM is trained to be a resource for use cases involving microbiome-based assistance. In other implementations, an LLM may be personalized to an individual user. In such implementations, the user may opt-in to allow the LLM access to all their stored personal health data 200 thereby allowing the LLM to better understand and anticipate user queries and intentions.

LLM 1630 continuously updates based on PHDB-centric user interactions. This dynamic learning process ensures that the LLM remains adaptive and responsive to evolving user needs and preferences.

In summary, the disclosed PHDB system, incorporating Large Language Model (LLM) machine learning technology, empowers users to share data directly with the PHDB-Related Computing Platform. The integration of LLM enhances the system's ability to comprehend and generate responses to user queries, creating a sophisticated and adaptive environment for users.

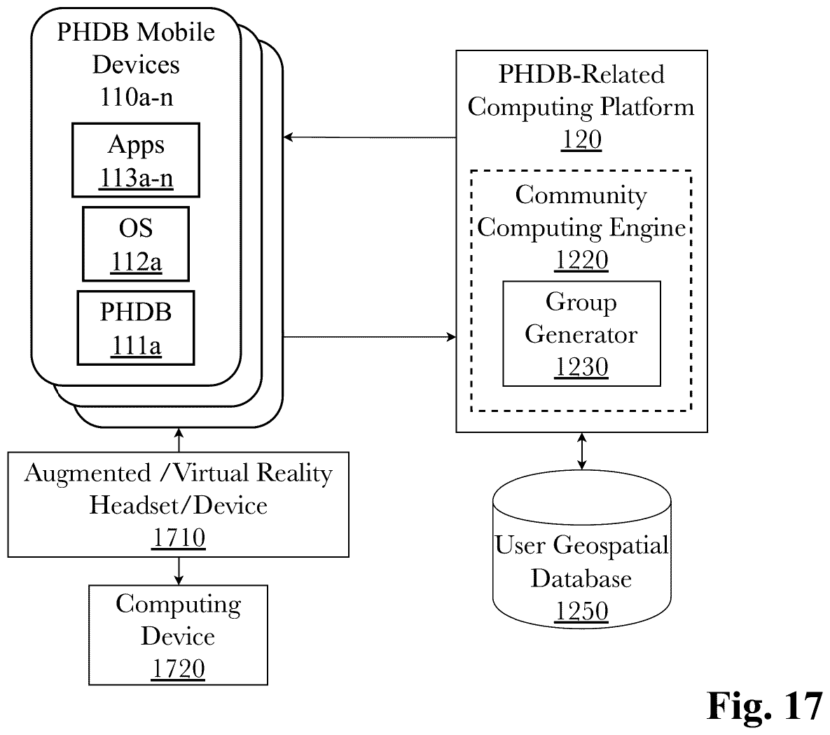

FIG. 17 is a system diagram showing an exemplary arrangement of a PHDB computing system utilizing Augmented Reality (AR) or Virtual Reality (VR) technologies, according to an aspect of the invention. Augmented reality or virtual reality headsets or devices 1710 are employed to share data with both a computing device 1720 and PHDB Mobile Devices 110a-n.

PHDB Mobile Devices 110a-n, comprising applications (Apps) 113a-n, an operating system (OS) 112a, and the PHDB application 111a, receive data shared by the AR or VR Headset or device. In some arrangements, PHDB mobile devices 110a-n and/or computing device 1720 may be configured to send control signals to AR/VR headset 1710 for controlling or otherwise manipulating the AR/VR environment. Subsequently, the PHDB mobile devices can transmit this data to the PHDB-related computing platform 120.

The PHDB-Related Computing Platform 120 encompasses a community computing engine 1220, which in turn includes a group generator 1230. This engine is pivotal in creating ad hoc local groups through its group generator and facilitates seamless data sharing within the community. The PHDB-related computing platform 120 then disseminates the shared data to the user geospatial database 1250.

In an exemplary use case, the data stored in PHDBs associated with AR/VR headset/device users may be used to identify playing groups for a virtual reality game. Group generator 1230 could obtain various user geospatial data to identify AR/VR users in a given area nearby a first player to initially identify a group to play with. Then, PHDB data and preferences could be obtained from the identified players to refine the group list based on matched preferences and statistical analysis thereof. The refined group may be presented to the AR/VR users such as by an invitation to join a playing group, or an invitation to meet at a physical location somewhere nearby (as determined by user defined preferences for distances willing to travel on foot, etc.) to participate in a group AR/VR session.

The utilization of AR or VR technologies enhances the user experience by providing an immersive interface for data sharing. This integration of spatial computing enriches user interactions and contributes to the creation of dynamic local groups, fostering collaborative and real-time data exchange within the PHDB ecosystem.

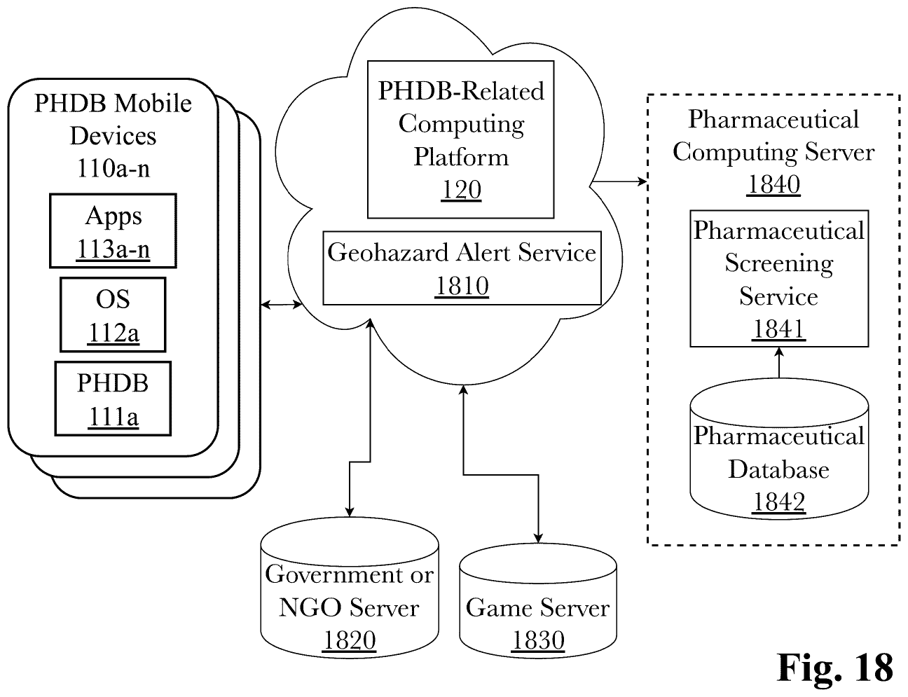

FIG. 18 is a system diagram showing an exemplary arrangement of a PHDB computing system utilizing a variety of possible third-party services and servers, according to an aspect of the invention. The PHDB mobile devices 110a-n may include Apps 113a-n, an operating system (OS) 112a, and the PHDB application 111a, and can engage in data sharing with the cloud infrastructure. This cloud includes the PHDB-related computing platform 120 and a geohazard alert service 1810.

The data shared within the cloud is then further transmitted to a variety of third-party services, enhancing the functionality and utility of the PHDB system. Notable third-party services include government or non-governmental organization (NGO) server 1820, a game server 1830, and a pharmaceutical computing server 1840.

Within the pharmaceutical computing server 1840, a pharmaceutical database 1842 is housed, facilitating the transfer of data to the pharmaceutical screening service 1841. This specialized service is designed to analyze and screen health-related data, contributing to the overall capabilities of the PHDB system in relation to pharmaceutical information.

Geohazard alert service 1810 is a service that provides timely information and warnings about geological hazards, such as earthquakes, landslides, volcanic eruptions, and tsunamis. These services use data from various sources, including seismic sensors, GPS stations, satellite imagery, game servers, governmental and NGOs, and geological surveys, to detect and monitor potential hazards and to issue alerts to the public and relevant authorities.

Government or NGO servers 1820 can host information or send out warnings about a geohazard (e.g., war, national security advisory, natural disaster, etc.) and the PHDB computing platform 120 scans known government/NGO servers for hosted information, and/or receives alerts. Users that are known to be in those locations where a geohazard has occurred, may be proactively alerted if the users have appropriate security rules and encryption practices that allow for it.

During geologically hazardous events where people are injured or need treatment, PHDB mobile devices can alert platform 120 of the types of treatment or medicine that people in the crisis need. This information can be communicated to the appropriate governmental organizations and NGOs to begin the process of aid procurement and may be further communicated to nearby pharmaceutical computing servers 1840 to begin processing the shipment of the required medications or other medical supplies.

This integration of third-party services extends the reach and applicability of the PHDB system, providing users with diverse functionalities ranging from government-related services, gaming applications, to pharmaceutical screenings.

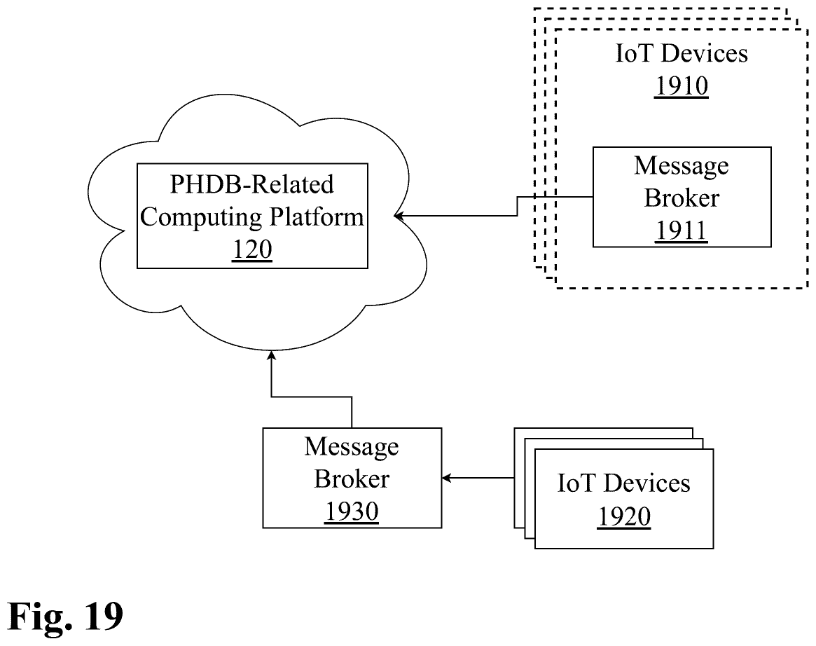

FIG. 19 is a system diagram showing an exemplary arrangement of a PHDB computing system utilizing Internet-of-Things (IoT) devices and associated technology, according to an aspect of the invention. IoT devices 1910, equipped with various sensors and technologies, form an integral part of the system architecture.

These IoT devices 1910 communicate with an embedded message broker 1911, serving as a central communication hub. The message broker facilitates the efficient and secure exchange of data between the IoT Devices 1910 and the PHDB-Related Computing Platform 120. This bi-directional data sharing ensures a seamless flow of information between the physical environment monitored by the IoT Devices and the computational capabilities of the PHDB system.

Furthermore, the message broker 1930 plays a critical role in managing distributed communication flow. It acts as an intermediary, collecting data from the IoT Devices 1920 and transmitting it to the cloud based PHDB-Related Computing Platform 120. This approach enhances the scalability and flexibility of the system, allowing for the integration of diverse IoT Devices with different communication protocols and data formats.

The data shared by the IoT Devices encompasses various health-related parameters, contributing to the comprehensive genomic health monitoring capabilities of the PHDB system. This integration of IoT technology enhances real-time data acquisition and expands the scope of genomic health-related information that can be incorporated into the PHDB.

### Detailed Description of Exemplary Aspects

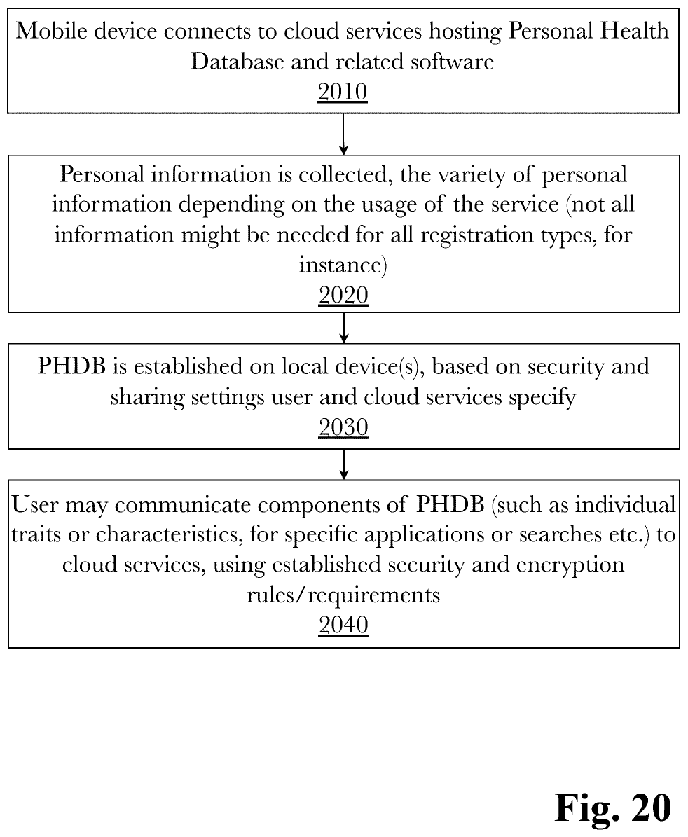

FIG. 20 is a method diagram showing exemplary steps taken for a user to register and establish a PHDB, according to an aspect of the invention. According to the aspect, the process begins at step 2010 when the user's mobile device establishes connection to cloud services 101 hosting the Personal Health Database and related software. This initial connection may be made responsive to the user downloading and storing a PHDB software application 111a on their PHDB-enabled mobile device 110a and opening the PHDB application for the first time as part of the onboarding process. Additionally, or alternatively, the user may establish the initial connection by accessing a PHDB associated website or web application on their computing device (e.g., personal computer or laptop). At a next step 2020 personal information is then collected, with the specific type and extent of information gathered depending on the intended usage of the service 2020. It's noteworthy that not all information may be required for every registration type, ensuring flexibility and user customization. Examples of the types of personal information that can be collected can include, but is in no way limited to, genomic data, microbiome data, phenotype data, biometric data, activity data, preference data including likes and dislikes, medical data, demographic data, physiological data, behavioral data, social media data, education data (e.g., highest level of education achieved), and/or the like. The personal information may be collected from a plurality of sources. For example, PHDB users can directly submit their personal information via PHDB application 111a stored and operating on their mobile device. Users may be guided through a process that can query the user for various types of personal information that the user may optionally choose to submit for storage in their PHDB. Mechanisms such as surveys or questionnaires can be used as a means to collect personal information. Additionally, or alternatively, PHDB users can select (i.e., provide consent to) various applications and/or third-party services for providing user-selected personal information that the applications and/or third-party services are in possession of. For example, a user can provide consent for a subset of their social media data to be uploaded to their PHDB. As another example, a user can consent to their medical provider to provide their electronic health record, or a subset thereof, for inclusion into their PHDB.

PHDB is established on local device(s), based on security and sharing settings specified by both the user and the cloud services 2030. This step ensures that the user has control over the security parameters and sharing preferences of their personal health data. The PHDB may be stored as an encrypted database on the user's local device.

To provide various functionality, a user may communicate components of PHDB (such as individual traits or characteristics, for specific applications or searches, etc.) to cloud services, using established security and encryption rules/requirements 2040. For example, components of PHDB may be encrypted (e.g., AES, homomorphic encryption, some combination thereof, etc.). Rules such as two-factor authentication and various access rules may be implemented to verify users who attempt to share personal information and to ensure that only user-selected individuals are able to receive the personal information.

Throughout these steps, the user is empowered to customize their PHDB based on their unique needs and preferences. The secure communication protocols and encryption mechanisms guarantee the confidentiality and integrity of the shared data, fostering a trustful and privacy aware environment for the user.

The disclosed method for registering and establishing a PHDB offers a user-centric approach, allowing individuals to tailor their health database while ensuring robust security measures during data transmission to and from cloud services.

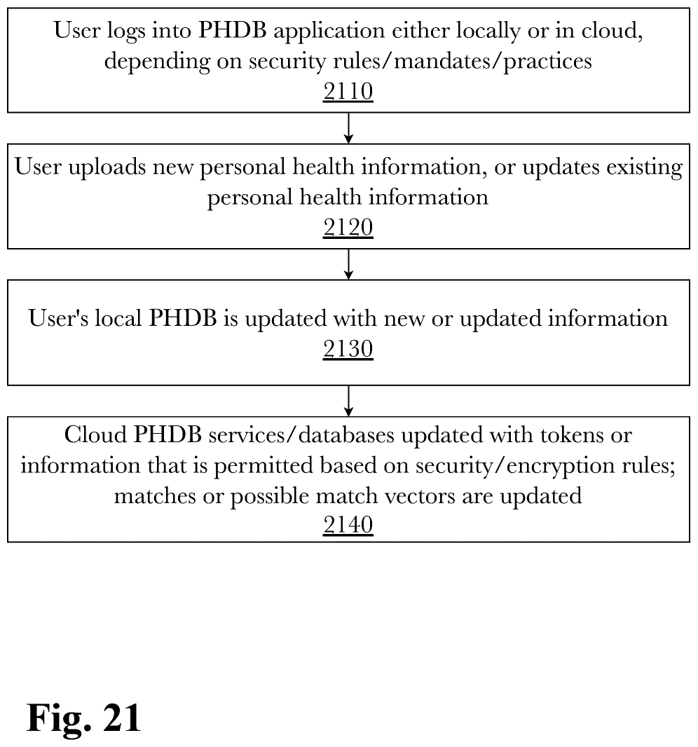

FIG. 21 is a method diagram showing exemplary steps taken for a user to update their data or information in a PHDB, according to an aspect of the invention. According to the aspect, the process begins at step 2110 as the user logs into PHDB application, either locally or in the cloud, based on security rules, mandates, or practices. For example, a user may first have to verify their identity before being allowed to edit or otherwise update their personal information such as by using biometric data to provide two-factor authentication. As another example, a user may establish rules that allow updates to their PHDB to be performed only when a user is logged into a specific device, or location, or certain time of the day. These are merely examples of the types of rules/restrictions that a user can optionally select to establish a level of PHDB security they are comfortable with.

The user then uploads new personal health information, or updates existing personal health information within the PHDB application 2120. The user's local PHDB is subsequently updated with the new or modified information 2130. This local update ensures that the user's device is current and reflective of the most recent health-related data. To maintain consistency across the PHDB ecosystem, the Cloud PHDB services and databases are updated with tokens (e.g., distributed ledger token) or information that is permitted based on security/encryption rules 2140. This ensures that the central repository of health data, accessible through cloud services, is synchronized with the user's updated information while adhering to established security protocols. The inclusion of this updated personal information may affect the types of matches or possible matches that have been determined for the user who updated their information. As such, the cloud based PHDB services may update match or possible match vectors to reflect the updated information.

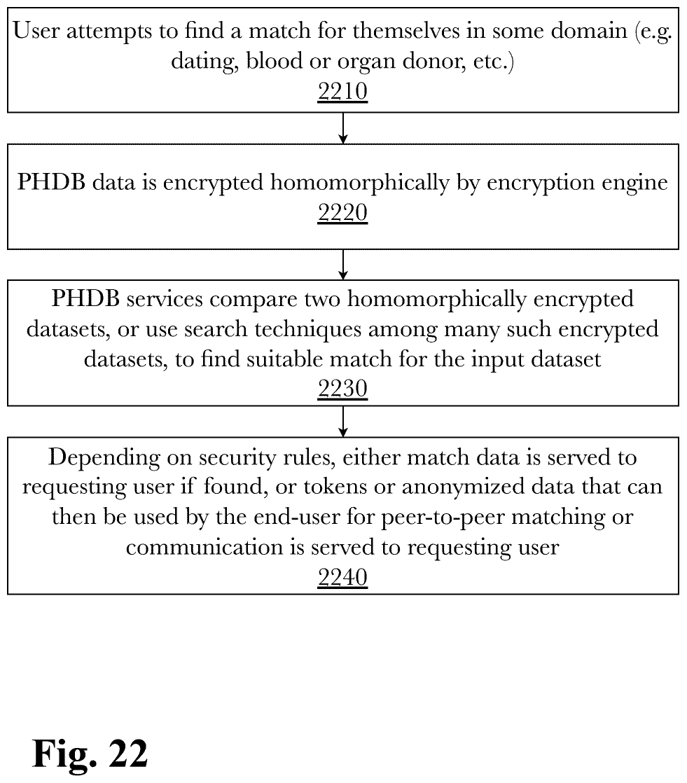

FIG. 22 is a method diagram showing exemplary steps taken for identifying a match using homomorphic encryption with a PHDB, according to an aspect of the invention. According to the aspect, the process begins at step 2210 as the user attempts to find a match for themselves in a specific domain, such as dating, blood or organ donor, gaming partner, etc. The PHDB data is then subjected to homomorphic encryption by an encryption engine at step 2220. In an embodiment, the homomorphic encryption scheme is partially homomorphic encryption. In an embodiment, the homomorphic encryption scheme is fully homomorphic encryption. This encryption process ensures that sensitive health-related information remains confidential while still allowing for meaningful comparisons and searches. At a next step 2230 PHDB services compare two homomorphically encrypted datasets, or use search techniques among multiple encrypted datasets, to identify a suitable match for the input dataset. For example, encrypted datasets may undergo a simple equality comparison, a similarity comparison (e.g., measuring the similarity between two encrypted vectors), or another type of comparison depending on the application, domain of interest, or some other limiting factor. This approach enables secure and privacy-preserving matching processes, safeguarding the integrity of the users' health data. In some embodiments, user PHDB profiles and preferences may be analyzed by a trained model and a score produced which is indicative of a potential match or a potential non-match.

Depending on security rules, if a match is found, match data is served to the requesting user. In some embodiments, the match data that is served to the requesting user may first be decrypted, if the involved users have allowed such sharing of personal information. Alternatively, tokens or anonymized data may be provided, allowing the end-user to engage in peer-to-peer matching or communication without revealing sensitive information directly 2240. This ensures that privacy is maintained during the matching process, and users have control over the level of detail shared with potential matches.

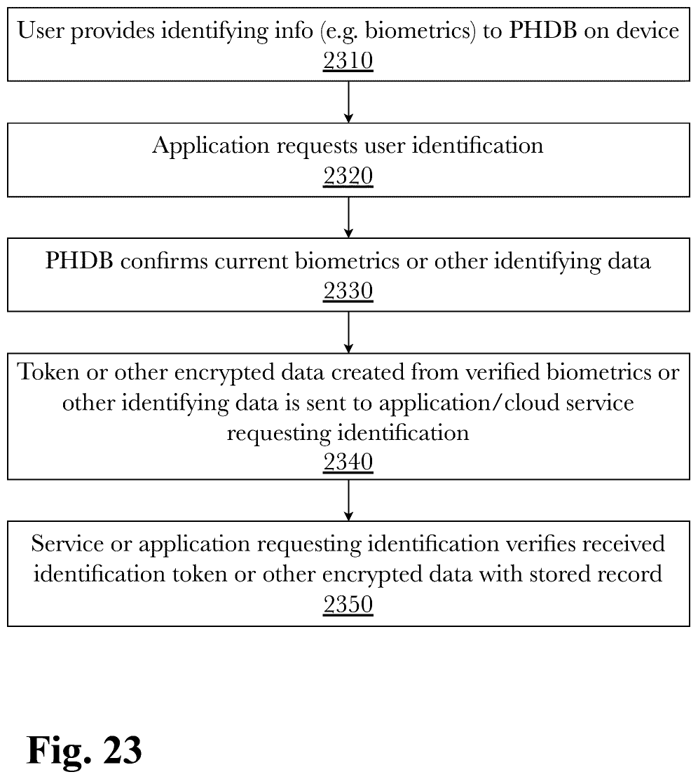

FIG. 23 is a method diagram showing exemplary steps taken for a user to authenticate themselves for access to cloud-based services using a PHDB, according to an aspect of the invention. According to the aspect, the authentication process begins at step 2310 when the user provides identifying info (e.g. biometrics) to PHDB on device 2310. The provided biometric data may be obtained from sensors/systems embedded in a PHDB-enabled mobile device 110a-n or from stand-alone biometric devices which can collect and transmit biometric data and may be integrated with a mobile device 110a-n or other computing device 115a-n.

When an external application requests user identification 2320, the PHDB is prompted to confirm the current biometrics or other identifying data associated with the user 2330. In an embodiment, biometric computing 800 may be leveraged to verify a user's identity using biometric data as a form of authentication. Biometric computing 800 can receive the user's provided identifying info, biometric data, and compare it to stored biometric templates to verify the identity of the user attempting to access the cloud-based services.

Following confirmation, at step 2340 a token or other encrypted data, derived from the verified biometrics or identifying data, is generated, and sent to the application/cloud service requesting identification. This token serves as a secure and privacy preserving means of verifying the user's identity. The service or application receiving the identification token verifies it by comparing it with stored records at step 2350. This verification process ensures that the user's identity is authenticated based on the information stored in the PHDB, without the need to expose sensitive biometric data directly.

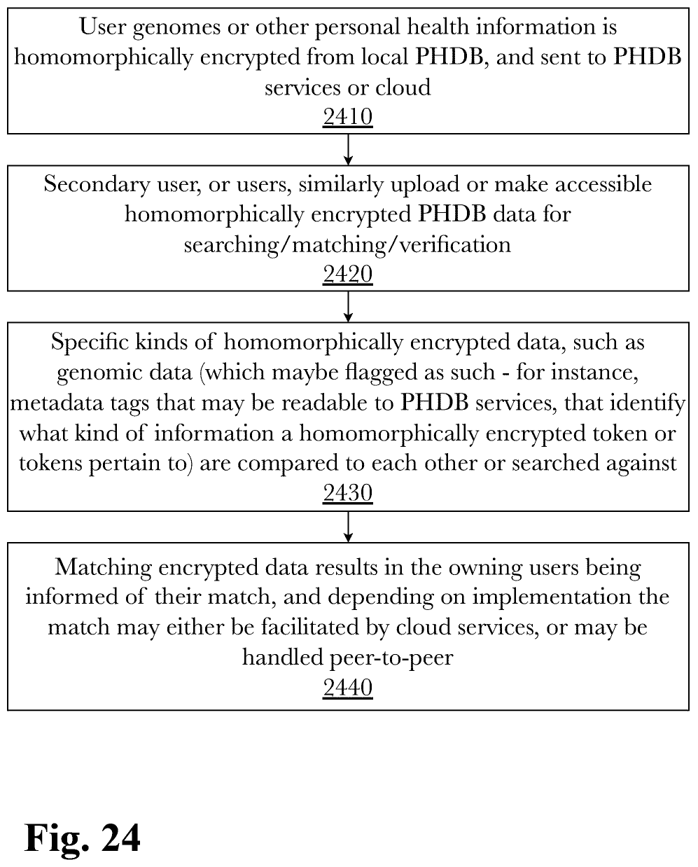

FIG. 24 is a method diagram showing exemplary steps taken for genome screening of a pair of individuals using their PHDBs, according to an aspect of the invention. According to the aspect, the process begins at step 2410 when the user genomic or other personal health information is homomorphically encrypted from their local PHDB and sent to PHDB services or cloud. Simultaneously, a secondary user, or multiple users, upload or make accessible homomorphically encrypted PHDB data for searching, matching, or verification 2420. All users can adjust access controls and permission for sharing with respect to the data stored in their PHDB. One such selection may be allowing other PHDB system users to perform homomorphic comparisons or other analysis of homomorphically encrypted data.

Specific kinds of homomorphically encrypted data, such as genomic data (which may be flagged as such—for instance, via metadata tags that may be readable to PHDB services, identifying what kind of information a homomorphically encrypted token or tokens pertain to), are then compared to each other or searched against 2430. Matching encrypted data results in the owning users being informed of their match. Depending on implementation, the match may either be facilitated by cloud services or handled peer-to-peer 2440. This process allows users to be notified of potentially significant genomic matches while preserving the security and privacy of their sensitive health information.

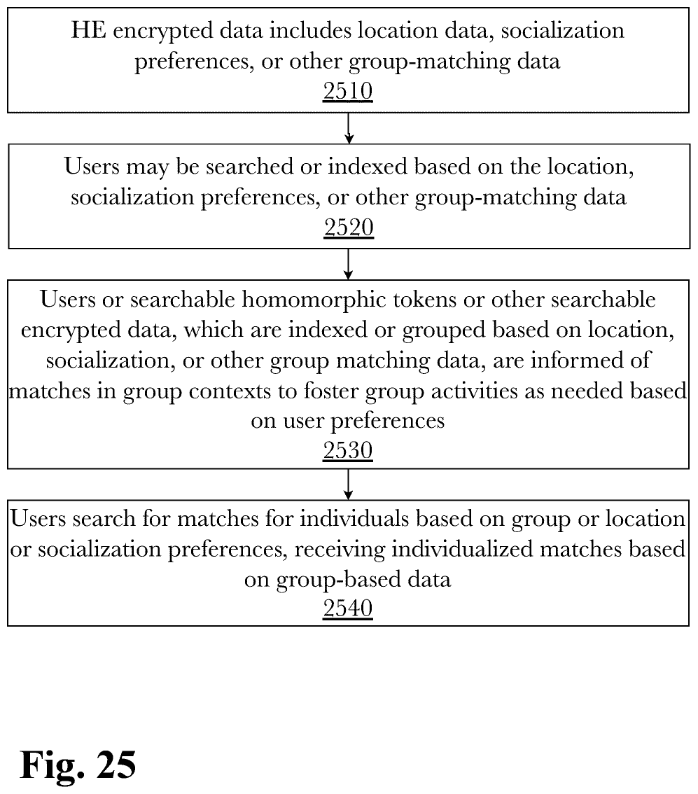

FIG. 25 is a method diagram showing exemplary steps taken for groups to be formed and users to be screened based on their group memberships via their PHDBs, according to an aspect of the invention. According to the aspect, the process begins at step 2510 when the PHDB system homomorphically encrypts (HE) data, including location data, socialization preferences, or other group-matching data. Users may be searched or indexed based on the location, socialization preferences, or other group-matching data 2520. The system may implement a database or indexing system to store and organize user data, ensuring that the indexing system can efficiently query and retrieve data based on different criteria, such as location or socialization preferences. In some implementations, a database with geospatial indexing capabilities may be utilized to facilitate location-based search.

According to an aspect of an embodiment, groups may be declaratively assigned wherein an individual can declare which groups they belong to (e.g., I am a veteran or member of this school or church, etc.). It should be appreciated that groups may be physical emergent groups and emergent digital groups. Physical emergent groups are groups that form in physical, real-world environments, such as communities, workplaces, or social gatherings. Physical emergent groups often arise spontaneously based on shared interests, needs, or circumstances. For example, a group of neighbors coming together to address a local issue would be considered a physical emergent group. Digital emergent groups are groups that form in digital or online environments, such as social media platforms, online forums, or multiplayer online games. Digital emergent groups can form around common interests, hobbies, or goals, and they often transcend geographical boundaries. For example, a group of individuals organizing a fundraiser on a crowdfunding platform would be considered a digital emergent group. Both types of groups can exhibit emergent properties, meaning that the group as a whole displays characteristics or behaviors that are not exhibited by any individual member. These properties can arise from the interactions and dynamics within the group, making them more than the sum of their parts. By analyzing these declared and emergent groups, the platform 120 can aid with broader association issues that can impact longevity factors for physical and mental fitness where long term data is needed (e.g., Alzheimer's).

At a next step 2530 users or searchable homomorphic tokens, and other searchable encrypted data, which are indexed or grouped based on location, socialization, or other group matching data, are informed of matching data, are informed of matches in group contexts. This fosters group activities as needed based on user preferences. For example, an individual may be seeking other people to play a VR-based video game with and may also be seeking opportunities to meet up with these other people for socialization outside of the VR video game. In such a use case, the individuals may be matched based on socialization preferences related to AR/VR gaming and may be further matched based on proximity to the individual who was seeking out the group.

Users can search for matches for individuals based on group, location, or socialization preferences and receive individualized matches based on group-based data at step 2540. That is to say, that given a matched group of people, users can then search for individual matches selected from the population of the matched group. These individual matches may be based on the group-based data and may not include other user preference data.

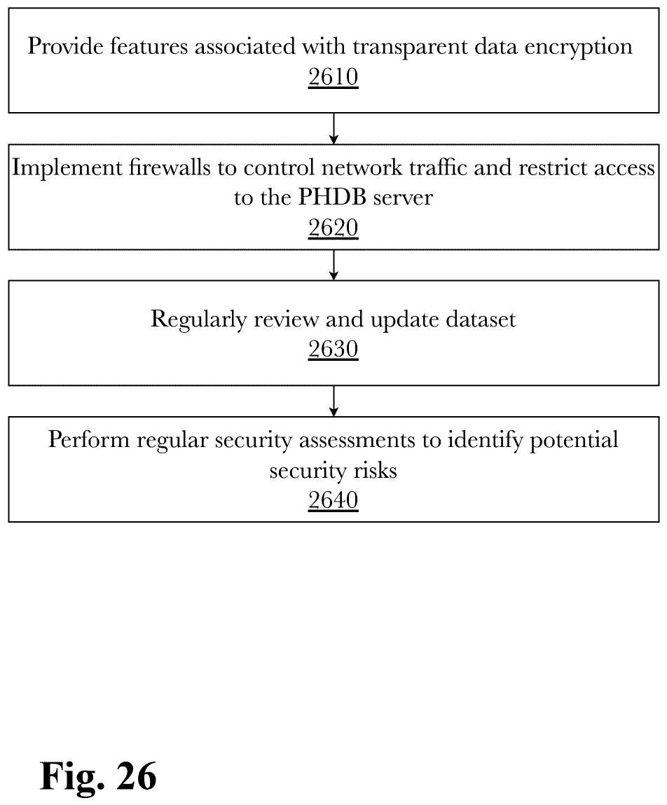

FIG. 26 is a method diagram showing exemplary steps taken on an oracle database to mitigate the risk of exploitation. According to an embodiment, the process utilizes homomorphic encryption for sensitive data, both in the cloud and at rest. Oracle provides features such as transparent data encryption for securing data as the database level 2610. Implement firewalls to control network traffic and restrict access to the personal health database server 2620. The oracle dataset can be regularly reviewed and updated to ensure that in the event of a security incident, the data can quickly be recovered 2630. Regular security assessments will be conducted, including penetration testing and vulnerability scanning to identify and address potential weaknesses in the oracle environment 2640.

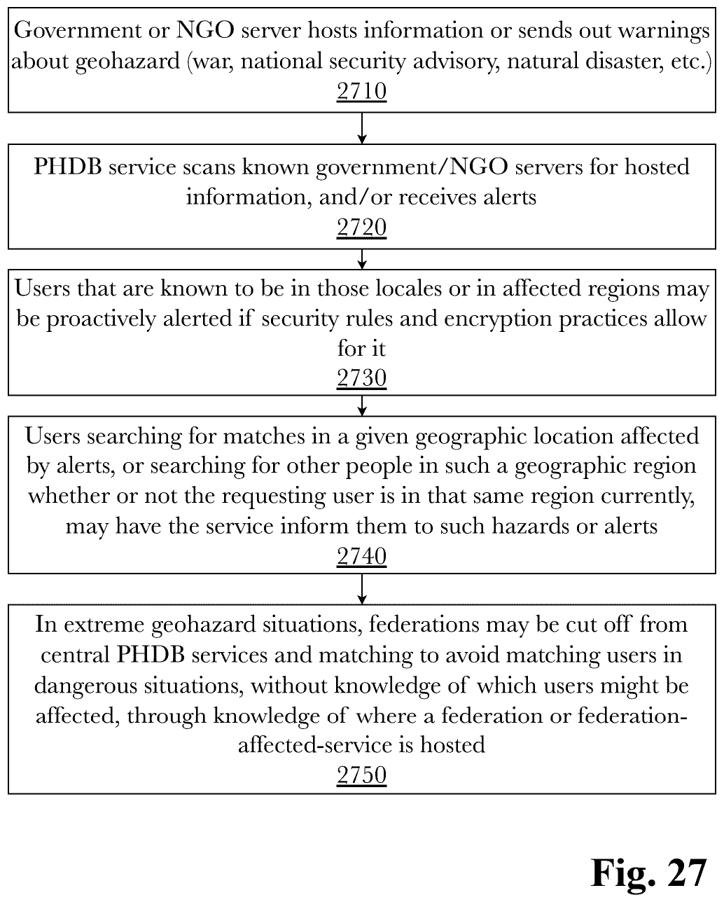

FIG. 27 is a method diagram showing exemplary steps taken for a PHDB service to alert or warn users based on geohazards and government or NGO alerts based on geographical regions, according to an aspect of the invention. According to the aspect, the process begins at step 2710 when a government or NGO server hosts information or sends out warnings about geohazard (war, national security advisory, natural disaster, etc.). The PHDB service 101 scans known government/NGO servers for hosted information, and/or receives alerts directly at step 2720. Users that are known to be in those locales or in affected regions may be proactively alerted, contingent upon security rules and encryption practices at step 2730. This proactive alerting ensures that users with potential exposure to geohazards are promptly informed, enhancing their situational awareness and safety.

According to some aspects, historic geolocation data may be used to cross reference known environmental hazards as it relates to subsequent potential health implications outside of alerts. Similar to how the Dept. of Veterans Affairs retroactively declared burn pit exposure for anyone deployed to a specific location or area. This could be extended to known oil spill areas, for example, or water quality issues (i.e., Flint, Michigan) where there is no “official” alert, but there was a known environmental event (e.g., oil spill in the gulf). Modeling and simulation may then be used to discover seemingly unrelated health issues. For example, would modeling the fluid dynamics around the use of Corexit 9527A (known to contain toxics harmful to red blood cells) in the Gulf turn up long term health impacts when combined with data from PHDB? Adding second and third order effects into the modeling would be possible (e.g., health issues related to dispersant impact of fish that were consumed). Such a system may be used to compare environmental exposure to fertility issues and make recommendations.

Users searching for matches in a given geographic location affected by alerts or searching for other people in such a geographic region, whether or not the requesting user is in that same region currently, may have the service inform them to such hazards or alerts at step 2740. This ensures that users actively seeking connections or information in specific regions are made aware of potential risks. In extreme geohazard situations, in an optional step 2750 federations may be cut off from central PHDB services and matching to avoid matching users in dangerous situations. This is done without specific knowledge of which users might be affected, through knowledge of where a federation or federation-affected-service is hosted. This proactive disconnection ensures the safety of users during critical situations.

According to some embodiments, if may be possible for different consortiums of individuals to share data related to their exposure to geohazards and/or environmental hazards with limited context specifications (e.g., a potential group of litigants for a chemical lawsuit or a group of concerned parents). In a use case, individuals in a consortium may opt-in to marketing pools and/or subscriptions (e.g., expert content like relevant journal papers on select omics elements referencing a subscriptions groups' characteristics). In another use case, consider citizens in the U.S. who get healthcare in another country or on a cruise where subsets of records may be more appropriate for sharing for limited time and/or utility.

It should be appreciated that individual users may select which of their data they can share with an NGO or governmental entity. It should be further appreciated that a “regulatory compliance” locker may be supported by platform 120 for records needed to be kept for liability and professional responsibility reasons but where the data is encrypted w/a key that is not available to healthcare providers in that system or office.

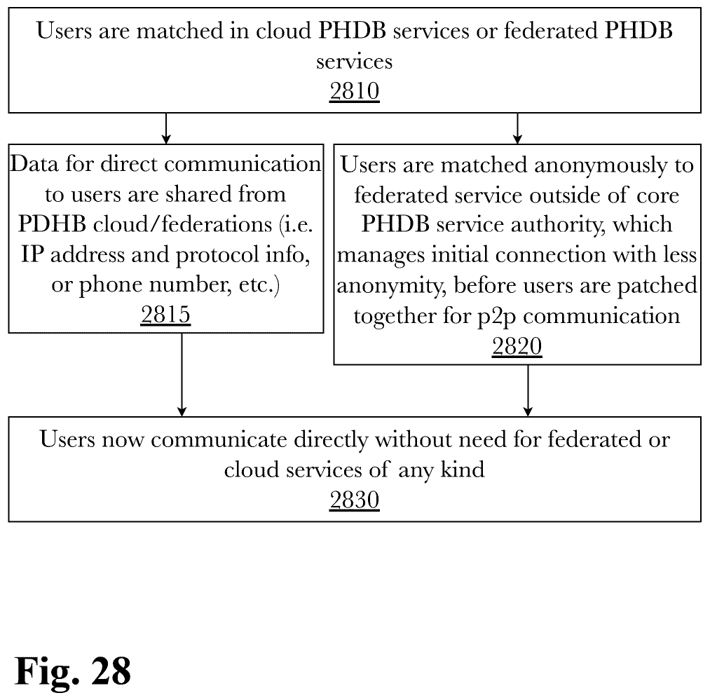

FIG. 28 is a method diagram showing exemplary steps taken for PHDB platform users to be paired for peer-to-peer communications, according to an aspect of the invention. According to the aspect, the process begins at step 2810 as users are matched in cloud PHDB services or federated PHDB services. Data for direct communication to users are shared from PDHB cloud/federations (i.e. IP address and protocol info, or phone number, etc.) 2815. Additionally, or alternatively, users are then matched anonymously to federated services outside of the core PHDB service authority. This federated service manages initial connection with less anonymity before users are seamlessly patched together for peer-to-peer communication at step 2820. Once the users are matched, they can communicate directly without need for federated or cloud services of any kind 2830. This enables users to establish direct and private communications, enhancing the efficiency and privacy of their interactions.

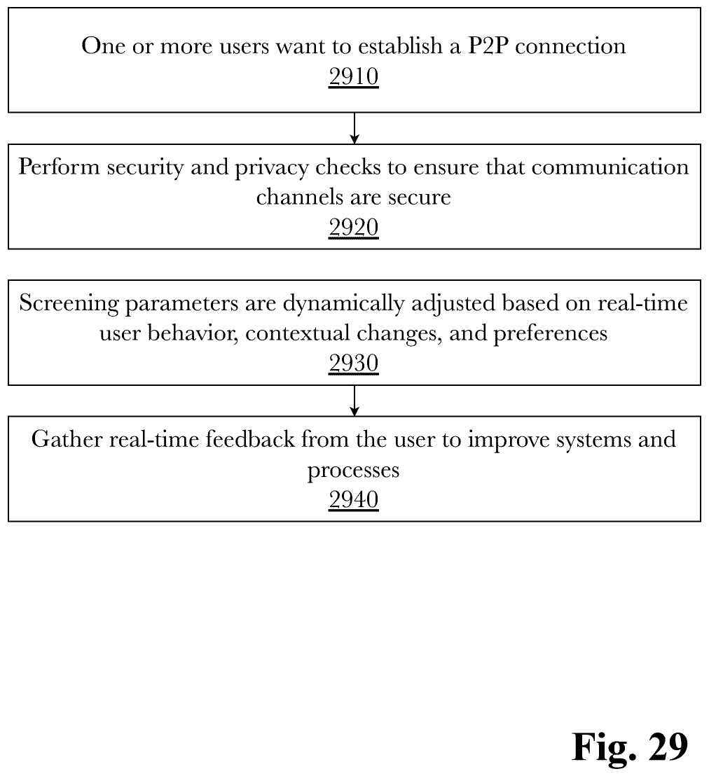

FIG. 29 is a method diagram showing exemplary steps taken for a pair of users to be screened for peer-to-peer communications, according to an aspect of the invention. According to the aspect, the process begins at step 2910 as one or both users express an interest in peer-to-peer communications. This may be done through a user interface provided by PHDB application 111a stored and operated on a PHDB-enabled mobile device or on some other computing device 115a, wherein the user interface can provide a means for one or two users can indicate their preferences and interests via PHDB. At step 2920, before establishing the peer-to-peer connection, the system will conduct security and privacy checks to ensure that communication channels are secure and user privacy is maintained throughout the interaction. This may involve verifying user identities (e.g., two-factor authentication, biometric authentication, token-based authentication, etc.), encrypting communication channels, and implementing access control measures (which may be specified by the user or by PHDB-related services)

At step 2930 the screening parameters are dynamically adjusted based on real-time user behavior, contextual changes, and any evolving preferences. The adaptive approach enhances the accuracy of the screening process. To facilitate dynamic screening the system may use machine learning algorithms to analyze user behavior and detect patterns that indicate the likelihood of malicious activity or inappropriate behavior. For example, the system can analyze the frequency and timing of messages, the content of messages, and the history of interactions. Furthermore, the system can take into account contextual information such as user preferences, location, and device information to adapt screening parameters. For example, the system might adjust screening parameters based on whether the user is accessing the system from a trusted location or device.

In an implementation, the system may provide functionality directed to real-time user behavior detection. This can include monitoring user interactions and events in real-time to detect patterns of behavior. This could include monitoring mouse movements, keyboard inputs, screen tapping/swiping/touching, and other user actions. In an embodiment, the system may use machine learning models to detect anomalies or patterns in user behavior that may indicate malicious activity. These models could be trained using labeled data to improve their accuracy. In an aspect, the system may create user profiles based on historical behavior and use these profiles to detect deviations from normal behavior. For example, if a user suddenly starts sending a large number of messages, this could be flagged as suspicious behavior.

As a last step 2940, during and after peer-to-peer communication, the system gathers real-time feedback from users. The feedback loop contributes to the continuous improvement of the screening algorithms, ensuring an optimized user experience.

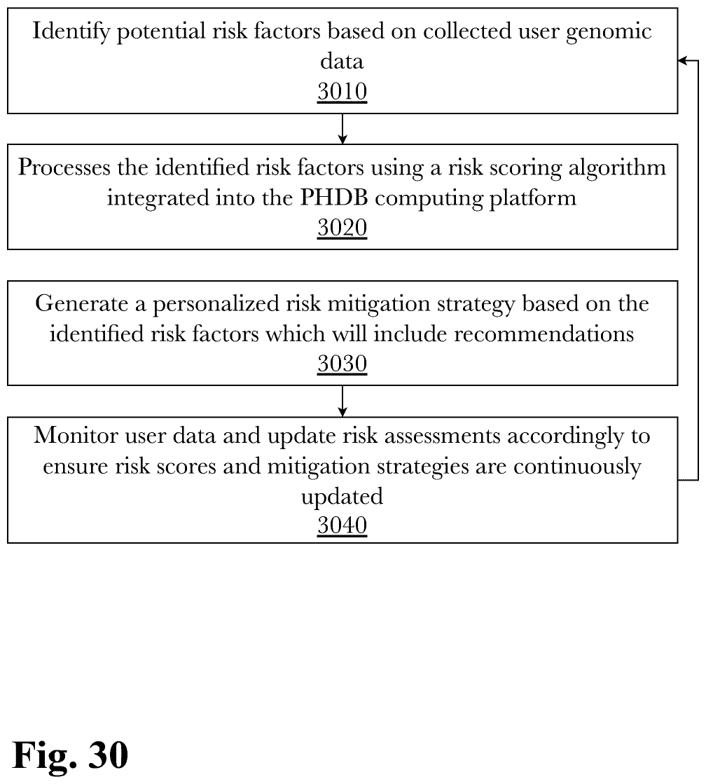

FIG. 30 is a method diagram showing exemplary steps taken for individuals to be screened and scored for risk and risk mitigation factors using their PHDB and the PHDB computing platform, according to an aspect of the invention. According to the aspect, the process begins at step 3010 when PHDB system identifies potential risk factors based on the collected user genomic data, which can span a range of health-related aspects, for example whether both the person who contributes the egg and the one who provides the sperm carries variants within the same gene to give rise to a child with cystic fibrosis, spinal muscular atrophy, sickle cell disease, and Tay-Sachs. The risk-factors may be chosen based on user defined preferences or other input or data that is derived or inferred from analysis of a plurality of PHDB data. These could include genetic variation associated with certain diseases or conditions. For example, a user can screen for a specific genetic disorder, phenotypic expression, behavioral condition, or any other preference relevant to the type of search/match being performed.

At a next step 3020 the identified risk factors can be processed by a risk scoring algorithm that may be integrated into the PHDB computing platform 120. Such a scoring algorithm may be developed and deployed using machine learning engine 1510. The scoring algorithm can be trained to assign scores to each risk factor considering the weight of each risk 3020. A weight may be assigned to each risk factor based on its importance or relevance to the risk being assessed. The weight could be based on scientific evidence, expert opinion, or other relevant factors. The weights may be assigned by a developer of the scoring algorithm, or the weights may be a learned feature of the scoring model itself. For example, a neural network may be developed to generate as output a plurality of risk scores for various identified risk factors, wherein the neural network learns to assign weights to each risk factor based on continuous learning on a large data corpus. The scoring algorithm may be validated, for example, using independent datasets or clinical studies to ensure its accuracy and reliability. In some implementations, feedback from domain experts (e.g., clinical psychologists, physicians, etc.) may be used to improve the scoring algorithms performance and fitness.

In some implementations, genomic data may be collected from users, such as whole genome sequencing or whole exome sequencing data, depending on the scope of the analysis. The genomic data may be analyzed to identify relevant genetic variations or mutations associated with identified risk factors. The scoring algorithm may, for each risk factor, calculate a risk score based on the presence or absence of the genetic variation or mutation associated with that risk factor. The score could be binary (0 or 1) or based on a scale, depending on the nature of the risk factor and the embodiment of the system. The system may then calculate a weighted sum of the risk scores for all the risk factors, using the weights assigned to each risk factor. This yields an overall risk score for the individual. In various embodiments, users or PHDB services may define threshold for the risk scores to categorize individuals into different risk categories (e.g., low, medium, high).

Additionally, the system may be configured to provide interpretation guidelines to help users understand their risk level and any recommended actions based on their risk score. At step 3030 the system generates a personalized risk mitigation strategy based on the identified risk factors (and risk score) which can include recommendations, for example, whether two users would be genetically compatible. For genetic compatibility between users, this could involve comparing genetic data to assess the likelihood of genetic compatibility and providing recommendations based on the results. The system may also take into account other factors that may impact risk mitigation recommendations, such as lifestyle factors, medical history, and environmental factors. Exemplary recommendations can include lifestyle changes, medical interventions, or genetic counseling. In at least one implementation, an LLM may be developed to receive an input comprising at least a set of identified risk factors and a risk score for one or more individuals and to generate as output a risk mitigation strategy.

At step 3040 the system continuously monitors user data and updates risk assessments accordingly to ensure risk scores and mitigation strategies are continuously updated to reflect current data. When new user data is discovered (e.g., a user has updated the personal information in their PHDB) the process may loop back to step 3010 wherein new potential risk factors may be identified based on the updated user data. The process then repeats, and a new risk score and risk mitigation strategy is created.

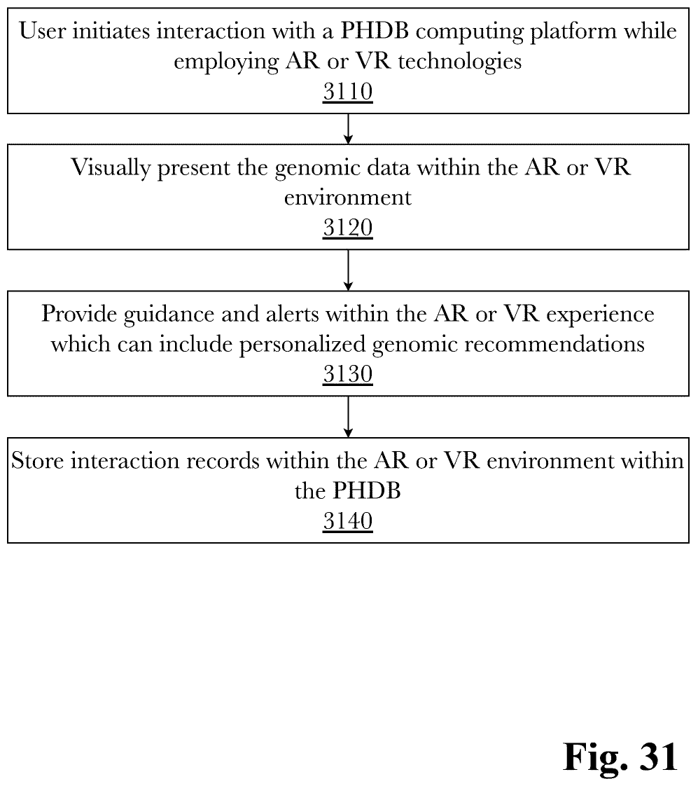

FIG. 31 is a method diagram showing exemplary steps taken for a PHDB computing platform to be utilized alongside AR or VR technologies, according to an aspect of the invention. According to the aspect, the process begins at step 3110 when a user initiates interaction with the PHDB computing platform 120 while employing AR or VR technologies. Initiation can occur through voice commands, gestures, or other AR/VR input methods. At step 3120 genomic data is visually presented within the AR or VR environment. This can include immersive visualizations of genomic metrics such as scanning another user that has opted into the genomic screening and them turning a color, for example, green for being clear with no issues and turning red if chances are there is not genetic compatibility.

At step 3130 PHDB computing platform provides guidance and alerts within the AR or VR experience which can include personalized genomic recommendations 3130. For example, in an augmented reality environment, PHDB computing platform 120 can overlay a virtual path which leads to a screened, genetically compatible user. Multiple paths may be overlayed, and color coordinated to indicate relative strengths of compatibility or some other matched factor. For example, a hot-cold scheme could be used wherein “cold” (low compatibility) paths may be indicated in a dark shade of blue, “hot” (high compatibility) paths may be indicated in a bright shade of red, and the range of compatibilities between low and high may be represented as color gradient shifting from dark blue to bright red. Guidance and alerts may be text-based and also displayed in the AR/VR environment

As a last step 3140 interaction records within the AR or VR environment will be stored within the PHDB to ensure a comprehensive record of the user's health-related AR or VR interactions.

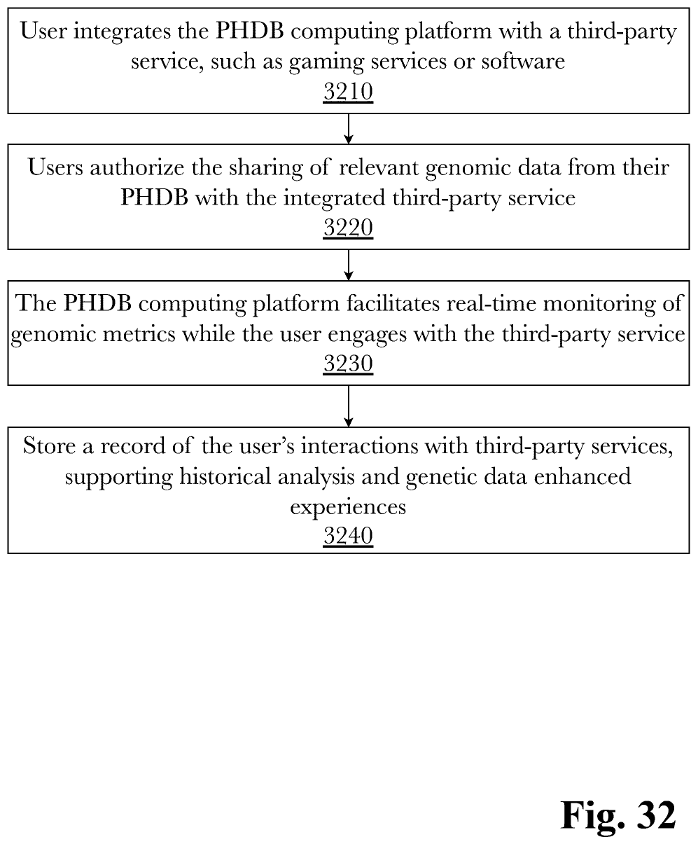

FIG. 32 is a method diagram showing exemplary steps taken for a PHDB computing platform to be used in tandem with third party services such as gaming services or software, according to an aspect of the invention. According to the aspect, the process begins at step 3210 when a user integrates the PHDB computing platform with a third-party service, such as gaming services or software. The integration can be initiated through user preferences, app settings, or dedicated integration features. Users may authorize the sharing of relevant genomic data (or other personal information) from their PHDB with the integrated third-party service at step 3220. To ensure that only authorized devices or users can access the data, integrated third-party services can use authentication and authorization mechanisms. This can include using digital certificates, passwords, or biometric authentication. The PHDB computing platform facilitates real-time monitoring of genomic metrics while the user engages with the third-party service at step 3230. This can include adaptive gameplay based on real-time genetic and health data, personalized challenges, or dynamic content tailored to the user's genetic profile. At step 3240 interaction records and data integration details are stored within the PHDB. This ensures a comprehensive record of the user's interactions with third-party services, supporting historical analysis and genetic data enhanced experiences.

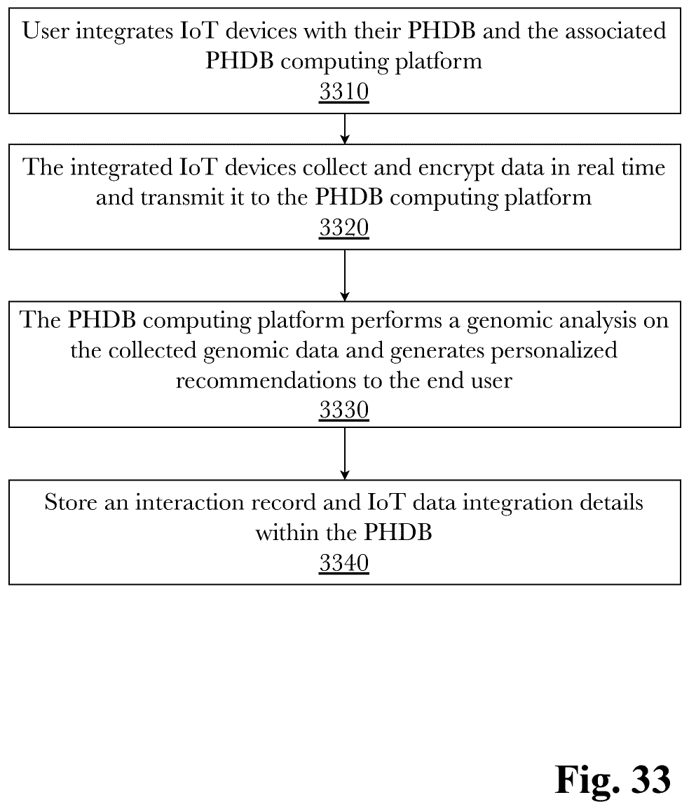

FIG. 33 is a method diagram showing exemplary steps taken for a user to integrate a plurality of IoT devices with their PHDB and a PHDB computing platform, according to an aspect of the invention. According to the aspect, the process begins at step 3310 when a user integrates IoT devices with their PHDB and the associated PHDB computing platform. The integration can be initiated through user preferences, app settings, or dedicated integration features. IoT devices may include smart kitchen appliances, wearables, or other connected devices capable of tracking information. IoT devices capture data using sensors or other data collection mechanisms. This data can include sensor readings, device status information, environmental data, and more. The integrated IoT devices may be configured to collect and encrypt data in real time and the data is transmitted to the PHDB computing platform at step 3320. Before encryption, data may undergo preprocessing to clean, filter, or format it for further processing or storage. Once the data is ready, it is encrypted to protect it from unauthorized access. There are several encryption techniques that can be used, including symmetric encryption and asymmetric encryption. For data transmission over networks, IoT devices can use TLS (transport layer security) to encrypt data in transit. TLS ensures that data is encrypted between the IoT device and the server or other devices it communicates with. To ensure that only authorized devices or users can access the data, IoT devices can use authentication and authorization mechanisms. This can include using digital certificates, passwords, or biometric authentication. The IoT devices may be configured to collect, encrypt, and transmit genomic data. The PHDB computing platform 120 performs a genomic analysis on the collected genomic data and generates personalized recommendations to the end user at step 3330. At step 3340 the system may store an interaction record and IoT data integration details within the PHDB to ensure a comprehensive record of the user's genetic information, IoT device interactions, and health-related insights.

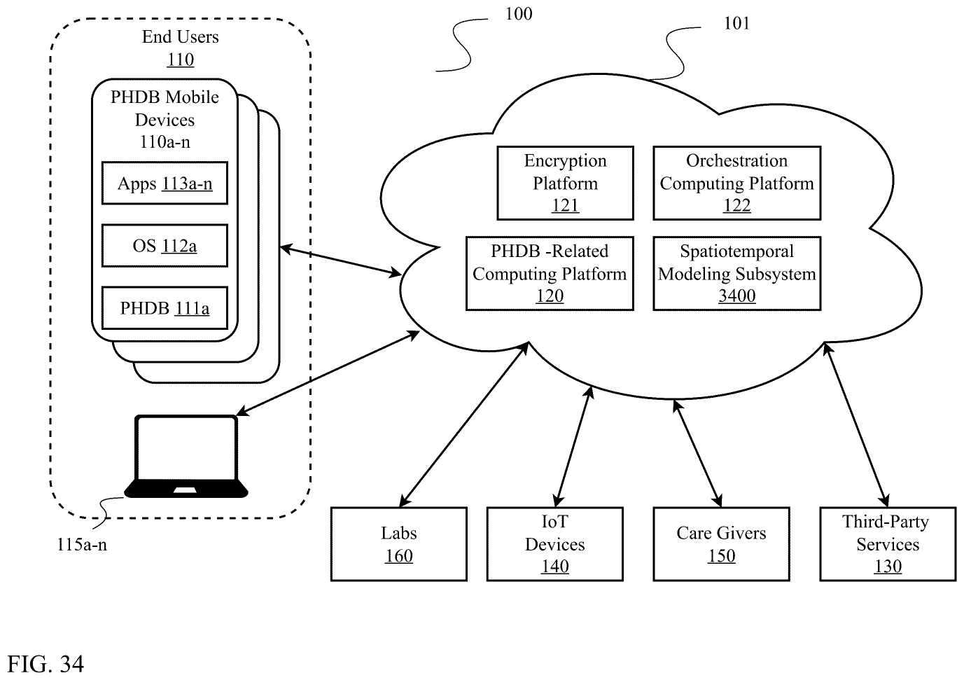

FIG. 34 is a block diagram showing an exemplary arrangement of a PHDB, where the PHDB system is configured for real time spatiotemporal modeling. The enhanced PHDB system introduces a significant advancement in health data management and analysis through the integration of a spatiotemporal modeling subsystem 3400. This new component interfaces seamlessly with the existing PHDB-related computing platform 120, expanding the system's capabilities to process and analyze health data across both spatial and temporal dimensions. The spatiotemporal modeling subsystem 3400 works in concert with the encryption platform 121 and the orchestration computing platform 122 to provide a comprehensive, secure, and dynamic approach to personal health data management.

The spatiotemporal modeling subsystem 3400 is designed to create and maintain a four-dimensional representation of an individual's health data. It processes information from various sources, including the PHDB mobile devices 110a-n, personal computers 115a-n, IoT devices 140, labs 160, third-party services 130, and care givers 150. This subsystem transforms the traditional static view of health records into a dynamic, evolving model that captures changes in health status over time and across different anatomical locations.

At its core, the spatiotemporal modeling subsystem 3400 employs advanced algorithms to construct a time-stabilized three-dimensional mesh of the human body. This mesh serves as a framework onto which various types of health data can be mapped. The subsystem can handle data at different resolution levels, allowing for analysis ranging from cellular-level information to whole-body overviews. By tagging multi-omics data to specific “cells” in the 3D mesh and tracking changes over time, the system provides a spatially and temporally resolved view of biological molecules within tissues or organisms.

The integration of this subsystem enhances the PHDB system's ability to perform sophisticated analyses. For instance, it enables the study of gene expression patterns in specific regions of the body over time, providing insights into how genetic factors interact with environmental influences to affect health outcomes. This capability is particularly valuable for understanding complex, multifactorial conditions that evolve over time, such as cancer progression or neurodegenerative diseases.

Furthermore, the spatiotemporal modeling subsystem 3400 supports advanced simulation and predictive modeling. By leveraging the comprehensive, four-dimensional health data, it can generate multiple simulation paths based on “seeds” extracted from the PHDB. This feature allows for parametric studies that explore the potential impacts of various factors, including imaging techniques, sampling methods, and environmental exposures. Such simulations can aid in diagnostics, treatment advisory, and treatment calibration, offering a more nuanced and personalized approach to healthcare.

The concept of “seeds” in the context of the PHDB system refers to specific data points or sets of parameters extracted from a user's personal health database that serve as starting points for simulations or analyses. These seeds are carefully selected snapshots of an individual's health status at a particular point in time, encompassing various types of data such as genomic information, current physiological measurements, lifestyle factors, and environmental exposures. The spatiotemporal modeling subsystem 3400 can utilize these seeds to initiate multiple simulation paths, allowing for the exploration of various “what-if” scenarios and potential health outcomes. For example, a seed might include a user's current cardiovascular health metrics, genetic predispositions, and lifestyle factors. The system could then generate simulations to predict how changes in diet, exercise, or medication might affect the user's heart health over time. By using seeds from different time points or with varied parameters, the system can conduct parametric studies to investigate the potential impacts of various factors on health outcomes. This approach enables a more personalized and predictive form of healthcare, where interventions can be tailored based on simulated outcomes derived from an individual's unique health profile. The use of seeds in this manner significantly enhances the PHDB system's capability to provide nuanced, forward-looking health insights and supports more informed decision-making for both users and healthcare providers.

The subsystem may also incorporate machine learning and AI processes for classifying individual cells and identifying cellular neighborhoods. These advanced analytical capabilities contribute to the creation of an enhanced 3D (or 4D) mesh that serves as an anchor for all available data. This approach is particularly beneficial for complex modeling techniques such as finite element analysis, fluid modeling, and fluid-structure interaction modeling, which are crucial for understanding the intricate dynamics of biological systems.

The spatiotemporal modeling subsystem 3400 ingests data from multiple sources within the PHDB ecosystem. It interfaces directly with the PHDB-related computing platform 120 to access the diverse types of data stored in users' personal health databases. This includes genomic data, microbiome data, phenotype data, biometric data, medical data, activity data, and snapshot data. The subsystem also receives real-time data streams from IoT devices 140 and wearables, which may be part of the PHDB mobile devices 110a-n. These data streams provide continuous updates on various physiological parameters, activity levels, and environmental exposures. Additionally, the subsystem can incorporate data from labs 160 and third-party services 130, which may include detailed medical imaging, test results, and specialized health assessments.

The data ingestion process is managed by the orchestration computing platform 122, which ensures that incoming data is properly formatted, validated, and securely transmitted to the spatiotemporal modeling subsystem. The encryption platform 121 plays a role in this process, ensuring that all data remains encrypted during transmission and storage. The spatiotemporal modeling subsystem 3400 is designed to work with homomorphically encrypted data, allowing it to perform complex computations and analyses without decrypting sensitive information, thus maintaining user privacy and data security.

Once the data is ingested, the spatiotemporal modeling subsystem processes it to create and update the 4D model of the user's health. This involves mapping each data point to its appropriate spatial location within the 3D body mesh and associating it with a specific time point. The subsystem employs sophisticated algorithms to interpolate between data points, creating a continuous representation of health parameters across space and time. It also uses machine learning techniques to classify cells, identify patterns, and make predictions based on the accumulated data.

End users can interact with the spatiotemporal modeling subsystem through various interfaces provided by the PHDB mobile devices 110a-n and personal computers 115a-n. The system offers intuitive visualization tools that allow users to explore their health data in a 4D space. Users can navigate through their body model, zooming in on specific organs or tissues, and moving forward or backward in time to observe changes in their health parameters. This interactive visualization can be particularly helpful for understanding the progression of chronic conditions or the effects of treatments over time.

For more advanced interactions, the system provides query tools that allow users to ask complex questions about their health data. For example, a user might query the system to show all instances where their blood pressure exceeded a certain threshold, with the results displayed as highlighted regions in the 4D model. Users can also set up alerts based on spatiotemporal patterns, such as notifications for rapid changes in a specific health parameter within a particular body region.

Healthcare providers and researchers, with appropriate permissions, can use more sophisticated tools to interact with the spatiotemporal modeling subsystem. They can run simulations, perform statistical analyses across populations, and use the system's predictive modeling capabilities to forecast potential health outcomes. The system also supports the creation of custom visualizations and reports, allowing healthcare providers to communicate complex health information to patients in an understandable and visually engaging manner.

Furthermore, the spatiotemporal modeling subsystem 3400 may integrate with augmented and virtual reality systems, enabling immersive interactions with the 4D health model. This can be particularly useful for patient education, surgical planning, or exploring complex physiological processes. Users can literally step inside a virtual representation of their body, gaining a unique perspective on their health data.

Through these varied interaction methods, the spatiotemporal modeling subsystem transforms the way users engage with their health data. It moves beyond static charts and isolated data points to provide a dynamic, holistic view of health that evolves over time and space. This rich, interactive experience has the potential to improve health literacy, encourage proactive health management, and facilitate more informed discussions between patients and healthcare providers.

This subsystem is able to implement a stabilized space-time process. This process enables more accurate and advanced modeling simulations by creating a reference “atlas” for single cells, cell groups (tissue/cellular neighborhoods), and other multi-omics information. It achieves this by simulating from an average mesh viewed as a stable point, then minimizing the dynamism inside the mesh over a finite time. This approach effectively creates a GPS-like system for the body, providing spatiotemporal locality of data that was previously unattainable in traditional medical record systems.

The spatiotemporal modeling subsystem 3400 is designed to support a wide range of analytical scenarios, from single cell/tissue analysis to multiple tissue groups and whole-body simulations. Its ability to parallelize parametric studies of different assumptions and perturbations of models makes it a powerful tool for exploring complex biological phenomena and their potential outcomes.

By incorporating this advanced spatiotemporal modeling capability, the PHDB system significantly enhances its utility for personalized medicine, disease prevention, and treatment optimization. It provides healthcare providers with a more comprehensive and dynamic view of patient health, enabling more informed decision-making and potentially leading to improved patient outcomes. Moreover, this system opens new avenues for medical research, offering unprecedented insights into the complex interplay between genetics, environment, and health over time and space.

In one embodiment, spatiotemporal modeling subsystem 3400 may incorporate a range of specialized modeling and simulation elements to provide a comprehensive, multi-scale representation of the subject's health status. These advanced modeling techniques allow for detailed analysis and prediction across various physiological levels, from molecular interactions to organ-system dynamics.

Spatiotemporal modeling subsystem 3400 may utilize Computational Fluid Dynamics (CFD) modeling to simulate and analyze fluid flows within the body. This is particularly crucial for understanding cardiovascular health, respiratory function, and other systems involving fluid dynamics. The CFD models can simulate blood flow through vessels, air flow in the lungs, or cerebrospinal fluid circulation. By incorporating patient-specific anatomical data, these models can predict areas of abnormal flow, potential for plaque formation in arteries, or efficiency of gas exchange in the lungs.

Fluid-Structure Interaction (FSI) simulations may extend the CFD capabilities by modeling the interplay between fluids and elastic structures in the body. This is essential for accurately representing phenomena such as arterial wall deformation under pulsatile blood flow, heart valve dynamics, or the mechanics of breathing. FSI models in the PHDB system can help predict the risk of aneurysm rupture, optimize artificial heart valve designs, or assess the impact of respiratory disorders on lung tissue.

At the molecular level, spatiotemporal modeling subsystem 3400 may incorporate molecular dynamics simulations. These simulations model the behavior of biomolecules based on physical principles, allowing for the study of drug-target interactions, ion channel function, or the effects of genetic mutations on protein structure. By integrating a patient's genetic data, these simulations can provide personalized insights into drug efficacy or the molecular basis of genetic disorders.

Protein folding simulations may further augment molecular-level modeling. Using algorithms, including but not limited to machine learning enhanced approaches like AlphaFold, the system can predict the three-dimensional structure of proteins based on their amino acid sequence. This capability is invaluable for understanding the impact of genetic variations on protein function, designing targeted therapies, or predicting the effects of post-translational modifications.

Spatiotemporal modeling subsystem 3400 is capable of cellular level simulations that form a bridge between molecular processes and tissue-level phenomena. These models can simulate intracellular signaling pathways, metabolic networks, or cell population dynamics. In the context of the PHDB system, cellular simulations can be used to model the progression of diseases like cancer, predict immune system responses, or optimize personalized treatment strategies at the cellular level.

Spatiotemporal modeling subsystem 3400 seamlessly integrates specialized modeling elements within its 4D framework. For instance, CFD and FSI simulations of blood flow can be mapped onto the patient's vascular anatomy over time, while molecular and cellular simulations provide insights into the underlying biological processes driving observed physiological changes.

To manage the computational complexity of these diverse simulation types, spatiotemporal modeling subsystem 3400 may utilize adaptive multi-scale modeling techniques. This approach allows for seamless transitions between different levels of detail, focusing computational resources where they are most needed based on the specific health query or analysis being performed.

In another example, in a patient with suspected coronary artery disease, the system could combine CFD simulations of coronary blood flow with cellular-level models of atherosclerosis progression and molecular simulations of lipid metabolism. This integrated approach could provide a comprehensive assessment of the patient's cardiovascular health, predict the likelihood of future cardiac events, and suggest personalized interventions based on the patient's unique physiological and genetic profile.

In one embodiment, the personal health database (PHDB) system leverages the existing spatiotemporal modeling subsystem 3400 to implement an advanced data sharing framework that provides users with control over their medical information. This framework enhances the capabilities of computing platform 120 by introducing granular data access control and spatiotemporal data release features. The system may utilize data preprocessor 5210 within the IoT processing hub 5100 to tag each data point with metadata, including information about its type, timestamp, associated conditions, originating provider, and sensitivity level. Orchestration computing platform 122 then creates and applies a set of rules based on user-defined sharing preferences to these tags, allowing for precise control over data accessibility. Spatiotemporal modeling subsystem 3400 incorporates this granular access control into its dynamic 3D and 4D representations of patient data, enabling context-aware data sharing.

Geospatial computing engine 1120 within community computing engine 1220 may be employed to implement location-based data release, using geofencing technology to enable or restrict data access based on the user's or data requester's location. Visualization and alerting subsystem 5240 of the IoT processing hub 5100 may be utilized to present relevant, aggregated data based on the spatiotemporal context of the data request. Computing platform 120 manages the continuous monitoring of spatiotemporal conditions, while encryption platform 121 ensures secure data transmission.

This embodiment leverages the existing architecture to provide a highly flexible, context-aware approach to medical data sharing, offering users unprecedented control over their health information while still enabling efficient and appropriate data sharing with healthcare providers and authorized applications, all within the secure and comprehensive framework of the PHDB system.

This framework significantly enhances the capabilities offered by current systems by introducing granular data access control and spatiotemporal data release features. The system allows users to define highly specific parameters for data sharing, moving beyond the concept of a simple “allow” or “deny” approach. Users can selectively share subsets of their medical information based on various criteria, including data type, time periods, condition-specific data, provider-specific sharing, and data sensitivity levels. This granular control is achieved through tagging each data point in the user's PHDB with metadata that includes information about its type, timestamp, associated conditions, originating provider, and sensitivity level. When a user configures their sharing preferences, the system creates a set of rules that are applied to these tags, allowing for precise control over data accessibility.

Building upon the granular access control, the system may incorporate a spatiotemporal data release mechanism. This feature allows for the dynamic release of medical information based on both spatial (location-based) and temporal (time-based) factors. The system may use geofencing technology to enable or restrict data access based on the user's or the data requester's location. Users can set specific time windows during which certain data is accessible, and the system allows for automatic data release triggered by specific events in the user's care journey. Additionally, users can set expiration dates for data access, after which the shared data becomes inaccessible. The system can also dynamically aggregate and present data based on the spatiotemporal context of the data request, providing relevant information tailored to specific healthcare scenarios.

The spatiotemporal data release functionality is implemented through a combination of GPS technology, secure time-stamping, and a real-time rules engine. The system continuously monitors the user's location and the current time, cross-referencing this information with the user's predefined sharing rules. This embodiment of the PHDB system thus provides a highly flexible, context-aware approach to medical data sharing, offering users unprecedented control over their health information while still enabling efficient and appropriate data sharing with healthcare providers and authorized applications. This approach not only enhances patient privacy and data security but also supports more personalized and context-appropriate care delivery, setting a new standard for patient-controlled health data management in the digital age.

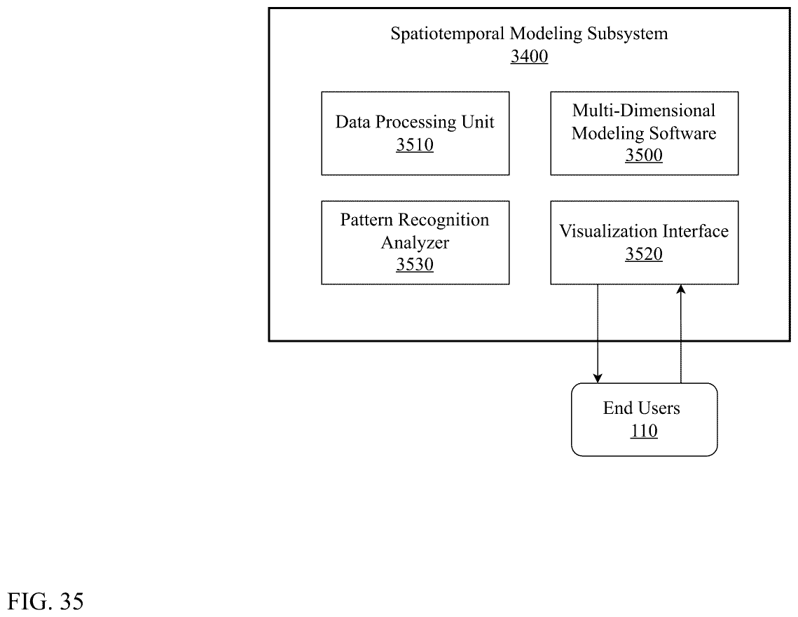

FIG. 35 is a diagram showing an exemplary subsystem of a PHDB system configured for real time spatiotemporal modeling, a spatiotemporal modeling subsystem. This subsystem comprises four key components: the Multi-Dimensional Modeling Software 3500, Data Processing Unit 3510, Visualization Interface 3520, and Pattern Recognition Analyzer 3530. These components work in concert to provide advanced spatiotemporal analysis and visualization of personal health data.

A Multi-Dimensional Modeling Software 3500 serves as the core engine for creating and manipulating 4D models of health data. It takes the diverse inputs from the PHDB system, including genomic data, microbiome information, phenotype data, and real-time sensor data from IoT devices, and constructs a comprehensive four-dimensional representation of the user's health status. This software implements advanced algorithms for spatial interpolation and temporal forecasting, allowing it to create continuous models from discrete data points across both space and time. It's capable of handling multi-scale modeling, seamlessly integrating data from the molecular level up to whole-body systems.

Working closely with the modeling software, a Data Processing Unit 3510 manages the ingestion, cleaning, and preliminary analysis of incoming data. This unit is responsible for ensuring data quality and consistency, performing necessary transformations, and preparing the data for integration into the 4D model. It handles the complex task of aligning data from various sources and timescales, from rapid physiological changes captured by wearable devices to slow-changing genomic data. The Data Processing Unit may also implement the concept of “seeds,” extracting key data points or parameter sets that serve as starting points for simulations and predictive modeling.

The Visualization Interface 3520 translates the complex 4D models into intuitive, interactive visual representations that can be easily understood by end users 110. This interface supports a range of visualization techniques, from traditional 2D graphs and 3D anatomical models to fully immersive virtual reality experiences. It allows users to navigate through their health data both spatially and temporally, zooming in on specific body regions or time periods of interest. The Visualization Interface is designed to be adaptable, capable of presenting information at various levels of complexity to suit the needs of different user groups, from patients to healthcare providers and researchers.

A Pattern Recognition Analyzer 3530 applies advanced machine learning and AI techniques to identify meaningful patterns, trends, and anomalies within the 4D health data. This component is crucial for extracting actionable insights from the vast amount of data processed by the system. It can detect subtle changes that might indicate the onset of disease, identify complex interactions between different health parameters, and predict future health trajectories based on current data and historical patterns. The Pattern Recognition Analyzer 3530 also plays a role in the system's predictive modeling capabilities, enabling it to simulate potential outcomes for different intervention strategies.

These components interact dynamically to provide a comprehensive spatiotemporal analysis of health data. The Multi-Dimensional Modeling Software 3500 continuously updates its models based on new data processed by the Data Processing Unit 3510. The Pattern Recognition Analyzer 3530 then scans these models to identify significant patterns or changes, which are subsequently visualized through the Visualization Interface 3520. This creates a feedback loop where insights generated by the Pattern Recognition Analyzer can inform further data processing and modeling strategies.

To illustrate how these components work together, consider the following example: a user of the PHDB system, has a family history of heart disease and is using the system to monitor their cardiovascular health. The user wears a smartwatch that continuously tracks their heart rate, blood pressure, and physical activity. The Data Processing Unit 3510 receives encrypted data streams from the user's smartwatch via the PHDB system's Encryption Platform 121. It cleans the data, handles any missing values, and prepares it for integration into the 4D model. The unit also processes periodic lab test results and data from the user's electronic health records.

The Multi-Dimensional Modeling Software 3500 takes this processed data and continuously updates the user's 4D health model. It creates a comprehensive spatiotemporal representation of the user's cardiovascular system, integrating real-time sensor data with her genetic information, lab results, and historical medical records. The Pattern Recognition Analyzer 3530 continuously scans the updated model, identifying trends and potential issues. For instance, it may detect a subtle but persistent increase in the user's resting heart rate over the past month, correlated with periods of elevated blood pressure.

Using the Visualization Interface 3520, the user can view their 4D cardiovascular health model. The user can see how their heart rate and blood pressure have changed over time, correlate them with her activity levels, and even zoom in on a 3D model of her heart to see how different parameters affect its function. The Pattern Recognition Analyzer 3530 may flag the concerning trend it has identified. This information is presented to the user through the Visualization Interface 3520, which shows a clear, easy-to-understand representation of the trend and its potential implications.

The user shares this information with her doctor during her next check-up. The doctor uses the Visualization Interface 3520 to explore the 4D model in more detail, gaining insights into the user's cardiovascular health that wouldn't be apparent from traditional medical records or individual test results. Based on these insights, the user and their doctor develop a proactive plan to address the concerning trends, including lifestyle modifications and closer monitoring. The PHDB system continues to track the user's progress, with the Spatiotemporal Modeling Subsystem providing ongoing analysis and visualization of her cardiovascular health.

The Spatiotemporal Modeling Subsystem 3400 interfaces with the broader PHDB system in several ways. It receives encrypted data from the Encryption Platform 121, ensuring that all operations maintain user privacy and data security. The Orchestration Computing Platform 122 manages the flow of data and commands between the Spatiotemporal Modeling Subsystem and other parts of the PHDB system, ensuring efficient and coordinated operation. The subsystem may also interacts with the Machine Learning Computing 360 component, leveraging advanced AI capabilities for pattern recognition and predictive modeling.

By integrating these sophisticated components, the Spatiotemporal Modeling Subsystem 3400 significantly enhances the capabilities of the PHDB system. It transforms static health records into dynamic, predictive models that can provide unprecedented insights into individual and population health. This system supports a wide range of applications, from personalized health management and precision medicine to large-scale epidemiological studies and health policy planning. The ability to visualize and analyze health data in four dimensions opens new avenues for understanding the complex interplay between genetics, environment, and health outcomes over time, potentially revolutionizing approaches to disease prevention, diagnosis, and treatment.

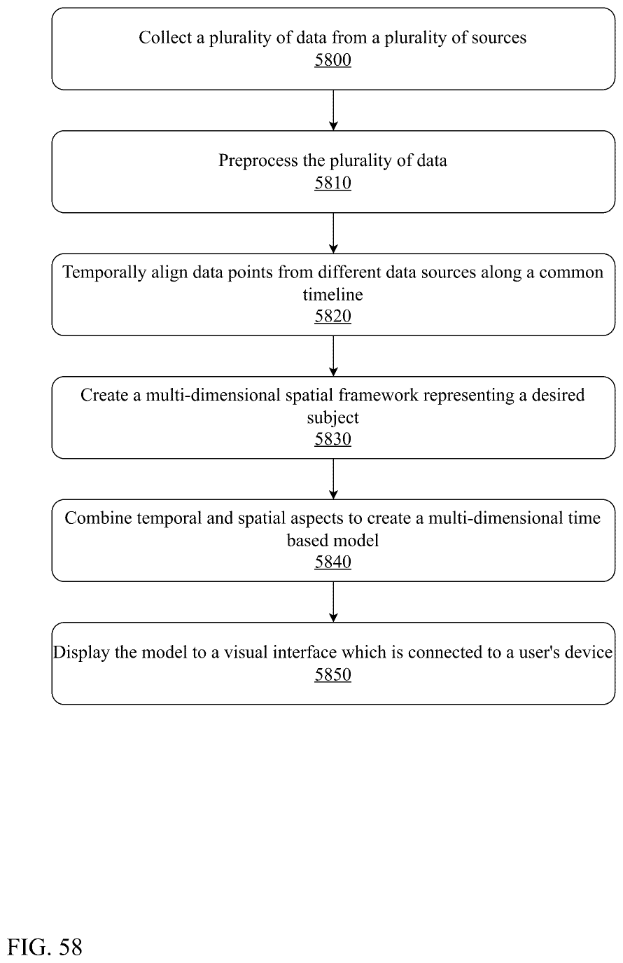

FIG. 58 is a flow diagram showing exemplary method for configuring a PHDB system for real time spatiotemporal modeling. This method leverages the capabilities of the Spatiotemporal Modeling Subsystem 3400 to provide users with a dynamic, multi-dimensional representation of their health data. In a first step 5800, the system collects a plurality of data from a plurality of sources. This step involves gathering diverse types of health-related information from various inputs connected to the PHDB system. For example, genomic data might be obtained from labs that conduct genetic testing, while real-time physiological data could come from IoT devices such as wearable fitness trackers or continuous glucose monitors. Additional data sources might include electronic health records from third-party services, manually entered information from users via PHDB mobile devices, and environmental data from external databases. This comprehensive data collection ensures that the resulting model provides a holistic view of the user's health status.

In a step 5810, the system preprocesses the plurality of data. This crucial step, primarily handled by a data processing unit, involves cleaning the raw data, handling missing values, and transforming the data into a consistent format suitable for integration into the spatiotemporal model. For instance, if a user's smartwatch fails to record heart rate data for a short period, the system might use interpolation techniques to estimate the missing values. This step also involves validating the data to ensure its accuracy and reliability. The preprocessing stage may also include the extraction of relevant features from complex data types, such as identifying specific genetic markers from genomic data or deriving activity levels from raw accelerometer data.

In a step 5820, the system temporally aligns data points from different data sources along a common timeline. This step is integral for creating a coherent temporal framework, given that different data sources often have varying sampling rates and collection times. For example, genomic data might be collected once and remain largely static, while heart rate data from a wearable device could be sampled multiple times per minute. The system must reconcile these different timescales, ensuring that all data points are correctly positioned in time relative to each other. This temporal alignment allows for accurate analysis of how different health parameters change and interact over time.

In a step 5830, the system creates a multi-dimensional spatial framework representing a desired subject. This step involves constructing a detailed spatial model of the user's body or the specific anatomical system under study. A multi-dimensional modeling software plays a role here, using advanced spatial modeling techniques to create a precise digital representation. For a full-body model, this might involve creating a detailed 3D anatomical framework. For more focused analyses, such as cardiovascular health monitoring, the spatial framework might provide a highly detailed model of the heart and circulatory system. This spatial framework serves as the foundation upon which various types of health data can be mapped.

In a step 5840, the system combines the temporal and spatial aspects to create a multi-dimensional time-based model. This step brings together the temporally aligned data from step 5820 and the spatial framework from step 5830 to construct a comprehensive 4D model. The resulting model allows for the visualization and analysis of health data across both space and time. For example, in the case of a user monitoring their recovery from a sports injury, the model might show how inflammation levels in the injured area change over time, correlated with pain levels, range of motion, and rehabilitation exercises.

In a step 5850, the system displays the model to a visual interface which is connected to a user's device. This final step leverages a visualization interface to present the complex 4D model in an intuitive, interactive format accessible to the user. The visual interface could be part of a PHDB mobile device, a personal computer, or even a virtual or augmented reality system. Users can interact with the model, zooming in on specific body areas, moving forward or backward in time, and exploring correlations between different health parameters. For instance, a user managing a chronic condition like diabetes could visualize how their blood glucose levels fluctuate throughout their body over time, correlating these changes with factors like diet, exercise, and medication.

Throughout this process, a pattern recognition analyzer continuously scans the developing model, identifying significant patterns, trends, or anomalies. These insights are incorporated into the visual display, helping to draw the user's attention to important aspects of their health data. This method enables the PHDB system to transform diverse, complex health data into a coherent, informative, and interactive spatiotemporal model. By providing users and healthcare providers with this comprehensive view of health data across both time and space, the system supports more informed decision-making, personalized treatment planning, and proactive health management.

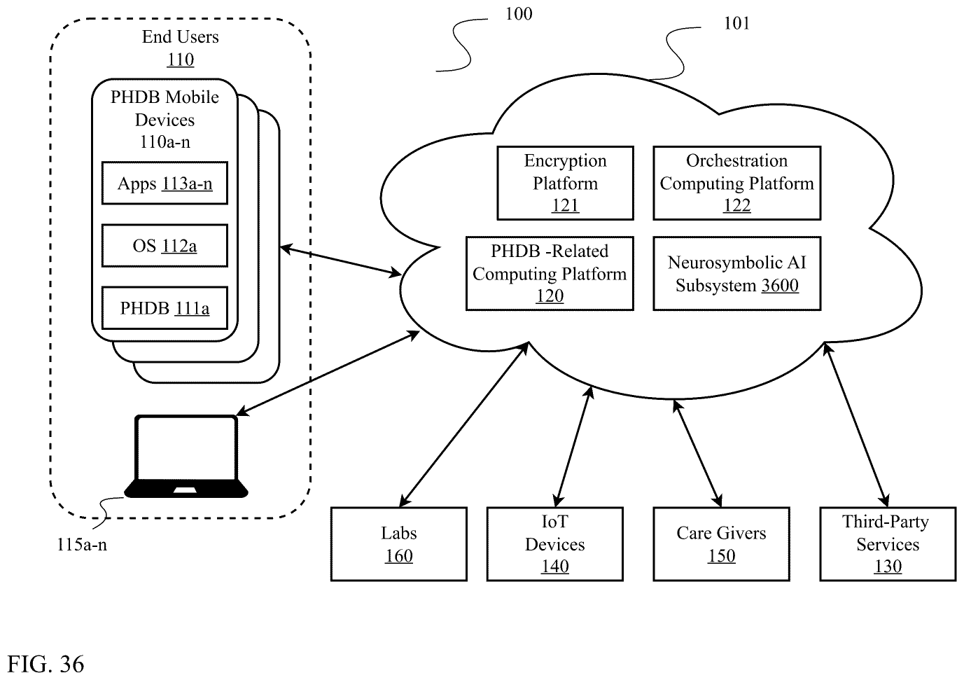

FIG. 36 is a block diagram showing an exemplary arrangement of a PHDB, where the PHDB system includes a neurosymbolic AI subsystem 3600. This addition represents a significant advancement in the system's ability to process, analyze, and derive insights from personal health data. Neurosymbolic AI subsystem 3600 seamlessly integrates with the existing PHDB-related computing platform 120, working in conjunction with other critical components such as the encryption platform 121 and the orchestration computing platform 122. This subsystem combines the strengths of neural networks and symbolic AI to provide more comprehensive, interpretable, and context-aware insights from the diverse range of personal health data collected by the PHDB system.

At its core, neurosymbolic AI subsystem 3600 processes data from various sources within the PHDB ecosystem. It receives input from PHDB mobile devices 110a-n, which include user applications apps 113a-n, the device's operating system OS 112a, and the local PHDB storage 111a. This allows the AI to analyze real-time data from users, including information manually entered through apps and data collected by device sensors.

The subsystem also interfaces with data from IoT devices 140, which might include wearable fitness trackers, smart scales, or home health monitoring equipment. This real-time data stream provides crucial information about users' daily activities, vital signs, and environmental factors affecting their health. Neurosymbolic AI subsystem 3600 can process information from labs 160, third-party services 130, and care givers 150. This might include genetic test results, electronic health records, or notes from healthcare providers. By integrating these diverse data sources, the AI can form a comprehensive view of a user's health status and history.

The neurosymbolic approach of this subsystem allows it to leverage both data-driven learning and rule-based reasoning. The neural network components excel at pattern recognition and handling unstructured data, such as identifying trends in a user's blood pressure readings or recognizing potential early signs of disease from medical imaging. Meanwhile, the symbolic AI components incorporate medical knowledge and logical rules, allowing for deductive reasoning based on established healthcare guidelines and protocols.

For example, consider a scenario where a user with type 2 diabetes is using the PHDB system to manage their condition. Neurosymbolic AI subsystem 3600 might analyze continuous glucose monitor data from an IoT device 140, physical activity levels from a fitness tracker, and dietary information entered through a PHDB mobile device app 113a-n. The neural network components could identify subtle patterns in how different foods and activities affect the user's glucose levels. Simultaneously, the symbolic AI components could apply rules about diabetes management, considering factors like the user's medication regimen, age, and other health conditions. By integrating these insights, the system could provide highly personalized recommendations for meal planning and physical activity to optimize glucose control.

One of the key benefits of this neurosymbolic approach is its ability to provide explainable AI-driven insights. Unlike pure neural network models that often function as “black boxes,” the neurosymbolic AI can offer clear explanations for its recommendations, tracing the logic back to both learned patterns and established medical knowledge. This transparency is crucial in healthcare applications, where understanding the reasoning behind AI-generated insights is essential for both users and healthcare providers.

Neurosymbolic AI subsystem 3600 also enhances the PHDB system's ability to adapt to new information. As new medical research becomes available, it can be incorporated into the symbolic reasoning components, allowing the system to quickly adjust its recommendations based on the latest evidence. At the same time, the neural network components continue to learn from individual user data, ensuring that insights remain personalized and relevant.

Security and privacy are paramount in handling sensitive health data. Neurosymbolic AI subsystem 3600 works in tandem with the encryption platform 121 to ensure that all data processing and analysis occur on encrypted data, maintaining user privacy and data security throughout the AI operations. The orchestration computing platform 122 plays a crucial role in managing the interactions between neurosymbolic AI subsystem 3600 and other components of the PHDB system. It ensures efficient data flow, coordinates processing tasks, and manages the distribution of AI-generated insights to relevant parts of the system, including back to users' PHDB mobile devices 110a-n or to healthcare providers via secure channels.

By integrating this advanced neurosymbolic AI subsystem 3600, the PHDB system significantly enhances its capabilities in personal health management. It can provide users with more accurate, explainable, and actionable health insights, supporting both day-to-day health management and complex medical decision-making. This approach has the potential to improve health outcomes, increase user engagement with their personal health data, and facilitate more effective communication between patients and healthcare providers.

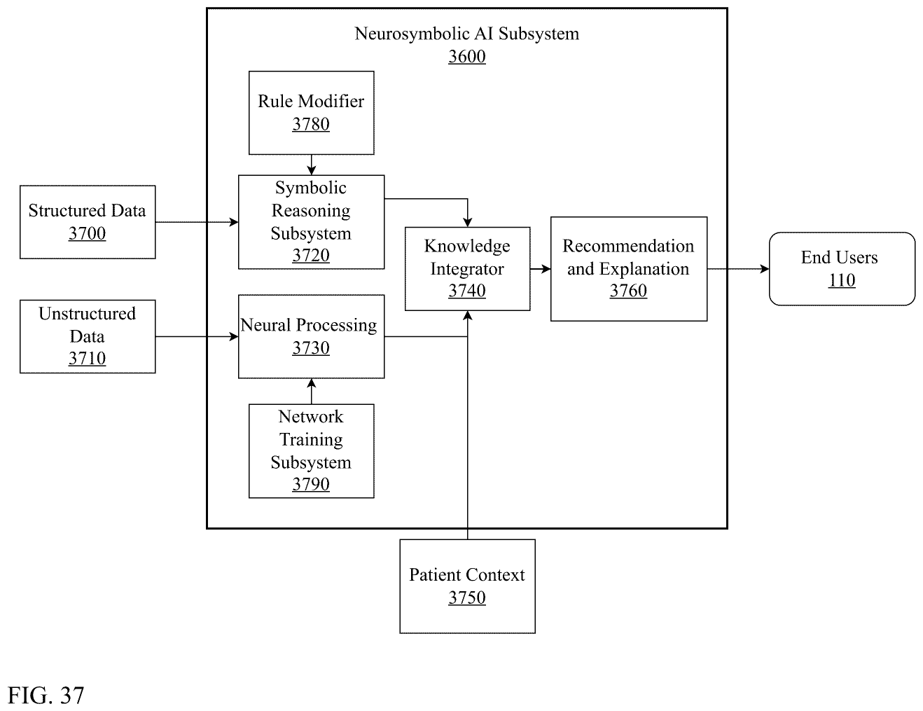

FIG. 37 is a diagram showing an exemplary subsystem of a PHDB system including a neurosymbolic A1 subsystem. Neurosymbolic AI is an approach that combines neural networks' pattern recognition capabilities with symbolic A1's logical reasoning, aiming to create more robust, interpretable, and adaptable artificial intelligence systems. The neurosymbolic AI subsystem begins with two types of input: structured data 3700 and unstructured data 3710. Structured data might include numerical health metrics, lab results, or clearly formatted electronic health records. Unstructured data could encompass free-text clinical notes, medical images, or raw sensor data from wearable devices.

A neural processing component 3730 primarily handles the unstructured data, leveraging deep learning techniques to extract meaningful features and patterns. For instance, it might process natural language in clinical notes to identify key health indicators or analyze medical images to detect potential abnormalities. This component's strength lies in its ability to learn complex patterns from large datasets without explicit programming. A symbolic reasoning subsystem 3720 operates on structured data and applies logical rules and domain knowledge to make inferences. This component embodies the system's ability to reason using established medical knowledge, guidelines, and protocols. For example, it might apply rules about drug interactions or use decision trees for diagnostic reasoning.

A knowledge integrator 3740 serves as the bridge between neural processing and symbolic reasoning. It combines insights from both approaches, allowing the system to leverage data-driven patterns and logical deductions simultaneously. This integration is key to the neurosymbolic approach, enabling more comprehensive and robust analysis than either method could achieve alone. Patient context 3750 ensures that all analyses and recommendations are tailored to the individual user's specific circumstances. It considers factors like the patient's medical history, lifestyle, preferences, and environmental factors, allowing for highly personalized insights. The recommendation and explanation component 3760 generates user-facing outputs. A crucial feature of neurosymbolic AI is its ability to provide explainable recommendations. Unlike “black box” neural networks, this system can trace its reasoning, showing how it arrived at a particular conclusion or recommendation by combining learned patterns and logical rules.

In some instances, new information comes to light which needs to be introduced to the AI subsystem to ensure results are as up to date as possible. A rule modifier 3780 allows the system to update its symbolic reasoning based on new information or patterns detected by the neural processing component. This adaptive capability ensures that the system can evolve its reasoning as it encounters new data or as medical knowledge advances. A network training subsystem 3790 continuously refines the neural processing component based on new data and feedback, ensuring that the system's pattern recognition capabilities improve over time.

This neurosymbolic approach offers several advantages in the context of personal health management. It can handle the complexity and variability of health data more effectively than traditional AI approaches. For instance, in managing a chronic condition like diabetes, the system can integrate data-driven insights about an individual's glucose responses to various foods with established nutritional guidelines and the user's personal preferences. This results in highly personalized dietary recommendations that are both medically sound and practical for the user to implement.

Moreover, the neurosymbolic AI subsystem enhances the PHDB system's ability to adapt to new information. As new medical research becomes available, it can be incorporated into the symbolic reasoning subsystem, allowing the system to quickly adjust its recommendations based on the latest evidence. Simultaneously, the neural processing component continues to learn from individual user data, ensuring that insights remain personalized and relevant. The system's ability to provide explainable AI is particularly crucial in healthcare applications. For example, if the system recommends a change in treatment for a user with hypertension, it can explain this recommendation by citing both the patterns it has observed in the user's blood pressure data (from neural processing) and the relevant medical guidelines it has applied (from symbolic reasoning).

By incorporating this neurosymbolic AI subsystem, the PHDB system can offer more nuanced, context-aware, and personalized health insights. It can handle complex, multi-faceted health scenarios, provide transparent and explainable recommendations, and adapt more effectively to new information and individual user needs. This approach represents a significant advancement in applying AI to personal health management, potentially improving health outcomes and enhancing user engagement with their personal health data.

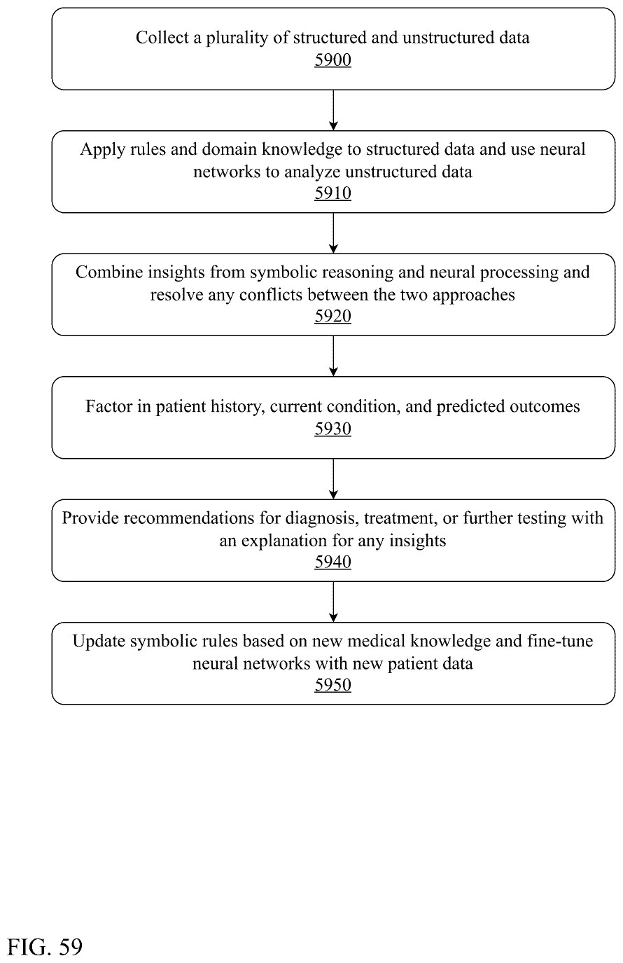

FIG. 59 is a flow diagram showing exemplary method for configuring a PHDB system with a neurosymbolic AI system. In a step 5900, a neurosymbolic AI subsystem collects a plurality of structured and unstructured data. This step involves gathering diverse types of health-related information from various sources within the PHDB ecosystem. Structured data might include numerical health metrics, lab results, or clearly formatted electronic health records from sources such as labs or third-party services. Unstructured data could encompass free-text clinical notes from care givers, medical images from diagnostic procedures, or raw sensor data from IoT devices and PHDB mobile devices. For example, structured data might include a patient's blood glucose levels over time, while unstructured data could be the text of a doctor's notes describing the patient's symptoms.

In a step 5910, the system applies rules and domain knowledge to structured data and uses neural networks to analyze unstructured data. This step leverages the strengths of both symbolic AI and neural networks. A symbolic reasoning subsystem operates on the structured data, applying logical rules and medical knowledge to make inferences. For instance, it might use established guidelines to assess whether a patient's cholesterol levels indicate a high risk of heart disease. Simultaneously, a neural processing component handles the unstructured data, using deep learning techniques to extract meaningful features and patterns. This could involve using natural language processing to identify key health indicators in clinical notes or applying computer vision algorithms to detect anomalies in medical images.

In a step 5920, the system combines insights from symbolic reasoning and neural processing and resolves any conflicts between the two approaches. This step is performed by a knowledge integrator, which serves as a bridge between the symbolic and neural components. It might, for example, reconcile a rule-based conclusion that a patient is at low risk for a certain condition with a pattern detected by the neural network suggesting otherwise. In resolving such conflicts, the system might prioritize one insight over the other based on confidence levels, or it might flag the discrepancy for human review.

In a step 5930, the system factors in patient history, current condition, and predicted outcomes. This step ensures that all analyses and recommendations are tailored to the individual user's specific circumstances. The patient context component (3750) plays a key role here, considering factors like the patient's medical history, lifestyle, preferences, and environmental factors. For instance, when assessing the risk of type 2 diabetes, the system would consider not just current blood sugar levels, but also family history, dietary habits, physical activity levels, and other relevant factors stored in the user's PHDB.

In a step 5940, the system provides recommendations for diagnosis, treatment, or further testing with an explanation for any insights. This step generates user-facing outputs that are both actionable and transparent. For example, if the system recommends that a user with persistent headaches should undergo further neurological testing, it would explain this recommendation by citing both the pattern of symptoms it has detected (from neural processing) and the relevant diagnostic guidelines it has applied (from symbolic reasoning). This explainability is a key advantage of the neurosymbolic approach, allowing users and healthcare providers to understand and trust the AI's recommendations.

In a step 5950, the system updates symbolic rules based on new medical knowledge and fine-tunes neural networks with new patient data. This final step ensures that the neurosymbolic AI subsystem remains current and continues to improve over time. A rule modifier updates the symbolic reasoning component with new medical knowledge, such as updated treatment guidelines or newly discovered drug interactions. Meanwhile, the network training subsystem refines the neural processing component based on new data and feedback, improving its pattern recognition capabilities. For instance, if a new study reveals a previously unknown risk factor for heart disease, this information could be incorporated into the symbolic rules. At the same time, as the system processes more user data, its neural networks might become better at detecting subtle patterns predictive of heart disease.

This method enables the PHDB system to provide highly personalized, context-aware, and explainable health insights. By combining the strengths of symbolic AI and neural networks, it can handle complex health scenarios, adapt to new information, and offer transparent recommendations. This approach represents a significant advancement in applying AI to personal health management, potentially improving health outcomes and enhancing user engagement with their personal health data.

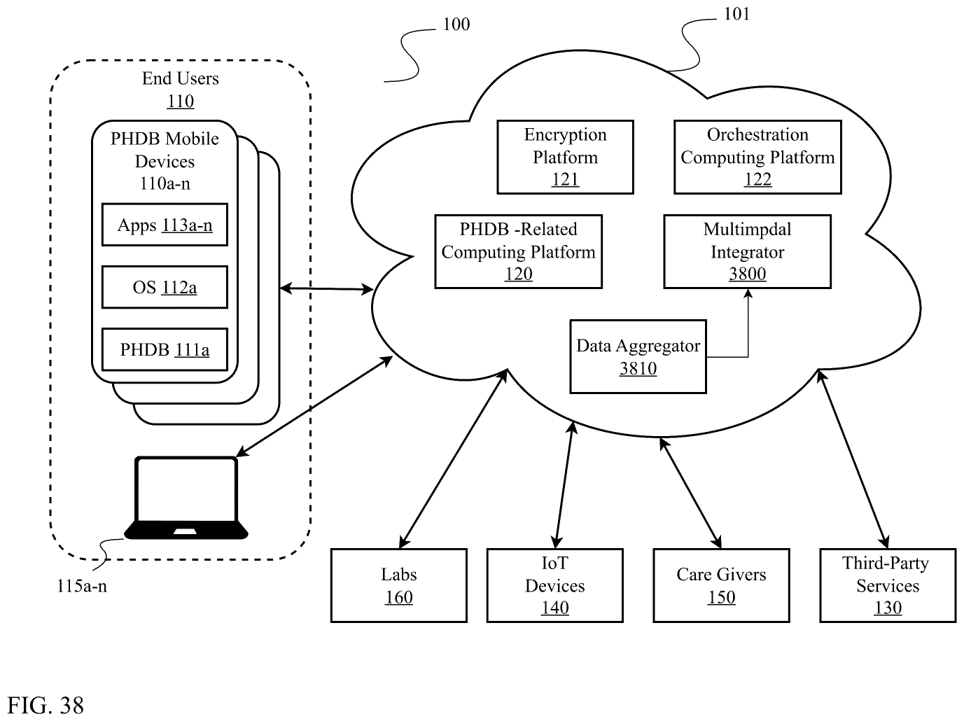

FIG. 38 is a block diagram showing an exemplary arrangement of a PHDB, where the PHDB system is configured for multimodal integration. In one embodiment, an enhanced PHDB system may incorporate a multimodal integrator 3800 and a data aggregator 3810 as key components within the cloud-based platforms 101. These additions represent a significant advancement in the system's ability to process, integrate, and analyze diverse types of personal health data.

A multimodal integrator 3800 is designed to combine and analyze data from various sources and modalities within the PHDB ecosystem. It interfaces with the PHDB-related computing platform 120, working in conjunction with other critical components such as the encryption platform 121 and the orchestration computing platform 122. This subsystem is capable of processing and integrating diverse data types, including structured data (like numerical health metrics and lab results), unstructured data (such as clinical notes and medical images), and real-time data streams from IoT devices 140.

A data aggregator 3810 works closely with the multimodal integrator, collecting and consolidating data from various sources within the PHDB system. It gathers information from PHDB mobile devices 110a-n, which include user applications apps 113a-n, the device's operating system OS 112a, and the local PHDB storage 111a. This allows the system to incorporate user-inputted data, device sensor readings, and locally stored health information.

Multimodal integrator 3800 and data aggregator 3810 also interface with data from labs 160, third-party services 130, and care givers 150. This might include genetic test results, electronic health records, or notes from healthcare providers. By consolidating and integrating these diverse data sources, the system can form a comprehensive view of a user's health status and history. One of the key strengths of this multimodal integration approach is its ability to handle complex, multifaceted health data. For example, in managing a chronic condition like diabetes, the system can integrate blood glucose readings from a continuous glucose monitor (an IoT device), dietary information logged through a mobile app, physical activity data from a fitness tracker, lab results showing HbA1c levels, and clinical notes from a healthcare provider. The multimodal integrator can analyze these diverse data types together, identifying correlations and patterns that might not be apparent when looking at each data source in isolation.

Multimodal integrator 3800 may employ a plurality of algorithms to align and synchronize data from different sources, which often have varying sampling rates and collection times. For instance, it might need to align high-frequency data from a heart rate monitor with less frequent blood pressure measurements and occasional lab test results. This temporal alignment is crucial for accurate analysis and interpretation of the data.

Furthermore, multimodal integrator 3800 can perform sophisticated analyses that leverage the strengths of different data types. For example, it might combine computer vision techniques to analyze medical images with natural language processing to interpret clinical notes, providing a more comprehensive understanding of a patient's condition than either data type could offer alone. Data aggregator 3810 plays a role in ensuring data quality and consistency. It can handle tasks such as de-duplication of data, resolution of conflicting information from different sources, and standardization of data formats. This is particularly important given the diverse range of data sources and formats in the PHDB ecosystem.

Security and privacy remain paramount in this system. Multimodal integrator 3800 and data aggregator 3810 work in tandem with encryption platform 121 to ensure that all data processing and analysis occur on encrypted data, maintaining user privacy and data security throughout the integration and analysis process.

Orchestration computing platform 122 manages the interactions between the multimodal integrator, data aggregator, and other components of the PHDB system. It ensures efficient data flow, coordinates processing tasks, and manages the distribution of integrated insights to relevant parts of the system, including back to users' PHDB mobile devices 110a-n or to healthcare providers via secure channels. By incorporating this advanced multimodal integration capability, the PHDB system significantly enhances its ability to provide comprehensive, nuanced insights into personal health. It can offer users a more holistic view of their health status, identify complex patterns and correlations across different aspects of health and lifestyle, and support more informed decision-making by both users and healthcare providers.

For instance, the system might detect a correlation between a user's sleep patterns (tracked by a wearable device), their stress levels (inferred from heart rate variability and app-logged mood data), and their blood pressure readings. By integrating and analyzing these diverse data streams, the system could provide insights and recommendations that address the interconnected nature of these health factors, potentially leading to more effective interventions and improved health outcomes. This multimodal integration approach represents a significant advancement in personal health management, enabling more comprehensive, personalized, and actionable health insights. It has the potential to improve health outcomes, increase user engagement with their personal health data, and facilitate more effective communication between patients and healthcare providers.

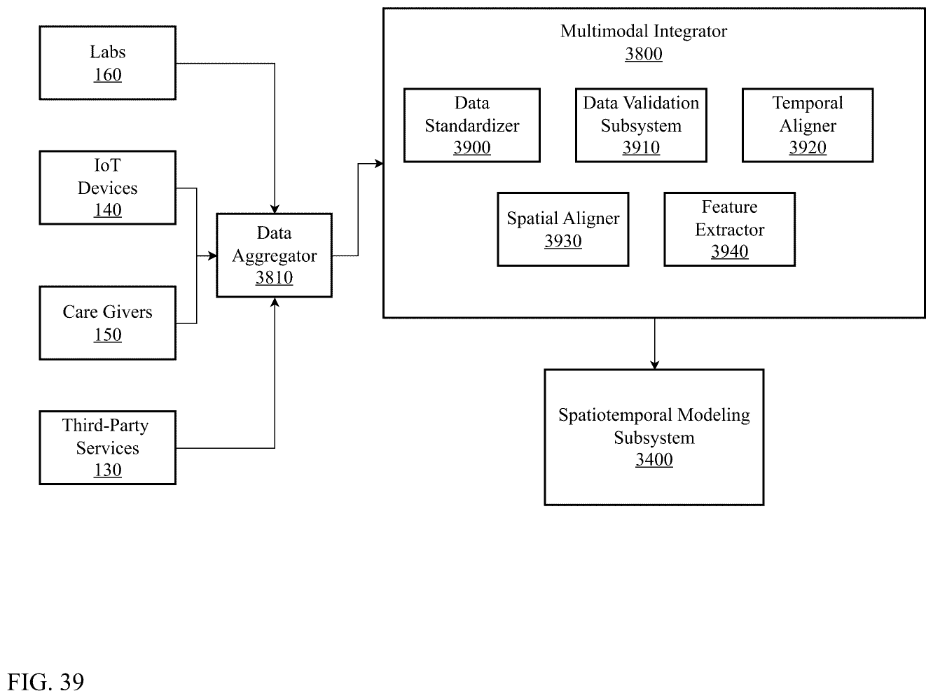

FIG. 39 is a diagram showing an exemplary subsystem of a PHDB system configured for multimodal integration, a multimodal integrator. Multimodal integrator 3800 begins with input from various sources, including labs 160, third-party services 130, care givers 150, and IoT devices 140. These diverse data streams are first collected and consolidated by data aggregator 3810, which serves as the initial point of contact for incoming information.

Once aggregated, the data passes through data standardizer 3900. This component plays a crucial role in harmonizing the various data formats and structures that come from different sources. For example, it might convert all date formats to a standard format, normalize units of measurement, or transform proprietary data structures into a common schema used within the PHDB system. This standardization is essential for enabling seamless integration and analysis of data from disparate sources.

The standardized data then moves to data validation subsystem 3910. This component performs critical quality checks to ensure the integrity and reliability of the data. It might flag outliers, detect missing values, and identify potential inconsistencies or errors in the data. For instance, if a blood pressure reading seems implausibly high or low, this subsystem would mark it for further review or potential correction.

After validation, the data proceeds to temporal aligner 3920 and spatial aligner 3930 components. The temporal aligner is responsible for synchronizing data points from different sources along a common timeline. This is particularly important when dealing with data that has varying sampling rates or collection times. For example, it might align high-frequency heart rate data from a wearable device with less frequent blood pressure measurements and occasional lab test results.

Spatial aligner 3930 ensures that data is correctly mapped to specific anatomical locations or physiological systems within the body. This is crucial for creating a coherent spatial representation of health data. For instance, it might map imaging data to specific organs or body regions, or associate sensor data from wearable devices with the appropriate body parts. Feature extractor 3940 then processes the aligned data to identify and extract relevant features or patterns. This component employs advanced algorithms, potentially including machine learning techniques, to distill key information from the complex, multimodal data. For example, it might extract features from ECG waveforms that are indicative of certain heart conditions, or identify patterns in movement data that suggest changes in gait or balance.

Finally, the processed and feature-rich data is passed to spatiotemporal modeling subsystem 3400. This subsystem, which may be integrated into a PHDB system, creates a comprehensive 4D model of the user's health, integrating both spatial and temporal aspects of the data.

The multimodal integrator's approach allows for sophisticated analyses that leverage the strengths of different data types. For instance, it can combine numerical data from lab tests, textual data from clinical notes, imaging data from diagnostic procedures, and continuous streams of sensor data from wearable devices to create a holistic view of a patient's health status. This system's ability to handle diverse data types and create a unified, spatiotemporal health model aligns well with the principles of neurosymbolic AI discussed in the new disclosure. While not explicitly a neurosymbolic AI system itself, the multimodal integrator provides the rich, integrated data that a neurosymbolic AI system could leverage for more advanced analysis and decision support.

For example, the standardized, validated, and aligned data from the multimodal integrator could serve as input for both the neural and symbolic components of a neurosymbolic AI system. The neural networks could process the unstructured elements like medical images or text from clinical notes, while the symbolic reasoning components could apply rules and domain knowledge to the structured elements like lab results or standardized diagnostic codes. Moreover, the feature extraction capabilities of the multimodal integrator could provide valuable input for the knowledge integrator component of a neurosymbolic AI system, helping to bridge the gap between data-driven insights and rule-based reasoning.

In practice, this system could enable highly sophisticated health analyses. For instance, it could integrate a patient's genetic data, longitudinal lab results, real-time glucose monitoring data, dietary logs, and physical activity data to provide a comprehensive view of their diabetes management. The system could identify subtle patterns in glucose fluctuations, correlate these with dietary choices and physical activity, and consider genetic factors that might influence treatment response. This integrated view could then inform personalized treatment recommendations, taking into account the full complexity of the patient's health status and lifestyle.

By providing this level of data integration and preprocessing, the multimodal integrator sets the stage for advanced AI analyses, including neurosymbolic approaches, that can generate more accurate, comprehensive, and personalized health insights. This represents a significant advancement in the application of AI to personal health management, potentially leading to improved health outcomes and more effective, personalized healthcare strategies.

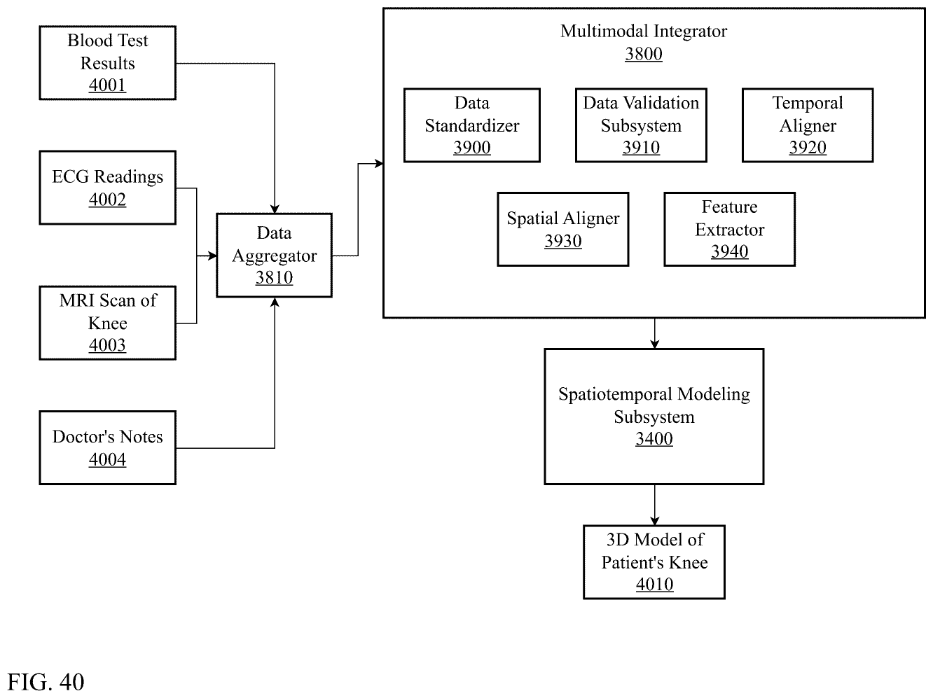

FIG. 40 is a diagram showing an exemplary embodiment of a PHDB system configured for multimodal integration, wherein the multimodal integrator generates a 3D model from incoming data. Illustrated is a practical use case of multimodal integrator 3800 within the PHDB system, demonstrating how diverse types of medical data are integrated to create a comprehensive 3D model of a patient's knee. This example showcases the system's ability to synthesize information from various sources into a coherent, spatially accurate representation. The process begins with the input of four distinct types of medical data: blood test results 4001, ECG readings 4002, an MRI scan of the knee 4003, and doctor's notes 4004. Each of these data types provides unique and valuable information about the patient's health status, particularly in relation to their knee condition.

Data aggregator 3810 serves as the initial point of contact for these diverse data streams. It collects and consolidates the information, preparing it for further processing. This step is crucial for ensuring that all relevant data is gathered in one place, regardless of its source or format. Next, the aggregated data passes through data standardizer 3900. This component plays a vital role in harmonizing the various data formats. For instance, it might convert date formats in the blood test results and doctor's notes to a standard format, ensure that all measurements in the MRI data use consistent units, and transform the ECG data into a standardized waveform representation.

The standardized data then moves to data validation subsystem 3910. This component performs critical quality checks to ensure the integrity and reliability of the data. It might flag any outliers in the blood test results, verify the completeness of the MRI scan data, and check for any inconsistencies between the doctor's notes and the other data sources. After validation, temporal aligner 3920 synchronizes the data points along a common timeline. This is particularly important for integrating the ECG readings, which might be collected over time, with the more static MRI scan data and the periodic blood test results. The temporal alignment allows for a dynamic understanding of how the patient's condition may have changed over time.

Spatial aligner 3930 then ensures that all the data is correctly mapped to the appropriate anatomical locations. This is crucial for creating an accurate 3D model of the knee. It would align the MRI scan data with a standard anatomical model, ensuring that structures like ligaments, cartilage, and bones are correctly positioned. Feature extractor 3940 processes the aligned data to identify and extract relevant features. For the knee model, this might involve identifying specific structures in the MRI scan, extracting relevant biomarkers from the blood test results, and identifying any mentions of knee-related symptoms or observations in the doctor's notes.

Finally, all the processed and feature-rich data is passed to spatiotemporal modeling subsystem 3400. This subsystem integrates all the information to create a comprehensive 3D model of the patient's knee 4010. The model would incorporate the structural information from the MRI scan, potentially color-coded or annotated based on information from the blood tests (e.g., highlighting areas of inflammation), and include markers or annotations based on the doctor's notes. This 3D knee model 4010 represents a powerful synthesis of the input data. It could show the current state of the knee joint, including any structural abnormalities visible in the MRI, areas of inflammation or damage indicated by blood markers, and specific points of concern noted by the doctor. The incorporation of ECG data might seem less directly relevant, but could be valuable for understanding the patient's overall health status or fitness level, which could impact treatment decisions for a knee condition.

The resulting 3D model serves as a comprehensive, visual representation of the patient's knee health. It could be used by healthcare providers to make more informed diagnostic and treatment decisions, or by the patient themselves to better understand their condition. For example, a surgeon could use this model to plan a knee surgery, taking into account not just the structural information from the MRI, but also the patient's overall health status as indicated by the blood tests and ECG.

This use case demonstrates the power of multimodal integration in creating a holistic view of a patient's health. By combining diverse data types—imaging, laboratory results, continuous monitoring data, and clinical observations—the system provides a more comprehensive and nuanced understanding than any single data source could offer alone. This integrated approach aligns with the principles of neurosymbolic AI, providing rich, contextualized data that could serve as input for more advanced AI analyses and decision support systems in healthcare.

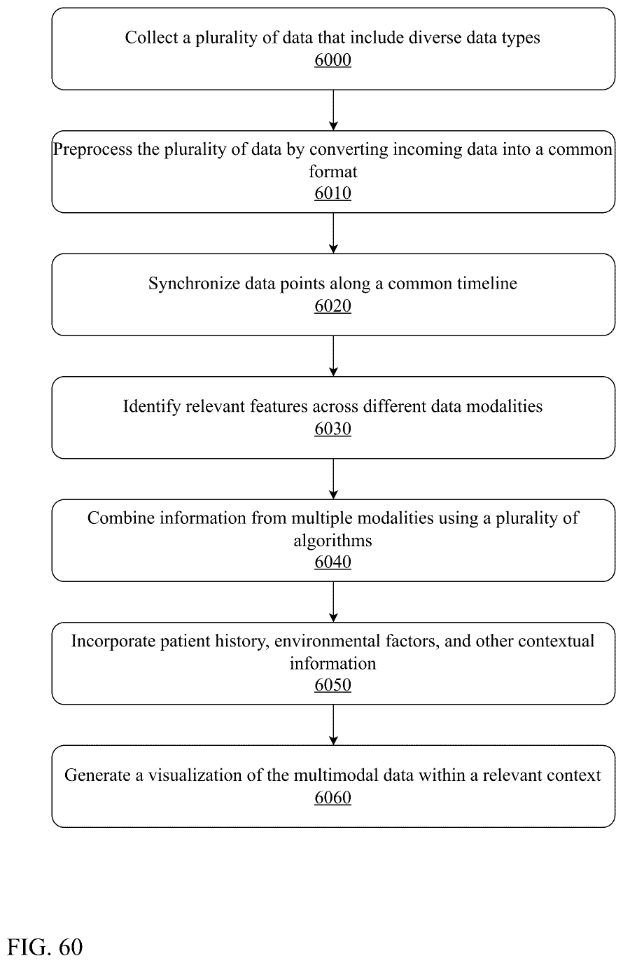

FIG. 60 is a flow diagram showing exemplary method for configuring a PHDB system with multimodal integration capabilities. In a first step 6000, the system collects a plurality of data that include diverse data types. This step involves gathering a wide range of health-related information from various sources within the PHDB ecosystem. For example, it might collect structured data like blood test results from labs, unstructured data like clinical notes from care givers, real-time sensor data from IoT devices such as wearable fitness trackers, and electronic health records from third-party services. A practical example could be collecting a user's genetic test results, daily blood glucose readings, physical activity data from a smartwatch, and their doctor's notes from recent check-ups.

In a step 6010, the system preprocesses the plurality of data by converting incoming data into a common format. A data standardizer ensures that all data, regardless of its source, is in a consistent format that can be easily integrated and analyzed. For instance, it might convert all date formats to a standard ISO format, normalize units of measurement (e.g., converting all weight measurements to kilograms), or transform proprietary data structures into a common schema used within the PHDB system. This standardization is essential for enabling seamless integration and analysis of data from disparate sources.

In a step 6020, the system synchronizes data points along a common timeline. This step, carried out by a temporal aligner, creates a coherent temporal framework for analysis. It involves aligning data with different sampling rates and collection times. For example, it might need to align high-frequency heart rate data collected every second from a wearable device, with daily weight measurements, weekly blood pressure readings, and monthly lab test results. This temporal alignment allows for accurate analysis of how different health parameters change and interact over time.

In a step 6030, the system identifies relevant features across different data modalities. A feature extractor uses advanced algorithms, potentially including machine learning techniques, to distill key information from the complex, multimodal data. For instance, it might extract features from ECG waveforms that are indicative of certain heart conditions, identify patterns in movement data that suggest changes in gait or balance, or derive mood indicators from the text of journal entries in a mental health app.

In a step 6040, the system combines information from multiple modalities using a plurality of algorithms. This step is at the core of the multimodal integration process. It might use techniques like sensor fusion to combine data from multiple IoT devices, apply natural language processing to extract insights from text-based data, and use machine learning algorithms to identify correlations between different data types. For example, it could combine blood glucose readings, dietary logs, physical activity data, and sleep patterns to provide a comprehensive view of factors affecting a diabetic patient's glucose control.

In a step 6050, the system incorporates patient history, environmental factors, and other contextual information. This step ensures that the integrated data is interpreted within the context of the individual user's specific circumstances. It might consider factors like the patient's medical history, known allergies, lifestyle factors, and even local environmental data like air quality or pollen counts. For instance, when analyzing a user's respiratory health data, the system might factor in their history of asthma, current medication regimen, local air quality data, and recent physical activity levels.

In a step 6060, the system generates a visualization of the multimodal data within a relevant context. This final step transforms the complex, integrated data into an intuitive, visual format that can be easily understood by users and healthcare providers. The visualization might take various forms depending on the nature of the data and the intended audience. For example, it could create a timeline showing the progression of a chronic condition alongside treatment changes and lifestyle factors, or a body map showing how different health parameters vary across different body systems. For a patient managing hypertension, it might generate a dashboard showing blood pressure trends over time, correlated with medication adherence, stress levels (inferred from heart rate variability), and dietary sodium intake.

This method enables the PHDB system to transform diverse, complex health data into a coherent, informative, and actionable format. By collecting, standardizing, aligning, and integrating data from multiple sources and modalities, it provides a comprehensive view of an individual's health status. This integrated approach supports more informed decision-making by both users and healthcare providers, potentially leading to more personalized and effective health management strategies. The system's ability to handle diverse data types and create unified, contextualized health models aligns well with the principles of neurosymbolic AI, setting the stage for more advanced, explainable AI analyses in healthcare.

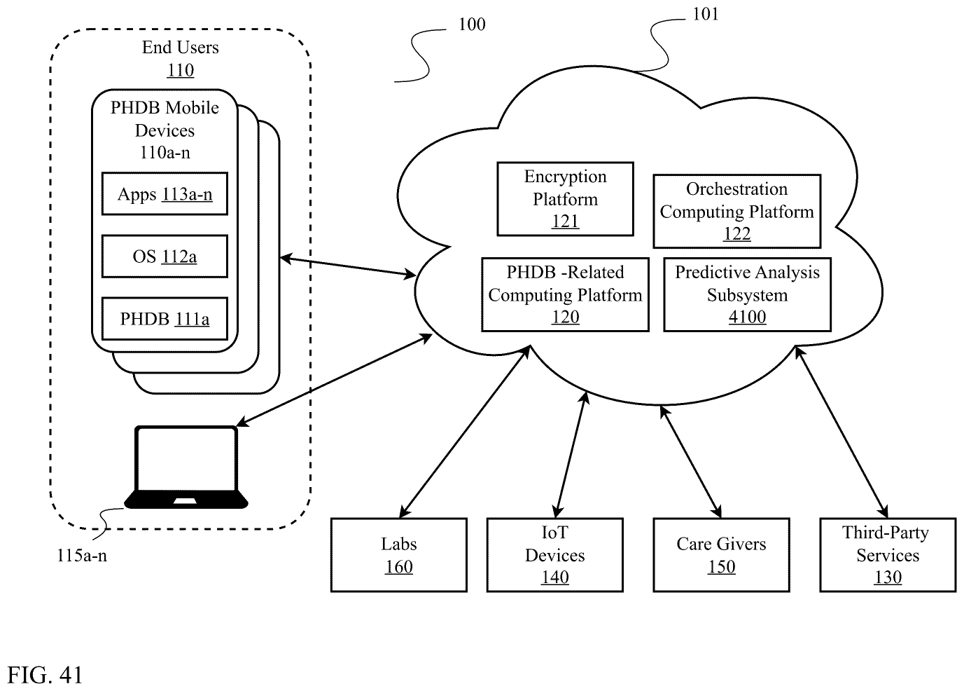

FIG. 41 is a block diagram showing an exemplary arrangement of a PHDB, where the PHDB system is configured for predictive analysis. Illustrated is an enhanced PHDB system that incorporates a predictive analysis subsystem 4100 as a key component within the cloud-based platforms 101. This addition represents a significant advancement in the system's ability to forecast health trends, anticipate potential health issues, and provide proactive recommendations for users.

Predictive analysis subsystem 4100 integrates seamlessly with the existing PHDB-related computing platform 120, working in conjunction with other critical components such as the encryption platform 121 and the orchestration computing platform 122. This subsystem is designed to analyze the vast amount of data collected and processed by the PHDB system to generate forward-looking insights and predictions about users' health.

Predictive analysis subsystem 4100 leverages data from various sources within the PHDB ecosystem. It processes information from PHBD mobile devices 110a-n, which include user applications apps 113a-n, the device's operating system OS 112a, and the local PHDB storage 111a. This allows the system to incorporate real-time and historical data directly from users, including manually entered information, device sensor readings, and locally stored health records. Furthermore, the predictive analysis subsystem interfaces with data from IoT devices 140, labs 160, third-party services 130, and care givers 150. This diverse range of data sources enables the system to create comprehensive predictive models that consider a wide array of factors influencing a user's health.

Predictive analysis subsystem 4100 may employ a plurality of machine learning algorithms and statistical modeling techniques to identify patterns, trends, and potential risk factors in users' health data. It can analyze both structured data (like numerical health metrics and lab results) and unstructured data (such as clinical notes and lifestyle information) to generate predictions. For example, the system might analyze a diabetic user's historical blood glucose levels, dietary habits, physical activity patterns, and medication adherence to predict future glucose trends and potential risks of complications. It could then generate personalized recommendations for lifestyle changes or medication adjustments to help prevent these predicted issues.

The predictive capabilities of this subsystem extend beyond individual health metrics to encompass broader health trends and potential disease risks. By analyzing genetic data, family history, lifestyle factors, and environmental data, the system can assess a user's risk for developing various health conditions over time. This could enable early interventions and preventive measures, potentially improving long-term health outcomes. Security and privacy remain paramount in this enhanced system. The predictive analysis subsystem works in tandem with the encryption platform 121 to ensure that all data processing and analysis occur on encrypted data, maintaining user privacy and data security throughout the predictive modeling process.

Orchestration computing platform 122 plays a role in managing the interactions between the predictive analysis subsystem and other components of the PHDB system. It ensures efficient data flow, coordinates processing tasks, and manages the distribution of predictive insights to relevant parts of the system, including back to users' PHDB mobile devices 110a-n or to healthcare providers via secure channels.

One of the key strengths of this predictive analysis approach is its ability to provide personalized, actionable insights to users. Rather than generic health advice, users can receive highly tailored recommendations based on their unique health profile, lifestyle, and predicted health trajectories. For instance, the system might predict an increased risk of heart disease based on a user's family history, current health metrics, and lifestyle factors, and then provide specific, personalized recommendations for diet, exercise, and preventive screenings.

Moreover, the predictive analysis subsystem can support healthcare providers in making more informed decisions about patient care. By providing predictions about potential health risks or treatment outcomes, it can help doctors develop more effective, personalized treatment plans. The integration of this predictive analysis capability represents a significant step towards proactive, preventive healthcare. By anticipating potential health issues before they become serious, the system can help users take timely action to maintain or improve their health. This approach has the potential to not only improve individual health outcomes but also to reduce the overall burden on healthcare systems by preventing or mitigating serious health conditions before they develop.

In summary, the addition of predictive analysis subsystem 4100 to the PHDB system marks a major advancement in personal health management. By leveraging the vast amount of diverse health data collected by the system, it enables more accurate, personalized, and forward-looking health insights. This predictive capability has the potential to transform how individuals manage their health, moving from a reactive approach to a proactive, preventive model of healthcare.

In one embodiment, predictive analysis subsystem 4100 may be designed to provide comprehensive insights from multiple stakeholder perspectives, specifically tailoring its outputs for payers, providers, and patients. This approach ensures that the system's predictive capabilities address the unique needs and concerns of each group while maintaining a cohesive view of the patient's health status.

For payers, predictive analysis subsystem 4100 may incorporate submodels focused on risk assessment, cost projections, and resource allocation. These submodels could analyze historical claims data, demographic information, and current health metrics to predict future healthcare utilization and associated costs. By leveraging machine learning algorithms trained on vast datasets of anonymized patient records, the system can identify high-risk individuals who may benefit from early interventions or care management programs, potentially reducing long-term healthcare expenses.

From the provider perspective, predictive analytics subsystem 4100 may offer submodels geared towards clinical decision support, treatment efficacy prediction, and patient outcome forecasting. These submodels integrate electronic health records, laboratory results, imaging studies, and the latest medical research to assist healthcare professionals in making informed decisions. For instance, the system might predict the likelihood of specific complications following a surgical procedure, allowing providers to tailor their treatment plans and post-operative care strategies accordingly.

For patients, predictive analysis subsystem 4100 may provide personalized health forecasts and lifestyle recommendations. Patient-centric submodels analyze individual health data, including but not limited to data from wearable devices and self-reported symptoms, to predict potential health risks and suggest preventive measures. These submodels are designed to be more accessible and actionable for patients, translating complex medical predictions into easy-to-understand insights and recommendations.

All these perspective-specific submodels share a common reference framework based on current and potential patient health states. This shared foundation ensures consistency across predictions and allows for seamless integration of insights from different perspectives. The common reference data includes may include but is not limited to, a standardized set of health state definitions, ranging from optimal wellness to various degrees of acute and chronic conditions, transition probabilities between health states, derived from large-scale population studies and continually updated with new data, and multidimensional impact assessments for each health state, including quality of life metrics, functional capacity measures, and economic implications.

By utilizing this common reference framework, the system can generate coherent predictions that are relevant and interpretable across all stakeholder groups. For example, a predicted transition to a specific health state would simultaneously inform the payer about potential cost implications, alert the provider to necessary interventions, and provide the patient with personalized guidance for health management.

The predictive analysis subsystem employs advanced machine learning techniques, including ensemble methods and deep learning architectures, to continuously refine its predictions based on new data inputs and outcomes. This adaptive approach allows the system to improve its accuracy over time and adapt to changing healthcare landscapes, emerging medical knowledge, and evolving patient populations.

Predictive analysis subsystem 4100 may incorporate explainable AI techniques to ensure that its predictions and recommendations are transparent and understandable to all stakeholders. This feature is particularly important in healthcare, where the reasoning behind predictions can be as crucial as the predictions themselves.

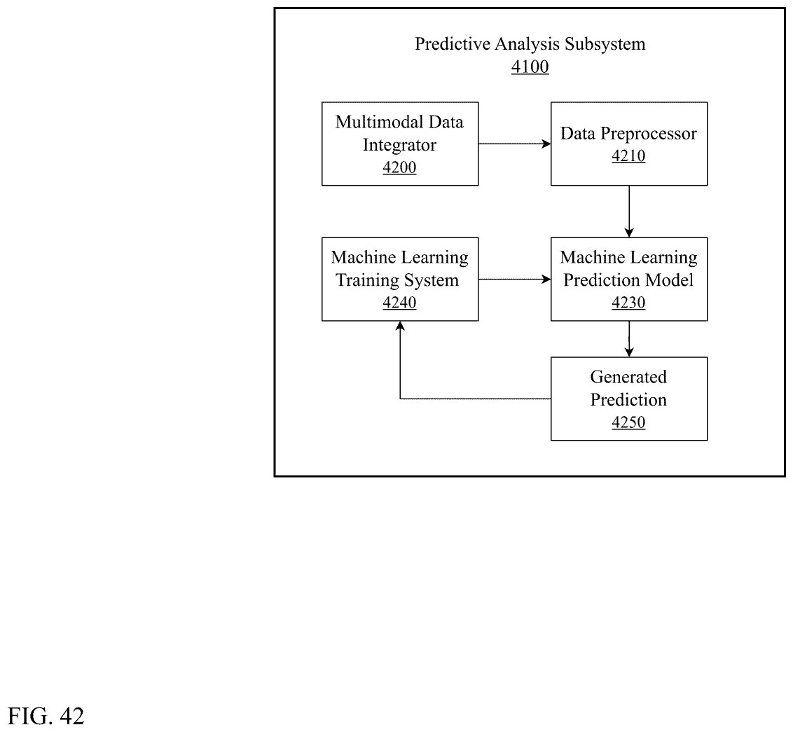

FIG. 42 is a diagram showing an exemplary subsystem of a PHDB system configured for predictive analysis, a predictive analysis subsystem. This subsystem is designed to leverage the diverse health data collected by the PHDB system to generate accurate and personalized health predictions.

The process begins with multimodal data integrator 4200, which serves as the entry point for the various types of data flowing into the predictive analysis subsystem. This component is responsible for bringing together data from multiple sources and modalities, such as structured data from lab results, unstructured data from clinical notes, time-series data from IoT devices, and imaging data from diagnostic procedures. The multimodal data integrator ensures that all these diverse data types are harmonized and prepared for further processing.

Next, data preprocessor 4210 takes the integrated data and performs necessary cleaning, normalization, and transformation operations. This step is crucial for ensuring the quality and consistency of the data that will be used for predictive modeling. The data preprocessor might handle tasks such as dealing with missing values, removing outliers, normalizing numerical data to a common scale, and encoding categorical variables. For example, it might normalize blood pressure readings from different devices to a standard scale, or convert textual descriptions of symptoms into structured, machine-readable formats. The preprocessed data then feeds into machine learning prediction model 4230. This component is at the heart of the predictive analysis subsystem, employing advanced algorithms to identify patterns and make predictions based on the input data. The prediction model could use various machine learning techniques, including but not limited to neural networks, random forests, or gradient boosting machines, depending on the specific prediction task and the nature of the available data.

Machine learning prediction model 4230 is capable of handling a wide range of predictive tasks. For instance, it might forecast future values of key health metrics (like blood glucose levels for diabetic patients), predict the risk of developing certain health conditions based on genetic and lifestyle factors, or estimate the likelihood of hospital readmission for patients with chronic conditions.

Working in tandem with the prediction model is a machine learning training system 4240. This component is responsible for continuously improving the prediction model's performance over time. It uses historical data and outcomes to train and refine the model, ensuring that predictions become more accurate as more data becomes available. The training system might employ techniques like cross-validation and hyperparameter tuning to optimize the model's performance. The output of this process is generated prediction 4250. These predictions could take various forms depending on the specific use case. They might be numerical (e.g., a predicted blood pressure reading for next month), categorical (e.g., a risk classification for developing heart disease), or more complex (e.g., a recommended treatment plan based on predicted outcomes of different interventions).

An important aspect of this subsystem, which aligns with the principles discussed in the new disclosure, is its ability to handle multimodal data and provide explainable predictions. The system doesn't just generate predictions, but also provides insights into which factors contributed most significantly to each prediction. This explainability is crucial in healthcare applications, where understanding the reasoning behind a prediction is often as important as the prediction itself. For example, if the system predicts an elevated risk of type 2 diabetes for a user, it might also indicate that this prediction is based on a combination of factors including recent trends in fasting glucose levels, family history, body mass index, and physical activity levels. This level of detail helps both users and healthcare providers understand the prediction and take appropriate actions.

Moreover, the predictive analysis subsystem can leverage the spatiotemporal modeling capabilities of the PHDB system to generate predictions that take into account both spatial (anatomical) and temporal aspects of health data. For instance, in predicting the progression of a condition like osteoarthritis, the system could consider not just overall trends in joint pain and mobility, but also how these factors vary across different joints and how they've changed over time. The predictive analysis subsystem represents a significant advancement in personalized healthcare. By integrating diverse data sources, employing advanced machine learning techniques, and generating explainable predictions, it enables a shift from reactive to proactive health management. Users can receive early warnings about potential health risks and personalized recommendations for preventive measures, while healthcare providers can make more informed decisions about patient care based on data-driven predictions of treatment outcomes.

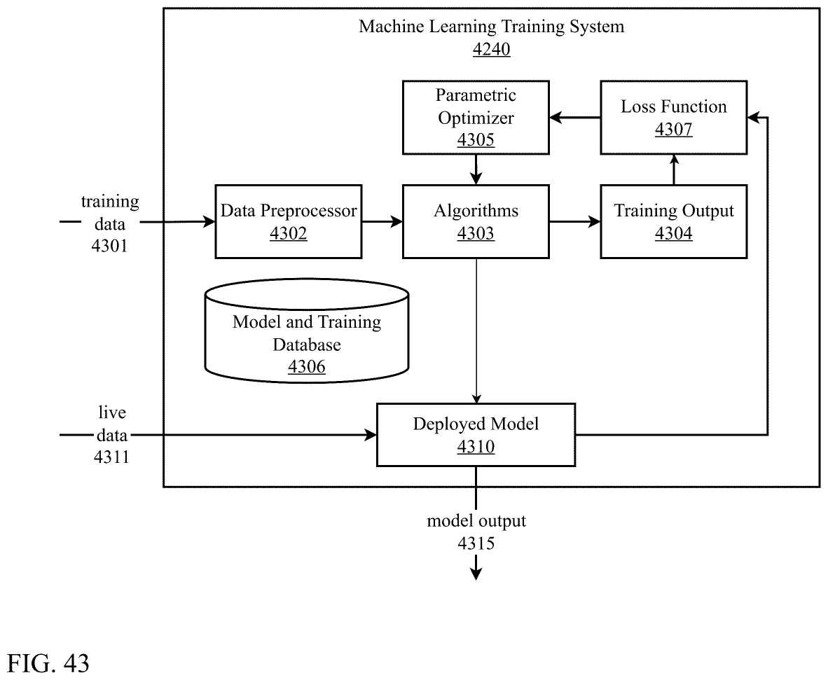

FIG. 43 is a diagram showing an exemplary subsystem of a PHDB system is configured for predictive analysis, a machine learning training system. According to the embodiment, machine learning training system 4240 may comprise a model training stage comprising a data preprocessor 4302, one or more machine and/or deep learning algorithms 4303, training output 4304, and a parametric optimizer 4305, and a model deployment stage comprising a deployed and fully trained model 4310 configured to perform tasks described herein such as processing medical data into a diagnoses or a recommendation. The machine learning training system 4240 may be used to train and deploy a plurality of deep learning architectures in order to support the services provided by the PHDB. In one embodiment, machine learning training system 4240 may be used to train the machine learning prediction model 4330. If the machine learning prediction model 4330 comprises a plurality of different deep learning architectures, the machine learning training system 4340 may train each of the deep learning architectures separately or together as a single system.

At the model training stage, a plurality of training data 4301 may be received by the machine learning training system 4240. Data preprocessor 4302 may receive the input data (e.g., IoT data, hospital records, patient data) and perform various data preprocessing tasks on the input data to format the data for further processing. For example, data preprocessing can include, but is not limited to, tasks related to data cleansing, data deduplication, data normalization, data transformation, handling missing values, feature extraction and selection, mismatch handling, and/or the like. Data preprocessor 4302 may also be configured to create training dataset, a validation dataset, and a test set from the plurality of input data 4301. For example, a training dataset may comprise 80% of the preprocessed input data, the validation set 10%, and the test dataset may comprise the remaining 10% of the data. The preprocessed training dataset may be fed as input into one or more machine and/or deep learning algorithms 4303 to train a predictive model for object monitoring and detection.

During model training, training output 4304 is produced and used to measure the accuracy and usefulness of the predictive outputs. During this process a parametric optimizer 4305 may be used to perform algorithmic tuning between model training iterations. Model parameters and hyperparameters can include, but are not limited to, bias, train-test split ratio, learning rate in optimization algorithms (e.g., gradient descent), choice of optimization algorithm (e.g., gradient descent, stochastic gradient descent, of Adam optimizer, etc.), choice of activation function in a neural network layer (e.g., Sigmoid, ReLu, Tanh, etc.), the choice of cost or loss function the model will use, number of hidden layers in a neural network, number of activation unites in each layer, the drop-out rate in a neural network, number of iterations (epochs) in a training the model, number of clusters in a clustering task, kernel or filter size in convolutional layers, pooling size, batch size, the coefficients (or weights) of linear or logistic regression models, cluster centroids, and/or the like. Parameters and hyperparameters may be tuned and then applied to the next round of model training. In this way, the training stage provides a machine learning training loop.

In some implementations, various accuracy metrics may be used by the machine learning training system 4240 to evaluate a model's performance. Metrics can include, but are not limited to, word error rate (WER), word information loss, speaker identification accuracy (e.g., single stream with multiple speakers), inverse text normalization and normalization error rate, punctuation accuracy, timestamp accuracy, latency, resource consumption, custom vocabulary, sentence-level sentiment analysis, multiple languages supported, cost-to-performance tradeoff, and personal identifying information/payment card industry redaction, to name a few. In one embodiment, the system may utilize a loss function 4307 to measure the system's performance. The loss function 4307 compares the training outputs with an expected output and determines how the algorithm needs to be changed in order to improve the quality of the model output. During the training stage, all outputs may be passed through the loss function 4307 on a continuous loop until the algorithms 4303 are in a position where they can effectively be incorporated into a deployed model 4315.

The test dataset can be used to test the accuracy of the model outputs. If the training model is establishing correlations that satisfy a certain criterion such as but not limited to quality of the correlations and amount of restored lost data, then it can be moved to the model deployment stage as a fully trained and deployed model 4310 in a production environment making predictions based on live input data 4311 (e.g., IoT data, hospital records, patient data). Further, model correlations and restorations made by deployed model can be used as feedback and applied to model training in the training stage, wherein the model is continuously learning over time using both training data and live data and predictions. A model and training database 4306 is present and configured to store training/test datasets and developed models. Database 4306 may also store previous versions of models.

According to some embodiments, the one or more machine and/or deep learning models may comprise any suitable algorithm known to those with skill in the art including, but not limited to: LLMs, generative transformers, transformers, supervised learning algorithms such as: regression (e.g., linear, polynomial, logistic, etc.), decision tree, random forest, k-nearest neighbor, support vector machines, Naïve-Bayes algorithm; unsupervised learning algorithms such as clustering algorithms, hidden Markov models, singular value decomposition, and/or the like. Alternatively, or additionally, algorithms 4303 may comprise a deep learning algorithm such as neural networks (e.g., recurrent, convolutional, long short-term memory networks, etc.).

In some implementations, the machine learning training system 4240 automatically generates standardized model scorecards for each model produced to provide rapid insights into the model and training data, maintain model provenance, and track performance over time. These model scorecards provide insights into model framework(s) used, training data, training data specifications such as chip size, stride, data splits, baseline hyperparameters, and other factors. Model scorecards may be stored in database(s) 4306.

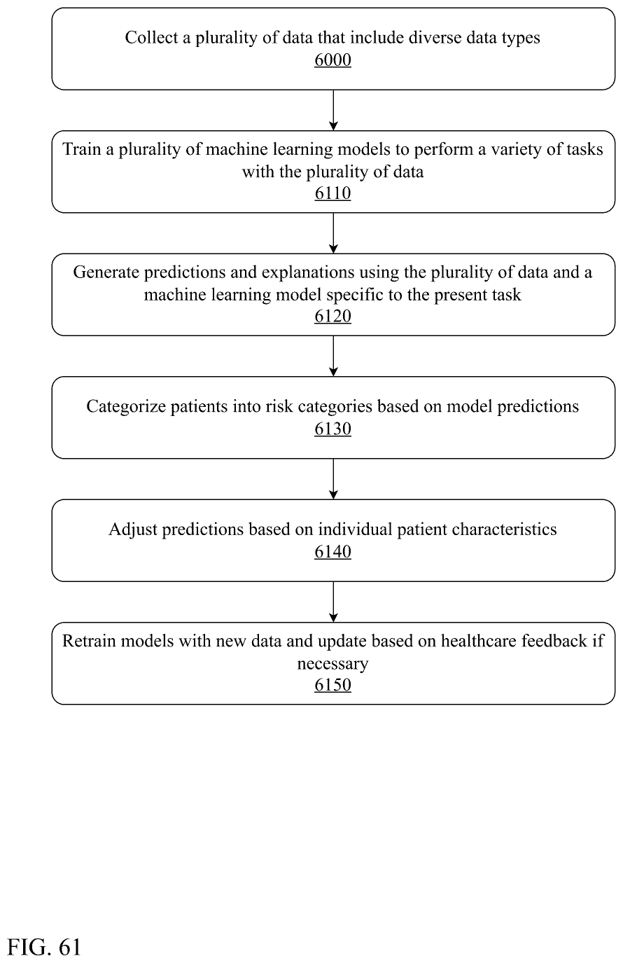

FIG. 61 is a flow diagram showing exemplary method for configuring a PHDB system for enhanced predictive analysis. In a first step 6000, the system collects a plurality of data that include diverse data types. This step involves gathering a wide range of health-related information from various sources within the PHDB ecosystem. The data types might include structured data like lab results from labs, unstructured data like clinical notes from care givers, real-time sensor data from IoT devices such as wearable fitness trackers, and electronic health records from third-party services. For example, for a patient with diabetes, the system might collect blood glucose readings, HbA1c test results, daily food logs, physical activity data from a smartwatch, and the patient's medical history including any complications or related conditions.

In a step 6110, the system trains a plurality of machine learning models to perform a variety of tasks with the plurality of data. This step involves using the collected data to develop and refine various predictive models, each designed for specific health-related tasks. The machine learning training system plays a crucial role here, employing techniques like cross-validation and hyperparameter tuning to optimize each model's performance. For instance, one model might be trained to predict future blood glucose levels based on historical data and current lifestyle factors. Another model could be trained to assess the risk of diabetes-related complications like neuropathy or retinopathy based on long-term trends in health metrics and genetic factors.

In a step 6120, the system generates predictions and explanations using the plurality of data and a machine learning model specific to the present task. This step involves applying the trained models to current data to produce forecasts and insights. The system doesn't just provide predictions, but also explanations for how these predictions were derived, aligning with the principle of explainable AI discussed in the new disclosure. For our diabetes example, the system might predict that a patient is at high risk of experiencing a hypoglycemic event in the next 24 hours based on recent blood glucose trends, insulin doses, and planned physical activity. Along with this prediction, it would provide an explanation detailing which factors contributed most significantly to this assessment.

In a step 6130, the system categorizes patients into risk categories based on model predictions. This step involves using the outputs of the predictive models to classify patients according to their level of risk for various health outcomes. These categories could be used to prioritize interventions or allocate healthcare resources more effectively. In our diabetes example, patients might be categorized into low, medium, and high-risk groups for developing complications like diabetic foot ulcers. This categorization could be based on factors such as blood glucose control, peripheral neuropathy symptoms, and foot care habits.

In a step 6140, the system adjusts predictions based on individual patient characteristics. This step ensures that the predictions and risk categorizations are tailored to each patient's unique circumstances. It might involve considering factors like age, gender, comorbidities, lifestyle, and personal preferences. For instance, while two patients might have similar blood glucose levels, their risk of complications could be adjusted based on factors like their level of physical activity, diet quality, or adherence to medication regimens.

In a step 6150, the system retrains models with new data and updates based on healthcare feedback if necessary. This final step allows for maintaining the accuracy and relevance of the predictive models over time. As new data becomes available, whether it's from ongoing patient monitoring, new research findings, or feedback from healthcare providers—the models are updated to incorporate this information. For example, if new research reveals a previously unknown risk factor for diabetic retinopathy, this information could be incorporated into the relevant predictive models. Similarly, if healthcare providers notice that the system's predictions for a certain subset of patients are consistently off, this feedback could be used to refine the models for improved accuracy.

This method enables the PHDB system to provide dynamic, personalized, and explainable health predictions. By continuously integrating diverse data sources, training specialized models, and adapting to new information and individual patient characteristics, it offers a powerful tool for proactive health management. The system's ability to not only predict health outcomes but also explain these predictions and categorize risks allows for more informed decision-making by both patients and healthcare providers. This approach aligns with the trend towards personalized, predictive, and preventive healthcare, potentially improving health outcomes and efficiency of care delivery.

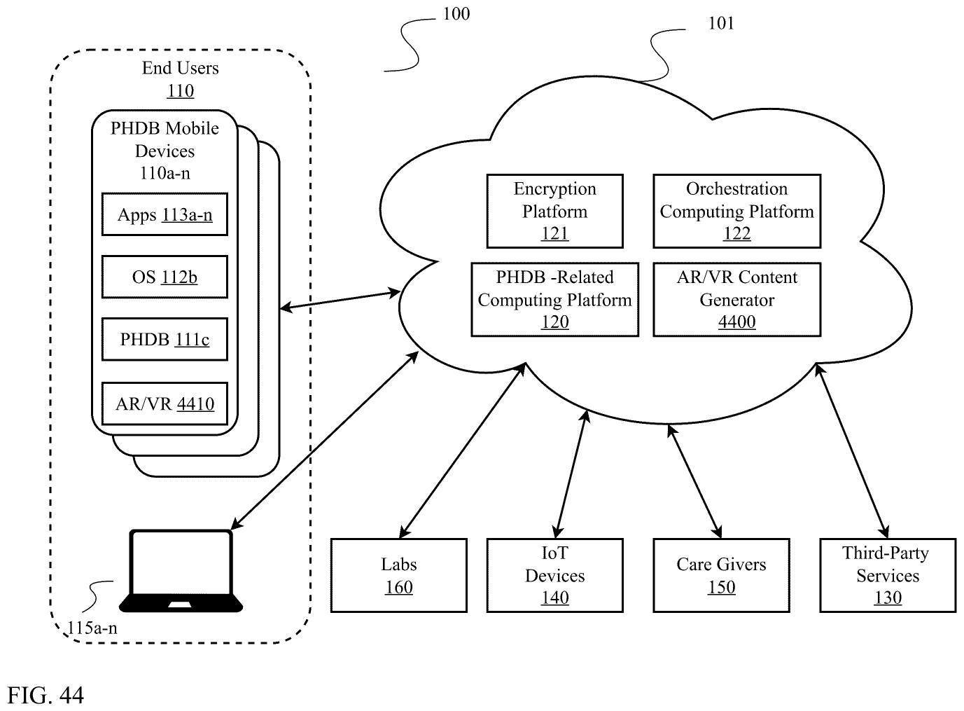

FIG. 44 is a block diagram showing an exemplary arrangement of a PHDB, where the PHDB system is configured to generate AR/VR content. This addition represents a significant advancement in the system's ability to visualize and interact with health data in immersive and intuitive ways. AR/VR content generator 4400 integrates seamlessly with the existing PHDB-related computing platform 120, working in conjunction with other critical components such as the encryption platform 121 and the orchestration computing platform 122. This subsystem is designed to transform the complex health data collected and processed by the PHDB system into immersive, three-dimensional visualizations and interactive experiences.

AR/VR content generator 4400 leverages data from various sources within the PHDB ecosystem. It processes information from PHDB mobile devices 110a-n, which include user applications apps 113a-n, the device's operating system (OS 112b), and the local PHDB storage 111c. This allows the system to incorporate real-time and historical data directly from users, including manually entered information, device sensor readings, and locally stored health records. Furthermore, the AR/VR content generator 4400 interfaces with data from IoT devices 140, labs 160, third-party services 130, and care givers 150. This diverse range of data sources enables the system to create comprehensive and detailed AR/VR representations that consider a wide array of factors influencing a user's health.

AR/VR devices 4410 work in tandem with the content generator to deliver these immersive experiences to end users 110. This could involve augmented reality applications on mobile devices, virtual reality headsets, or other forms of mixed reality technology. For example, the system might generate a three-dimensional model of a user's cardiovascular system based on their latest health data. In an AR application, users could use their mobile devices to project this model into their physical space, allowing them to walk around and examine it from different angles. In a VR environment, users could be immersed in a virtual representation of their own body, navigating through different organ systems and observing how various health metrics affect their physiology.

AR/VR content generator 4400 employs advanced algorithms to translate complex medical data into visually intuitive representations. It can create dynamic visualizations that change in real-time based on incoming data from IoT devices or updates to the user's health records. For instance, a user with diabetes might see a real-time visualization of how their blood glucose levels affect different parts of their body, with the visualization updating as new glucose readings come in from a continuous glucose monitor. Security and privacy remain paramount in this enhanced system. The AR/VR content generator works in tandem with the encryption platform 121 to ensure that all data processing and visualization creation occur on encrypted data, maintaining user privacy and data security throughout the AR/VR content generation process.

The orchestration computing platform 122 plays a crucial role in managing the interactions between AR/VR content generator 4400 and other components of the PHDB system. It ensures efficient data flow, coordinates processing tasks, and manages the distribution of AR/VR content to relevant parts of the system, including back to users' PHDB mobile devices 110a-n or to healthcare providers via secure channels. One of the key strengths of this AR/VR approach is its ability to make complex health information more accessible and understandable to users. Rather than trying to interpret numerical data or text-based reports, users can interact with visual representations of their health data in a more intuitive and engaging way. This could potentially lead to better understanding of health conditions, improved adherence to treatment plans, and more informed health-related decision making.

Moreover, AR/VR content generator 4400 can support healthcare providers in explaining diagnoses, treatment options, and potential outcomes to patients. By providing immersive, interactive visualizations, it can help bridge the communication gap between medical professionals and patients, leading to better informed consent and shared decision-making. The integration of AR/VR capabilities represents a significant step towards more engaging and intuitive health management. By allowing users to visualize and interact with their health data in immersive environments, the system can potentially increase user engagement, improve health literacy, and facilitate better communication between patients and healthcare providers.

The addition of AR/VR content generator 4400 and AR/VR 4410 devices to the PHDB system marks a major advancement in personal health data visualization and interaction. By leveraging the vast amount of diverse health data collected by the system and transforming it into immersive AR/VR experiences, it enables more intuitive, engaging, and potentially more effective health management. This AR/VR capability has the potential to transform how individuals understand and interact with their health data, moving from abstract numbers and reports to tangible, interactive visualizations.

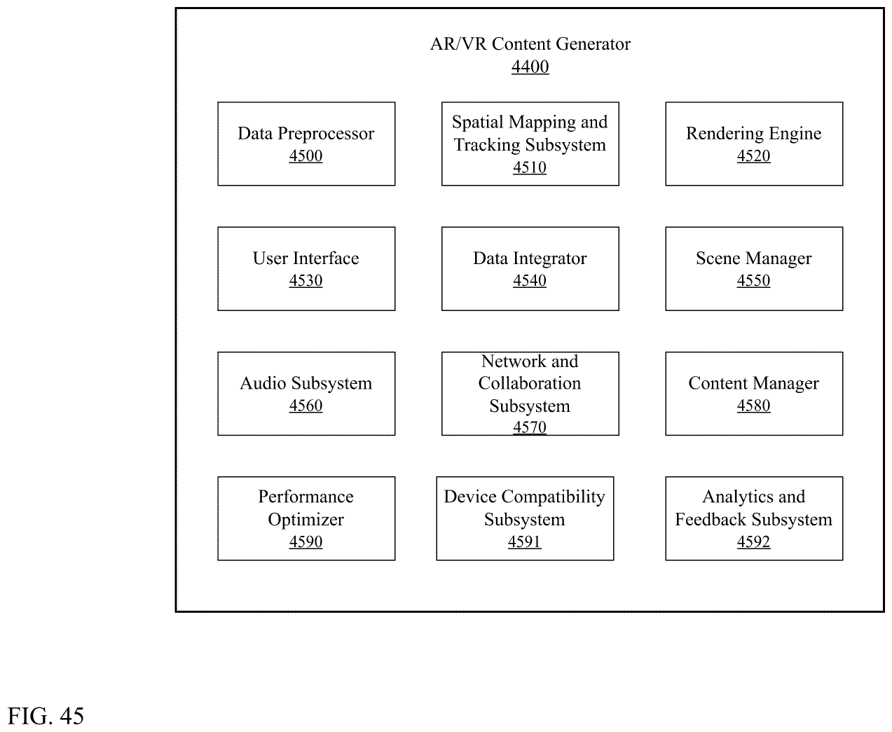

FIG. 45 is a diagram showing an exemplary subsystem of a PHDB system configured for AR/VR content generation, an AR/VR content generator. Illustrated is a detailed view of AR/VR content generator 4400, illustrating its internal components and the flow of data through the system. This subsystem is designed to transform the diverse health data collected by the PHDB system into immersive and interactive AR/VR experiences. The process begins with a data preprocessor 4500, which prepares the incoming health data for AR/VR content generation. This component handles tasks such as data cleaning, normalization, and formatting to ensure that the information is suitable for 3D visualization. For example, it might convert 2D medical imaging data into 3D models or transform time-series data from IoT devices into a format suitable for dynamic visualizations.

A spatial mapping and tracking subsystem 4510 is responsible for understanding the user's physical environment (in AR applications) and tracking the user's movements in both AR and VR scenarios. This component enables the accurate placement of virtual objects in the real world for AR applications and ensures that the VR environment responds correctly to the user's movements. A rendering engine 4520 is the core component for creating the visual elements of the AR/VR experience. It generates the 3D models, textures, and animations that represent the user's health data. For instance, it might create a detailed 3D model of a user's heart, complete with animations showing blood flow based on the user's latest cardiovascular data.

A user interface 4530 component manages how users interact with the AR/VR content. It handles inputs such as gestures, voice commands, or controller inputs, and determines how these should affect the virtual environment. This component is crucial for creating an intuitive and engaging user experience. A data integrator 4540 works to combine data from various sources within the PHDB system into a coherent AR/VR representation. It might, for example, integrate real-time data from IoT devices with historical health records and genetic information to create a comprehensive health visualization.

A scene manager 4550 oversees the overall structure and organization of the AR/VR environment. It manages the placement of virtual objects, handles level-of-detail adjustments for optimal performance, and controls how different elements of the visualization interact with each other. An audio subsystem 4560 generates spatial audio to enhance the immersive experience. This could include creating sound effects that correspond to visualized health data or providing audio explanations of what the user is seeing.

A network and collaboration subsystem 4570 enables multi-user AR/VR experiences. This could allow healthcare providers to join a patient's AR/VR session remotely, facilitating more intuitive and engaging telemedicine consultations. A content manager 4580 oversees the creation, storage, and delivery of AR/VR content. It ensures that the right content is available for each user based on their health data and the specific visualization they're accessing.

A performance optimizer 4590 works to ensure smooth and responsive AR/VR experiences across a range of devices. It might employ techniques like dynamic resolution scaling or foveated rendering to maintain high frame rates even on less powerful devices. A device compatibility subsystem 4591 ensures that the AR/VR content can be experienced on a variety of devices, from high-end VR headsets to smartphones using mobile AR. It adapts the content and interactions based on the capabilities of the user's device. Finally, an analytics and feedback subsystem 4592 collects data on how users interact with the AR/VR content. This information can be used to improve the visualizations over time, making them more intuitive and effective.

AR/VR content generator 4400 represents a significant advancement in how individuals can interact with their health data. By transforming complex medical information into immersive, interactive 3D visualizations, it has the potential to greatly enhance health literacy and engagement. For example, a patient with diabetes could step into a virtual environment where they can see a life-sized model of their own body, observing in real-time how their blood glucose levels affect different organs. They could interact with this model, perhaps fast-forwarding to see predicted outcomes based on different treatment options.

The system's ability to integrate diverse data sources allows for highly personalized and comprehensive health visualizations. It could combine genetic data, current health metrics, environmental factors, and lifestyle information to create a holistic representation of a user's health status and potential future scenarios. Moreover, the collaborative aspects of this system open up new possibilities for patient-provider interactions. A healthcare provider could join a patient in the virtual environment, using the immersive visualization to explain complex medical concepts or treatment plans in a more intuitive way. By making health data more accessible and engaging through AR/VR technology, this system has the potential to empower users to take a more active role in managing their health, potentially leading to better health outcomes and a more proactive approach to healthcare.

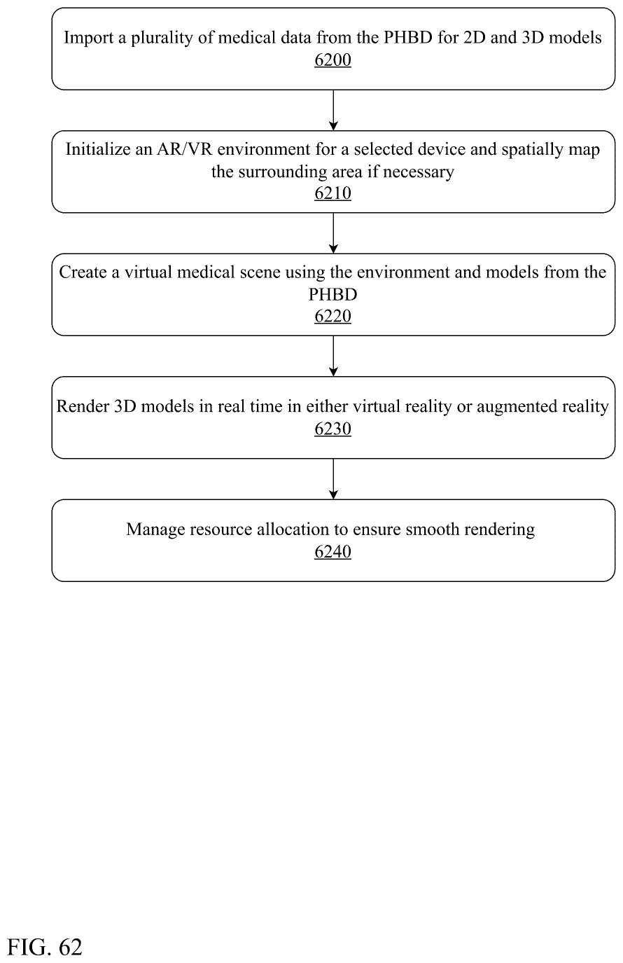

FIG. 62 is a flow diagram showing exemplary method for configuring a PHDB system for generating AR/VR content. In a first step 6200, the system imports a plurality of medical data from the PHDB for 2D and 3D models. This step involves retrieving relevant health data from the user's personal health database, which may include a wide range of information such as medical imaging results, lab test data, real-time sensor data from IoT devices, and historical health records. The data preprocessor plays a role here, converting this diverse data into formats suitable for 2D and 3D visualization. For example, it might transform MRI scans into 3D models of specific organs, convert time-series data from a continuous glucose monitor into a format suitable for dynamic visualization, or prepare EKG data for a 2D graph that can be placed within the 3D environment.

In a step 6210, the system initializes an AR/VR environment for a selected device and spatially maps the surrounding area if necessary. This step, primarily handled by the spatial mapping and tracking subsystem and the device compatibility subsystem, involves setting up the AR/VR experience based on the user's device capabilities and the chosen mode (AR or VR). For AR applications, this includes scanning and mapping the user's physical environment to enable accurate placement of virtual objects. For instance, if the user is using a smartphone for an AR experience, the system might use the device's camera to understand the layout of the room, allowing it to place a virtual 3D model of the user's circulatory system in a specific location. For VR applications, this step involves initializing the virtual environment where the health data will be visualized.

In a step 6220, the system creates a virtual medical scene using the environment and models from the PHDB. This step is where the scene manager and rendering engine work together to construct the immersive health visualization. The system takes the 2D and 3D models created from the user's health data and places them within the initialized AR/VR environment. For example, in a VR scenario for a patient with heart disease, this might involve creating a life-sized, 3D model of the patient's heart based on their latest cardiac imaging results, surrounded by floating 2D graphs showing trends in blood pressure and cholesterol levels. In an AR scenario, it might involve overlaying a 3D model of the patient's respiratory system onto their body, visible through their smartphone camera.

In a step 6230, the system renders 3D models in real time in either virtual reality or augmented reality. This step is primarily executed by the rendering engine, which generates the visual representation of the health data models within the AR/VR environment. The rendering is done in real-time, allowing for dynamic updates based on incoming data or user interactions. For instance, in our heart disease example, the 3D heart model might pulse in sync with the patient's actual heart rate, changing color to indicate blood oxygenation levels based on real-time data from a wearable device. The user interface component also plays a role here, enabling users to interact with the rendered models through gestures, voice commands, or controller inputs.

In a step 6240, the system manages resource allocation to ensure smooth rendering. This final step is handled by the performance optimizer and involves continuously monitoring and adjusting the AR/VR experience to maintain optimal performance across different devices. This might include techniques like dynamic resolution scaling, where the detail of the rendered models is adjusted based on the processing power of the user's device, or foveated rendering in VR, where areas of the display that the user isn't directly looking at are rendered in lower detail to save processing power. For example, on a high-end VR headset, the system might render a highly detailed, fully animated model of the circulatory system, while on a smartphone AR app, it might provide a simpler, less resource-intensive visualization.

Throughout this process, the system leverages other components like the audio subsystem to provide spatial audio cues enhancing the immersive experience, the network and collaboration subsystem to enable shared experiences with healthcare providers, and the analytics and feedback subsystem to gather data on user interactions for future improvements. This method enables the PHDB system to transform complex health data into intuitive, immersive AR/VR experiences. By leveraging advanced visualization techniques and real-time rendering, it allows users to interact with their health information in novel ways, potentially improving understanding, engagement, and ultimately, health outcomes. The system's ability to adapt to different devices and manage resources efficiently ensures that these powerful visualizations are accessible to a wide range of users, from those with high-end VR setups to those using everyday smartphones.

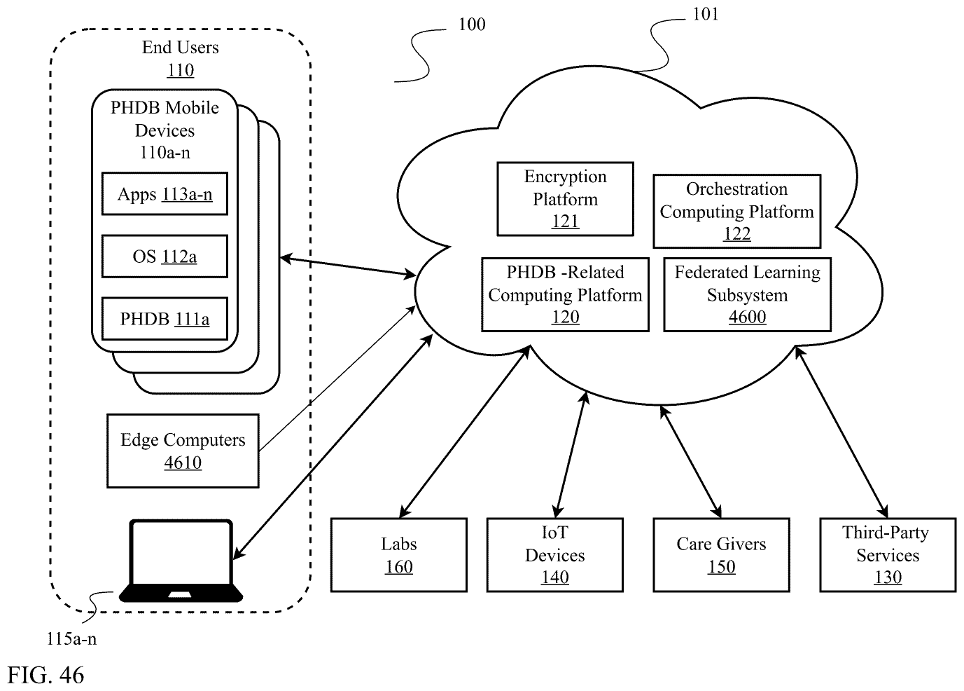

FIG. 46 is a block diagram showing an exemplary arrangement of a PHDB, where the PHDB system is configured for federated learning. This addition represents a significant advancement in the system's ability to learn from distributed data while maintaining user privacy and data security. Federated learning subsystem 4600 integrates seamlessly with the existing PHDB-related computing platform 120, working in conjunction with other critical components such as the encryption platform 121 and the orchestration computing platform 122. This subsystem is designed to enable machine learning across decentralized data stored on multiple devices or servers without the need to exchange raw data.

Federated learning subsystem 4600 leverages data from various sources within the PHDB ecosystem. It processes information from PHDB mobile devices 110a-n, which include user applications apps 113a-n, the device's operating system (OS 112a), and the local PHDB storage 111a. This allows the system to learn from user data while keeping that data local to the user's device, enhancing privacy and data security. Furthermore, the federated learning subsystem interfaces with data from IoT devices 140, labs 160, third-party services 130, and care givers 150. This diverse range of data sources enables the system to create comprehensive and robust machine learning models that consider a wide array of factors influencing users' health.

Edge computers 4610 play a crucial role in this federated learning approach. These could be the users' personal devices, local servers, or other computational resources located close to the data source. The edge computers perform local computations on the user's data, updating a shared model without transmitting the raw data to a central server. For example, the system might develop a model to predict the risk of cardiovascular disease. Instead of collecting all users' health data in a central location, the federated learning subsystem would send the current model to each user's device. The device would then use the user's local data to improve the model, sending back only the model updates, not the raw data. The central system would aggregate these updates to improve the overall model.

Federated learning subsystem 4600 employs advanced algorithms to aggregate model updates from multiple sources, ensuring that the global model improves without compromising individual user data. It can handle various machine learning tasks, from simple regression models to complex deep learning networks, adapting its approach based on the specific health prediction or analysis task at hand. Security and privacy remain paramount in this enhanced system. The federated learning subsystem works in tandem with the encryption platform 121 to ensure that all model updates are encrypted during transmission, maintaining user privacy and data security throughout the learning process. Moreover, techniques like differential privacy can be implemented to add noise to the model updates, further protecting individual user data from potential inference attacks.

Orchestration computing platform 122 plays a crucial role in managing the interactions between the federated learning subsystem, edge computers, and other components of the PHDB system. It ensures efficient coordination of the federated learning process, manages the distribution of model updates, and oversees the aggregation of these updates into the global model. One of the key strengths of this federated learning approach is its ability to learn from a diverse and large dataset while respecting data privacy regulations and user preferences. For instance, it could enable the development of more accurate and personalized health prediction models by learning from users across different geographic regions, age groups, and health conditions, all without centralizing sensitive health data.

The federated learning subsystem 4600 can support continuous learning and model improvement. As users interact with their PHDB mobile devices and generate new health data, the local models on their devices can be updated, contributing to the ongoing refinement of the global model. This ensures that the system's predictive capabilities remain current and adapt to changing health trends and user behaviors.

The integration of federated learning represents a significant step towards more privacy-preserving and decentralized machine learning in healthcare. By allowing the system to learn from diverse data sources without centralizing sensitive information, it enables the development of more robust and generalizable health models while maintaining strong privacy guarantees. In summary, the addition of federated learning subsystem 4600 and edge computers 4610 to the PHDB system marks a major advancement in privacy-preserving machine learning for health applications. By enabling learning from decentralized data sources, it allows the system to leverage large and diverse datasets for improved health predictions and insights, all while maintaining strong privacy protections for individual users. This capability has the potential to transform how health-related machine learning models are developed and deployed, enabling more personalized and effective health management while respecting user privacy.

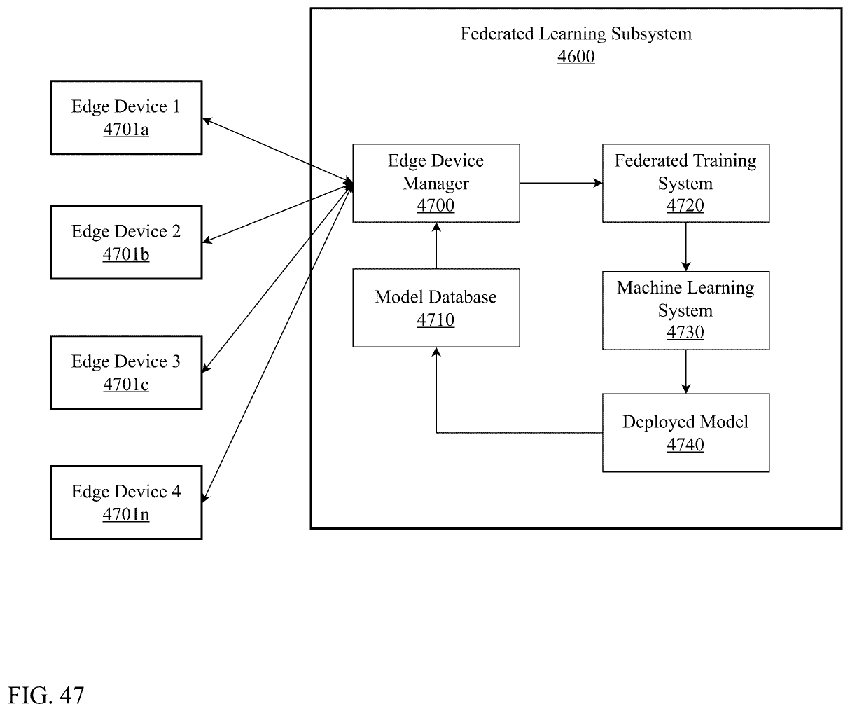

FIG. 47 is a diagram showing an exemplary subsystem of a PHDB system configured for federated learning, a federated learning subsystem. This subsystem is designed to enable collaborative learning across distributed data sources while preserving data privacy and security. Federated learning subsystem 4600 interfaces with multiple edge devices (4701a, 4701b, 4701c, 4701n). These edge devices could represent individual users' PHDB mobile devices 110a-n, local servers, or other computational resources close to the data source. Each edge device contains a local copy of the user's personal health database and performs computations on this local data.

An edge device manager 4700 is responsible for coordinating communication between the federated learning subsystem and the edge devices. It manages the distribution of model updates to the edge devices and the collection of locally computed updates. This component ensures that the federated learning process runs smoothly across all participating devices, handling issues such as device availability, network connectivity, and synchronization of model versions. A model database 4710 stores various versions of the machine learning models being trained through the federated learning process. This includes the current global model, historical versions for tracking progress, and potentially different models for various health-related tasks. For example, it might store separate models for predicting cardiovascular risk, diabetes progression, and mental health status.

A federated training system 4720 is at the heart of the federated learning process. It implements the federated averaging algorithm or other federated learning techniques to aggregate model updates from the edge devices. This component ensures that the global model improves based on the collective knowledge from all participating devices without centralizing the raw data. For instance, in training a model to predict the risk of type 2 diabetes, this system would aggregate the model updates computed on individual users' devices based on their local health data, lifestyle information, and genetic factors. A machine learning system 4730 is responsible for the core machine learning operations. It handles tasks such as model initialization, hyperparameter tuning, and evaluation of model performance. This system can support various types of machine learning models, from simple linear regressions to complex deep neural networks, adapting to the specific requirements of different health prediction tasks.

Deployed model 4740 represents the current best version of the global model, which has been trained through the federated learning process. This model is what gets distributed to the edge devices for making predictions or for further training. For example, a deployed model for predicting heart disease risk would be sent to users' devices, where it can make personalized predictions based on the user's local health data without that data ever leaving the device. This federated learning approach offers several advantages in the context of the PHDB system. It allows the system to learn from a diverse and large dataset, potentially improving the accuracy and generalizability of health predictions. For instance, it could develop more robust models for rare diseases by learning from cases across a wide geographic area without centralizing sensitive patient data.

Moreover, this approach strongly aligns with data privacy regulations and user preferences. By keeping raw data on users' devices and only sharing model updates, it minimizes the risk of data breaches or unauthorized access to sensitive health information. This is particularly crucial given the sensitive nature of the data stored in personal health databases. The federated learning subsystem also supports continuous learning. As users interact with their PHDB mobile devices and generate new health data, their local models can be updated, contributing to the ongoing refinement of the global model. This ensures that the system's predictive capabilities remain current and adapt to changing health trends and user behaviors. Furthermore, this approach can handle the heterogeneity of data across different users and devices. The federated training system can implement techniques to deal with non-IID (Independent and Identically Distributed) data, a common challenge in federated learning where different users may have very different data distributions.

In practice, this system could enable sophisticated health analyses without compromising user privacy. For example, it could develop a model to predict the likelihood of adverse drug reactions by learning from users' medication histories, genetic data, and reported side effects, all while keeping this sensitive information local to each user's device. The federated learning subsystem represents a significant advancement in privacy-preserving machine learning for health applications. By enabling collaborative learning from decentralized data sources, it allows the PHDB system to leverage large and diverse datasets for improved health predictions and insights, all while maintaining strong privacy protections for individual users. This capability has the potential to transform how health-related machine learning models are developed and deployed, enabling more personalized and effective health management while respecting user privacy.

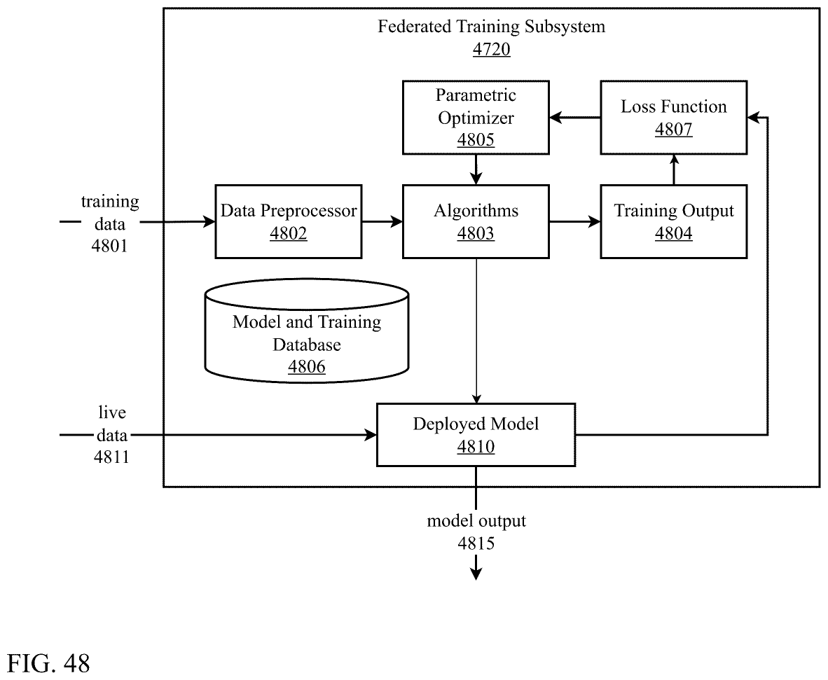

FIG. 48 is a diagram showing an exemplary subsystem of a PHDB system configured for federated learning, a federated training subsystem. According to the embodiment, federated training subsystem 4720 may comprise a model training stage comprising a data preprocessor 4802, one or more machine and/or deep learning algorithms 4803, training output 4804, and a parametric optimizer 4805, and a model deployment stage comprising a deployed and fully trained model 4810 configured to perform tasks described herein such as processing medical data into medical diagnoses or recommendations. The federated training subsystem 4720 may be used to train and deploy a plurality of deep learning architectures in order to support the services provided by the PHDB. In one embodiment, the federated training subsystem 4720 may be used to train the machine learning prediction model 4830. If the machine learning prediction model 4830 comprises a plurality of different deep learning architectures, the machine learning training system 4840 may train each of the deep learning architectures separately or together as a single system.

At the model training stage, a plurality of training data 4801 may be received by the federated training subsystem 4820. Data preprocessor 4802 may receive the input data (e.g., IoT data, hospital records, and patient data) and perform various data preprocessing tasks on the input data to format the data for further processing. For example, data preprocessing can include, but is not limited to, tasks related to data cleansing, data deduplication, data normalization, data transformation, handling missing values, feature extraction and selection, mismatch handling, and/or the like. Data preprocessor 4802 may also be configured to create training dataset, a validation dataset, and a test set from the plurality of input data 4801. For example, a training dataset may comprise 80% of the preprocessed input data, the validation set 10%, and the test dataset may comprise the remaining 10% of the data. The preprocessed training dataset may be fed as input into one or more machine and/or deep learning algorithms 4803 to train a predictive model for object monitoring and detection.

During model training, training output 4804 is produced and used to measure the accuracy and usefulness of the predictive outputs. During this process a parametric optimizer 4805 may be used to perform algorithmic tuning between model training iterations. Model parameters and hyperparameters can include, but are not limited to, bias, train-test split ratio, learning rate in optimization algorithms (e.g., gradient descent), choice of optimization algorithm (e.g., gradient descent, stochastic gradient descent, of Adam optimizer, etc.), choice of activation function in a neural network layer (e.g., Sigmoid, ReLu, Tanh, etc.), the choice of cost or loss function the model will use, number of hidden layers in a neural network, number of activation units in each layer, the drop-out rate in a neural network, number of iterations (epochs) in a training the model, number of clusters in a clustering task, kernel or filter size in convolutional layers, pooling size, batch size, the coefficients (or weights) of linear or logistic regression models, cluster centroids, and/or the like. Parameters and hyperparameters may be tuned and then applied to the next round of model training. In this way, the training stage provides a machine learning training loop.

In some implementations, various accuracy metrics may be used by the federated training subsystem 4720 to evaluate a model's performance. Metrics can include, but are not limited to, word error rate (WER), word information loss, speaker identification accuracy (e.g., single stream with multiple speakers), inverse text normalization and normalization error rate, punctuation accuracy, timestamp accuracy, latency, resource consumption, custom vocabulary, sentence-level sentiment analysis, multiple languages supported, cost-to-performance tradeoff, and personal identifying information/payment card industry redaction, to name a few. In one embodiment, the system may utilize a loss function 4807 to measure the system's performance. The loss function 4807 compares the training outputs with an expected output and determines how the algorithm needs to be changed in order to improve the quality of the model output. During the training stage, all outputs may be passed through the loss function 4807 on a continuous loop until the algorithms 4803 are in a position where they can effectively be incorporated into a deployed model 4815.

The test dataset can be used to test the accuracy of the model outputs. If the training model is establishing correlations that satisfy a certain criterion such as but not limited to quality of the correlations and amount of restored lost data, then it can be moved to the model deployment stage as a fully trained and deployed model 4810 in a production environment making predictions based on live input data 4811 (e.g., IoT data, hospital records, patient data). Further, model correlations and restorations made by deployed model can be used as feedback and applied to model training in the training stage, wherein the model is continuously learning over time using both training data and live data and predictions. A model and training database 4806 is present and configured to store training/test datasets and developed models. Database 4806 may also store previous versions of models.

According to some embodiments, the one or more machine and/or deep learning models may comprise any suitable algorithm known to those with skill in the art including, but not limited to: LLMs, generative transformers, transformers, supervised learning algorithms such as: regression (e.g., linear, polynomial, logistic, etc.), decision tree, random forest, k-nearest neighbor, support vector machines, Naïve-Bayes algorithm; unsupervised learning algorithms such as clustering algorithms, hidden Markov models, singular value decomposition, and/or the like. Alternatively, or additionally, algorithms 4803 may comprise a deep learning algorithm such as neural networks (e.g., recurrent, convolutional, long short-term memory networks, etc.).

In some implementations, the federated training subsystem 4840 automatically generates standardized model scorecards for each model produced to provide rapid insights into the model and training data, maintain model provenance, and track performance over time. These model scorecards provide insights into model framework(s) used, training data, training data specifications such as chip size, stride, data splits, baseline hyperparameters, and other factors. Model scorecards may be stored in database(s) 4806.

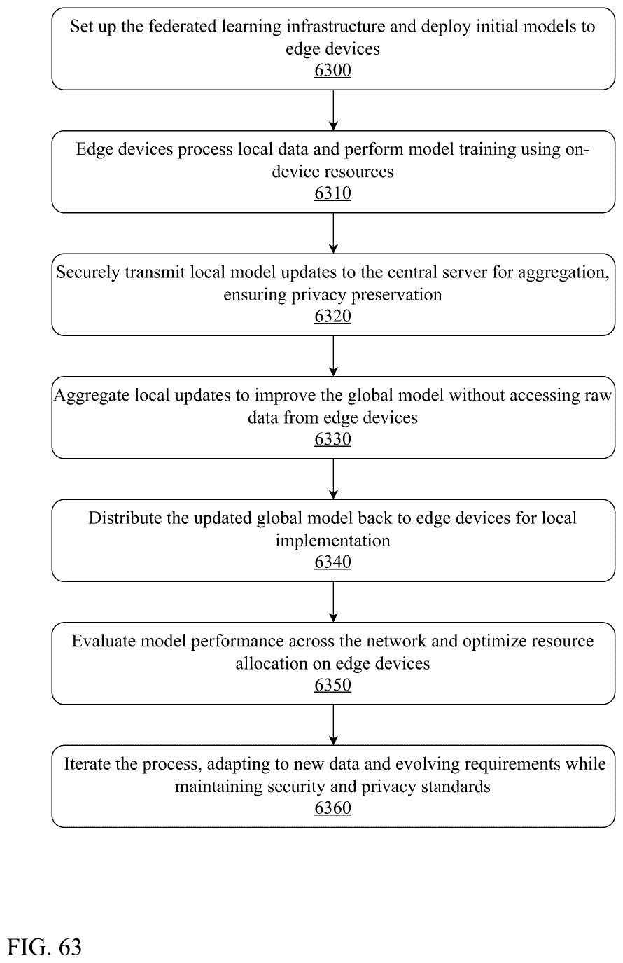

FIG. 63 is a flow diagram showing exemplary method for configuring a PHDB system for federated learning and edge device computing. In a first step 6300, the system sets up the federated learning infrastructure and deploys initial models to edge devices. This step involves initializing the federated learning subsystem and preparing the edge devices for participation in the learning process. The edge device manager coordinates the distribution of initial models from the model database to the various edge devices. For example, if developing a model to predict cardiovascular risk, an initial model based on general medical knowledge might be deployed to all participating PHDB mobile devices.

In a step 6310, edge devices process local data and perform model training using on-device resources. This step leverages the computational capabilities of individual devices to train the model on local data without sharing raw information. The machine learning system provides the necessary algorithms for this local training. For instance, a user's device might update the cardiovascular risk model based on their personal health data, including their blood pressure readings, cholesterol levels, exercise habits, and genetic information stored in their local PHDB.

In a step 6320, the system securely transmits local model updates to the central server for aggregation, ensuring privacy preservation. This step is crucial for maintaining user privacy while enabling collaborative learning. The edge device manager oversees this process, ensuring that only model updates, not raw data, are transmitted. These updates are encrypted using the encryption platform before transmission. Continuing our cardiovascular risk example, the user's device would send only the changes to the model parameters, not the underlying health data used to calculate these changes.

In a step 6330, the system aggregates local updates to improve the global model without accessing raw data from edge devices. This step is performed by the federated training system, which implements federated averaging or similar algorithms to combine updates from multiple devices into a single, improved global model. For our cardiovascular risk model, this might involve averaging the changes to model weights suggested by thousands of individual devices, potentially revealing population-level insights without exposing individual data.

In a step 6340, the system distributes the updated global model back to edge devices for local implementation. The edge device manager coordinates this distribution, ensuring that all participating devices receive the latest version of the model. In our example, all users would now have access to an improved cardiovascular risk prediction model that has learned from a diverse population, potentially offering more accurate and personalized risk assessments.

In a step 6350, the system evaluates model performance across the network and optimizes resource allocation on edge devices. This step involves assessing how well the model is performing on various devices and adjusting the federated learning process accordingly. The machine learning system might compute performance metrics on a held-out validation set on each device, with aggregated results informing future training strategies. For resource optimization, the system might adjust the frequency of model updates or the complexity of computations based on device capabilities and battery life.

In a step 6360, the system iterates the process, adapting to new data and evolving requirements while maintaining security and privacy standards. This final step ensures that the federated learning process is continuous and responsive to changing conditions. As users interact with their PHDB mobile devices and generate new health data, the local models are updated, contributing to the ongoing refinement of the global model. The system might also adapt to new types of data or emerging health concerns. For example, if new research reveals a previously unknown risk factor for cardiovascular disease, the system could adapt the model architecture to incorporate this new information in future training iterations.

Throughout this process, the PHDB system maintains high standards of data security and user privacy. By keeping raw data on users' devices and only sharing encrypted model updates, it minimizes the risk of data breaches or unauthorized access to sensitive health information. This approach allows the system to learn from a diverse and large dataset, potentially improving the accuracy and generalizability of health predictions, while respecting individual privacy and complying with data protection regulations. This method enables the PHDB system to leverage collective knowledge for improved health predictions and insights, all while maintaining strong privacy protections for individual users. It represents a significant advancement in privacy-preserving machine learning for health applications, potentially transforming how health-related AI models are developed and deployed in a world increasingly concerned with data privacy and security.

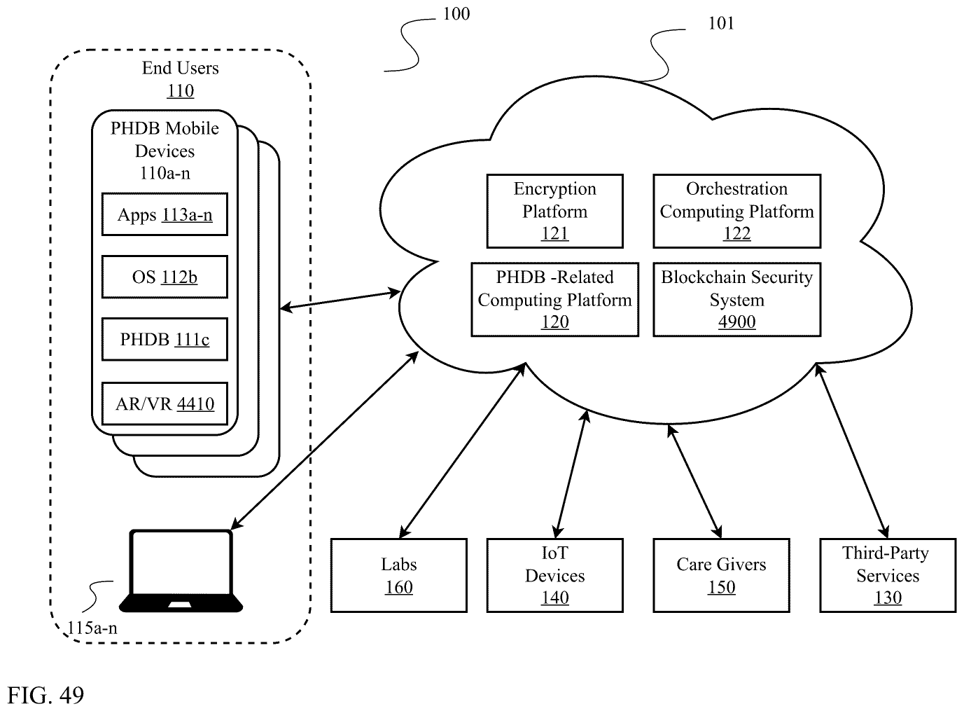

FIG. 49 is a block diagram showing an exemplary arrangement of a PHDB, where the PHDB system is secured using a blockchain security system. This addition represents a significant advancement in the system's ability to ensure data integrity, transparency, and security while maintaining user privacy. Blockchain security system 4900 integrates seamlessly with the existing PHDB-related computing platform 120, working in conjunction with other critical components such as the encryption platform 121 and the orchestration computing platform 122. This subsystem is designed to provide an immutable, distributed ledger for recording and verifying transactions and data changes within the PHDB ecosystem.

Blockchain security system 4900 interacts with data from various sources within the PHDB network. It processes information from PHDB mobile devices 110a-n, which include user applications apps 113a-n, the device's operating system (OS 112b), and the local PHDB storage 111c. This allows the system to create a secure, verifiable record of all data interactions and changes, enhancing trust and transparency in the handling of sensitive health information. Furthermore, the blockchain security system interfaces with data from IoT devices 140, labs 160, third-party services 130, and care givers 150. This comprehensive integration ensures that all data flows within the PHDB ecosystem are securely recorded and verifiable.

Blockchain security system 4900 employs advanced cryptographic techniques to create a chain of data blocks, each containing a cryptographic hash of the previous block, a timestamp, and transaction data. This structure makes it extremely difficult to alter any record without altering all subsequent blocks, providing a high level of data integrity and tamper-resistance. For example, when a user updates their health record through their PHDB mobile device (110a-n), blockchain security system 4900 could create a new block containing a hash of the update, timestamp, and a reference to the previous version of the record. This creates an auditable trail of all changes to the user's health data, without exposing the sensitive details of those changes.

The system can also implement smart contracts, which are self-executing contracts with the terms of the agreement directly written into code. These could be used to automate and secure various processes within the PHDB system, such as managing access permissions, executing data sharing agreements, or triggering alerts based on specific health data changes. Security and privacy remain paramount in this enhanced system. The blockchain security system works in tandem with the encryption platform 121 to ensure that while the fact of a transaction is recorded on the blockchain, the content of that transaction remains encrypted and private. This allows for verification of data integrity without compromising data confidentiality.

Orchestration computing platform 122 plays a crucial role in managing the interactions between the blockchain security system and other components of the PHDB system. It ensures efficient coordination of blockchain operations, manages the creation and validation of new blocks, and oversees the execution of smart contracts. One of the key strengths of this blockchain approach is its ability to create a transparent, verifiable record of all data transactions while maintaining strict privacy controls. For instance, it could provide an immutable audit trail of who has accessed a user's health records, when, and for what purpose, without revealing the content of those records. This can be particularly valuable in scenarios requiring regulatory compliance or in cases where patients want to maintain control over their health data.

Blockchain security system 4900 can support more efficient and secure data sharing between different entities within the healthcare ecosystem. For example, it could facilitate secure sharing of anonymized health data for research purposes, with all data access and usage recorded on the blockchain for full transparency. The integration of AR/VR 4410 with the blockchain security system opens up possibilities for secure, immersive visualization of health data and its associated audit trails. Users could potentially navigate a visual representation of their health data history in a virtual environment, with each data point securely linked to its blockchain record.

In summary, the addition of the blockchain security system 4900 to the PHDB system marks a major advancement in securing and verifying health data transactions. By providing an immutable, distributed ledger for recording data changes and access, it enhances trust, transparency, and security within the PHDB ecosystem. This capability has the potential to transform how health data is managed and shared, ensuring data integrity and user control while facilitating more efficient collaboration in healthcare.

In one embodiment, blockchain security system 4900 offers both distributed ledger technology (DLT) options and traditional database solutions for secure data management and auditing. This hybrid approach allows organizations to choose the most appropriate technology based on their specific needs, regulatory requirements, and existing infrastructure. When configured to use DLT, blockchain security system 4900 leverages the inherent benefits of blockchain technology, such as immutability, transparency, and decentralized trust. In this mode, blockchain security system 4900 creates a tamper-resistant record of all data transactions, access logs, and system changes. Each block in the chain contains a cryptographic hash of the previous block, a timestamp, and transaction data, ensuring a verifiable and unalterable audit trail.

Alternatively, blockchain security system 4900 can be configured to use traditional database technologies for organizations that prefer or require conventional data persistence methods. In this configuration, the system employs robust relational or NoSQL databases, depending on the specific data structures and query patterns required. These databases are optimized for high-performance transaction processing and complex querying capabilities.

Regardless of the chosen technology (DLT or traditional database), the blockchain security system 4900 implements key features to ensure data integrity and comprehensive auditing. Blockchain security system 4900 maintains immutable audit logs, whether using blockchain or a traditional database, recording all data access, modifications, and system events. In the traditional database setup, this may be achieved through write-once-read-many (WORM) storage techniques and cryptographic signing of log entries.

All data entries and modifications are cryptographically signed, allowing for verification of data origin and integrity. In the blockchain configuration, this is inherent to the technology. For traditional databases, the system implements digital signatures and hash chains to provide similar assurances. Identity and access management controls are implemented, recording all user actions and system access attempts. This may include multi-factor authentication and role-based access control, which are consistently applied across both DLT and traditional database configurations.

Blockchain security system 4900 ensures that all sensitive data is encrypted both at rest and in transit, using industry-standard encryption algorithms. The encryption keys are managed securely, with key rotation policies in place to enhance long-term security. Each transaction or data modification is timestamped, providing a chronological record of all system activities. In the blockchain configuration, this is part of the block structure. For traditional databases, the system implements secure timestamping services to ensure the accuracy and immutability of time records.

In the blockchain configuration, smart contracts automate and enforce data handling rules. For traditional databases, equivalent functionality is achieved through stored procedures and triggers, which encapsulate and enforce business logic and data integrity rules. When using DLT, blockchain security system 4900 may leverage distributed consensus mechanisms to validate transactions across multiple nodes. For traditional database setups, the system can be configured with database replication and distributed consensus protocols to achieve similar levels of data verification and resilience.

Blockchain security system 4900 may provide comprehensive audit reporting, generating detailed logs of all data access, modifications, and system events. These reports are consistent across both DLT and traditional database configurations, ensuring that organizations can meet their audit and compliance requirements regardless of the underlying technology. Both configurations support configurable data retention policies and secure archiving mechanisms, allowing organizations to maintain historical records while managing storage efficiency.

Blockchain security system 4900 is designed to integrate with existing healthcare information systems, electronic health records (EHRs), and health information exchanges (HIEs). This interoperability is maintained regardless of whether DLT or traditional database technology is used. By offering this flexible approach, blockchain security system 4900 accommodates various organizational needs and regulatory landscapes. Organizations can choose the technology that best fits their requirements while still benefiting from robust security measures and comprehensive audit capabilities. This adaptability ensures that the PHDB system can be deployed in diverse healthcare environments, from cutting-edge research institutions leveraging blockchain technology to established healthcare providers preferring traditional database solutions.

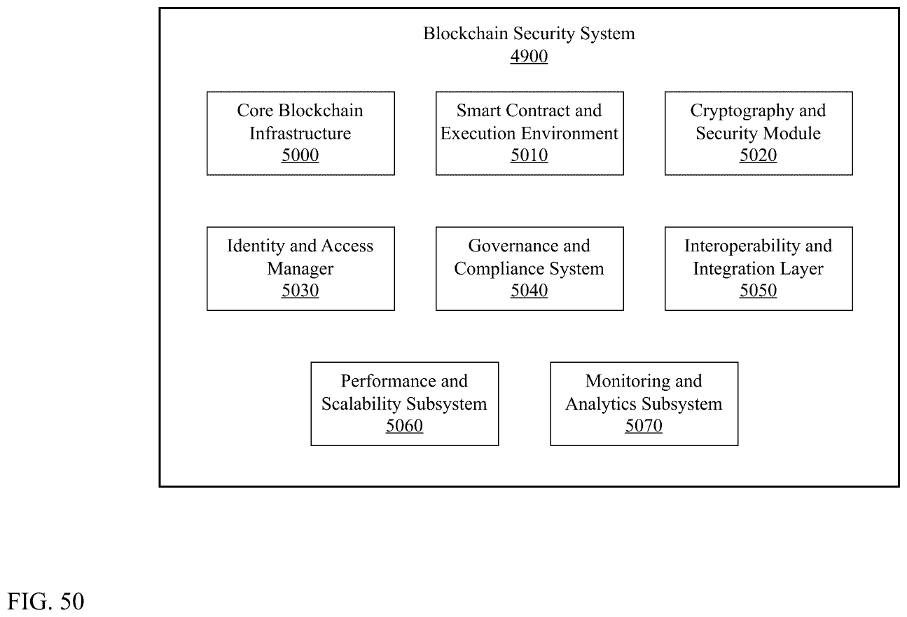

FIG. 50 is a diagram showing an exemplary subsystem of a PHDB with blockchain security, a blockchain security system. This system is designed to enhance data security, integrity, and transparency while maintaining user privacy. Blockchain infrastructure 5000 forms the foundation of the security system. It implements the distributed ledger technology, creating and maintaining a chain of cryptographically linked blocks. Each block contains a hash of the previous block, a timestamp, and transaction data, ensuring an immutable record of all data interactions within the PHDB system. For example, when a user updates their health record through their PHDB mobile device 110a-n, a new block is created containing a hash of this update, preserving the chronology and integrity of the data.

A smart contract and execution environment 5010 enables the creation and execution of self-executing contracts with predefined rules. These smart contracts can automate various processes within the PHDB system, such as managing data access permissions or triggering alerts based on specific health data changes. For instance, a smart contract could automatically grant temporary access to a user's health data to an emergency care provider, with this access revoked once the emergency situation is resolved. A cryptography and security module 5020 is responsible for implementing advanced encryption techniques to secure data and transactions on the blockchain. It works in conjunction with the encryption platform 121 to ensure that while transaction records are visible on the blockchain, the content of these transactions remains encrypted and private. This module might employ techniques like zero-knowledge proofs to verify the validity of transactions without revealing their contents.

An identity and access manager 5030 oversees user authentication and authorization within the blockchain network. It ensures that only authorized entities can access or modify data, creating a secure environment for sensitive health information. This component might implement decentralized identity solutions, allowing users to have more control over their digital identities and how their personal information is shared. A governance and compliance system 5040 ensures that all operations within the blockchain network adhere to predefined rules and regulatory requirements. It manages the implementation of consensus mechanisms, voting systems for network upgrades, and compliance checks for data protection regulations like HIPAA. This system could, for example, enforce rules about how long certain types of health data can be stored or who can access specific categories of information.

An interoperability and integration layer 5050 facilitates communication between the blockchain security system and other components of the PHDB ecosystem, including external systems. It ensures that the blockchain can interact seamlessly with various data sources, from IoT devices 140 to third-party services 130. This layer might implement standards like HL7 FHIR to ensure interoperability with other healthcare systems. A performance and scalability subsystem 5060 is crucial for maintaining the efficiency of the blockchain network as it grows. It might implement solutions like sharding or layer-2 protocols to handle increased transaction volumes without compromising speed or security. This is particularly important in a healthcare context where rapid access to data can be critical.

A monitoring and analytics subsystem 5070 provides real-time insights into the operation of the blockchain network. It can detect anomalies that might indicate security threats, track network performance, and generate reports for auditing purposes. This subsystem could, for instance, alert system administrators to unusual patterns of data access that might indicate a breach attempt. This blockchain security system offers several advantages within the PHDB ecosystem. It provides an immutable audit trail of all data transactions, enhancing transparency and accountability in health data management. For example, patients could verify who has accessed their health records and when, without the risk of this access log being tampered with.

The system also supports more secure and efficient data sharing. Researchers could access anonymized health data for studies, with all data access recorded on the blockchain, ensuring transparency and compliance with data use agreements. Smart contracts could automate the process of granting and revoking access, reducing administrative overhead and potential human errors. Moreover, the blockchain security system can enhance the reliability of health data. By creating an immutable record of all data changes, it becomes possible to track the provenance of health information, which is crucial for ensuring data quality in clinical decision-making.

The integration with other PHDB components, such as the AR/VR system 4410, opens up possibilities for innovative data visualization and interaction. Users could navigate a visual representation of their health data history in a virtual environment, with each data point securely linked to its blockchain record, providing an intuitive way to understand their health data and its security. In summary, the blockchain security system 4900 represents a significant advancement in securing health data within the PHDB ecosystem. By providing a transparent, immutable, and secure platform for managing health data transactions, it enhances trust, ensures data integrity, and facilitates more efficient and secure collaboration in healthcare, all while maintaining the privacy and confidentiality of sensitive health information.

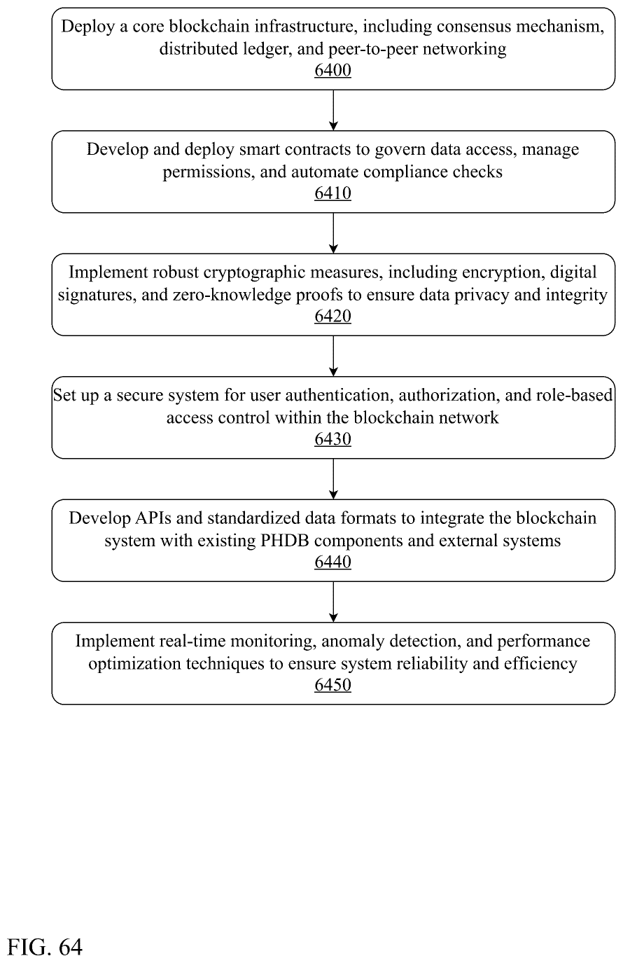

FIG. 64 is a flow diagram showing exemplary method for configuring a PHDB system with a blockchain security system. This method enhances the security, transparency, and integrity of personal health data within the PHDB ecosystem. In a first step 6400, the system deploys a core blockchain infrastructure, including consensus mechanism, distributed ledger, and peer-to-peer networking. This step establishes the foundational elements of the blockchain system, ensuring a decentralized and tamper-resistant network for storing and managing health data. A consensus mechanism, such as Proof of Stake or Practical Byzantine Fault Tolerance, ensures agreement on the state of the blockchain across all nodes. A distributed ledger maintains a complete and immutable record of all transactions, while peer-to-peer networking enables direct communication between nodes without the need for intermediaries.

In a step 6410, the system develops and deploys smart contracts to govern data access, manage permissions, and automate compliance checks. Smart contracts are self-executing contracts with the terms of the agreement directly written into code. In the context of the PHDB system, these smart contracts can automate and secure various processes such as managing access permissions, executing data sharing agreements, or triggering alerts based on specific health data changes. This step ensures that data access and usage comply with predefined rules and regulations, enhancing both security and efficiency.

In a step 6420, the system implements robust cryptographic measures, which may include encryption, digital signatures, and zero-knowledge proofs to ensure data privacy and integrity. Encryption protects the confidentiality of sensitive health data, while digital signatures verify the authenticity and origin of data. Zero-knowledge proofs allow for the verification of certain information without revealing the underlying data, which is particularly useful in healthcare scenarios where privacy is paramount. These cryptographic measures work in tandem with the blockchain infrastructure to provide a high level of security and privacy for personal health data.

In a step 6430, the system sets up a secure system for user authentication, authorization, and role-based access control within the blockchain network. This step ensures that only authorized users can access specific parts of the blockchain and perform certain actions. Role-based access control allows for fine-grained permission management, ensuring that healthcare providers, patients, and other stakeholders have appropriate access levels to health data. This system may incorporate multi-factor authentication and biometric verification for enhanced security.

In a step 6440, the system develops APIs and standardized data formats to integrate the blockchain system with existing PHDB components and external systems. This step is crucial for ensuring interoperability between the blockchain security system and other elements of the PHDB ecosystem, as well as external healthcare systems. Standardized data formats, such as FHIR (Fast Healthcare Interoperability Resources), facilitate seamless data exchange and integration. APIs enable secure and efficient communication between the blockchain and other systems, allowing for real-time data updates and queries.

In a step 6450, the system implements real-time monitoring, anomaly detection, and performance optimization techniques to ensure system reliability and efficiency. This final step focuses on maintaining the health and performance of the blockchain security system. Real-time monitoring allows for immediate detection of potential security threats or system issues. Anomaly detection algorithms can identify unusual patterns or behaviors that may indicate security breaches or data tampering. Performance optimization techniques ensure that the blockchain system operates efficiently, handling high transaction volumes without compromising speed or security. By implementing these steps, the PHDB system leverages blockchain technology to create a secure, transparent, and efficient environment for managing personal health data. This blockchain security system enhances data integrity, improves trust among stakeholders, and supports more effective collaboration in healthcare while maintaining strict privacy and compliance standards.

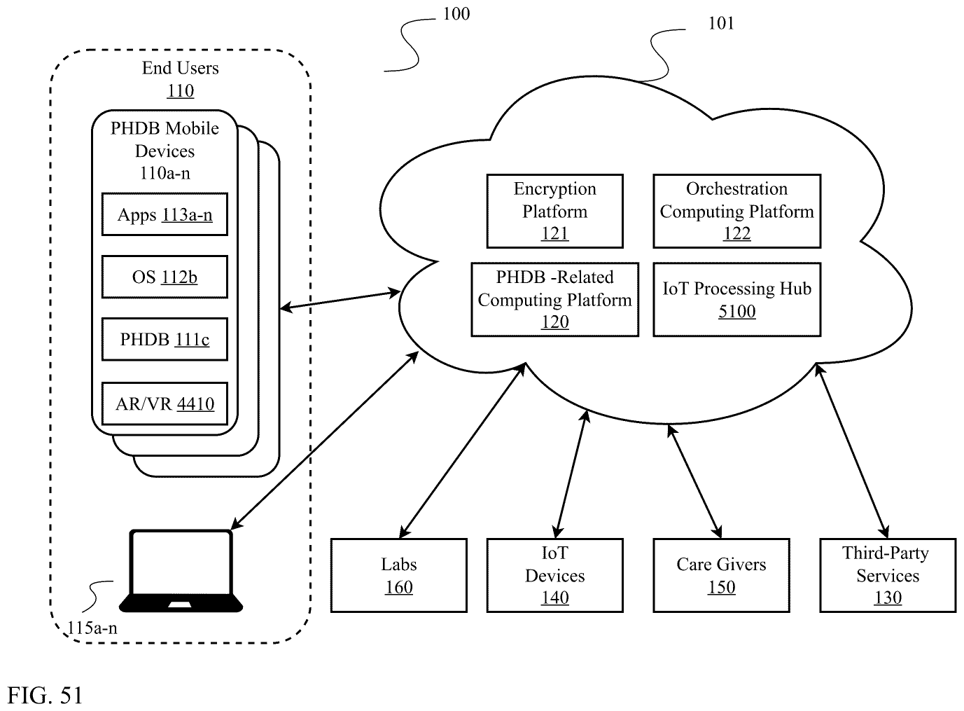

FIG. 51 is a block diagram showing an exemplary arrangement of a PHDB, where the PHDB system is configured with an IoT data processing hub. The system 100 comprises various interconnected components that work together to provide a comprehensive personal health data management solution. At the core of the system is the cloud-based platforms 101, which houses several key components including the PHDB-related computing platform 120, the encryption platform 121, and the orchestration computing platform 122.

A significant addition to this system is IoT processing hub 5100, which serves as a central node for managing and processing data from various Internet of Things (IoT) devices 140. The IoT processing hub 5100 is designed to handle the vast amounts of data generated by connected health devices, wearables, and other IoT sensors. It acts as an intermediary between the IoT devices 140 and the core PHDB system, performing initial data processing, aggregation, and analysis before transmitting relevant information to the PHDB-related computing platform 120. IoT processing hub 5100 enhances the system's capability to handle real-time data streams, enabling more efficient and timely health monitoring and analysis. It can perform edge computing tasks, reducing latency and bandwidth usage by processing data closer to its source. This is particularly beneficial for applications requiring real-time responses, such as continuous glucose monitoring or heart rate tracking.

End users 110 interact with the system through PHDB mobile devices 110a-n, which contain applications (apps) 113a-n, an operating system (OS) 112b, and a local PHDB 111c. These devices communicate with the cloud-based platforms 101, sending and receiving data as needed. The system also integrates with personal computers 115a-n, providing additional access points for users. The PHDB system interfaces with various external entities, including labs 160, third-party services 130, and care givers 150. These connections allow for the integration of diverse data sources, enhancing the comprehensiveness of the personal health database. The encryption platform 121 ensures that all data transmissions, both from IoT devices and other sources, are securely encrypted, maintaining patient privacy and data integrity.

Orchestration computing platform 122 plays a crucial role in coordinating the various components of the system, including managing data flows between the IoT processing hub 5100, the PHDB-related computing platform 120, and other system elements. It ensures efficient and secure data exchange, optimizing system performance and resource utilization. In one embodiment, the system may integrate AR/VR capabilities 4410. This component allows for immersive visualization of health data, potentially enabling more intuitive interactions with personal health information and enhanced telemedicine experiences. The AR/VR integration could leverage data processed by the IoT hub to create real-time, data-rich visual representations of a user's health status.

IoT processing hub 5100 significantly enhances the PHDB system's ability to handle diverse and high-volume data streams from IoT devices. It enables more sophisticated real-time health monitoring, predictive analytics, and personalized health insights by efficiently processing and integrating data from various connected devices. This addition makes the PHDB system more robust and capable of supporting advanced applications in personalized medicine, remote patient monitoring, and proactive health management.

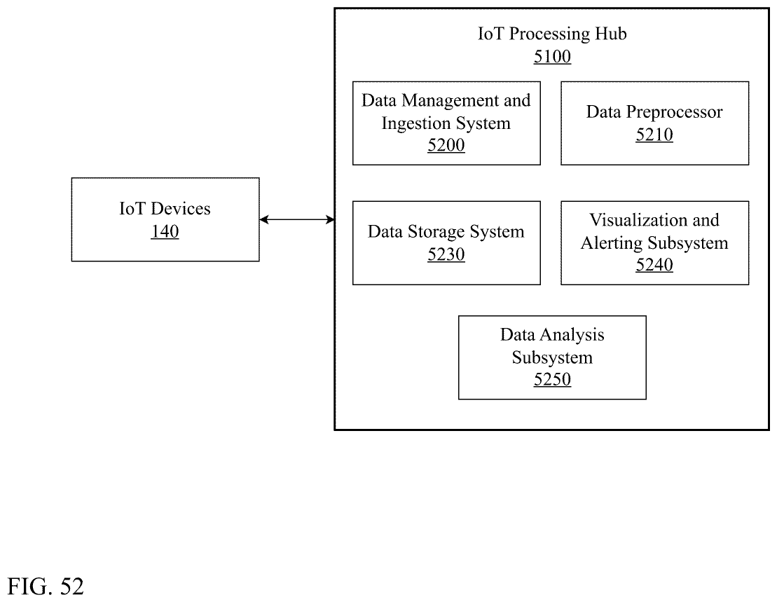

FIG. 52 is a diagram showing an exemplary subsystem of a PHDB with IoT processing, an IoT processing hub. IoT Processing hub 5100, is designed to efficiently manage, process, and analyze data from various IoT devices 140, enhancing the PHDB system's capability to handle real-time health data streams. IoT processing hub 5100 comprises of several interconnected subsystems, each playing a crucial role in the data processing pipeline. At the entry point of the hub is a data management and ingestion system 5200. This subsystem is responsible for receiving and managing the influx of data from diverse IoT devices 140. It handles the initial reception of data, ensuring proper data formatting and organization. The data management and ingestion system 5200 may employ techniques like data streaming and batch processing to efficiently handle both real-time and historical data from devices such as wearable fitness trackers, continuous glucose monitors, smart scales, and other health monitoring equipment.

Once the data is ingested, it moves to a data preprocessor 5210. This subsystem is critical for ensuring data quality and consistency. It performs tasks such as data cleaning, normalization, and transformation. For example, if different IoT devices measure the same parameter (like heart rate) in slightly different ways, data preprocessor 5210 would normalize these measurements to a standard format. It may also handle tasks like removing outliers, filling in missing values, and converting units to ensure consistency across the dataset. The processed data is then stored in data storage system 5230. This system is designed to efficiently store and retrieve large volumes of health data. It may utilize a combination of storage technologies, such as high-speed databases for recent data and more cost-effective long-term storage for historical data. The storage system ensures that data is readily available for analysis while maintaining data integrity and security.

A data analysis subsystem 5250 is the brain of IoT processing hub 5100. It applies various analytical techniques, including machine learning algorithms, to extract meaningful insights from the IoT data. This subsystem could perform tasks such as trend analysis, anomaly detection, and predictive modeling. For instance, it might analyze a user's continuous glucose monitor data alongside their activity and diet information to predict potential hypoglycemic events.

A visualization and alerting subsystem 5240 is responsible for presenting the analyzed data in an understandable format and generating alerts when necessary. This subsystem could create dynamic dashboards for healthcare providers or patients, showing real-time health metrics and trends. It would also be responsible for sending notifications or alerts based on predefined rules or anomalies detected by the analysis subsystem.

These subsystems work in close coordination with each other. For example, data analysis subsystem 5250 might request specific datasets from data storage system 5230, process this data, and then pass the results to the visualization and alerting subsystem 5240 for display or alert generation. Similarly, data preprocessor 5210 might flag certain data points for immediate analysis, prompting data analysis subsystem 5250 to perform real-time computations.

IoT processing hub 5100 significantly enhances the PHDB system's ability to handle the complex, high-volume data streams generated by modern IoT health devices. By efficiently processing and analyzing this data, it enables more sophisticated real-time health monitoring, predictive analytics, and personalized health insights. This aligns with the new disclosure's emphasis on comprehensive data integration and analysis for improved patient care and outcomes. The hub's ability to handle diverse data types and perform edge computing aligns with the concept of distributed intelligence mentioned in the new disclosure, allowing for more efficient and responsive health monitoring and decision support.

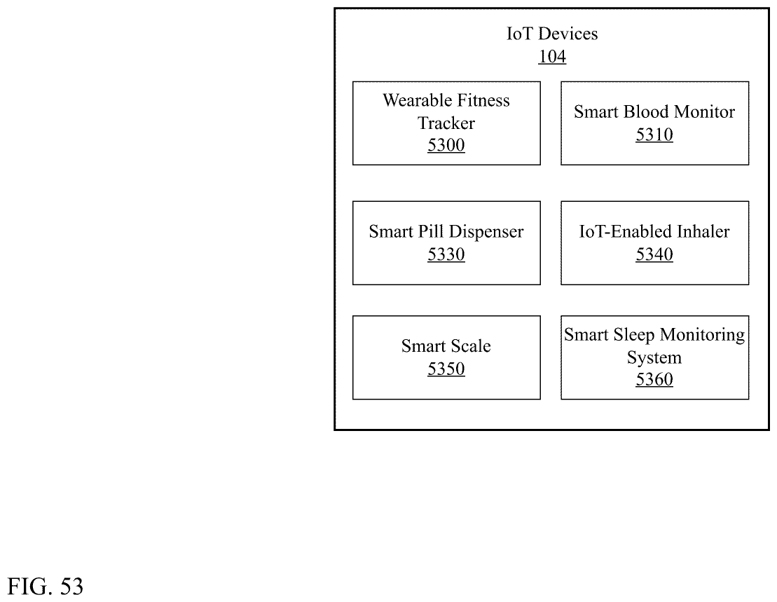

FIG. 53 is a diagram showing exemplary IoT devices that may be connected to an IoT processing hub. These devices, collectively represented as component 140, showcase the diverse range of Internet of Things (IoT) sensors and smart devices that can contribute valuable health data to the PHDB system. In one embodiment, IoT device may comprise one of the devices mention in the figure. A wearable fitness tracker 5300 typically includes sensors for monitoring various health metrics such as heart rate, steps taken, calories burned, and sleep patterns. For example, a smartwatch might continuously track a user's heart rate and physical activity throughout the day, providing insights into their overall fitness and cardiovascular health.

A smart blood monitor 5310 represents devices designed for measuring blood-related health metrics. This could include continuous glucose monitors for diabetes management, which provide real-time blood sugar readings, or blood pressure monitors that can regularly measure and record a user's blood pressure throughout the day. A smart pill dispenser 5330 is another IoT device shown. These devices can be programmed to dispense medications at specific times and can track whether a patient has taken their prescribed doses. This technology is particularly valuable for managing complex medication regimens and improving medication adherence. An IoT-enabled inhaler 5340 is a specialized device for individuals with respiratory conditions such as asthma or COPD. These smart inhalers can track usage patterns, measure the effectiveness of medication delivery, and even record environmental factors that may trigger respiratory issues.

A smart scale 5350 is also depicted, which goes beyond just measuring weight. Modern smart scales can often measure body composition, including metrics like body fat percentage, muscle mass, and bone density. Some advanced models can even measure heart rate and assess arterial health. The smart sleep monitoring system 5360 represents devices designed to track and analyze sleep patterns. This could include smart mattresses or bedside devices that monitor sleep duration, quality, breathing patterns, and even detect sleep disorders like sleep apnea.

In addition to these devices shown in the figure, there are numerous other IoT devices that could be integrated into the PHDB system via the IoT processing hub. These might include smart thermometers for tracking body temperature, UV exposure monitors to help prevent skin damage, hydration sensors to ensure proper fluid intake, and posture correction devices to improve ergonomics and prevent musculoskeletal issues. Other potential devices could include smart clothing with embedded sensors for comprehensive body monitoring, IoT-enabled blood coagulation testing devices for patients on anticoagulation therapy, and smart contact lenses capable of measuring intraocular pressure for glaucoma management. Environmental sensors, while not worn on the body, could also provide valuable data to the PHDB system. These might include air quality monitors, pollen sensors, or even smart water quality testers, all of which can have significant impacts on an individual's health.

The diverse array of IoT devices illustrated and described demonstrates the potential for comprehensive, real-time health monitoring and data collection. By integrating data from these various sources, the IoT processing hub can provide a holistic view of an individual's health status, enabling more personalized and proactive healthcare management within the PHDB system.

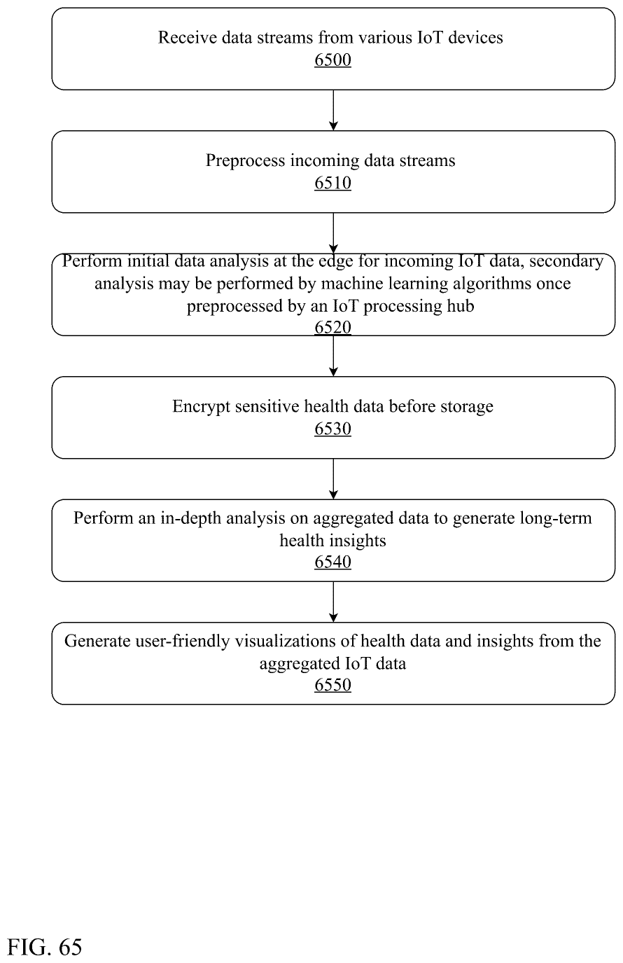

FIG. 65 is a flow diagram showing exemplary method for configuring a PHDB system with an IoT processing hub. This method illustrates the key steps in handling data from Internet of Things (IoT) devices within the Personal Health Database (PHDB) ecosystem. In a first step 6500, the system receives data streams from various IoT devices. This initial step involves the ingestion of diverse data types from a wide array of IoT health devices such as wearable fitness trackers, smart blood monitors, IoT-enabled inhalers, smart scales, and sleep monitoring systems. The IoT processing hub acts as a central point for collecting these real-time data streams, which may include metrics like heart rate, blood glucose levels, medication adherence, weight, and sleep patterns.

In a step 6510, the system preprocesses the incoming data streams. This step involves cleaning, normalizing, and transforming the raw data into a standardized format suitable for further analysis. The preprocessing may include tasks such as removing noise, handling missing values, correcting for device-specific biases, and aligning timestamps across different data sources. This step ensures that the data from various IoT devices is consistent and compatible for integrated analysis.

In a step 6520, the system performs initial data analysis at the edge for incoming IoT data, with secondary analysis potentially performed by machine learning algorithms once preprocessed by an IoT processing hub. Edge computing allows for real-time processing of time-sensitive data, reducing latency and bandwidth usage. This step might involve quick calculations or basic anomaly detection. The secondary analysis, leveraging more complex machine learning algorithms, can provide deeper insights once the data has been aggregated and preprocessed by the IoT hub.

In a step 6530, the system encrypts sensitive health data before storage. This step aids in maintaining patient privacy and complying with healthcare data protection regulations. The encryption process ensures that even if unauthorized access occurs, the sensitive health information remains protected. This step may involve using advanced encryption standards and potentially leveraging blockchain technology for enhanced security and data integrity.

In a step 6540, the system performs an in-depth analysis on aggregated data to generate long-term health insights. This step goes beyond the initial edge analysis, utilizing the full power of the PHDB system's analytics capabilities. It may involve applying sophisticated machine learning models, statistical analysis, and pattern recognition algorithms to identify trends, predict potential health issues, and generate personalized health recommendations. This analysis could combine IoT data with other health records in the PHDB for a more comprehensive understanding of the patient's health status.

In a step 6550, the system generates user-friendly visualizations of health data and insights from the aggregated IoT data. This final step focuses on presenting the complex health data and derived insights in an easily understandable format. Visualizations could include interactive dashboards, trend graphs, and personalized health reports. These visual representations help patients and healthcare providers to quickly grasp important health trends and make informed decisions.

This method enables the PHDB system to effectively integrate and utilize the wealth of data generated by IoT health devices. By systematically processing, analyzing, and presenting this data, the system can provide more comprehensive, real-time, and personalized health insights, ultimately supporting better health outcomes and more proactive healthcare management.

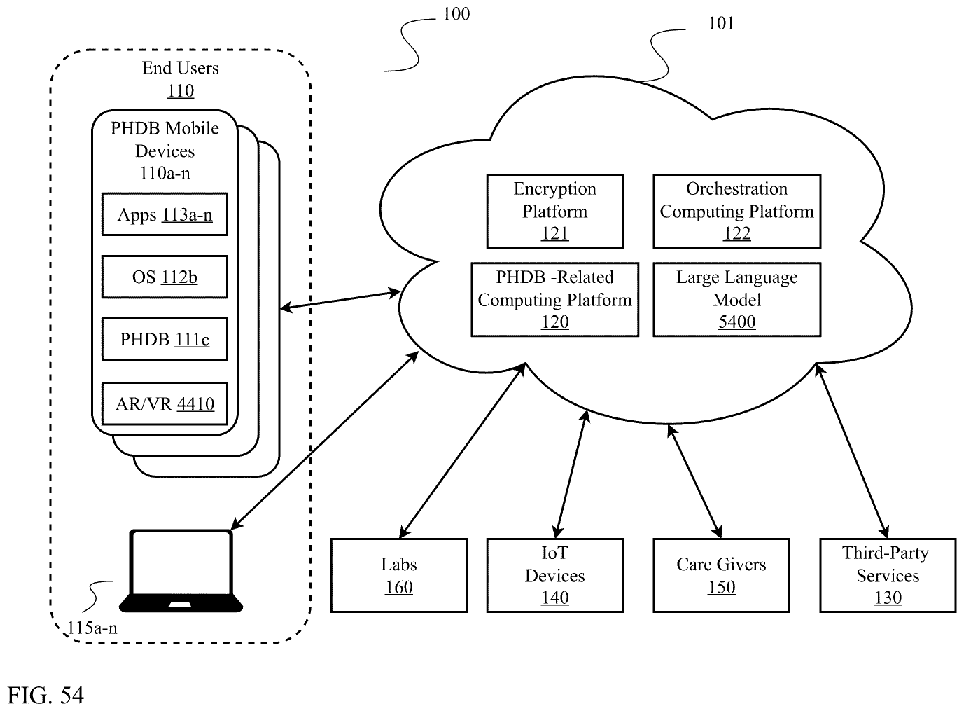

FIG. 54 is a block diagram showing an exemplary arrangement of a PHDB, where the PHDB system is configured with a Large Language Model. The system 100 comprises various interconnected components that work together to provide a comprehensive personal health data management solution enhanced by advanced natural language processing capabilities. At the core of the system is the cloud-based platforms 101, which houses several key components including the PHDB-related computing platform 120, the encryption platform 121, and the orchestration computing platform 122.

In one embodiment, a significant addition to this system is a large language model (LLM) 5400, which serves as a sophisticated natural language processing and generation engine. LLM 5400 is designed to understand and generate human-like text, enabling more intuitive interactions with the PHDB system and providing advanced capabilities for medical data analysis, interpretation, and communication. It can process and generate natural language related to medical information, patient records, and healthcare queries, enhancing the system's ability to provide personalized health insights and support medical decision-making.

End users 110 interact with the system through PHDB mobile devices 110a-n, which contain applications (apps) 113a-n, an operating system (OS) 112b, and a local PHDB 111c. These devices can now leverage LLM 5400 to provide more sophisticated and context-aware responses to user queries, offer personalized health advice, and interpret complex medical information in user-friendly language. The system also integrates with personal computers 115a-n, providing additional access points for users to interact with the LLM-enhanced PHDB system. The PHDB system interfaces with various external entities, including labs 160, third-party services 130, and care givers 150. LLM 5400 can assist in interpreting lab results, integrating information from third-party services, and facilitating more effective communication between care givers and patients. It can process and generate natural language summaries of complex medical data, making it easier for both healthcare providers and patients to understand and act upon health information.

Encryption platform 121 ensures that all data processed by the LLM, including sensitive medical information, is securely encrypted, maintaining patient privacy and data integrity. The LLM itself may incorporate privacy-preserving techniques to process sensitive information without compromising confidentiality. The orchestration computing platform 122 plays a crucial role in coordinating the various components of the system, including managing data flows between LLM 5400, the PHDB-related computing platform 120, and other system elements. It ensures that the LLM is efficiently utilized across different applications and use cases within the PHDB ecosystem.

In another embodiment, the system can be further expanded with AR/VR capabilities 4410. LLM 5400 can enhance these immersive experiences by generating natural language descriptions and explanations of visualized health data, making complex medical concepts more accessible and understandable in virtual or augmented reality environments.

The integration of LLM 5400 into the PHDB system significantly enhances its capabilities in natural language understanding and generation. This enables more intuitive user interactions, improves the interpretation and communication of medical information, and supports advanced applications in personalized medicine, medical research, and healthcare decision support. The LLM can process vast amounts of medical literature and patient data, providing up-to-date and context-aware information to both healthcare providers and patients, ultimately contributing to better health outcomes and more efficient healthcare delivery.

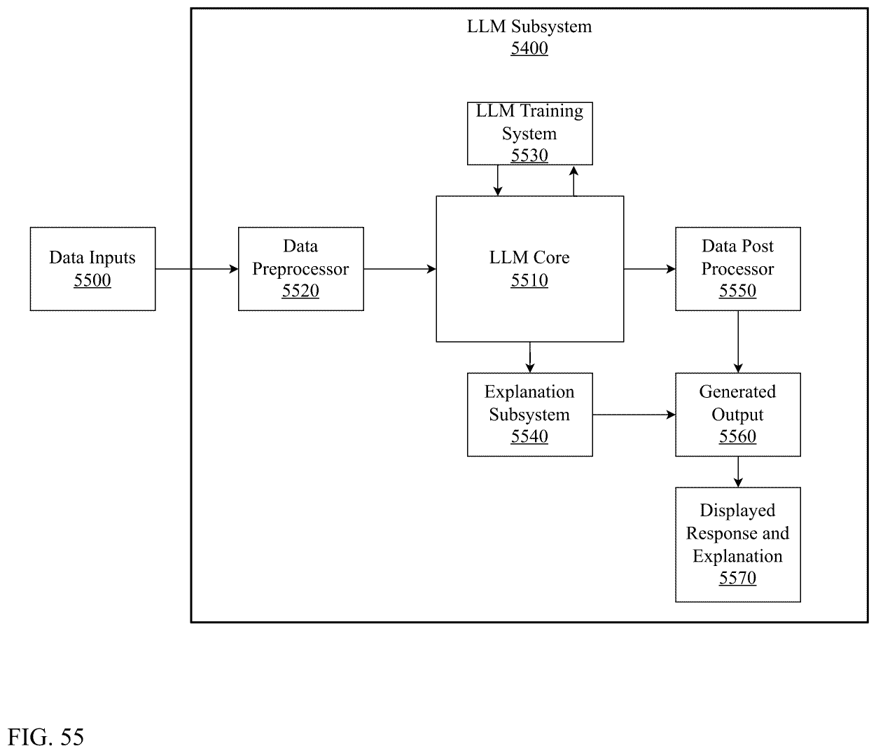

FIG. 55 is a diagram showing exemplary neural network architecture for a Large Language Model that has been integrated into the PHDB system. This figure provides a detailed look at the internal components of LLM subsystem 5400, illustrating how it processes information and generates responses within the context of personal health data management. The LLM subsystem begins with data inputs 5500, which represent the various sources of information fed into the model. In the PHDB context, these inputs could include patient health records, medical literature, clinical guidelines, and real-time data from IoT devices. The data inputs are crucial for providing the LLM with the necessary context and information to generate accurate and relevant responses.

A data preprocessor 5520 is responsible for preparing the input data for processing by the LLM core. This component may handle tasks such as tokenization, normalization of medical terms, and formatting of structured data into a form suitable for the LLM. For example, it might convert raw lab results into a standardized format or translate colloquial health descriptions into formal medical terminology. At the heart of the subsystem is LLM core 5510. This is the main neural network architecture that processes the prepared input data. Based on the new disclosure, this could be a transformer-based model similar to GPT-4, specifically trained on medical data. In other embodiments, the LLM may utilize a Variational Autoencoder or diffusion model architecture as its core. The LLM core performs the primary language understanding and generation tasks, leveraging its training on vast amounts of medical text to provide contextually relevant and accurate responses.

An LLM training system 5530 is responsible for the continuous improvement and updating of the LLM core. This system might employ techniques like federated learning, as mentioned in the new disclosure, allowing the model to learn from decentralized data sources without compromising patient privacy. It could also incorporate new medical knowledge and adapt to evolving healthcare practices over time. An explanation subsystem 5540 is a critical component for ensuring transparency and trust in the LLM's outputs. This subsystem generates explanations for the LLM's decisions and recommendations, aligning with the emphasis on explainable AI in healthcare as discussed in the new disclosure. For instance, if the LLM suggests a particular diagnosis, this subsystem would provide the reasoning behind this suggestion, citing relevant symptoms, test results, or medical literature.

A data post processor 5550 takes the raw output from the LLM core and refines it for final presentation. This might involve formatting the response for different user interfaces, translating technical medical terms into lay language when appropriate, or integrating the LLM's output with other data visualizations in the PHDB system. A generated output 5560 represents the final product of the LLM subsystem's processing. This could be a diagnosis suggestion, a treatment recommendation, or an interpretation of complex medical data. Finally, a displayed response and explanation 5570 is what the end-user sees. This combines the generated output with the explanation provided by the explanation subsystem, ensuring that users not only receive the LLM's response but also understand the reasoning behind it.

These components work together in a coordinated manner. For example, as data flows in through inputs 5500 and is preprocessed 5520, LLM core 5510 processes it and generates a response. Simultaneously, the explanation subsystem 5540 analyzes the LLM's decision-making process. Data post processor 5550 then combines the generated output and explanation into a coherent response, which is finally displayed to the user. This architecture allows for sophisticated natural language processing capabilities within the PHDB system, enabling more intuitive interaction with complex medical data, supporting clinical decision-making, and enhancing patient understanding of their health information, all while maintaining transparency and explainability in line with ethical AI practices in healthcare.

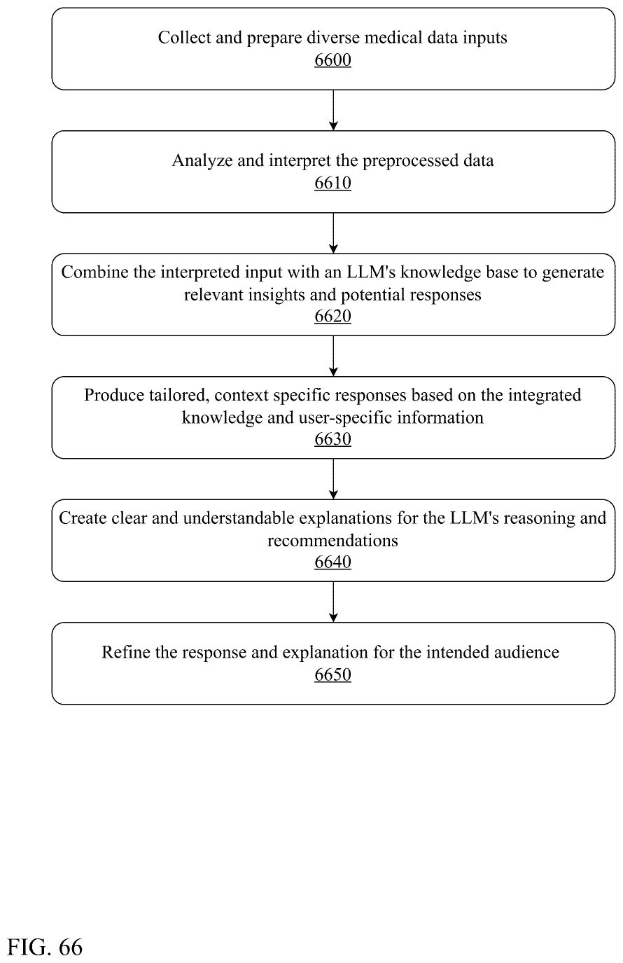

FIG. 66 is a flow diagram showing exemplary method for configuring a PHDB system with an integrated Large Language Model. This method outlines the key steps in processing medical data and generating personalized, context-aware responses using advanced natural language processing capabilities. In a first step 6600, the system collects and prepares diverse medical data inputs. This step involves gathering a wide range of medical information, including patient health records, medical literature, clinical guidelines, and real-time data from IoT devices. The data is then preprocessed, which may include tasks such as tokenization, normalization of medical terms, and formatting of structured data into a form suitable for the LLM. This step ensures that the LLM has access to comprehensive and properly formatted data for accurate analysis and response generation.

In a step 6610, the system analyzes and interprets the preprocessed data. The LLM, leveraging its training on vast amounts of medical text, processes the input data to understand the context, identify key medical concepts, and interpret the information in light of the specific query or task at hand. This step may involve techniques like named entity recognition to identify medical terms, relationship extraction to understand connections between different pieces of information, and semantic analysis to grasp the overall meaning and intent of the input.

In a step 6620, the system combines the interpreted input with the LLM's extensive knowledge base to generate relevant insights and potential responses. This step leverages the LLM's deep understanding of medical knowledge, which it has acquired through training on large volumes of medical literature and clinical data. The LLM uses this knowledge to contextualize the input, draw connections to relevant medical concepts, and formulate potential responses or recommendations. This process may involve complex reasoning, such as considering differential diagnoses, treatment options, or potential drug interactions.

In a step 6630, the system produces tailored, context-specific responses based on the integrated knowledge and user-specific information. This step focuses on personalizing the output to the specific user or situation. The LLM takes into account factors such as the user's medical history, current health status, and any specific circumstances mentioned in the input. This personalization ensures that the generated response is not only medically accurate but also relevant and applicable to the individual user's situation.

In a step 6640, the system creates clear and understandable explanations for the LLM's reasoning and recommendations. This step is crucial for ensuring transparency and building trust in the LLM's outputs. The system generates explanations that detail the logic behind its conclusions, citing relevant medical knowledge, patient-specific factors, and any other considerations that influenced its response. These explanations are designed to be comprehensible to the intended audience, whether it's a healthcare professional or a patient.

In a step 6650, the system refines the response and explanation for the intended audience. This step involves tailoring the language, detail level, and presentation of the LLM's output to suit the specific recipient. For example, if the output is intended for a patient, medical jargon might be simplified and more context provided. If it's for a healthcare provider, more technical language and detailed medical reasoning might be included. This step ensures that the LLM's insights are communicated effectively and appropriately to different users within the PHDB system.

By implementing these steps, the PHDB system leverages the power of advanced LLM technology to provide personalized, context-aware, and explainable medical insights and recommendations. This method enhances the system's ability to process complex medical information, support clinical decision-making, and improve patient understanding of health-related information, all while maintaining transparency and adaptability to different user needs.

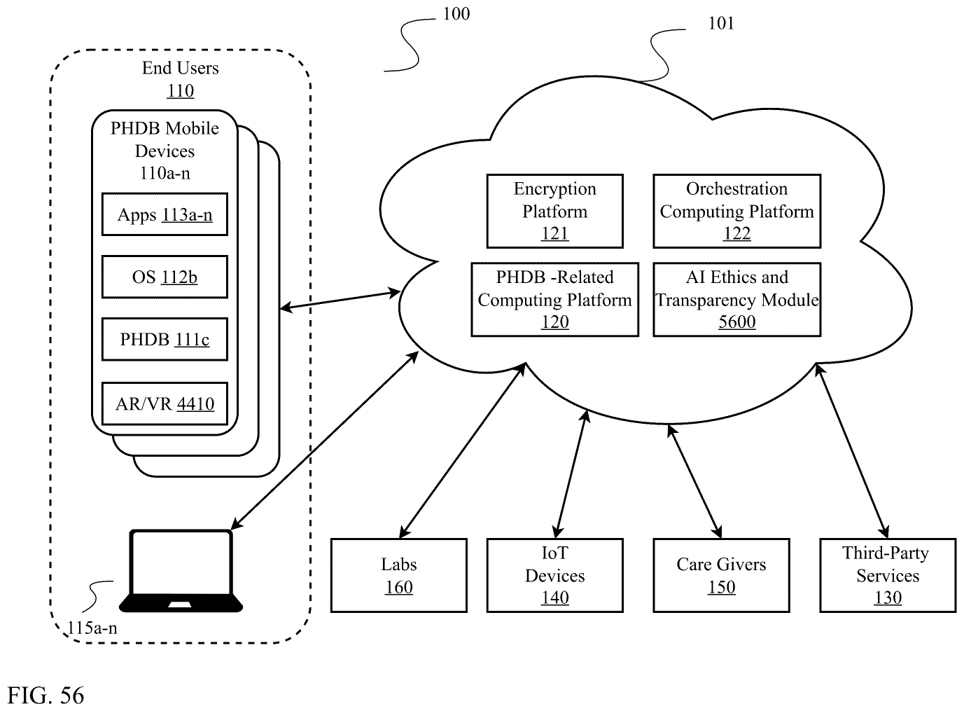

FIG. 56 is a block diagram showing an exemplary arrangement of a PHDB, where the PHDB system is configured for maintaining ethical AI usage and transparent machine learning models. System 100 comprises various interconnected components that work together to provide a comprehensive personal health data management solution with a strong emphasis on ethical AI practices and transparency. In one embodiment, an AI ethics and transparency module 5600 may be added to the PHDB system which serves as a component for ensuring responsible and accountable AI use within the PHDB ecosystem. This module is designed to address the ethical considerations and transparency requirements associated with AI in healthcare, as highlighted in the new disclosure. It works to ensure that AI-driven decisions and recommendations within the PHDB system are explainable, unbiased, and align with established ethical guidelines and regulatory requirements.

End users 110 interact with the system through PHDB mobile devices 110a-n, which contain applications (apps) 113a-n, an operating system (OS) 112b, and a local PHDB 111c. The AI ethics and transparency module 5600 enhances these interactions by providing clear explanations for AI-generated insights and recommendations, ensuring that users understand how their data is being used and interpreted. This module may also implement features for user consent management and privacy protection, giving users greater control over their health data.

The PHDB system interfaces with various external entities, including labs 160, third-party services 130, and care givers 150. The AI ethics and transparency module 5600 plays a role in managing the ethical implications of data sharing and AI-driven analysis across these interfaces. It may implement audit trails for data access and usage, ensure compliance with healthcare regulations like HIPAA, and provide transparency reports on how AI models are trained and applied to patient data. The encryption platform 121 works in conjunction with the AI ethics and transparency module 5600 to ensure that all data processing, including AI operations, maintains the highest standards of data security and privacy. This collaboration is essential for building trust in the AI-driven aspects of the PHDB system.

Orchestration computing platform 122 coordinates the activities of various system components, including the AI ethics and transparency module 5600. It ensures that ethical considerations and transparency measures are integrated into all relevant processes within the PHDB ecosystem, from data collection and analysis to the presentation of results to users and healthcare providers. A1 ethics and transparency module 5600 significantly enhances the PHDB system's trustworthiness and accountability. It implements features such as bias detection and mitigation, ensures fairness in AI-driven health recommendations across diverse patient populations, and provides mechanisms for continuous ethical assessment of AI operations. This module may also facilitate stakeholder feedback integration, allowing for ongoing refinement of ethical AI practices based on input from users, healthcare providers, and ethicists.

By incorporating AI ethics and transparency module 5600, the PHDB system sets a new standard for responsible A1 use in healthcare. It addresses critical concerns about AI transparency, fairness, and accountability, as emphasized in the new disclosure. This integration ensures that as the system leverages advanced AI capabilities for improving health outcomes and efficiency, it does so in a manner that is ethical, explainable, and aligned with the best interests of patients and healthcare providers.

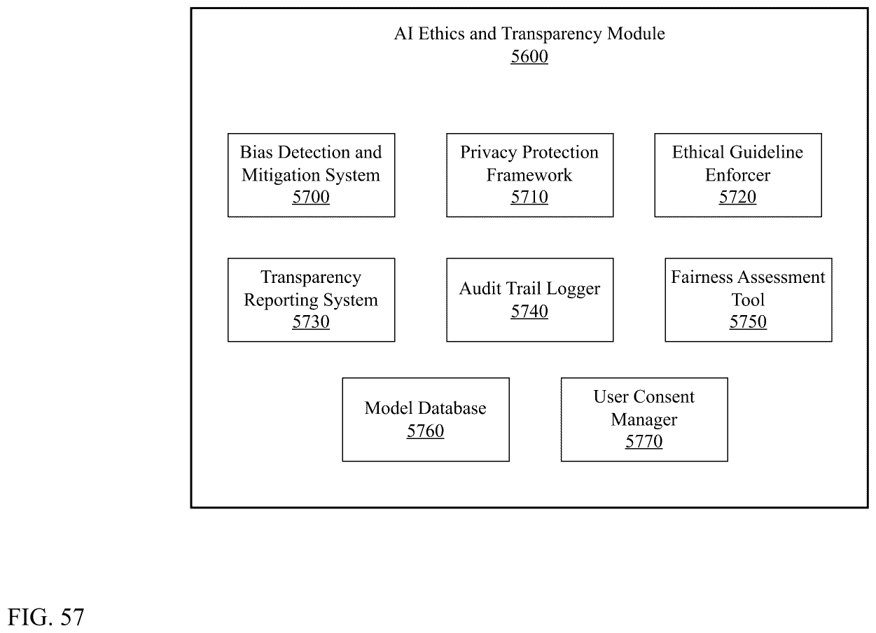

FIG. 57 is a diagram showing an exemplary subsystem of a PHDB, an ethics and transparency subsystem. Illustrated is a detailed view of the AI Ethics and Transparency Module 5600, showcasing its various subsystems that work together to ensure ethical and transparent AI operations within the PHDB system. A bias detection and mitigation system 5700 is a component that continuously monitors AI outputs for potential biases. It might use statistical techniques to identify if the AI system is consistently providing different outcomes for different demographic groups. For example, it could detect if a diagnostic AI is more likely to misdiagnose certain conditions in specific ethnic groups. Once detected, this system would work with other components to mitigate these biases, perhaps by adjusting the AI model or flagging the issue for human review.

A privacy protection framework 5710 ensures that all AI operations comply with data privacy regulations and protect sensitive patient information. This subsystem might implement techniques like differential privacy or homomorphic encryption, as mentioned in the new disclosure, to allow data analysis without compromising individual privacy. It would work closely with the user consent manager 5770 to ensure that data usage aligns with user permissions. An ethical guideline enforcer 5720 implements and maintains a set of predefined ethical rules for AI operations. These guidelines might be based on established medical ethics principles and AI ethics frameworks. For instance, it could ensure that A1 recommendations always prioritize patient well-being over cost considerations. This subsystem would interact with all other components to ensure their operations align with these ethical guidelines.

A transparency reporting system 5730 generates comprehensive reports on AI model development, training data, and decision-making processes. These reports could include information on model architectures, training datasets (with privacy considerations), and performance metrics. This system would work closely with the model database 5760 to access necessary information about AI models and their development history. An audit trail logger 5740 maintains a detailed record of all AI actions and decisions. This is crucial for accountability and can be invaluable in case of disputes or when reviewing past decisions. It might log every instance of AI-assisted diagnosis, including the input data, the AI's recommendation, and the final decision made by healthcare providers. A fairness assessment tool 5750 evaluates AI outputs to ensure equitable treatment across different patient groups. It might use various fairness metrics to assess if the AI system is providing consistent quality of care regardless of factors like age, gender, or socioeconomic status. This tool would work closely with the bias detection and mitigation system 5700 to address any identified fairness issues.

A model database 5760 stores information about all AI models used in the PHDB system, including their versions, training data characteristics, and performance metrics. This database is crucial for maintaining model lineage and supporting the transparency reporting system 5730. It also facilitates model updates and version control, ensuring that only approved and ethically validated models are in use. A user consent manager 5770 handles all aspects of user permissions for data usage and AI operations. It ensures that users are properly informed about how their data will be used and obtains necessary consents. This subsystem may interact closely with the privacy protection framework 5710 to enforce user-defined data usage limits.

These subsystems work in concert to create a comprehensive ethical and transparent AI environment. For example, when a new AI model is deployed, the ethical guideline enforcer 5720 would first check its compliance with established guidelines. Bias detection and mitigation system 5700 and fairness assessment tool 5750 would continuously monitor its outputs for potential issues. Transparency reporting system 5730 would generate reports on its operation, while the audit trail logger 5740 would record all its actions. The privacy protection framework 5710 and user consent manager 5770 would ensure all data usage complies with user permissions and privacy regulations.

This modular approach allows for comprehensive management of AI ethics and transparency, addressing the key concerns highlighted in the new disclosure about responsible AI use in healthcare. It ensures that as the PHDB system leverages advanced AI capabilities, it does so in a manner that is ethical, explainable, and respectful of patient rights and privacy.

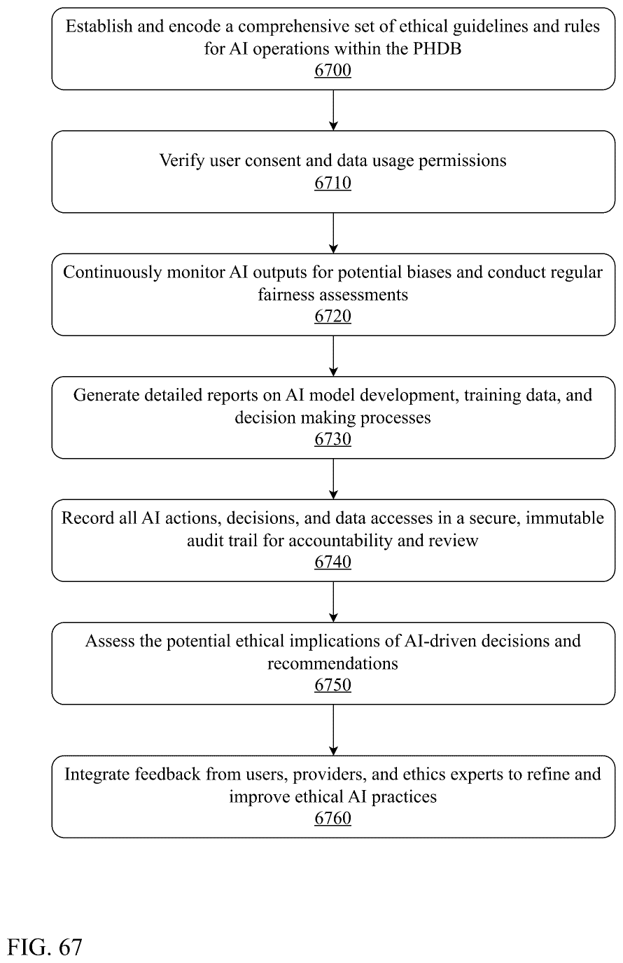

FIG. 67 is a flow diagram showing exemplary method for configuring a PHDB system for ethical AI detection and transparent AI model use. This method outlines the key steps in ensuring ethical A1 operations, maintaining transparency, and fostering trust in AI-driven healthcare solutions. In a first step 6700, the system establishes and encodes a comprehensive set of ethical guidelines and rules for AI operations within the PHDB. This step involves developing a robust framework of ethical principles tailored to the healthcare context. These guidelines may include principles such as respect for patient autonomy, beneficence, non-maleficence, and justice. The system encodes these guidelines into machine-readable formats, allowing for automated ethical checks throughout the AI operation pipeline. This step ensures that all AI activities within the PHDB system are grounded in a strong ethical foundation.

In a step 6710, the system verifies user consent and data usage permissions. This step is crucial for maintaining patient privacy and adhering to data protection regulations such as HIPAA. The system implements robust mechanisms to obtain, record, and verify user consent for various data usage scenarios. It also ensures that AI operations only access and process data in accordance with the permissions granted by users. This step may involve the use of advanced cryptographic techniques, such as homomorphic encryption, to enable data analysis while preserving privacy.

In a step 6720, the system continuously monitors AI outputs for potential biases and conducts regular fairness assessments. This ongoing process involves analyzing AI decisions and recommendations to detect any systematic biases, such as disparities in diagnosis or treatment recommendations across different demographic groups. The system employs sophisticated statistical techniques and fairness metrics to assess the equitability of A1 outputs. When biases are detected, the system triggers mitigation processes, which may include model retraining, data augmentation, or human expert review.

In a step 6730, the system generates detailed reports on AI model development, training data, and decision-making processes. These transparency reports provide comprehensive information about the AI models used within the PHDB system, including their architecture, training methodologies, and performance metrics. The reports also detail the characteristics of the training data, ensuring transparency about potential limitations or biases in the data. This step is crucial for building trust among users and healthcare providers, as it allows for scrutiny and validation of the AI systems.

In a step 6740, the system records all AI actions, decisions, and data accesses in a secure, immutable audit trail for accountability and review. This audit trail serves as a comprehensive log of all AI operations within the PHDB system. It captures details such as the input data used, the AI model version, the generated output, and any human interventions. The immutability of this audit trail, potentially implemented using blockchain technology, ensures that the record cannot be tampered with, providing a reliable source for retrospective analysis, regulatory compliance, and dispute resolution.

In a step 6750, the system assesses the potential ethical implications of AI-driven decisions and recommendations. This step involves a proactive evaluation of the broader impacts of AI outputs on patient care, healthcare resource allocation, and societal health outcomes. The system may employ scenario analysis and ethical impact assessment tools to anticipate and mitigate potential negative consequences of AI-driven decisions. This process ensures that the AI system not only adheres to ethical guidelines in its operations but also considers the long-term and systemic effects of its recommendations.

In a step 6760, the system integrates feedback from users, providers, and ethics experts to refine and improve ethical AI practices. This final step establishes a continuous improvement loop for the AI Ethics and Transparency Module. The system collects and analyzes feedback from various stakeholders, including patients, healthcare providers, and AI ethics experts. This feedback is used to update ethical guidelines, improve transparency mechanisms, and enhance the overall performance of the AI system in terms of fairness and accountability. This step ensures that the PHDB system remains responsive to evolving ethical considerations and societal expectations regarding AI in healthcare.

By implementing these steps, the PHDB system establishes a robust framework for ethical AI operations and transparency. This method ensures that AI-driven healthcare solutions within the PHDB ecosystem are not only effective and efficient but also trustworthy, fair, and aligned with societal values and ethical standards. The continuous monitoring, reporting, and improvement processes embedded in this method contribute to the responsible development and deployment of AI in healthcare, fostering trust among users and advancing the ethical use of AI technologies in medical applications.

In one embodiment, the personal health database (PHDB) system described well-positioned to support and integrate data related to virus-based delivery vectors for gene therapies and genetic modifications. These advanced therapeutic approaches, which utilize viral vectors such as adeno-associated viruses (AAVs) or lentiviruses to deliver genetic material, can be seamlessly incorporated into the comprehensive 4D spatiotemporal model of an individual's health status.

The system's ability to collect and preprocess diverse data types, including genomic data, allows for the integration of information specific to virus-based delivery vectors. This may include data on the viral vector used, the genetic payload it carries, and the targeted tissues or cells. The multi-dimensional spatial framework representing the subject's anatomy can be leveraged to visualize and track the distribution and effects of these viral vectors within the body over time. This is particularly valuable for monitoring the efficacy and potential side effects of gene therapies delivered through viral vectors.

The real-time analysis capabilities of the PHDB system can be extended to monitor the expression of genes delivered by viral vectors, tracking their integration and activity within the subject's genome. The system's predictive modeling can be enhanced to forecast the long-term outcomes of virus-based gene therapies, taking into account the individual's unique genetic profile and physiological state. This integration of virus-based delivery vector data into the PHDB system supports personalized medicine approaches, enabling healthcare providers to tailor gene therapies more precisely and monitor their effects with unprecedented detail and accuracy.

In another embodiment, the process of converting diverse health data into a “common format” within the PHDB system involves a series of data transformations that go beyond simple standardization. This process may include schematization, normalization, and semantification of the data, ensuring that information from various sources can be integrated seamlessly into the spatiotemporal model.

Schematization involves mapping the incoming data to a predefined data structure that accommodates the diverse types of health information. This step ensures that data from different sources, such as genomic sequencing results, imaging studies, and real-time health metrics, can be organized in a consistent and interoperable manner. Normalization further refines this data, adjusting for variations in measurement units or scales, and resolving inconsistencies to create a unified representation of the user's health information.

The semantification process adds another layer of sophistication by attaching meaningful context to the data. This involves mapping the normalized data to standardized medical ontologies and terminologies, enabling the system to understand the relationships between different health concepts. Additionally, the system can perform vectorization of the semantically enriched data, converting text-based information into numerical vectors that can be processed by machine learning algorithms. This vectorization supports advanced analysis techniques and facilitates the generation of Retrieval-Augmented Generation (RAG) content on demand, providing users with personalized, context-aware health insights.

The PHDB system is designed to proactively identify and integrate relevant public or shared data elements that are pertinent to the user's health profile. This feature acts as an automated enrichment process, continuously scanning and caching genetics studies, clinical trials, and research findings that overlap with the user's genomic data or other health characteristics. For instance, post-sequencing, the system can automatically note all genetic studies that are relevant to the user's genome, creating a personalized knowledge base that is constantly updated.

This automated enrichment extends to providing ongoing alerts about developments in medical science that are directly relevant to the user's health profile. Similar to how one might set up news alerts for topics of interest, the PHDB system keeps users informed about the latest scientific advancements, Phase 3 clinical trial results, and drug approvals that are specifically relevant to their omics data and lifestyle information. This feature ensures that users and their healthcare providers have access to the most current and personalized scientific information, supporting more informed decision-making and potentially earlier access to cutting-edge treatments or interventions.

### Exemplary Computing Environment

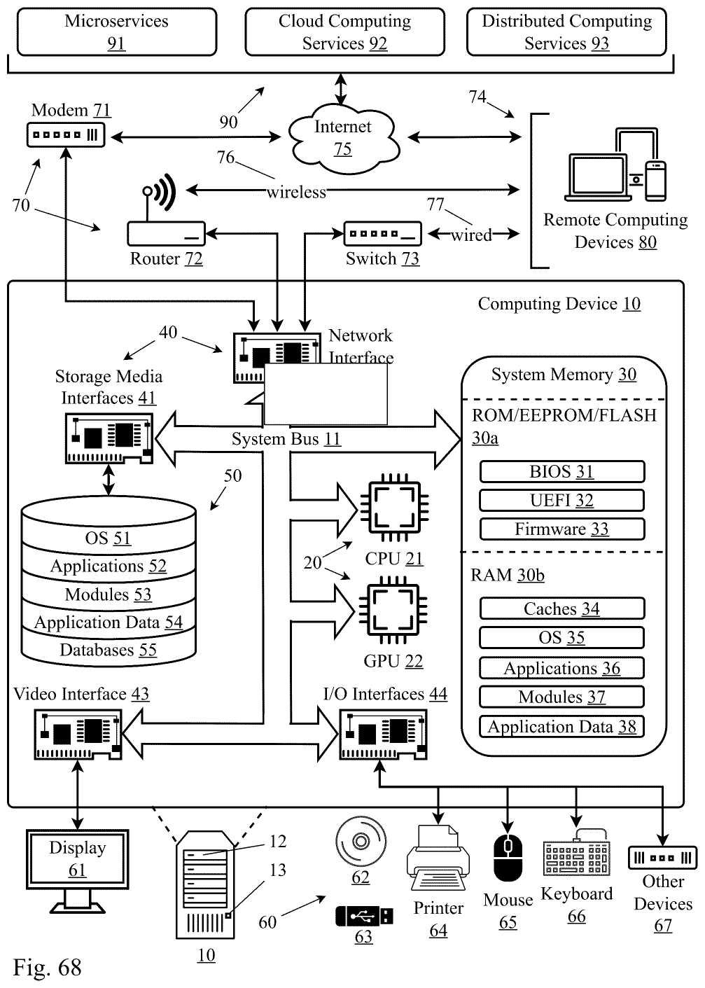

FIG. 68 illustrates an exemplary computing environment on which an embodiment described herein may be implemented, in full or in part. This exemplary computing environment describes computer-related components and processes supporting enabling disclosure of computer-implemented embodiments. Inclusion in this exemplary computing environment of well-known processes and computer components, if any, is not a suggestion or admission that any embodiment is no more than an aggregation of such processes or components. Rather, implementation of an embodiment using processes and components described in this exemplary computing environment will involve programming or configuration of such processes and components resulting in a machine specially programmed or configured for such implementation. The exemplary computing environment described herein is only one example of such an environment and other configurations of the components and processes are possible, including other relationships between and among components, and/or absence of some processes or components described. Further, the exemplary computing environment described herein is not intended to suggest any limitation as to the scope of use or functionality of any embodiment implemented, in whole or in part, on components or processes described herein.

The exemplary computing environment described herein comprises a computing device 10 (further comprising a system bus 11, one or more processors 20, a system memory 30, one or more interfaces 40, one or more non-volatile data storage devices 50), external peripherals and accessories 60, external communication devices 70, remote computing devices 80, and cloud-based services 90.

System bus 11 couples the various system components, coordinating operation of and data transmission between those various system components. System bus 11 represents one or more of any type or combination of types of wired or wireless bus structures including, but not limited to, memory busses or memory controllers, point-to-point connections, switching fabrics, peripheral busses, accelerated graphics ports, and local busses using any of a variety of bus architectures. By way of example, such architectures include, but are not limited to, Industry Standard Architecture (ISA) busses, Micro Channel Architecture (MCA) busses, Enhanced ISA (EISA) busses, Video Electronics Standards Association (VESA) local busses, a Peripheral Component Interconnects (PCI) busses also known as a Mezzanine busses, or any selection of, or combination of, such busses. Depending on the specific physical implementation, one or more of the processors 20, system memory 30 and other components of the computing device 10 can be physically co-located or integrated into a single physical component, such as on a single chip. In such a case, some or all of system bus 11 can be electrical pathways within a single chip structure.

Computing device may further comprise externally-accessible data input and storage devices 12 such as compact disc read-only memory (CD-ROM) drives, digital versatile discs (DVD), or other optical disc storage for reading and/or writing optical discs 62; magnetic cassettes, magnetic tape, magnetic disk storage, or other magnetic storage devices; or any other medium which can be used to store the desired content and which can be accessed by the computing device 10. Computing device may further comprise externally accessible data ports or connections 12 such as serial ports, parallel ports, universal serial bus (USB) ports, and infrared ports and/or transmitter/receivers. Computing device may further comprise hardware for wireless communication with external devices such as IEEE 1394 (“Firewire”) interfaces, IEEE 802.11 wireless interfaces, BLUETOOTH® wireless interfaces, and so forth. Such ports and interfaces may be used to connect any number of external peripherals and accessories 60 such as visual displays, monitors, and touch-sensitive screens 61, USB solid state memory data storage drives (commonly known as “flash drives” or “thumb drives”) 63, printers 64, pointers and manipulators such as mice 65, keyboards 66, and other devices 67 such as joysticks and gaming pads, touchpads, additional displays and monitors, and external hard drives (whether solid state or disc-based), microphones, speakers, cameras, and optical scanners.

Processors 20 are logic circuitry capable of receiving programming instructions and processing (or executing) those instructions to perform computer operations such as retrieving data, storing data, and performing mathematical calculations. Processors 20 are not limited by the materials from which they are formed or the processing mechanisms employed therein, but are typically comprised of semiconductor materials into which many transistors are formed together into logic gates on a chip (i.e., an integrated circuit or IC). The term processor includes any device capable of receiving and processing instructions including, but not limited to, processors operating on the basis of quantum computing, optical computing, mechanical computing (e.g., using nanotechnology entities to transfer data), and so forth. Depending on configuration, computing device 10 may comprise more than one processor. For example, computing device 10 may comprise one or more central processing units (CPUs) 21, each of which itself has multiple processors or multiple processing cores, each capable of independently or semi-independently processing programming instructions. Further, computing device 10 may comprise one or more specialized processors such as a graphics processing unit (GPU) 22 configured to accelerate processing of computer graphics and images via a large array of specialized processing cores arranged in parallel.

System memory 30 is processor-accessible data storage in the form of volatile and/or nonvolatile memory. System memory 30 may be either or both of two types: non-volatile memory and volatile memory. Non-volatile memory 30a is not erased when power to the memory is removed and includes memory types such as read only memory (ROM), electronically-erasable programmable memory (EEPROM), and rewritable solid state memory (commonly known as “flash memory”). Non-volatile memory 30a is typically used for long-term storage of a basic input/output system (BIOS) 31, containing the basic instructions, typically loaded during computer startup, for transfer of information between components within computing device, or a unified extensible firmware interface (UEFI), which is a modern replacement for BIOS that supports larger hard drives, faster boot times, more security features, and provides native support for graphics and mouse cursors. Non-volatile memory 30a may also be used to store firmware comprising a complete operating system 35 and applications 36 for operating computer-controlled devices. The firmware approach is often used for purpose-specific computer-controlled devices such as appliances and Internet-of-Things (IoT) devices where processing power and data storage space is limited. Volatile memory 30b is erased when power to the memory is removed and is typically used for short-term storage of data for processing. Volatile memory 30b includes memory types such as random-access memory (RAM), and is normally the primary operating memory into which the operating system 35, applications 36, program modules 37, and application data 38 are loaded for execution by processors 20. Volatile memory 30b is generally faster than non-volatile memory 30a due to its electrical characteristics and is directly accessible to processors 20 for processing of instructions and data storage and retrieval. Volatile memory 30b may comprise one or more smaller cache memories which operate at a higher clock speed and are typically placed on the same IC as the processors to improve performance.

Interfaces 40 may include, but are not limited to, storage media interfaces 41, network interfaces 42, display interfaces 43, and input/output interfaces 44. Storage media interface 41 provides the necessary hardware interface for loading data from non-volatile data storage devices 50 into system memory 30 and storage data from system memory 30 to non-volatile data storage device 50. Network interface 42 provides the necessary hardware interface for computing device 10 to communicate with remote computing devices 80 and cloud-based services 90 via one or more external communication devices 70. Display interface 43 allows for connection of displays 61, monitors, touchscreens, and other visual input/output devices. Display interface 43 may include a graphics card for processing graphics-intensive calculations and for handling demanding display requirements. Typically, a graphics card includes a graphics processing unit (GPU) and video RAM (VRAM) to accelerate display of graphics. One or more input/output (I/O) interfaces 44 provide the necessary support for communications between computing device 10 and any external peripherals and accessories 60. For wireless communications, the necessary radio-frequency hardware and firmware may be connected to I/O interface 44 or may be integrated into I/O interface 44.

Non-volatile data storage devices 50 are typically used for long-term storage of data. Data on non-volatile data storage devices 50 is not erased when power to the non-volatile data storage devices 50 is removed. Non-volatile data storage devices 50 may be implemented using any technology for non-volatile storage of content including, but not limited to, CD-ROM drives, digital versatile discs (DVD), or other optical disc storage; magnetic cassettes, magnetic tape, magnetic disc storage, or other magnetic storage devices; solid state memory technologies such as EEPROM or flash memory; or other memory technology or any other medium which can be used to store data without requiring power to retain the data after it is written. Non-volatile data storage devices 50 may be non-removable from computing device 10 as in the case of internal hard drives, removable from computing device 10 as in the case of external USB hard drives, or a combination thereof, but computing device will typically comprise one or more internal, non-removable hard drives using either magnetic disc or solid-state memory technology. Non-volatile data storage devices 50 may store any type of data including, but not limited to, an operating system 51 for providing low-level and mid-level functionality of computing device 10, applications 52 for providing high-level functionality of computing device 10, program modules 53 such as containerized programs or applications, or other modular content or modular programming, application data 54, and databases 55 such as relational databases, non-relational databases, object oriented databases, BOSQL databases, and graph databases.

Applications (also known as computer software or software applications) are sets of programming instructions designed to perform specific tasks or provide specific functionality on a computer or other computing devices. Applications are typically written in high-level programming languages such as C++, Java, and Python, which are then either interpreted at runtime or compiled into low-level, binary, processor-executable instructions operable on processors 20. Applications may be containerized so that they can be run on any computer hardware running any known operating system. Containerization of computer software is a method of packaging and deploying applications along with their operating system dependencies into self-contained, isolated units known as containers. Containers provide a lightweight and consistent runtime environment that allows applications to run reliably across different computing environments, such as development, testing, and production systems.

The memories and non-volatile data storage devices described herein do not include communication media. Communication media are means of transmission of information such as modulated electromagnetic waves or modulated data signals configured to transmit, not store, information. By way of example, and not limitation, communication media includes wired communications such as sound signals transmitted to a speaker via a speaker wire, and wireless communications such as acoustic waves, radio frequency (RF) transmissions, infrared emissions, and other wireless media.

External communication devices 70 are devices that facilitate communications between computing devices and either remote computing devices 80, or cloud-based services 90, or both. External communication devices 70 include, but are not limited to, data modems 71 which facilitate data transmission between computing device and the Internet 75 via a common carrier such as a telephone company or internet service provider (ISP), routers 72 which facilitate data transmission between computing device and other devices, and switches 73 which provide direct data communications between devices on a network. Here, modem 71 is shown connecting computing device 10 to both remote computing devices 80 and cloud-based services 90 via the Internet 75. While modem 71, router 72, and switch 73 are shown here as being connected to network interface 42, many different network configurations using external communication devices 70 are possible. Using external communication devices 70, networks may be configured as local area networks (LANs) for a single location, building, or campus, wide area networks (WANs) comprising data networks that extend over a larger geographical area, and virtual private networks (VPNs) which can be of any size but connect computers via encrypted communications over public networks such as the Internet 75. As just one exemplary network configuration, network interface 42 may be connected to switch 73 which is connected to router 72 which is connected to modem 71 which provides access for computing device 10 to the Internet 75. Further, any combination of wired 77 or wireless 76 communications between and among computing device 10, external communication devices 70, remote computing devices 80, and cloud-based services 90 may be used. Remote computing devices 80, for example, may communicate with computing device through a variety of communication channels 74 such as through switch 73 via a wired 77 connection, through router 72 via a wireless connection 76, or through modem 71 via the Internet 75. Furthermore, while not shown here, other hardware that is specifically designed for servers may be employed. For example, secure socket layer (SSL) acceleration cards can be used to offload SSL encryption computations, and transmission control protocol/internet protocol (TCP/IP) offload hardware and/or packet classifiers on network interfaces 42 may be installed and used at server devices.

In a networked environment, certain components of computing device 10 may be fully or partially implemented on remote computing devices 80 or cloud-based services 90. Data stored in non-volatile data storage device 50 may be received from, shared with, duplicated on, or offloaded to a non-volatile data storage device on one or more remote computing devices 80 or in a cloud computing service 92. Processing by processors 20 may be received from, shared with, duplicated on, or offloaded to processors of one or more remote computing devices 80 or in a distributed computing service 93. By way of example, data may reside on a cloud computing service 92, but may be usable or otherwise accessible for use by computing device 10. Also, certain processing subtasks may be sent to a microservice 91 for processing with the result being transmitted to computing device 10 for incorporation into a larger processing task. Also, while components and processes of the exemplary computing environment are illustrated herein as discrete units (e.g., OS 51 being stored on non-volatile data storage device 51 and loaded into system memory 35 for use) such processes and components may reside or be processed at various times in different components of computing device 10, remote computing devices 80, and/or cloud-based services 90.

In an implementation, the disclosed systems and methods may utilize, at least in part, containerization techniques to execute one or more processes and/or steps disclosed herein. Containerization is a lightweight and efficient virtualization technique that allows you to package and run applications and their dependencies in isolated environments called containers. One of the most popular containerization platforms is Docker, which is widely used in software development and deployment. Containerization, particularly with open-source technologies like Docker and container orchestration systems like Kubernetes, is a common approach for deploying and managing applications. Containers are created from images, which are lightweight, standalone, and executable packages that include application code, libraries, dependencies, and runtime. Images are often built from a Dockerfile or similar, which contains instructions for assembling the image. Dockerfiles are configuration files that specify how to build a Docker image. Systems like Kubernetes also support containerd or CRI-O. They include commands for installing dependencies, copying files, setting environment variables, and defining runtime configurations. Docker images are stored in repositories, which can be public or private. Docker Hub is an exemplary public registry, and organizations often set up private registries for security and version control using tools such as Hub, JFrog Artifactory and Bintray, Github Packages or Container registries. Containers can communicate with each other and the external world through networking. Docker provides a bridge network by default but can be used with custom networks. Containers within the same network can communicate using container names or IP addresses.

Remote computing devices 80 are any computing devices not part of computing device 10. Remote computing devices 80 include, but are not limited to, personal computers, server computers, thin clients, thick clients, personal digital assistants (PDAs), mobile telephones, watches, tablet computers, laptop computers, multiprocessor systems, microprocessor based systems, set-top boxes, programmable consumer electronics, video game machines, game consoles, portable or handheld gaming units, network terminals, desktop personal computers (PCs), minicomputers, main frame computers, network nodes, virtual reality or augmented reality devices and wearables, and distributed or multi-processing computing environments. While remote computing devices 80 are shown for clarity as being separate from cloud-based services 90, cloud-based services 90 are implemented on collections of networked remote computing devices 80.

Cloud-based services 90 are Internet-accessible services implemented on collections of networked remote computing devices 80. Cloud-based services are typically accessed via application programming interfaces (APIs) which are software interfaces which provide access to computing services within the cloud-based service via API calls, which are pre-defined protocols for requesting a computing service and receiving the results of that computing service. While cloud-based services may comprise any type of computer processing or storage, three common categories of cloud-based services 90 are microservices 91, cloud computing services 92, and distributed computing services 93.

Microservices 91 are collections of small, loosely coupled, and independently deployable computing services. Each microservice represents a specific computing functionality and runs as a separate process or container. Microservices promote the decomposition of complex applications into smaller, manageable services that can be developed, deployed, and scaled independently. These services communicate with each other through well-defined application programming interfaces (APIs), typically using lightweight protocols like HTTP, gRPC, or message queues such as Kafka. Microservices 91 can be combined to perform more complex processing tasks.

Cloud computing services 92 are delivery of computing resources and services over the Internet 75 from a remote location. Cloud computing services 92 provide additional computer hardware and storage on as needed or subscription basis. Cloud computing services 92 can provide large amounts of scalable data storage, access to sophisticated software and powerful server-based processing, or entire computing infrastructures and platforms. For example, cloud computing services can provide virtualized computing resources such as virtual machines, storage, and networks, platforms for developing, running, and managing applications without the complexity of infrastructure management, and complete software applications over the Internet on a subscription basis.

Distributed computing services 93 provide large-scale processing using multiple interconnected computers or nodes to solve computational problems or perform tasks collectively. In distributed computing, the processing and storage capabilities of multiple machines are leveraged to work together as a unified system. Distributed computing services are designed to address problems that cannot be efficiently solved by a single computer or that require large-scale computational power. These services enable parallel processing, fault tolerance, and scalability by distributing tasks across multiple nodes.

Although described above as a physical device, computing device 10 can be a virtual computing device, in which case the functionality of the physical components herein described, such as processors 20, system memory 30, network interfaces 40, and other like components can be provided by computer-executable instructions. Such computer-executable instructions can execute on a single physical computing device, or can be distributed across multiple physical computing devices, including being distributed across multiple physical computing devices in a dynamic manner such that the specific, physical computing devices hosting such computer-executable instructions can dynamically change over time depending upon need and availability. In the situation where computing device 10 is a virtualized device, the underlying physical computing devices hosting such a virtualized computing device can, themselves, comprise physical components analogous to those described above, and operating in a like manner. Furthermore, virtual computing devices can be utilized in multiple layers with one virtual computing device executing within the construct of another virtual computing device. Thus, computing device 10 may be either a physical computing device or a virtualized computing device within which computer-executable instructions can be executed in a manner consistent with their execution by a physical computing device. Similarly, terms referring to physical components of the computing device, as utilized herein, mean either those physical components or virtualizations thereof performing the same or equivalent functions.

The skilled person will be aware of a range of possible modifications of the various aspects described above. Accordingly, the present invention is defined by the claims and their equivalents.

What is claimed is:

1. A computer-implemented method executed on a platform for a personal health database platform with spatiotemporal modeling, the computer-implemented method comprising:
collecting a plurality of data that include a plurality of data types from multiple sources;
preprocessing the plurality of data by converting the plurality of data into a common format and temporally aligning data points from different data sources along a common timeline;
creating a multi-dimensional spatial framework representing a desired subject, wherein the spatial framework comprises a detailed digital representation of a subject's anatomy;
generating contextualized insight data from raw observational and sensor data with spatiotemporal tagging;
combining the common timeline and spatial framework to create a plurality of multi-dimensional time-based models or simulations, wherein the models or simulations integrate the preprocessed data with the spatial framework to construct a comprehensive 4D representation of the subject's health status;
performing batch, microbatched or streaming near real-time analysis on the plurality of multi-dimensional time-based models or simulations to identify patterns, anomalies, potential health issues, and corresponding therapies or treatments;
generating predictions and forecasts of the subject's future anatomical and physiological states based on the analysis of the plurality of multi-dimensional time-based models or simulations;
displaying the plurality of multi-dimensional time-based models or simulations and analysis results through an audible, haptic, or visual interface connected to a user device, wherein the display includes interactive visualizations or descriptions of health data, insights, or scenarios derived from the plurality of multi-dimensional time-based models or simulations; and
updating the plurality of multi-dimensional time-based models or simulations with new data inputs to maintain an accurate and current representation of the subject's health status over time.

2. The computer-implemented method of claim 1, wherein the plurality of data types comprise genomic data, proteomic data, metabolomics data, metagenomics data, epigenomic data, imaging data, clinical data, and real-time health metrics.

3. A computing system for a personal health database platform with spatiotemporal modeling, the computing system comprising:
one or more hardware processors configured for:
collecting a plurality of data that include a plurality of data types from multiple sources;
preprocessing the plurality of data by converting the plurality of data into a common format and temporally aligning data points from different data sources along a common timeline;
creating a multi-dimensional spatial framework representing a desired subject, wherein the spatial framework comprises a detailed digital representation of a subject's anatomy;
generating contextualized insight data from raw observational and sensor data with spatiotemporal tagging;
combining the common timeline and spatial framework to create a plurality of multi-dimensional time-based models or simulations, wherein the models or simulations integrate the preprocessed data with the spatial framework to construct a comprehensive 4D representation of the subject's health status;
performing batch, microbatched or streaming near real-time analysis on the plurality of multi-dimensional time-based models or simulations to identify patterns, anomalies, potential health issues, and corresponding therapies or treatments;
generating predictions and forecasts of the subject's future anatomical and physiological states based on the analysis of the plurality of multi-dimensional time-based models or simulations;
displaying the plurality of multi-dimensional time-based models or simulations and analysis results through an audible, haptic, or visual interface connected to a user device, wherein the display includes interactive visualizations or descriptions of health data, insights, or scenarios derived from the plurality of multi-dimensional time-based models or simulations; and
updating the plurality of multi-dimensional time-based models or simulations with new data inputs to maintain an accurate and current representation of the subject's health status over time.

4. The computing system of claim 3, wherein the plurality of data types comprise genomic data, proteomic data, metabolomics data, metagenomics data, epigenomic data, imaging data, clinical data, and real-time health metrics.

5. A system for a personal health database platform with spatiotemporal modeling, comprising one or more computers with executable instructions that, when executed, cause the system to:
collect a plurality of data that include a plurality of data types from multiple sources;
preprocess the plurality of data by converting the plurality of data into a common format and temporally aligning data points from different data sources along a common timeline;
create a multi-dimensional spatial framework representing a desired subject, wherein the spatial framework comprises a detailed digital representation of a subject's anatomy;
generate contextualized insight data from raw observational and sensor data with spatiotemporal tagging;
combine the common timeline and spatial framework to create a plurality of multi-dimensional time-based models or simulations, wherein the models or simulations integrate the preprocessed data with the spatial framework to construct a comprehensive 4D representation of the subject's health status;
perform batch, microbatched or streaming near real-time analysis on the plurality of multi-dimensional time-based models or simulations to identify patterns, anomalies, potential health issues, and corresponding therapies or treatments;
generate predictions and forecasts of the subject's future anatomical and physiological states based on the analysis of the plurality of multi-dimensional time-based models or simulations;
display the plurality of multi-dimensional time-based models or simulations and analysis results through an audible, haptic, or visual interface connected to a user device, wherein the display includes interactive visualizations or descriptions of health data, insights, or scenarios derived from the plurality of multi-dimensional time-based models or simulations; and
update the plurality of multi-dimensional time-based models or simulations with new data inputs to maintain an accurate and current representation of the subject's health status over time.

6. The system of claim 5, wherein the plurality of data types comprise genomic data, proteomic data, metabolomics data, metagenomics data, epigenomic data, imaging data, clinical data, and real-time health metrics.

7. Non-transitory, computer-readable storage media having computer instructions embodied thereon that, when executed by one or more processors of a computing system employing a system for a personal health database platform with spatiotemporal modeling, cause the computing system to:
collect a plurality of data that include a plurality of data types from multiple sources;
preprocess the plurality of data by converting the plurality of data into a common format and temporally aligning data points from different data sources along a common timeline;
create a multi-dimensional spatial framework representing a desired subject, wherein the spatial framework comprises a detailed digital representation of a subject's anatomy;
generate contextualized insight data from raw observational and sensor data with spatiotemporal tagging;
combine the common timeline and spatial framework to create a plurality of multi-dimensional time-based models or simulations, wherein the models or simulations integrate the preprocessed data with the spatial framework to construct a comprehensive 4D representation of the subject's health status;
perform batch, microbatched or streaming near real-time analysis on the plurality of multi-dimensional time-based models or simulations to identify patterns, anomalies, potential health issues, and corresponding therapies or treatments;
generate predictions and forecasts of the subject's future anatomical and physiological states based on the analysis of the plurality of multi-dimensional time-based models or simulations;
display the plurality of multi-dimensional time-based models or simulations and analysis results through an audible, haptic, or visual interface connected to a user device, wherein the display includes interactive visualizations or descriptions of health data, insights, or scenarios derived from the plurality of multi-dimensional time-based models or simulations; and
update the plurality of multi-dimensional time-based models or simulations with new data inputs to maintain an accurate and current representation of the subject's health status over time.

8. The media of claim 7, wherein the plurality of data types comprise genomic data, proteomic data, metabolomics data, metagenomics data, epigenomic data, imaging data, clinical data, and real-time health metrics.
